(19) United States
(12) Patent Application Publication
(10) Pub. No.: US 2025/0353863 A1
(43) Pub. Date: Nov. 20, 2025

(54) TRICYCLIC SPIROLACTAM COMPOUNDS WITH ANTIMYCOBACTERIAL ACTIVITY

(71) Applicants: INSTITUT NATIONAL DE LA SANTE ET DE LA RECHERCHE MEDICALE, Paris (FR); UNIVERSITÉ DE LILLE, Lille (FR); INSTITUT PASTEUR DE LILLE, Lille Cedex (FR)

(72) Inventors: Nicolas WILLAND, Lille Cedex (FR); Théo ABDESSELAM-HATTABI, Lille (FR); Benoit DEPREZ, Lille (FR); Baptiste VILLEMAGNE, Lille (FR); Salia TANGARA, Lille Cedex (FR); Sushovan DAM, Lille (FR); Marion FLIPO, Lille Cedex (FR); Léo FAÏON, Lille (FR); Ruben Christiaan HARTKOORN, Lille Cedex (FR)

(21) Appl. No.: 18/871,334

(22) PCT Filed: Jun. 7, 2023
(86) PCT No.: PCT/EP2023/065174

§ 371 Date: Dec. 3, 2024

(30) Foreign Application Priority Data
Jun. 7, 2022 (EP) 22305823.1

(51) Int. Cl. C07D 498/20; A61K 31/438; A61K 31/444; A61K 31/4545; A61K 31/4709; A61K 31/4747; A61K 31/537; A61K 31/5386; A61K 31/55; A61K 45/06; A61P 31/06; C07D 498/10

(52) U.S. Cl. CPC C07D 498/20; A61K 31/438; A61K 31/444; A61K 31/4545; A61K 31/4709; A61K 31/4747; A61K 31/537; A61K 31/5386; A61K 31/55; A61K 45/06; A61P 31/06; C07D 498/10

(57) ABSTRACT

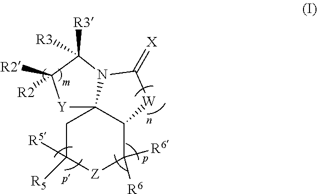

The present invention concerns new tricyclic spirolactams (TriSLas) compounds and their use as a drug, in particular for the prevention and/or treatment of a mycobacterial infection or for the treatment of a disease caused by infection with a Mycobacterium. The tuberculosis drug development pipeline requires further supplementation with additional candidates, ideally acting on novel targets (to minimize cross-resistance) and impacting on drug-tolerant bacilli to shorten treatment. The inventors of the present invention have identified new tricyclic spirolactams (TriSLas) compounds with particular activity against M. tuberculosis.

### FIELD OF INVENTION

The present invention concerns new tricyclic spirolactams (TriSLas) compounds and their use as a drug, in particular for the prevention and/or treatment of a mycobacterial infection or for the prevention and/or treatment of a disease caused by infection with a Mycobacterium.

### BACKGROUND OF THE INVENTION

While global efforts to eradicate tuberculosis (TB) by improving drug accessibility and compliance have significantly decreased deaths by 29% over the last 2 decades, TB remains the leading cause of death by an infectious disease worldwide (World Health Organization. Global tuberculosis report 2021. (2021)). With a minimum of 6-months of multidrug therapy, current TB treatment is notoriously lengthy, a feature largely attributed to the difficulty of eliminating phenotypically drug-tolerant sub-populations of the causative bacteria Mycobacterium tuberculosis (Mtb) (Connolly, L. E., Edelstein, P. H. & Ramakrishnan, L. Why is long-term therapy required to cure tuberculosis? PLOS Med. 4, 435-442 (2007)). Regrettably, escalating infections by multidrug resistant (MDR) TB infections (483,000 cases of rifampicin-resistant TB reported in 2020 (World Health Organization. Global tuberculosis report 2021. (2021)) as well as extensively drug resistant (XDR) TB (12,350 cases reported in 2019 (World Health Organization. Global tuberculosis report 2020. (2020))) require even longer therapy with less efficient and tolerated second-line drugs. In recognition of this global health problem, the WHO has placed TB at the highest critical global priority of antibiotic-resistant bacteria for the development of new antibiotics (World Health Organization. Global Priority List Of Antibiotic-Resistant Bacteria To Guide Research, Discovery And Development Of New Antibiotics. (2017) doi: 10.1016/S1473-3099 (09) 70222-1).

Concerted efforts to find alternative and better antibiotics against drug-sensitive and -resistant TB have led to the approval of two novel classes of anti-TB drugs, the ATP synthase inhibitor bedaquiline (Palomino, J. C. & Martin, A. TMC207 becomes bedaquiline, a new anti-TB drug. Future Microbiol. 8, 1071-1080 (2013)), and the two nitroimidazole prodrugs delamanid (Ryan, N. J. & Lo, J. H. Delamanid: First global approval. Drugs 74, 1041-1045 (2014)) and pretomanid (Keam, S. J. Pretomanid: First Approval. Drugs 79, 1797-1803 (2019)). Additional anti-TB molecules are at various levels of clinical and pre-clinical drug development that may further feed our treatment options in the future (Tiberi, S. et al. Tuberculosis: progress and advances in development of new drugs, treatment regimens, and host-directed therapies. Lancet Infect. Dis. 18, e183-e198 (2018); Tornheim, J. A. & Dooley, K. E. The global landscape of tuberculosis therapeutics. Annu. Rev. Med. 70, 105-120 (2019); J Libardo, M. D., Boshoff, H. I. & Barry, C. E. The present state of the tuberculosis drug development pipeline. Curr. Opin. Pharmacol. 42, 81-94 (2018)). Despite these increased efforts, it is clear that the TB drug development pipeline requires further supplementation with additional candidates, ideally acting on novel targets (to minimize cross-resistance) and impacting on drug-tolerant bacilli to shorten treatment.

The inventors of the present invention identified novel tricyclic spirolactams (TriSLas) compounds with particular activity against M. tuberculosis.

### DESCRIPTION OF THE INVENTION

The present invention thus relates to a compound of formula (I):

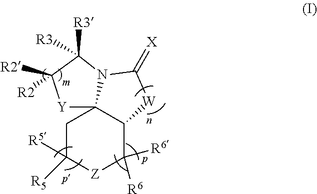

In which:
W is:
   O; or
   CR₄R_{4′}
X is:
   O; or.
   S
Y is:
   O; or
   N
Z is:
   NR₁; or
   CHNRaRb;
p has a value of 0 or 1;
p′ has a value of 1 or 2;
m and n, identical or different have a value of 1 or 2; with at least one W being CR₄R_{4′};
R₁ is chosen from:
   linear or branched —(C₁-C₆)alkyl-(C₆-C₁₀)aryl, said alkyl being optionally substituted by one or more halogen atoms and said aryl being optionally substituted by one or more:
      halogen atom,
      —OH,
      linear or branched —(C₁-C₆)alkyl,
      linear or branched —(C₁-C₆)halogenoalkyl,
      linear or branched —(C₁-C₆)alkoxy,
      linear or branched —(C₁-C₆)halogenoalkoxy,
      —(C₆-C₁₀)aryl,
      —O—(C₆-C₁₀)aryl;
      heterocycle comprising 3 to 10 members and having at least one heteroatom chosen from O, N, or S; said heterocycle being optionally substituted by one or more:
         —(C₆-C₁₀)aryl optionally substituted by one or more linear or branched —(C₁-C₆)halogenoalkoxy; and
         —O—(C₆-C₁₀)aryl optionally substituted by one or more linear or branched —(C₁-C₆)halogenoalkoxy;
   —(C₆-C₁₀)aryl optionally substituted by one or more linear or branched —(C₁-C₆)halogenoalkyl or —(C₁-C₆)halogenoalkoxy;
   linear or branched —(C₁-C₆)alkyl-(C₆-C₁₀) heteroaryl, said heteroaryl having at least one heteroatom chosen from O, N, or S and being optionally substituted by one or more:
      linear or branched —(C₁-C₆)alkyl,
      linear or branched —(C₁-C₆)halogenoalkyl,
      —(C₃-C₆)cycloalkyl;
   linear or branched —(C₁-C₆)alkyl-(C₃-C₆)cycloalkyl;
   —(C₃-C₆)cycloalkyl;
   linear or branched —(C₁-C₆)alkyl-heterocycle, said heterocycle comprising 3 to 10 members and having at least one heteroatom chosen from O, N, or S;
   —S(O)₂—(C₆-C₁₀)aryl, said aryl being optionally substituted by one or more linear or branched —(C₁-C₆)halogenoalkyl;
   S(O)₂—(C₁-C₆)alkyl-(C₆-C₁₀)aryl, said aryl being optionally substituted by one or more linear or branched —(C₁-C₆)halogenoalkyl;
   —C(O)—R′—(C₆-C₁₀)aryl, said aryl being optionally substituted by one or more linear or branched —(C₁-C₆)halogenoalkyl, R′ being chosen from:
      linear or branched —(C₁-C₆)alkyl, said alkyl being optionally substituted by one or more halogen atoms,
      linear or branched —O—(C₁-C₆)alkyl, said alkyl being optionally substituted by one or more halogen atoms;
Ra and Rb, identical or different, are chosen from H, —(C₆-C₁₀)aryl and linear or branched —(C₁-C₆)alkyl-(C₆-C₁₀)aryl, said aryl being optionally substituted by one or more linear or branched —(C₁-C₆)halogenoalkyl;
R₂ and R₂′ are —H;
R₃ and R₃′, identical or different are chosen from:
   —H;
   a linear or branched —(C₁-C₆)alkyl;
   a linear or branched —(C₂-C₆)alkenyl;
   a linear or branched —(C₁-C₆)halogenoalkyl; or
   a —(C₃-C₆)cycloalkyl;
or R₂ or R₂′ is linked together with R₃ or R₃′ to form a cycloalkyl or aryl; said cycloalkyl and aryl comprising 3 to 6 members
R₄ and R₄′, identical or different are chosen from:
   —H;
   a halogen atom;
   a linear or branched —(C₁-C₆)alkyl; and
   a linear or branched —(C₂-C₆)alkenyl;
R₅ and R₅′ identical or different are chosen from H, linear or branched —(C₁-C₃)alkyl or form together ═O;
R₆ and R₆′, different, identical or different are chosen from H, linear or branched —(C₁-C₃)alkyl or form together ═O;
   or its pharmaceutically acceptable salts.

The present invention further relates to a pharmaceutical composition comprising a compound of formula (I) as defined herein.

It also relates to a compound according to the invention or to a pharmaceutical composition according to the invention for use as a drug, in particular for the prevention and/or treatment of a mycobacterial infection or for the treatment of a disease caused by infection with a Mycobacterium, more particularly wherein the mycobacterial infection is a Mycobacterium tuberculosis infection.

It further relates to a compound or a pharmaceutical composition according to the invention in combination with at least one other anti-mycobacterial agent and to their use as mentioned above.

Unless specified otherwise, the terms used hereabove or hereafter as regards to the compounds of formula (I) have the meaning ascribed to them below:
“halogen” refers to fluorine, chlorine, bromine or iodine atom, in particular fluorine or chlorine atom.
“alkyl” represents an aliphatic-hydrocarbon group which may be straight or branched, having 1 to 6, especially 1 to 3 carbon atoms in the chain (C₁-C₆)alkyl or (C₁-C₃)alkyl, unless specified otherwise. In particular, alkyl groups have 1 to 3 carbon atoms in the chain (C₁-C₃)alkyl. Branched means that one or more alkyl groups such as methyl, ethyl or propyl are attached to a linear alkyl chain. Exemplary alkyl groups include methyl, ethyl, n-propyl, i-propyl, n-butyl, t-butyl, i-25 butyl, n-pentyl, n-hexyl, in particular isobutyl, isopropyl, propyl, methyl or ethyl. As mentioned, said alkyl can be substituted by one or more halogen atoms.
“halogenoalkyl” represents an aliphatic-hydrocarbon group which may be straight or branched, having 1 to 6, especially 1 to 3 carbon atoms in the chain (C₁-C₆)halogenoalkyl or (C₁-C₃)halogenoalkyl and in which one or more hydrogen atoms has been replaced by a halogen atom such as fluorine, chlorine, bromine or iodine atom, in particular by one or more fluorine atoms. Exemplary halogenoalkyl include trifluoromethyl or difluoroethyl.
“(C₃-C₆)cycloalkyl” refers to a saturated monocyclic or bicyclic non-aromatic hydrocarbon ring of 3 to 6 carbon atoms, which can comprise one or more unsaturation. Specific examples of monocyclic cycloalkyl groups include cyclopropyl, cyclobutyl, cyclopentyl, cyclopentenyl, cyclohexyl. Preferably, the cycloalkyl group is cyclohexyl, cyclobutyl or cyclopentyl.
“alkoxy” represents an alkyl group as previously defined singular bonded to oxygen. Examples of linear or branched (C₁-C₆)alkoxy or (C₁-C₃)alkoxy includes methoxy (CH₃O) and ethoxy (CH₃CH₂O).
   “halogenoalkoxy” represents an halogenoalkyl group as previously defined bonded to oxygen. Examples of linear or branched (C₁-C₆)halogenoalkoxy or (C₁-C₃)halogenoalkoxy includes trifluoromethoxy (CF₃O).
“alkenyl” refers to an aliphatic hydrocarbon group containing a carbon-carbon double bond and which may be straight or branched having 2 to 6 carbon atoms in the chain (C₂-C₆)alkenyl, unless specified otherwise. Preferred alkenyl groups have 2 to 3 carbon atoms in the chain (C₂-C₃)alkenyl. Exemplary alkenyl groups include ethenyl, n-propenyl, i-propenyl, n-butenyl, i-butenyl, in particular ethenyl or propenyl.
“aryl” refers to an aromatic monocyclic or multicyclic hydrocarbon ring system of 6 to 10 carbon atoms, preferably of 6 carbon atoms. Exemplary aryl groups include phenyl, naphthyl, biphenyl, in particular phenyl. Said aryl or phenyl can be substituted by one or more halogen atoms such as fluorine, bromine, iodine or chlorine, in particular chlorine or fluorine; —OH; one or more (C₁-C₆)alkyl or (C₁-C₃)alkyl such as a methyl, said alkyl being optionally substituted by one to 3 fluorine atoms such as trifluoromethyl; one or more (C₁-C₃)alkoxy such as methoxy, said alkoxy being optionally substituted by one or more fluorine atoms such as trifluoromethoxy; one-(C₆-C₁₀)aryl, in particular phenyl; one-O—(C₆-C₁₀)aryl, in particular, —O-phenyl; a heterocycle as defined below.
“linear or branched (C₁-C₆)alkyl-(C₆-C₁₀)aryl” means that the aryl is linked to the alkyl group by a carbon of the alkyl group; alkyl and aryl group are as defined previously; in particular —(C₁-C₃)alkyl-(C₆-C₁₀)aryl and more particularly (C₁-C₃)alkyl-phenyl are contemplated, more particularly benzyl, ethylphenyl difluoroethylphenyl or propylphenyl. Phenyl can be substituted as above mentioned, in particular by a halogen atom such as fluorine or chlorine, in particular chlorine; a methyl, a trifluoromethyl, a methoxy or trifluorometoxy.
“heterocycle” or “heterocycloalkyl” refers to a saturated or partially unsaturated non aromatic stable 3 to 10-membered mono, bi or multicyclic rings. Hetero atoms can be O, S or N, in particular N. In particular, each ring comprises from 1 to 3 hetero atoms. Suitable heterocycles are also disclosed in the Handbook of Chemistry and Physics, 76th Edition, CRC Press, Inc., 1995-1996, pages 225 to 226, the disclosure of which is hereby incorporated by reference. Examples of heterocycloalkyl include, but are not limited to piperazine, tetrahydropyrane, tetrahydrofurane, diazepane, piperidine, pyrrolidine, morpholine, azetidine, diazabicyclo octanyl, diazabicycloheptanyl, azabicyclohexanyl, in particular piperazine, tetrahydropyrane, tetrahydrofurane. Said heterocycle is optionally substituted by a (C₆-C₁₀)aryl, in particular phenyl, substituted by one or more (C₁-C₆)halogenoalkyl, in particular trifluoromethoxy; or by a —O—(C₆-C₁₀)aryl, in particular phenyl, substituted by one or more (C₁-C₆)halogenoalkyl, in particular trifluoromethoxy.
“—O—(C₆-C₁₀)aryl” means that the aryl group is linked to the atom of oxygen by a carbon of the aryl group.
“linear or branched (C₁-C₆)alkyl-heterocycle” means that the heterocycle is linked to the alkyl group by a carbon of the alkyl group; alkyl and heterocycle are as defined previously.
“heteroaryl” refers to a 5 to 10, aromatic mono-, bi- or multicyclic ring wherein at least one member of the ring is a hetero atom. Hetero atoms can be O, S or N, in particular N. In particular, each ring comprises from 1 to 3 hetero atoms. Examples include pyrrolyl, pyridyl, oxadiazol, thiazol, oxazol, triazol, pyrazolyl, pyrimidinyl, pyrazinyl, indolyl, quinolyl, imidazolyl, in particular pyridyl, quinolyl, imidazolyl and indolyl. Said heteroaryl can be substituted by one or more (C₁-C₆)alkyl or (C₁-C₃)alkyl such as a methyl, said alkyl being optionally substituted by one to 3 fluorine atoms such as trifluoromethyl; one or more (C₃-C₆)cycloalkyl such as cyclopentyl.
“—S(O)₂—(C₆-C₁₀)aryl” means that the aryl group is linked to the atom of sulfur by a carbon of the aryl group; in particular-S(O)₂—(C₆-C₁₀)aryl is S(O)₂-phenyl. Phenyl can be substituted as above mentioned, in particular by (C₁-C₆)halogenoalkyl such as trifluoromethyl.
“—S(O)₂—(C₁-C₆)alkyl-(C₆-C₁₀)aryl” means that the alkyl group is linked to the atom of sulfur by a carbon of the alkyl group; in particular-S(O)₂—(C₁-C₆)alkyl-(C₆-C₁₀)aryl is S(O)₂—(C₁-C₃)alkyl-phenyl. Phenyl can be substituted as above mentioned, in particular by (C₁-C₆)halogenoalkyl such as trifluoromethyl.
“—C(O)—R′—(C₆-C₁₀)aryl” means that the aryl group is linked to the R′ by a carbon of the aryl group, said R′ being also linked to the carbon of the C(═O); with R′ chosen from linear or branched —(C₁-C₆)alkyl, said alkyl being optionally substituted by one or more halogen atoms; linear or branched —O—(C₁-C₆)alkyl, said alkyl being optionally substituted by one or more halogen atoms.
   The term “substituted” generally refers to, unless specified otherwise, a substitution with one or more substituents, which may be identical or different, and which are identified herein.

As mentioned above, W can not be O two times, so that at least one the two W is CR₄R₄′. The compounds of formula (I) as described herein can be provided in the form of a free base or in the form of addition salts with acids, which also form part of the invention. These salts are advantageously prepared with pharmaceutically acceptable acids, but salts with other acids, useful for example for the purification or for the isolation of the compounds of formula (I) as described herein, also form part of the invention.

As used herein, the expression “pharmaceutically acceptable” refers to those compounds, materials, excipients, compositions or dosage forms which are, within the scope of sound medical judgment, suitable for contact with the tissues of human beings and animals without excessive toxicity, irritation, allergic response or other problem complications commensurate with a reasonable benefit/risk ratio.

As used herein, “pharmaceutically acceptable salts” refer to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like, including mono, di or tri-salts thereof; and the salts prepared from organic acids such as formic, acetic, propionic, succinic, tartaric, citric, methanesulfonic, trifluoromethanesulfonic, benzenesulfonic, trifluoroacetic, glucoronic, glutamic, benzoic, salicylic, toluenesulfonic, oxalic, fumaric, maleic, lactic and the like. Further addition salts include ammonium salts such as tromethamine, meglumine, epolamine, etc., metal salts such as sodium, potassium, calcium, zinc or magnesium.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, non-aqueous media like ether, dioxane, ethyl acetate, ethanol, isopropanol, or acetonitrile are preferred. Lists of suitable salts are found in Remington's Pharmaceutical Sciences, 20th ed., Mack Publishing Company, Easton, PA, 2000, the disclosure of which is hereby incorporated by reference.

### Compounds

As mentioned, the compounds according to the invention are compounds of formula (I):

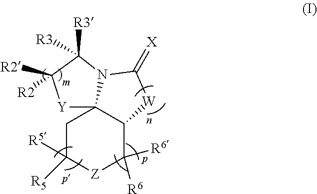

In which:
W is:
   O; or
   CR₄R_{4′}
X is:
   O; or
   S
Y is:
   O; or
   N
Z is:
   NR₁; or
   CHNRaRb;
p has a value of 0 or 1;
p′ has a value of 1 or 2;
m and n, identical or different have a value of 1 or 2; with at least one W being CR₄R_{4′};
R₁ is chosen from:
   linear or branched —(C₁-C₆)alkyl-(C₆-C₁₀)aryl, said alkyl being optionally substituted by one or more halogen atoms and said aryl being optionally substituted by one or more:
      halogen atom,
      —OH,
      linear or branched —(C₁-C₆)alkyl,
      linear or branched —(C₁-C₆)halogenoalkyl,
      linear or branched —(C₁-C₆)alkoxy,
      linear or branched —(C₁-C₆)halogenoalkoxy,
      —(C₆-C₁₀)aryl,
      —O—(C₆-C₁₀)aryl;
      heterocycle comprising 3 to 10 members and having at least one heteroatom chosen from O, N, or S; said heterocycle being optionally substituted by one or more:
         —(C₆-C₁₀)aryl optionally substituted by one or more linear or branched —(C₁-C₆)halogenoalkoxy; and
         —O—(C₆-C₁₀)aryl optionally substituted by one or more linear or branched —(C₁-C₆)halogenoalkoxy;
   —(C₆-C₁₀)aryl optionally substituted by one or more linear or branched (C₁-C₆)halogenoalkyl or —(C₁-C₆)halogenoalkoxy;
   linear or branched —(C₁-C₆)alkyl-(C₆-C₁₀) heteroaryl, said heteroaryl having at least one heteroatom chosen from O, N, or S and being optionally substituted by one or more:
      linear or branched —(C₁-C₆)alkyl,
      linear or branched —(C₁-C₆)halogenoalkyl,
      —(C₃-C₆)cycloalkyl;
   linear or branched —(C₁-C₆)alkyl-(C₃-C₆)cycloalkyl;
   —(C₃-C₆)cycloalkyl;
   linear or branched —(C₁-C₆)alkyl-heterocycle, said heterocycle comprising 3 to 10 members and having at least one heteroatom chosen from O, N, or S;
   —S(O)₂—(C₆-C₁₀)aryl, said aryl being optionally substituted by one or more linear or branched —(C₁-C₆)halogenoalkyl;
   S(O)₂—(C₁-C₆)alkyl-(C₆-C₁₀)aryl, said aryl being optionally substituted by one or more linear or branched —(C₁-C₆)halogenoalkyl;
   —C(O)—R′—(C₆-C₁₀)aryl, said aryl being optionally substituted by one or more linear or branched —(C₁-C₆)halogenoalkyl, R′ being chosen from:
      linear or branched —(C₁-C₆)alkyl, said alkyl being optionally substituted by one or more halogen atoms,
      linear or branched —O—(C₁-C₆)alkyl, said alkyl being optionally substituted by one or more halogen atoms;
Ra and Rb, identical or different, are chosen from H, —(C₆-C₁₀)aryl and linear or branched —(C₁-C₆)alkyl-(C₆-C₁₀)aryl, said aryl being optionally substituted by one or more linear or branched —(C₁-C₆)halogenoalkyl;
R₂ and R₂′ are H
R₃ and R₃′, identical or different are chosen from:
   —H;
   a linear or branched —(C₁-C₆)alkyl;
   a linear or branched —(C₂-C₆)alkenyl;
   a linear or branched —(C₁-C₆)halogenoalkyl; or
   a —(C₃-C₆)cycloalkyl;
or R₂ or R₂′ is linked together with R₃ or R₃′ to form a cycloalkyl or aryl; said cycloalkyl and aryl comprising 3 to 6 members;
R₄ and R₄′, identical or different are chosen from:
   —H;
   a halogen atom;
   a linear or branched —(C₁-C₆)alkyl; and
   a linear or branched —(C₂-C₆)alkenyl;
R₅ and R₅′ identical or different are chosen from H, linear or branched —(C₁-C₃)alkyl or form together ═O;
R₆ and R₆′, different, identical or different are chosen from H, linear or branched —(C₁-C₃)alkyl or form together ═O;
   or its pharmaceutically acceptable salts.

In one embodiment, Z is NR₁ with R₁ chosen from:
linear or branched —(C₁-C₃)alkyl-phenyl, said alkyl being optionally substituted by one or more halogen atoms, in particular fluorine, and said phenyl being optionally substituted by one or more:
   halogen atom,
   —OH,
   linear or branched —(C₁-C₃)alkyl, in particular methyl,
   linear or branched —(C₁-C₃)halogenoalkyl, in particular trifluoromethyl,
   linear or branched —(C₁-C₃)alkoxy, in particular methoxy;
   linear or branched —(C₁-C₃)halogenoalkoxy, in particular trifluoromethoxy;
   phenyl,
   —O-phenyl;
   an heterocycle comprising 3 to 10 members and having at least one heteroatom chosen from N; said heterocycle being optionally substituted by one or more:
      phenyl optionally substituted by one or more linear or branched —(C₁-C₃)halogenoalkoxy, in particular trifluoromethoxy; and
      —O-phenyl optionally substituted by one or more linear or branched —(C₁-C₃)halogenoalkoxy, in particular trifluoromethoxy;
phenyl optionally substituted by one or more linear or branched —(C₁-C₃)halogenoalkyl or —(C₁-C₃)halogenoalkoxy, in particular trifluoromethyl or trifluoromethoxy;
linear or branched —(C₁-C₃)alkyl-(C₆-C₁₀) heteroaryl, said heteroaryl having at least one heteroatom chosen from N and being optionally substituted by one or more:
   linear or branched —(C₁-C₃)alkyl, in particular methyl,
   linear or branched —(C₁-C₃)halogenoalkyl, in particular trifluoromethyl,
   —(C₃-C₆)cycloalkyl;
linear or branched —(C₁-C₃)alkyl-(C₃-C₆)cycloalkyl;
—(C₃-C₆)cycloalkyl;
linear or branched —(C₁-C₃)alkyl-heterocycle, said heterocycle comprising 3 to 10 members and having at least one heteroatom chosen from O or N;
—S(O)₂-phenyl, said phenyl being optionally substituted by one or more linear or branched —(C₁-C₃)halogenoalkyl, in particular trifluoromethyl;
—S(O)₂—(C₁-C₃)alkyl-phenyl, said phenyl being optionally substituted by one or more linear or branched —(C₁-C₃)halogenoalkyl, in particular trifluoromethyl;
—C(O)—R′-phenyl, said phenyl being optionally substituted by one or more linear or branched —(C₁-C₃)halogenoalkyl, in particular trifluoromethoxy, with R′ being chosen from:
   linear or branched —(C₁-C₃)alkyl, said alkyl being optionally substituted by one or more halogen atoms,
   linear or branched —O—(C₁-C₃)alkyl.

Alternatively, Z is CHNRaRb with Ra and Rb, identical or different, chosen from H, phenyl and linear or branched —(C₁-C₃)alkylphenyl, said phenyl being optionally substituted by one or more linear or branched —(C₁-C₃)halogenoalkyl, in particular by one trifluoromethyl.

In particular:
R₂ and R₂′, are —H; and/or
R₃ and R₃′, identical or different are chosen from:
   —H;
   a linear or branched —(C₁-C₆)alkyl;
   a linear or branched —(C₂-C₃)alkenyl;
   a linear or branched —(C₁-C₃)halogenoalkyl, in particular trifluoromethyl; or
   a —(C₃-C₆)cycloalkyl;
or R₂ or R₂′ is linked together with R₃ or R₃′ to form a cycloalkyl or aryl; said cycloalkyl and aryl comprising 3 to 6 members; and/or
R₄ and R₄′, identical or different are chosen from:
   —H;
   a halogen atom;
   a linear or branched —(C₁-C₃)alkyl; and
   a linear or branched —(C₂-C₃)alkenyl; and/or
R₅ and R₅′ are H or form together ═O; and/or
R₆ and R₆′ are H or form together ═O.

More particularly, said compound if chosen from:
(3S,7aR,11aR)-9-[(4-fluorophenyl)methyl]-3-isopropyl-2,3,6,7,7a,8,10,11-octahydrooxazolo[2,3-j][1,6]naphthyridin-5-one;
(3S,7aR,11aR)-3-isopropyl-9-[[4-(trifluoromethyl)phenyl]methyl]-2,3,6,7,7a,8,10,11-octahydrooxazolo[2,3-j][1,6]naphthyridin-5-one;
(3S,7aR,11aR)-3-isopropyl-9-[[4-(trifluoromethoxy)phenyl]methyl]-2,3,6,7,7a,8,10,11-octahydrooxazolo[2,3-j][1,6]naphthyridin-5-one;
(3S,7aR,11aR)-9-[(4-chlorophenyl)methyl]-3-isopropyl-2,3,6,7,7a,8,10,11-octahydrooxazolo[2,3-j][1,6]naphthyridin-5-one;
(3S,7aR,11aR)-9-[(3,4-dichlorophenyl)methyl]-3-isopropyl-2,3,6,7,7a,8,10,11-octahydrooxazolo[2,3-j][1,6]naphthyridin-5-one;
(3S,7aR,11aR)-3-isopropyl-9-(p-tolylmethyl)-2,3,6,7,7a,8,10,11-octahydrooxazolo[2,3-j][1,6]naphthyridin-5-one;
(3S,7aR,11aR)-3-isopropyl-9-[(4-methoxyphenyl)methyl]-2,3,6,7,7a,8,10,11-octahydrooxazolo[2,3-j][1,6]naphthyridin-5-one;
(3S,7aR,11aR)-9-[(4-hydroxyphenyl)methyl]-3-isopropyl-2,3,6,7,7a,8,10,11-octahydrooxazolo[2,3-j][1,6]naphthyridin-5-one;
(3S,7aR,11aR)-9-[[4-chloro-3-(trifluoromethyl)phenyl]methyl]-3-isopropyl-2,3,6,7,7a,8,10,11-octahydrooxazolo[2,3-j][1,6]naphthyridin-5-one;
(3S,7aR,11aR)-9-[[2,4-bis(trifluoromethyl)phenyl]methyl]-3-isopropyl-2,3,6,7,7a,8,10,11-octahydrooxazolo[2,3-j][1,6]naphthyridin-5-one;
(3S,7aR,11aR)-3-isopropyl-9-[(4-phenylphenyl)methyl]-2,3,6,7,7a,8,10,11-octahydrooxazolo[2,3-j][1,6]naphthyridin-5-one;
(3S,7aR,11aR)-3-isopropyl-9-[(3-phenoxyphenyl)methyl]-2,3,6,7,7a,8,10,11-octahydrooxazolo[2,3-j][1,6]naphthyridin-5-one;
(3S,7aR,11aR)-3-isopropyl-9-[[4-[4-[4-(trifluoromethoxy)phenoxy]-1-piperidyl]phenyl]methyl]-2,3,6,7,7a,8,10,11-octahydrooxazolo[2,3-j][1,6]naphthyridin-5-one;
(3S,7aR,11aR)-3-isopropyl-9-[[4-[4-[4-(trifluoromethoxy)phenyl]-1-piperidyl]phenyl]methyl]-2,3,6,7,7a,8,10,11-octahydrooxazolo[2,3-j][1,6]naphthyridin-5-one;
(3S,7aR,11aR)-3-isopropyl-9-[[4-(1-piperidyl)phenyl]methyl]-2,3,6,7,7a,8,10,11-octahydrooxazolo[2,3-j][1,6]naphthyridin-5-one;
(3S,7aR,11aR)-3-isopropyl-9-(2-pyridylmethyl)-2,3,6,7,7a,8,10,11-octahydrooxazolo[2,3-j][1,6]naphthyridin-5-one;
(3S,7aR,11aR)-3-isopropyl-9-(3-pyridylmethyl)-2,3,6,7,7a,8,10,11-octahydrooxazolo[2,3-j][1,6]naphthyridin-5-one;
(3S,7aR,11aR)-3-isopropyl-9-(4-pyridylmethyl)-2,3,6,7,7a,8,10,11-octahydrooxazolo[2,3-j][1,6]naphthyridin-5-one;
(3S,7aR,11aR)-3-isopropyl-9-[[6-(trifluoromethyl)-3-pyridyl]methyl]-2,3,6,7,7a,8,10,11-octahydrooxazolo[2,3-j][1,6]naphthyridin-5-one;
(3S,7aR,11aR)-3-isopropyl-9-[[5-(trifluoromethyl)-2-pyridyl]methyl]-2,3,6,7,7a,8,10,11-octahydrooxazolo[2,3-j][1,6]naphthyridin-5-one;
(3S,7aR,11aR)-3-isopropyl-9-(2-quinolylmethyl)-2,3,6,7,7a,8,10,11-octahydrooxazolo[2,3-j][1,6]naphthyridin-5-one;
(3S,7aR,11aR)-3-isopropyl-9-(8-quinolylmethyl)-2,3,6,7,7a,8,10,11-octahydrooxazolo[2,3-j][1,6]naphthyridin-5-one;
(3S,7aR,11aR)-3-isopropyl-9-(6-quinolylmethyl)-2,3,6,7,7a,8,10,11-octahydrooxazolo[2,3-j][1,6]naphthyridin-5-one;
(3S,7aR,11aR)-9-[(2-cyclopropyl-4-quinolyl)methyl]-3-isopropyl-2,3,6,7,7a,8,10,11-octahydrooxazolo[2,3-j][1,6]naphthyridin-5-one;
(3S,7aR,11aR)-3-isopropyl-9-[[2-(trifluoromethyl)-4-quinolyl]methyl]-2,3,6,7,7a,8,10,11-octahydrooxazolo[2,3-j][1,6]naphthyridin-5-one;
(3S,7aR,11aR)-3-isopropyl-9-[(1-methylimidazol-2-yl)methyl]-2,3,6,7,7a,8,10,11-octahydrooxazolo[2,3-j][1,6]naphthyridin-5-one;
(3S,7aR,11aR)-3-isopropyl-9-[(3-methyl-1H-indol-2-yl)methyl]-2,3,6,7,7a,8,10,11-octahydrooxazolo[2,3-j][1,6]naphthyridin-5-one;
(3S,7aR,11aR)-3-isopropyl-9-[(1-methylindol-2-yl)methyl]-2,3,6,7,7a,8,10,11-octahydrooxazolo[2,3-j][1,6]naphthyridin-5-one;
(3S,7aR,11aR)-9-(cyclohexylmethyl)-3-isopropyl-2,3,6,7,7a,8,10,11-octahydrooxazolo[2,3-j][1,6]naphthyridin-5-one;
(3S,7aR,11aR)-9-cyclobutyl-3-isopropyl-2,3,6,7,7a,8,10,11-octahydrooxazolo[2,3-j][1,6]naphthyridin-5-one;
(3S,7aR,11aR)-9-cyclohexyl-3-isopropyl-2,3,6,7,7a,8,10,11-octahydrooxazolo[2,3-j][1,6]naphthyridin-5-one;
(3S,7aR,11aR)-3-isopropyl-9-(tetrahydropyran-4-ylmethyl)-2,3,6,7,7a,8,10,11-octahydrooxazolo[2,3-j][1,6]naphthyridin-5-one;
(3S,7aR,11aR)-3-isopropyl-9-(tetrahydropyran-2-ylmethyl)-2,3,6,7,7a,8,10,11-octahydrooxazolo[2,3-j][1,6]naphthyridin-5-one;
(3S,7aR,11aR)-3-isopropyl-9-(tetrahydrofuran-2-ylmethyl)-2,3,6,7,7a,8,10,11-octahydrooxazolo[2,3-j][1,6]naphthyridin-5-one;
(3S,7aR,11aR)-3-isopropyl-9-phenyl-2,3,6,7,7a,8,10,11-octahydrooxazolo[2,3-j][1,6]naphthyridin-5-one;
(3S,7aR,11aR)-3-isopropyl-9-[4-(trifluoromethyl)phenyl]-2,3,6,7,7a,8,10,11-octahydrooxazolo[2,3-j][1,6]naphthyridin-5-one;
(3S,7aR,11aR)-3-isopropyl-9-(2-phenylethyl)-2,3,6,7,7a,8,10,11-octahydrooxazolo[2,3-j][1,6]naphthyridin-5-one;
(3S,7aR,11aR)-3-isopropyl-9-[2-[4-(trifluoromethyl)phenyl]ethyl]-2,3,6,7,7a,8,10,11-octahydrooxazolo[2,3-j][1,6]naphthyridin-5-one;
(3S,7aR,11aR)-3-isopropyl-9-[3-[4-(trifluoromethyl)phenyl]propyl]-2,3,6,7,7a,8,10,11-octahydrooxazolo[2,3-j][1,6]naphthyridin-5-one;
(3S,7aR,11aR)-3-isopropyl-9-(1-phenylethyl)-2,3,6,7,7a,8,10,11-octahydrooxazolo[2,3-j][1,6]naphthyridin-5-one;
(3S,7aR,11aR)-9-[2,2-difluoro-2-[4-(trifluoromethyl)phenyl]ethyl]-3-isopropyl-2,3,6,7,7a,8,10,11-octahydrooxazolo[2,3-j][1,6]naphthyridin-5-one;
(3S,7aR,11aR)-3-isopropyl-9-(2-phenylacetyl)-2,3,6,7,7a,8,10,11-octahydrooxazolo[2,3-j][1,6]naphthyridin-5-one;
(3S,7aR,11aR)-3-isopropyl-9-[2-[4-(trifluoromethyl)phenyl]acetyl]-2,3,6,7,7a,8,10,11-octahydrooxazolo[2,3-j][1,6]naphthyridin-5-one;
(3S,7aR,11aR)-3-isopropyl-9-[3-[4-(trifluoromethyl)phenyl]propanoyl]-2,3,6,7,7a,8,10,11-octahydrooxazolo[2,3-j][1,6]naphthyridin-5-one;
(3S,7aR,11aR)-3-isopropyl-9-4-[[4-(trifluoromethyl)phenyl]butanoyl]-2,3,6,7,7a,8,10,11-octahydrooxazolo[2,3-j][1,6]naphthyridin-5-one;
(3S,7aR,11aR)-9-[2,2-difluoro-2-[4-(trifluoromethyl)phenyl]acetyl]-3-isopropyl-2,3,6,7,7a,8,10,11-octahydrooxazolo[2,3-j][1,6]naphthyridin-5-one;
benzyl (3S,7aR,11aR)-3-isopropyl-5-oxo-2,3,6,7,7a,8,10,11-octahydrooxazolo[2,3-j][1,6]naphthyridine-9-carboxylate;
[4-(trifluoromethyl)phenyl]methyl (3S,7aR,11aR)-3-isopropyl-5-oxo-2,3,6,7,7a,8,10,11-octahydrooxazolo[2,3-j][1,6]naphthyridine-9-carboxylate;
2-[4-(trifluoromethyl)phenyl]ethyl (3S,7aR,11aR)-3-isopropyl-5-oxo-2,3,6,7,7a,8,10,11-octahydrooxazolo[2,3-j][1,6]naphthyridine-9-carboxylate;
3-[4-(trifluoromethyl)phenyl]propyl (3S,7aR,11aR)-3-isopropyl-5-oxo-2,3,6,7,7a,8,10,11-octahydrooxazolo[2,3-j][1,6]naphthyridine-9-carboxylate;
(3S,7aR,11aR)-3-isopropyl-9-[4-(trifluoromethyl)phenyl]sulfonyl-2,3,6,7,7a,8,10,11-octahydrooxazolo[2,3-j][1,6]naphthyridin-5-one;
(3S,7aR,11aR)-3-isopropyl-9-[[4-(trifluoromethyl)phenyl]methylsulfonyl]-2,3,6,7,7a,8,10,11-octahydrooxazolo[2,3-j][1,6]naphthyridin-5-one;
(3S,7aR,11aR)-3-isopropyl-9-[2-[4-(trifluoromethyl)phenyl]ethylsulfonyl]-2,3,6,7,7a,8,10,11-octahydrooxazolo[2,3-j][1,6]naphthyridin-5-one;
(3S,7aR,11aR)-3-isopropyl-9-[3-[4-(trifluoromethyl)phenyl]propylsulfonyl]-2,3,6,7,7a,8,10,11-octahydrooxazolo[2,3-j][1,6]naphthyridin-5-one;
(3S,7aR,11aR)-9-benzyl-3-[(1S)-1-methylpropyl]-2,3,6,7,7a,8,10,11-octahydrooxazolo[2,3-j][1,6]naphthyridin-5-one;
(3S,7aS,11aS)-9-benzyl-3-[(1S)-1-methylpropyl]-2,3,6,7,7a,8,10,11-octahydrooxazolo[2,3-j][1,6]naphthyridin-5-one;
(3S,7aR,11aR)-9-benzyl-3-[(1R)-1-methylpropyl]-2,3,6,7,7a,8,10,11-octahydrooxazolo[2,3-j][1,6]naphthyridin-5-one;
(3S,7aR,11aR)-9-benzyl-3-tert-butyl-2,3,6,7,7a,8,10,11-octahydrooxazolo[2,3-j][1,6]naphthyridin-5-one;
(7aR,11aR)-9-benzyl-3,3-dimethyl-6,7,7a,8,10,11-hexahydro-2H-oxazolo[2,3-j][1,6]naphthyridin-5-one;
(7aS,11aS)-9-benzyl-3,3-dimethyl-6,7,7a,8,10,11-hexahydro-2H-oxazolo[2,3-j][1,6]naphthyridin-5-one;
(7aR,11aR)-9-benzylspiro[6,7,7a,8,10,11-hexahydro-2H-oxazolo[2,3-j][1,6]naphthyridine-3,1′-cyclohexane]-5-one;
(7aS,11aS)-9-benzylspiro[6,7,7a,8,10,11-hexahydro-2H-oxazolo[2,3-j][1,6]naphthyridine-3,1′-cyclohexane]-5-one;
(3S,7aR,11aR)-9-benzyl-3-cyclopropyl-2,3,6,7,7a,8,10,11-octahydrooxazolo[2,3-j][1,6]naphthyridin-5-one;
(3S,7aR,11aR)-9-[[4-(trifluoromethyl)phenyl]methyl]-3-vinyl-2,3,6,7,7a,8,10,11-octahydrooxazolo[2,3-j][1,6]naphthyridin-5-one;
(3S,7aR,11aR)-9-benzyl-3-ethyl-2,3,6,7,7a,8,10,11-octahydrooxazolo[2,3-j][1,6]naphthyridin-5-one;
(3S,7aS,11aS)-9-benzyl-3-ethyl-2,3,6,7,7a,8,10,11-octahydrooxazolo[2,3-j][1,6]naphthyridin-5-one;
(3R,7aR,11aR)-3-(trifluoromethyl)-9-[[4-(trifluoromethyl)phenyl]methyl]-2,3,6,7,7a,8,10,11-octahydrooxazolo[2,3-j][1,6]naphthyridin-5-one
(1R,6R,11S,16S)-4-benzyl-17-oxa-4,10-diazatetracyclo[8.7.0.01,6.011,16]heptadecan-9-one;
(1S,6S,11R,16R)-4-benzyl-17-oxa-4,10-diazatetracyclo[8.7.0.01,6.011,16]heptadecan-9-one;
(1R,6R,11S,16S)-4-[[4-(trifluoromethoxy)phenyl]methyl]-17-oxa-4,10-diazatetracyclo[8.7.0.01,6.011,16]heptadecan-9-one;
(1S,6S,11R,16R)-4-[[4-(trifluoromethoxy)phenyl]methyl]-17-oxa-4,10-diazatetracyclo[8.7.0.01,6.011,16]heptadecan-9-one;
(8aR,12aR)-10-[[4-(trifluoromethyl)phenyl]methyl]spiro[2,4,7,8,8a,9,11,12-octahydro-[1,3]oxazino[2,3-j][1,6]naphthyridine-3,1′-cyclobutane]-6-one;
(8aS,12aS)-10-[[4-(trifluoromethyl)phenyl]methyl]spiro[2,4,7,8,8a,9,11,12-octahydro-[1,3]oxazino[2,3-j][1,6]naphthyridine-3,1′-cyclobutane]-6-one;
(3R,8aR,12aR)-3-isopropyl-10-[[4-(trifluoromethyl)phenyl]methyl]-3,4,7,8,8a,9,11,12-octahydro-2H-[1,3]oxazino[2,3-j][1,6]naphthyridin-6-one;
(3S,8aS,12aS)-3-isopropyl-10-[[4-(trifluoromethyl)phenyl]methyl]-3,4,7,8,8a,9,11,12-octahydro-2H-[1,3]oxazino[2,3-j][1,6]naphthyridin-6-one;
(3R,8aR,12aR)-3-ethyl-10-[[4-(trifluoromethyl)phenyl]methyl]-3,4,7,8,8a,9,11,12-octahydro-2H-[1,3]oxazino[2,3-j][1,6]naphthyridin-6-one;
(3S,8aS,12aS)-3-ethyl-10-[[4-(trifluoromethyl)phenyl]methyl]-3,4,7,8,8a,9,11,12-octahydro-2H-[1,3]oxazino[2,3-j][1,6]naphthyridin-6-one;
(4R,8aR,12aR)-4-isopropyl-10-[[4-(trifluoromethyl)phenyl]methyl]-3,4,7,8,8a,9,11,12-octahydro-2H-[1,3]oxazino[2,3-j][1,6]naphthyridin-6-one;
(1S,6R,11S,16S)-4-benzyl-4,10,17-triazatetracyclo[8.7.0.01,6.011,16]heptadecan-9-one;
(1R,4S,8R)-10-benzyl-4-isopropyl-2-oxa-5,10-diazatricyclo[6.4.0.01,5]dodecan-6-one;
(1R,4S,8R)-4-isopropyl-10-[[4-(trifluoromethyl)phenyl]methyl]-2-oxa-5,10-diazatricyclo[6.4.0.01,5]dodecan-6-one;
(3S,7aR,11aR)-9-benzyl-3-isopropyl-2,3,6,7,7a,8,10,11-octahydrooxazolo[2,3-j][1,6]naphthyridine-5-thione;
(1R,4S,9R)-4-isopropyl-11-[[4-(trifluoromethyl)phenyl]methyl]-2,8-dioxa-5,11-diazatricyclo[7.4.0.01,5]tridecan-6-one;
(1R,4S,8R)-4-isopropyl-10-[[4-(trifluoromethyl)phenyl]methyl]-2-oxa-5,10-diazatricyclo[6.4.0.01,5]dodecan-6-one;
(1R,4R,9R)-4-isopropyl-11-[[4-(trifluoromethyl)phenyl]methyl]-2-oxa-6,11-diazatricyclo[7.4.0.01,6]tridecan-7-one;
(1S,4S,9S)-4-isopropyl-11-[[4-(trifluoromethyl)phenyl]methyl]-2-oxa-6,11-diazatricyclo[7.4.0.01,6]tridecan-7-one;
(1R,4R,9R)-4-ethyl-11-[[4-(trifluoromethyl)phenyl]methyl]-2-oxa-6,11-diazatricyclo[7.4.0.01,6]tridecan-7-one;
(1R,4R,9R)-4-ethyl-11-[[4-(trifluoromethyl)phenyl]methyl]-2-oxa-6,11-diazatricyclo[7.4.0.01,6]tridecan-7-one;
(3S,7aR,11aR)-3-isopropyl-9-[[4-(trifluoromethyl)phenyl]methyl]-3,6,7,7a,8,11-hexahydro-2H-oxazolo[2,3-j][1,6]naphthyridine-5,10-dione;
(3S,7aS,11aR)-3-isopropyl-9-[[4-(trifluoromethyl)phenyl]methyl]-3,6,7,7a, 10,11-hexahydro-2H-oxazolo[2,3-j][1,6]naphthyridine-5,8-dione;
(3S,7aR,9S,11aR)-9-[bis[[4-(trifluoromethyl)phenyl]methyl]amino]-3-isopropyl-3,6,7,7a,8,9,10,11-octahydro-2H-oxazolo[2,3-j]quinolin-5-one;
(3S,7aR,9R,11aR)-9-[bis[[4-(trifluoromethyl)phenyl]methyl]amino]-3-isopropyl-3,6,7,7a,8,9,10,11-octahydro-2H-oxazolo[2,3-j]quinolin-5-one;
(3S,7aR,9S,11 aR)-3-isopropyl-9-[[4-(trifluoromethyl)phenyl]methylamino]-3,6,7,7a,8,9,10,11-octahydro-2H-oxazolo[2,3-j]quinolin-5-one;
(3S,7aR,9R,11aR)-3-isopropyl-9-[[4-(trifluoromethyl)phenyl]methylamino]-3,6,7,7a,8,9,10,11-octahydro-2H-oxazolo[2,3-j]quinolin-5-one;
(3S,7aR,9S,11aR)-3-isopropyl-9-[4-(trifluoromethyl) anilino]-3,6,7,7a,8,9,10,11-octahydro-2H-oxazolo[2,3-j]quinolin-5-one;
(3S,7aR,9S,11aR)-3-isopropyl-9-[4-(trifluoromethyl) anilino]-3,6,7,7a,8,9,10,11-octahydro-2H-oxazolo[2,3-j]quinolin-5-one;
(4S,9R)-4-isopropyl-11-[[4-(trifluoromethyl)phenyl]methyl]-2-oxa-5,11-diazatricyclo[7.5.0.01,5]tetradecan-6-one;
(1R,4S,9S)-4-isopropyl-10-[[4-(trifluoromethyl)phenyl]methyl]-2-oxa-5,10-diazatricyclo[7.3.0.01,5]dodecan-6-one;
(3S,6R,7aR,11aR)-3-isopropyl-6-methyl-9-[[4-(trifluoromethyl)phenyl]methyl]-2,3,6,7,7a,8,10,11-octahydrooxazolo[2,3-j][1,6]naphthyridin-5-one;
(3S,6R,7aR,11aR)-6-ethyl-3-isopropyl-9-[[4-(trifluoromethyl)phenyl]methyl]-2,3,6,7,7a,8,10,11-octahydrooxazolo[2,3-j][1,6]naphthyridin-5-one;
(3S,6R,7aR,11aR)-6-allyl-3-isopropyl-9-[[4-(trifluoromethyl)phenyl]methyl]-2,3,6,7,7a,8,10,11-octahydrooxazolo[2,3-j][1,6]naphthyridin-5-one;
(3S,6R,7aR,11aR)-6-isobutyl-3-isopropyl-9-[[4-(trifluoromethyl)phenyl]methyl]-2,3,6,7,7a,8,10,11-octahydrooxazolo[2,3-j][1,6]naphthyridin-5-one;
(3S,6S,7aR,11aR)-3-isopropyl-6-methyl-9-[[4-(trifluoromethyl)phenyl]methyl]-2,3,6,7,7a,8,10,11-octahydrooxazolo[2,3-j][1,6]naphthyridin-5-one;
(3S,6S,7aR,11aR)-6-ethyl-3-isopropyl-9-[[4-(trifluoromethyl)phenyl]methyl]-2,3,6,7,7a,8,10,11-octahydrooxazolo[2,3-j][1,6]naphthyridin-5-one;
(3S,6S,7aR,11aR)-6-allyl-3-isopropyl-9-[[4-(trifluoromethyl)phenyl]methyl]-2,3,6,7,7a,8,10,11-octahydrooxazolo[2,3-j][1,6]naphthyridin-5-one;
(3S,7aR,11aR)-6-fluoro-3-isopropyl-9-[[4-(trifluoromethyl)phenyl]methyl]-2,3,6,7,7a,8,10,11-octahydrooxazolo[2,3-j][1,6]naphthyridin-5-one;
(3S,7aR,11aR)-6,6-difluoro-3-isopropyl-9-[[4-(trifluoromethyl)phenyl]methyl]-3,7,7a,8,10,11-hexahydro-2H-oxazolo[2,3-j][1,6]naphthyridin-5-one;
   or its pharmaceutically acceptable salts.

In one embodiment, said compound of formula (I) is characterized in that:
p and p′ have a value of 1; and/or
X is O; and/or
Y is O; and/or
m has a value of 1; and/or
n has a value of 1 and W is CR₄R₄′; and/or
Z is NR₁ with R₁ chosen from:
   benzyl, said benzyl being substituted by one or more halogen atom, linear-(C₁-C₆)halogenoalkyl, linear-(C₁-C₆)halogenoalkoxy,
   linear or branched —CH₃—(C₆-C₁₀) heteroaryl, said heteroaryl having at least one heteroatom chosen from O, N, or S, in particular said heteroaryl being a piperidine, and being optionally substituted by one or more linear-(C₁-C₆)halogenoalkyl (C₁-C₃)alkyl or —(C₃-C₆)cycloalkyl,
   linear or branched —(C₁-C₆)alkyl-(C₆-C₁₀)aryl, said aryl being in particular phenyl, and said phenyl being optionally substituted by linear-(C₁-C₃)halogenoalkyl, or
Z is CHNRaRb with Ra and Rb, identical being linear or branched —(C₁-C₃)alkylphenyl, said phenyl being optionally substituted by one or more linear or branched —(C₁-C₃)halogenoalkyl, in particular by one trifluoromethyl; and/or

R₂ and R₂′ are identical and are H; and/or
R₃ and R₃′ are different and chosen from H and branched —(C₁-C₆) alkyl; and/or
R₄ and R₄′ are identical and are H; and/or
R₅ and R₅′ are H; and/or
R₆ and R₆′ are H.

More particularly, said compound if chosen from:
(3S,7aR,11aR)-3-isopropyl-9-[[4-(trifluoromethyl)phenyl]methyl]-2,3,6,7,7a,8,10,11 octahydrooxazolo[2,3-j][1,6]naphthyridin-5-one
(3S,7aR,11aR)-3-isopropyl-9-[[4-(trifluoromethoxy)phenyl]methyl]-2,3,6,7,7a,8,10,11-octahydrooxazolo[2,3-j][1,6]naphthyridin-5-one
(3S,7aR,11aR)-9-[(4-chlorophenyl)methyl]-3-isopropyl-2,3,6,7,7a,8,10,11-octahydrooxazolo[2,3-j][1,6]naphthyridin-5-one
(3S,7aR,11aR)-9-[(3,4-dichlorophenyl)methyl]-3-isopropyl-2,3,6,7,7a,8,10,11-octahydrooxazolo[2,3-j][1,6]naphthyridin-5-one
(3S,7aR,11aR)-9-[[4-chloro-3-(trifluoromethyl)phenyl]methyl]-3-isopropyl-2,3,6,7,7a,8,10,11-octahydrooxazolo[2,3-j][1,6]naphthyridin-5-one
(3S,7aR,11aR)-9-[[2,4-bis(trifluoromethyl)phenyl]methyl]-3-isopropyl-2,3,6,7,7a,8,10,11-octahydrooxazolo[2,3-j][1,6]naphthyridin-5-one
(3S,7aR,11aR)-9-[(2-cyclopropyl-4-quinolyl)methyl]-3-isopropyl-2,3,6,7,7a,8,10,11-octahydrooxazolo[2,3-j][1,6]naphthyridin-5-one
(3S,7aR,11aR)-3-isopropyl-9-[[2-(trifluoromethyl)-4-quinolyl]methyl]-2,3,6,7,7a,8,10,11-octahydrooxazolo[2,3-j][1,6]naphthyridin-5-one
(3S,7aR,11aR)-3-isopropyl-9-[(1-methylindol-2-yl)methyl]-2,3,6,7,7a,8,10,11-octahydrooxazolo[2,3-j][1,6]naphthyridin-5-one
(3S,7aR,11aR)-3-isopropyl-9-[2-[4-(trifluoromethyl)phenyl]ethyl]-2,3,6,7,7a,8,10,11-octahydrooxazolo[2,3-j][1,6]naphthyridin-5-one
(3S,7aR,11aR)-3-isopropyl-9-[3-[4-(trifluoromethyl)phenyl]propyl]-2,3,6,7,7a,8,10,11-octahydrooxazolo[2,3-j][1,6]naphthyridin-5-one
(3S,7aR,9S,11aR)-9-[bis[[4-(trifluoromethyl)phenyl]methyl]amino]-3-isopropyl-3,6,7,7a,8,9,10,11-octahydro-2H-oxazolo[2,3-j]quinolin-5-one or its pharmaceutically acceptable salts.

In particular, in the context of the present invention, the following compounds which are described and named as follows in Aurora Building Blocks 9″, 4 Apr. 2022 (2022 Apr. 4), Aurora Fine Chemicals Ltd, XP055972899, are not covered:
(1S,8R)-9-[(3-hydroxyphenyl)methyl]-2-oxa-5,9-diazatricyclo[6.3.0.0 (1,5)]undecan-6-one;
(1S, 4R, 8R)-9-[(4-methoxy-2,5-dimethylphenyl)methyl]-4-(propan-2-yl)-2-oxa-5,9-diazatricyclo[6.3.0.0 (1,5)]undecan-6-one;
(1S, 8R)-9-([4-(2,2,2-trifluoroethyl)phenyl]methyl)-2-oxa-5,9-diazatricyclo[6.3.0.0 (1,5)]undecan-6-one;
(1S, 4R, 8R)-9-[(5-chloro-2-fluorophenyl)methyl]-4-(propan-2-yl)-2-oxa-5,9-diazatricyclo[6.3.0.0 (1,5)]undecan-6-one;
(1S, 4R, 8R)-9-[(3-methylphenyl)methyl]-4-(propan-2-yl)-2-(oxa-5,9-diazatricyclo[6.3.0.0 (1,5)]undecan-6-one;
(1S, 4R, 8R)-4-(propan-2-yl)-9-[(2,4,6-trimethylphenyl)methyl]-2-oxa-5,9-diazatricyclo[6.3.0.0 (1,5)]undecan-6-one;
(1S, 4R, 8R)-4-(propan-2-yl)-9-[(2,3,4-trimethoxyphenyl)methyl]-2-oxa-5,9-diazatricyclo[6.3.0.0 (1,5)]undecan-6-one; and
(1S, 8R)-9-[(3-chloro-2-fluorophenyl)methyl]-2-oxa-5,9-diazatricyclo[6.3.0.0 (1,5)]undecan-6-one.

As previously mentioned, the present invention also relates to a combination of (a) a compound of Formula (I) as defined herein and (b) at least one other anti-mycobacterial agent.

The anti-mycobacterial agent is as defined below.

Compounds provided herein can be formulated into pharmaceutical compositions, optionally by admixture with one or more pharmaceutically acceptable excipients.

The present invention thus also relates to a pharmaceutical composition comprising a compound of formula (I) as defined herein, and a pharmaceutically acceptable excipient.

In one embodiment, said pharmaceutical composition further comprises at least one other anti-mycobacterial agent.

Anti-mycobacterial agents are well known in the art. Antimycobacterial, or antituberculosis, agents include rifampin, rifabutin, isoniazid, ethambutol, streptomycin, amikacin, kanamycin, moxifloxacin, pyrazinamide, bedaquiline, linezolid, sutezolid, nitroimidazole. Antimycobacterial agents are most commonly prescribed today in multidrug combinations.

In one embodiment, the pharmaceutical composition comprises two, three, four, five, six or seven additional anti-tuberculosis agents. For example, in the treatment of multidrug-resistant tuberculosis, it is common that combinations of four or more drugs are administered to patients. For example, in the treatment of drug-sensitive tuberculosis, it is common that combinations of three or four drugs are administered to patients.

Such compositions may be prepared for use in oral administration, particularly in the form of tablets or capsules, in particular orodispersible (lyoc) tablets; or parenteral administration, particularly in the form of liquid solutions, suspensions or emulsions.

It may be prepared by any of the methods well known in the pharmaceutical art, for example, as described in Remington: The Science and Practice of Pharmacy, 20th ed.; Gennaro, A. R., Ed.; Lippincott Williams & Wilkins: Philadelphia, PA, 2000.

Pharmaceutically compatible binding agents and/or adjuvant materials can be included as part of the composition. Oral compositions will generally include an inert diluent carrier or an edible carrier. They can be administered in unit dose forms, wherein the term “unit dose” means a single dose which is capable of being administered to a patient, and which can be readily handled and packaged, remaining as a physically and chemically stable unit dose comprising either the active compound itself, or as a pharmaceutically acceptable composition.

The tablets, pills, powders, capsules, troches and the like can contain one or more of any of the following ingredients, or compounds of a similar nature: a binder such as microcrystalline cellulose, or gum tragacanth; a diluent such as starch or lactose; a disintegrant such as starch and cellulose derivatives; a lubricant such as magnesium stearate; a glidant such as colloidal silicon dioxide; a sweetening agent such as sucrose or saccharin; or a flavoring agent such as peppermint, or methyl salicylate. Capsules can be in the form of a hard capsule or soft capsule, which are generally made from gelatin blends optionally blended with plasticizers, as well as a starch capsule. In addition, dosage unit forms can contain various other materials that modify the physical form of the dosage unit, for example, coatings of sugar, shellac, or enteric agents. Other oral dosage forms syrup or elixir may contain sweetening agents, preservatives, dyes, colorings, and flavorings. In addition, the active compounds may be incorporated into fast dissolve, modified-release or sustained-release preparations and formulations, and wherein such sustained-release formulations are preferably bi-modal.

Liquid preparations for administration include sterile aqueous or non-aqueous solutions, suspensions, and emulsions. The liquid compositions may also include binders, buffers, preservatives, chelating agents, sweetening, flavoring and coloring agents, and the like. Non-aqueous solvents include alcohols, propylene glycol, polyethylene glycol, acrylate copolymers, vegetable oils such as olive oil, and organic esters such as ethyl oleate. Aqueous carriers include mixtures of alcohols and water, hydrogels, buffered media, and saline. In particular, biocompatible, biodegradable lactide polymer, lactide/glycolide copolymer, or polyoxyethylene-polyoxypropylene copolymers may be useful excipients to control the release of the active compounds. Intravenous vehicles can include fluid and nutrient replenishers, electrolyte replenishers, such as those based on Ringer's dextrose, and the like.

### Compounds for Use

As already mentioned, the present invention also relates to a compound of formula (I) as defined herein for use as a drug.

In particular, said compound can be used to prevent and/or treat a mycobacterial infection.

Mycobacterial infections are well known in the art. A mycobacterial infection is one caused by infection with a Mycobacterium.

The Mycobacterium may be a member of one of the following groups of Mycobacterium: Mycobacterium tuberculosis complex (MTC), Mycobacterium avium comp/ex (MAC), Mycobacterium gordonae clade, Mycobacterium kansasii clade, Mycobacterium chelonae clade, Mycobacterium fortuitum clade, Mycobacterium parafortuitum clade or Mycobacterium vaccae clade, Mycobacterium marinum and Mycobacterium abscessus. The Mycobacterium may also be Mycobacterium ulcerans or Mycobacterium leprae.

In particular, the Mycobacterium is a member of the Mycobacterium tuberculosis complex (MTC). Members of Mycobacterium tuberculosis complex (MTC) include Mycobacterium tuberculosis, Mycobacterium africanum, Mycobacterium bovis, Mycobacterium bovis BCG, Mycobacterium canetti, Mycobacterium caprae, Mycobacterium microti, Mycobacterium marinum, Mycobacterium abscessus and Mycobacterium pinnipedii.

These mycobacteria are causative agents of human and animal tuberculosis. Mycobacterium tuberculosis is the major cause of human tuberculosis.

Still particularly, the infection is a Mycobacterium tuberculosis infection. In other words, the mycobacterial infection is caused by infection with Mycobacterium tuberculosis.

In one embodiment, the Mycobacterium tuberculosis is multidrug-resistant.

In another aspect, the invention relates to a compound of Formula (I) as described herein, or a pharmaceutically acceptable salt thereof, for use in the treatment of a disease caused by infection with a Mycobacterium.

In particular, the Mycobacterium is selected from those hereinbefore described.

For example, the mycobacterial infection may be caused by infection with a Mycobacterium selected from the following: Mycobacterium tuberculosis, Mycobacterium africanum, Mycobacterium bovis, Mycobacterium bovis BCG, Mycobacterium canetti, Mycobacterium caprae, Mycobacterium microti, Mycobacterium pinnipedii, Mycobacterium avium, Mycobacterium avium paratuberclosis, Mycobacterium avium silaticum, Mycobacterium avium hominissuis, Mycobacterium columbiense, Mycobacterium indicus pranii, Mycobacterium asiaticum, Mycobacterium gordonae, Mycobacterium gastri, Mycobacterium kansasii, Mycobacterium abscessus, Mycobacterium bolletii, Mycobacterium chlonae, include Mycobacterium boenickei, Mycobacterium brisbanense, Mycobacterium cosmeticum, Mycobacterium fortuitum, Mycobacterium fortuitum subspecies acetamidolyticum, Mycobacterium houstonense, Mycobacterium mageritense, Mycobacterium neworleansense, Mycobacterium peregrinum, Mycobacterium porcinum, Mycobacterium senegalense, Mycobacterium septicum, Mycobacterium austroafricanum, Mycobacterium diernhoferi, Mycobacterium frederiksbergense, Mycobacterium hodleri, Mycobacterium neoaurum, Mycobacterium parafortuitum, Mycobacterium ulcerans, Mycobacterium marinum, Mycobacterium abscessus and Mycobacterium leprae.

Diseases caused by infection with a Mycobacterium include, but are not limited to, tuberculosis (e.g. from Mycobacterium tuberculosis), leprosy (e.g. from Mycobacterium leprae), Johne's disease (e.g. from Mycobacterium avium subspecies paratuberculosis), Buruli or Bairnsdale ulcer (e.g. from Mycobacterium ulcerans), Crohn's disease (e.g. from Mycobacterium avium subspecies paratuberculosis), pulmonary disease or pulmonary infection, pneumonia, bursa, synovial, tendon sheaths, localized abscess, lymphadenitis, skin and soft tissue infections, Lady Windermere syndrome (e.g. from Mycobacterium avium complex (MAC)), MAC lung disease, disseminated Mycobacterium avium complex (DMAC), disseminated Mycobacterium avium intracellulare complex (DMAIC), hot-tub lung (e.g. from Mycobacterium avium complex), MAC mastitis, MAC pyomyositis, or granuloma disease.

In particular, in the context of the invention, the disease is tuberculosis.

In one embodiment, the invention thus also relates to a method of treatment of a mycobacterial infection in a subject in need thereof, said treatment comprising administering to said subject a therapeutically effective amount of a compound of Formula (I) as described in this section, or pharmaceutically acceptable salt thereof.

As described herein, a mycobacterial infection is one caused by infection with a Mycobacterium. The Mycobacterium is as hereinbefore described.

In one embodiment, the invention relates to a method of treatment of a Mycobacterium tuberculosis infection.

In another embodiment, the invention relates to a method of treatment of a disease caused by infection with a Mycobacterium in a subject in need thereof, said treatment comprising administering to said subject a therapeutically effective amount of a compound of Formula (I), or a pharmaceutically acceptable salt thereof.

In particular in the context of the invention, the disease is tuberculosis.

In one embodiment, said compound is used in combination with at least one other anti-mycobacterial agent.

Anti-mycobacterial agents are well known in the art. Antimycobacterial, or antituberculosis, agents include rifampin, rifabutin, isoniazid, ethambutol, streptomycin, amikacin, kanamycin, moxifloxacin, pyrazinamide, bedaquiline, linezolid, sutezolid, nitroimidazole.

Antimycobacterial agents are most commonly prescribed today in multidrug combinations.

In one embodiment, the combination comprises two, three, four, five, six or seven additional anti-tuberculosis agents. For example, in the treatment of multidrug-resistant tuberculosis, it is common that combinations of four or more drugs are administered to patients. For example, in the treatment of drug-sensitive tuberculosis, it is common that combinations of three or four drugs are administered to patients.

The terms “treat”, “treating”, “treated” or “treatment”, as used in the context of the invention, refer to therapeutic treatment wherein the object is to eliminate or lessen symptoms. Beneficial or desired clinical results include, but are not limited to, elimination of symptoms, alleviation of symptoms, diminishment of extent of condition, stabilized (i.e., not worsening) state of condition, delay or slowing of progression of the condition.

The terms “prevent”, “prevention”, “preventing” or “prevented”, as used in the context of the present invention, refer to the prevention of the onset, recurrence or spread of a disease or disorder or infection, or of one or more symptoms thereof. In certain embodiments, the terms refer to the treatment with or administration of a compound provided herein prior to the onset of symptoms, particularly to patients at risk of disease or disorder or infection provided herein. The terms encompass the inhibition or reduction of a symptom of the particular disease or disorder or infection. Subjects with familial history of a disease or disorder or infection in particular are candidates for preventive regimens in certain embodiments. In addition, subjects who have a history of recurring symptoms are also potential candidates for the prevention. In this regard, the term “prevention” may be interchangeably used with the term “prophylactic treatment”.

In particular, the subject in need of a treatment against mycobacterial infection or a disease caused by infection with a Mycobacterium is a subject afflicted with such disease or infection.

In the context of the present invention, the identification of the subjects who are in need of treatment of herein-described diseases and conditions is conducted as above mentioned and is well within the ability and knowledge of the man skilled in the art. A clinician skilled in the art can readily identify, by the above-mentioned technics, those subjects who are in need of such treatment.

A therapeutically effective amount can be readily determined by the attending diagnostician, as one skilled in the art, by the use of conventional techniques and by observing results obtained under analogous circumstances. In determining the therapeutically effective amount, a number of factors are considered by the attending diagnostician, including, but not limited to: the species of subject; its size, age, and general health; the specific disease involved; the degree of involvement or the severity of the disease; the response of the individual subject; the particular compound administered; the mode of administration; the bioavailability characteristic of the preparation administered; the dose regimen selected; the use of concomitant medication; and other relevant circumstances.

As used herein, an «effective amount” refers to an amount which is effective in reducing, eliminating, treating or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment and chronic use.

The term “patient” or “subject” refers to a warm-blooded animal such as a mammal, in particular a human, male or female, unless otherwise specified, which is afflicted with, or has the potential to be afflicted with one or more diseases and conditions described herein.

In particular, the compounds of the combination according to the invention are administered separately, sequentially or simultaneously.

The amount of the compound according to the invention, which is required to achieve the desired biological effect, will vary depending upon a number of factors, including the dosage of the drug to be administered, the chemical characteristics (e.g. hydrophobicity) of the compounds employed, the potency of the compounds, the type of resistance, the state of resistance in the patient, and the route of administration.

Compounds provided herein can be formulated into pharmaceutical compositions, optionally by admixture with one or more pharmaceutically acceptable excipients.

Such compositions may be prepared for use in oral administration, particularly in the form of tablets or capsules, in particular orodispersible (lyoc) tablets; or parenteral administration, particularly in the form of liquid solutions, suspensions or emulsions.

It may be prepared by any of the methods well known in the pharmaceutical art, for example, as described in Remington: The Science and Practice of Pharmacy, 20^{th} ed.; Gennaro, A. R., Ed.; Lippincott Williams & Wilkins: Philadelphia, PA, 2000. Pharmaceutically compatible binding agents and/or adjuvant materials can be included as part of the composition. Oral compositions will generally include an inert diluent carrier or an edible carrier. They can be administered in unit dose forms, wherein the term “unit dose” means a single dose which is capable of being administered to a patient, and which can be readily handled and packaged, remaining as a physically and chemically stable unit dose comprising either the active compound itself, or as a pharmaceutically acceptable composition.

The tablets, pills, powders, capsules, troches and the like can contain one or more of any of the following ingredients, or compounds of a similar nature: a binder such as microcrystalline cellulose, or gum tragacanth; a diluent such as starch or lactose; a disintegrant such as starch and cellulose derivatives; a lubricant such as magnesium stearate; a glidant such as colloidal silicon dioxide; a sweetening agent such as sucrose or saccharin; or a flavoring agent such as peppermint, or methyl salicylate. Capsules can be in the form of a hard capsule or soft capsule, which are generally made from gelatin blends optionally blended with plasticizers, as well as a starch capsule. In addition, dosage unit forms can contain various other materials that modify the physical form of the dosage unit, for example, coatings of sugar, shellac, or enteric agents. Other oral dosage forms syrup or elixir may contain sweetening agents, preservatives, dyes, colorings, and flavorings. In addition, the active compounds may be incorporated into fast dissolve, modified-release or sustained-release preparations and formulations, and wherein such sustained-release formulations are preferably bi-modal.

Liquid preparations for administration include sterile aqueous or non-aqueous solutions, suspensions, and emulsions. The liquid compositions may also include binders, buffers, preservatives, chelating agents, sweetening, flavoring and coloring agents, and the like. Non-aqueous solvents include alcohols, propylene glycol, polyethylene glycol, acrylate copolymers, vegetable oils such as olive oil, and organic esters such as ethyl oleate. Aqueous carriers include mixtures of alcohols and water, hydrogels, buffered media, and saline. In particular, biocompatible, biodegradable lactide polymer, lactide/glycolide copolymer, or polyoxyethylene-polyoxypropylene copolymers may be useful excipients to control the release of the active compounds. Intravenous vehicles can include fluid and nutrient replenishers, electrolyte replenishers, such as those based on Ringer's dextrose, and the like.

Examples of modes of administration include parenteral e.g. subcutaneous, intramuscular, intravenous, intradermal, as well as oral administration.

### Process of Preparation

The present invention is also concerned with the process of preparation of the compounds of formula (I) as described herein.

The compounds of the present invention may be prepared in a number of ways well known to those skilled in the art. The compounds can be synthesized, for example, by application or adaptation of the methods described below, or variations thereon as appreciated by the skilled artisan. The appropriate modifications and substitutions will be readily apparent and well known or readily obtainable from the scientific literature to those skilled in the art.

It will be appreciated that the compounds of the present invention may contain one or more asymmetrically substituted carbon atoms, and may be isolated in optically active or racemic forms. Thus, all diastereomeric forms of a structure are intended, unless the specific stereochemistry—is specifically indicated. It is well known in the art how to prepare and isolate such optically active forms.

For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

Compounds of the present invention may be prepared by a variety of synthetic routes. The reagents and starting materials are commercially available, or readily synthesized by well-known techniques by one of ordinary skill in the arts. All substituents, unless otherwise indicated, are as previously defined.

In the reactions described hereinafter, it may be necessary to protect reactive functional groups, for example hydroxyl, amino, imino, thio or carboxy groups, where these are desired in the final product, to avoid their unwanted participation in the reactions. Conventional protecting groups may be used in accordance with standard practice, for examples see T. W. Greene and P. G. M. Wuts in Protective Groups in Organic Chemistry, 4th ed. (2007), John Wiley & Sons Inc., 1999; J. F. W. McOmie in Protective Groups in Organic Chemistry, Plenum Press, 1973.

The compound thus prepared may be recovered from the reaction mixture by conventional means. For example, the compounds may be recovered by distilling off the solvent from the reaction mixture or, if necessary, after distilling off the solvent from the reaction mixture, pouring the residue into water followed by extraction with a water-immiscible organic solvent and distilling off the solvent from the extract. Additionally, the product can, if desired, be further purified by various well-known techniques, such as recrystallization, reprecipitation or the various chromatography techniques, notably column chromatography or preparative thin layer chromatography.

The reactions can be carried out by the skilled person by applying or adapting the methods illustrated in the examples hereinafter.

In particular, compounds of formula (I) can be prepared according to protocols mentioned in the experimental part below.

Further, the process of the invention may also comprise the additional step of isolating the compound of formula (I). This can be done by the skilled person by any of the known conventional means, such as the recovery methods described above.

Generally, the starting products are commercially available mainly from Aldrich or Acros or other typical chemicals supplier or may be obtained by applying or adapting any known methods or those described in the examples.

In the context of the present invention, it should be understood that “a compound for use for the prevention and/or treatment of” is equivalent to “the use of a compound for the prevention and/or treatment of” and to “the use of a compound for the manufacture of a medicament for the prevention and/or treatment of”.

The invention will be further illustrated by the following examples.

### EXAMPLES

### Part A—Synthesis of Compounds According to the Invention

The following compounds according to the invention have been prepared.

TABLE 1
compounds according to the invention

| No | IUPAC name | Structure |
|---|---|---|
| 1 | (3S,7aR,11aR)-9-[(4- fluorophenyl)methyl]-3-isopropyl- 2,3,6,7,7a,8,10,11-octahydrooxazolo[2,3- j][1,6]naphthyridin-5-one | 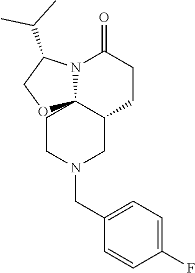 [SMILES: CC(C)[C@H]1CO[C@]23CCN(Cc4ccc(F)cc4)C[C@H]2CCC(=O)N13] |
| 2 | (3S,7aR,11aR)-3-isopropyl-9-[[4- (trifluoromethyl)phenyl]methyl]- 2,3,6,7,7a,8,10,11-octahydrooxazolo[2,3- j][1,6]naphthyridin-5-one | 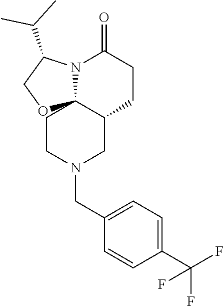 [SMILES: CC(C)[C@H]1CO[C@]23CCN(Cc4ccc(C(F)(F)F)cc4)C[C@H]2CCC(=O)N13] |
| 3 | (3S,7aR,11aR)-3-isopropyl-9-[[4- (trifluoromethoxy)phenyl]methyl]- 2,3,6,7,7a,8,10,11-octahydrooxazolo[2,3- j][1,6]naphthyridin-5-one | 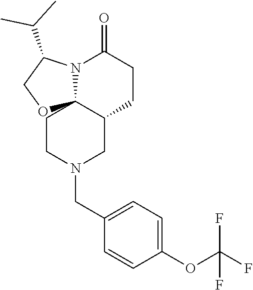 [SMILES: CC(C)[C@H]1CO[C@]23CCN(Cc4ccc(OC(F)(F)F)cc4)C[C@H]2CCC(=O)N13] |
| 4 | (3S,7aR,11aR)-9-[(4- chlorophenyl)methyl]-3-isopropyl- 2,3,6,7,7a,8,10,11-octahydrooxazolo[2,3- j][1,6]naphthyridin-5-one | 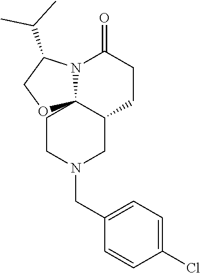 [SMILES: CC(C)[C@H]1CO[C@]23CCN(Cc4ccc(Cl)cc4)C[C@H]2CCC(=O)N13] |
| 5 | (3S,7aR,11aR)-9-[(3,4- dichlorophenyl)methyl]-3-isopropyl- 2,3,6,7,7a,8,10,11-octahydrooxazolo[2,3- j][1,6]naphthyridin-5-one | 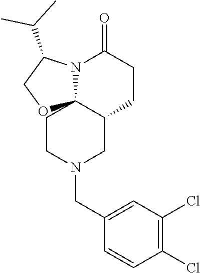 [SMILES: CC(C)[C@H]1CO[C@]23CCN(Cc4ccc(Cl)c(Cl)c4)C[C@H]2CCC(=O)N13] |
| 6 | (3S,7aR,11aR)-3-isopropyl-9-(p- tolylmethyl)-2,3,6,7,7a,8,10,11- octahydrooxazolo[2,3-j][1,6]naphthyridin- 5-one | 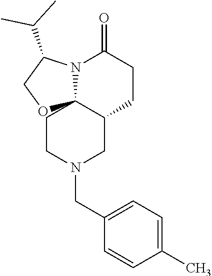 [SMILES: Cc1ccc(CN2CC[C@]34OC[C@H](C(C)C)N3C(=O)CC[C@@H]4C2)cc1] |
| 7 | (3S,7aR,11aR)-3-isopropyl-9-[(4- methoxyphenyl)methyl]- 2,3,6,7,7a,8,10,11-octahydrooxazolo[2,3- j][1,6]naphthyridin-5-one | 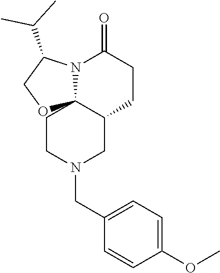 [SMILES: COc1ccc(CN2CC[C@]34OC[C@H](C(C)C)N3C(=O)CC[C@@H]4C2)cc1] |
| 8 | (3S,7aR,11aR)-9-[(4- hydroxyphenyl)methyl]-3-isopropyl- 2,3,6,7,7a,8,10,11-octahydrooxazolo[2,3- j][1,6]naphthyridin-5-one | 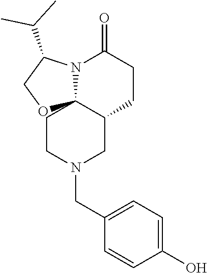 [SMILES: CC(C)[C@H]1CO[C@]23CCN(Cc4ccc(O)cc4)C[C@H]2CCC(=O)N13] |
| 9 | (3S,7aR,11aR)-9-[[4-chloro-3- (trifluoromethyl)phenyl]methyl]-3- isopropyl-2,3,6,7,7a,8,10,11- octahydrooxazolo[2,3-j][1,6]naphthyridin- 5-one | 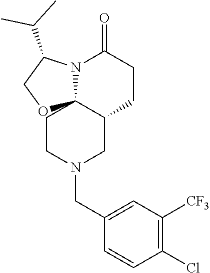 [SMILES: CC(C)[C@H]1CO[C@]23CCN(Cc4ccc(Cl)c(C(F)(F)F)c4)C[C@H]2CCC(=O)N13] |
| 10 | (3S,7aR,11aR)-9-[[2,4- bis(trifluoromethyl)phenyl]methyl]-3- isopropyl-2,3,6,7,7a,8,10,11- octahydrooxazolo[2,3-j][1,6]naphthyridin- 5-one | 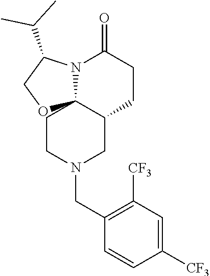 [SMILES: CC(C)[C@H]1CO[C@]23CCN(Cc4ccc(C(F)(F)F)cc4C(F)(F)F)C[C@H]2CCC(=O)N13] |
| 11 | (3S,7aR,11aR)-3-isopropyl-9-[(4- phenylphenyl)methyl]-2,3,6,7,7a,8,10,11- octahydrooxazolo[2,3-j][1,6]naphthyridin- 5-one | 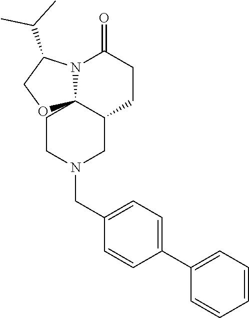 [SMILES: CC(C)[C@H]1CO[C@]23CCN(Cc4ccc(-c5ccccc5)cc4)C[C@H]2CCC(=O)N13] |
| 12 | (3S,7aR,11aR)-3-isopropyl-9-[(3- phenoxyphenyl)methyl]- 2,3,6,7,7a,8,10,11-octahydrooxazolo[2,3- j][1,6]naphthyridin-5-one | 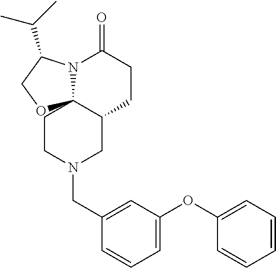 [SMILES: CC(C)[C@H]1CO[C@]23CCN(Cc4cccc(Oc5ccccc5)c4)C[C@H]2CCC(=O)N13] |
| 13 | (3S,7aR,11aR)-3-isopropyl-9-[[4-[4-[4- (trifluoromethoxy)phenoxy]-1- piperidyl]phenyl]methyl]- 2,3,6,7,7a,8,10,11-octahydrooxazolo[2,3- j][1,6]naphthyridin-5-one | 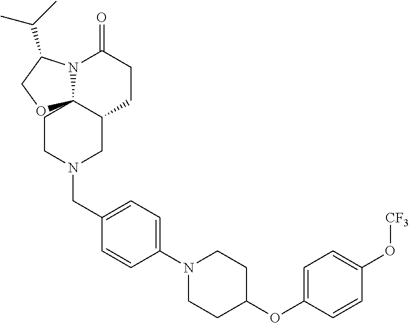 [SMILES: CC(C)[C@H]1CO[C@]23CCN(Cc4ccc(N5CCC(Oc6ccc(OC(F)(F)F)cc6)CC5)cc4)C[C@H]2CCC(=O)N13] |
| 14 | (3S,7aR,11aR)-3-isopropyl-9-[[4-[4-[4- (trifluoromethoxy)phenyl]-1- piperidyl]phenyl]methyl]- 2,3,6,7,7a,8,10,11-octahydrooxazolo[2,3- j][1,6]naphthyridin-5-one | 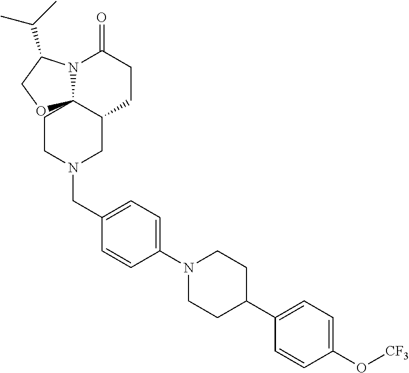 [SMILES: CC(C)[C@H]1CO[C@]23CCN(Cc4ccc(N5CCC(c6ccc(OC(F)(F)F)cc6)CC5)cc4)C[C@H]2CCC(=O)N13] |
| 15 | (3S,7aR,11aR)-3-isopropyl-9-[[4-(1- piperidyl)phenyl]methyl]- 2,3,6,7,7a,8,10,11-octahydrooxazolo[2,3- j][1,6]naphthyridin-5-one | 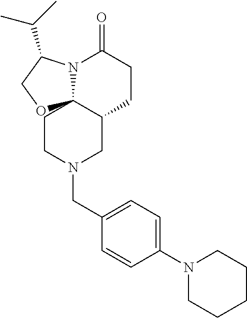 [SMILES: CC(C)[C@H]1CO[C@]23CCN(Cc4ccc(N5CCCCC5)cc4)C[C@H]2CCC(=O)N13] |
| 16 | (3S,7aR,11aR)-3-isopropyl-9-(2- pyridylmethyl)-2,3,6,7,7a,8,10,11- octahydrooxazolo[2,3-j][1,6]naphthyridin- 5-one | 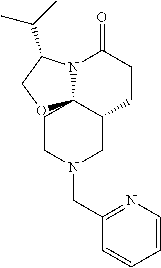 [SMILES: CC(C)[C@H]1CO[C@]23CCN(Cc4ccccn4)C[C@H]2CCC(=O)N13] |
| 17 | (3S,7aR,11aR)-3-isopropyl-9-(3- pyridylmethyl)-2,3,6,7,7a,8,10,11- octahydrooxazolo[2,3-j][1,6]naphthyridin- 5-one | 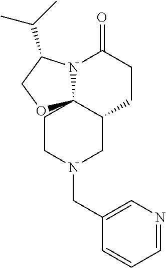 [SMILES: CC(C)[C@H]1CO[C@]23CCN(Cc4cccnc4)C[C@H]2CCC(=O)N13] |
| 18 | (3S,7aR,11aR)-3-isopropyl-9-(4- pyridylmethyl)-2,3,6,7,7a,8,10,11- octahydrooxazolo[2,3-j][1,6]naphthyridin- 5-one | 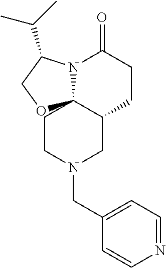 [SMILES: CC(C)[C@H]1CO[C@]23CCN(Cc4ccncc4)C[C@H]2CCC(=O)N13] |
| 19 | (3S,7aR,11aR)-3-isopropyl-9-[[6- (trifluoromethyl)-3-pyridyl]methyl]- 2,3,6,7,7a,8,10,11-octahydrooxazolo[2,3- j][1,6]naphthyridin-5-one | 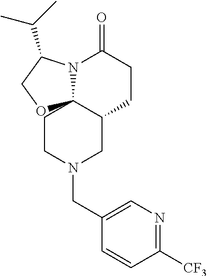 [SMILES: CC(C)[C@H]1CO[C@]23CCN(Cc4ccc(C(F)(F)F)nc4)C[C@H]2CCC(=O)N13] |
| 20 | (3S,7aR,11aR)-3-isopropyl-9-[[5- (trifluoromethyl)-2-pyridyl]methyl]- 2,3,6,7,7a,8,10,11-octahydrooxazolo[2,3- j][1,6]naphthyridin-5-one | 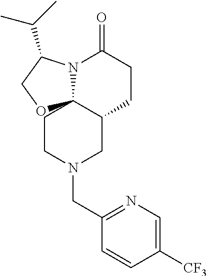 [SMILES: CC(C)[C@H]1CO[C@]23CCN(Cc4ccc(C(F)(F)F)cn4)C[C@H]2CCC(=O)N13] |
| 21 | (3S,7aR,11aR)-3-isopropyl-9-(2- quinolylmethyl)-2,3,6,7,7a,8,10,11- octahydrooxazolo[2,3-j][1,6]naphthyridin- 5-one | 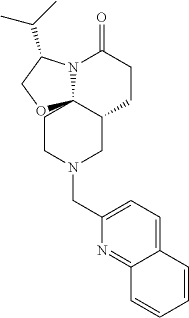 [SMILES: CC(C)[C@H]1CO[C@]23CCN(Cc4ccc5ccccc5n4)C[C@H]2CCC(=O)N13] |
| 22 | (3S,7aR,11aR)-3-isopropyl-9-(8- quinolylmethyl)-2,3,6,7,7a,8,10,11- octahydrooxazolo[2,3-j][1,6]naphthyridin- 5-one | 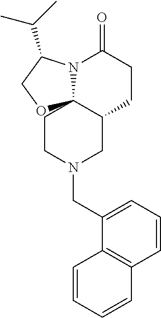 [SMILES: CC(C)[C@H]1CO[C@]23CCN(Cc4cccc5ccccc45)C[C@H]2CCC(=O)N13] |
| 23 | (3S,7aR,11aR)-3-isopropyl-9-(6- quinolylmethyl)-2,3,6,7,7a,8,10,11- octahydrooxazolo[2,3-j][1,6]naphthyridin- 5-one | 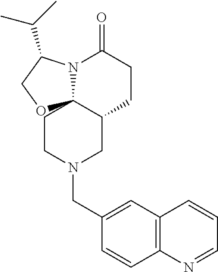 [SMILES: CC(C)[C@H]1CO[C@]23CCN(Cc4ccc5ncccc5c4)C[C@H]2CCC(=O)N13] |
| 24 | (3S,7aR,11aR)-9-[(2-cyclopropyl-4- quinolyl)methyl]-3-isopropyl- 2,3,6,7,7a,8,10,11-octahydrooxazolo[2,3- j][1,6]naphthyridin-5-one | 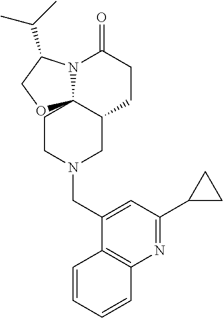 [SMILES: CC(C)[C@H]1CO[C@]23CCN(Cc4cc(C5CC5)nc5ccccc45)C[C@H]2CCC(=O)N13] |
| 25 | (3S,7aR,11aR)-3-isopropyl-9-[[2- (trifluoromethyl)-4-quinolyl]methyl]- 2,3,6,7,7a,8,10,11-octahydrooxazolo[2,3- j][1,6]naphthyridin-5-one | 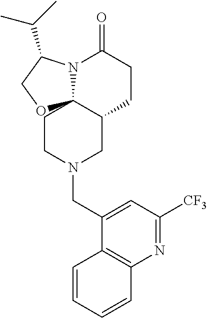 [SMILES: CC(C)[C@H]1CO[C@]23CCN(Cc4cc(C(F)(F)F)nc5ccccc45)C[C@H]2CCC(=O)N13] |
| 26 | (3S,7aR,11aR)-3-isopropyl-9-[(1- methylimidazol-2-yl)methyl]- 2,3,6,7,7a,8,10,11-octahydrooxazolo[2,3- j][1,6]naphthyridin-5-one | 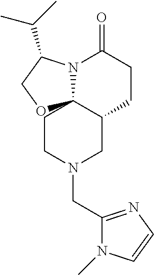 [SMILES: CC(C)[C@H]1CO[C@]23CCN(Cc4nccn4C)C[C@H]2CCC(=O)N13] |
| 27 | (3S,7aR,11aR)-3-isopropyl-9-[(3-methyl- 1H-indol-2-yl)methyl]-2,3,6,7,7a,8,10,11- octahydrooxazolo[2,3-j][1,6]naphthyridin- 5-one | 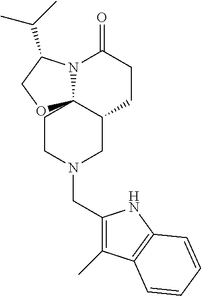 [SMILES: Cc1c(CN2CC[C@]34OC[C@H](C(C)C)N3C(=O)CC[C@@H]4C2)[nH]c2ccccc12] |
| 28 | (3S,7aR,11aR)-3-isopropyl-9-[(1- methylindol-2-yl)methyl]- 2,3,6,7,7a,8,10,11-octahydrooxazolo[2,3- j][1,6]naphthyridin-5-one | 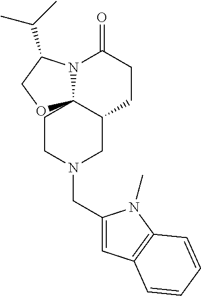 [SMILES: CC(C)[C@H]1CO[C@]23CCN(Cc4cc5ccccc5n4C)C[C@H]2CCC(=O)N13] |
| 29 | (3S,7aR,11aR)-9-(cyclohexylmethyl)-3- isopropyl-2,3,6,7,7a,8,10,11- octahydrooxazolo[2,3-j][1,6]naphthyridin- 5-one | 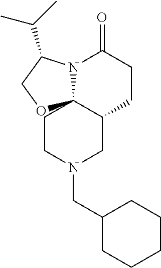 [SMILES: CC(C)[C@H]1CO[C@]23CCN(CC4CCCCC4)C[C@H]2CCC(=O)N13] |
| 30 | (3S,7aR,11aR)-9-cyclobutyl-3-isopropyl- 2,3,6,7,7a,8,10,11-octahydrooxazolo[2,3- j][1,6]naphthyridin-5-one | 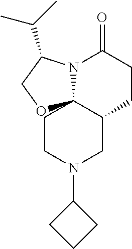 [SMILES: CC(C)[C@H]1CO[C@]23CCN(C4CCC4)C[C@H]2CCC(=O)N13] |
| 31 | (3S,7aR,11aR)-9-cyclohexyl-3-isopropyl- 2,3,6,7,7a,8,10,11-octahydrooxazolo[2,3- j][1,6]naphthyridin-5-one | 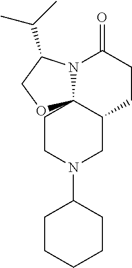 [SMILES: CC(C)[C@H]1CO[C@]23CCN(C4CCCCC4)C[C@H]2CCC(=O)N13] |
| 32 | (3S,7aR,11aR)-3-isopropyl-9- (tetrahydropyran-4-ylmethyl)- 2,3,6,7,7a,8,10,11-octahydrooxazolo[2,3- j][1,6]naphthyridin-5-one | 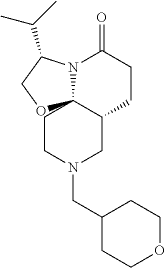 [SMILES: CC(C)[C@H]1CO[C@]23CCN(CC4CCOCC4)C[C@H]2CCC(=O)N13] |
| 33 | (3S,7aR,11aR)-3-isopropyl-9- (tetrahydropyran-2-ylmethyl)- 2,3,6,7,7a,8,10,11-octahydrooxazolo[2,3- j][1,6]naphthyridin-5-one | 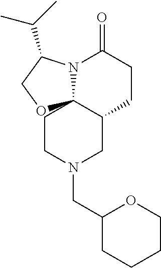 [SMILES: CC(C)[C@H]1CO[C@]23CCN(CC4CCCCO4)C[C@H]2CCC(=O)N13] |
| 34 | (3S,7aR,11aR)-3-isopropyl-9- (tetrahydrofuran-2-ylmethyl)- 2,3,6,7,7a,8,10,11-octahydrooxazolo[2,3- j][1,6]naphthyridin-5-one | 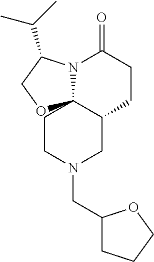 [SMILES: CC(C)[C@H]1CO[C@]23CCN(CC4CCCO4)C[C@H]2CCC(=O)N13] |
| 35 | (3S,7aR,11aR)-3-isopropyl-9-phenyl- 2,3,6,7,7a,8,10,11-octahydrooxazolo[2,3- j][1,6]naphthyridin-5-one | 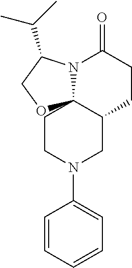 [SMILES: CC(C)[C@H]1CO[C@]23CCN(c4ccccc4)C[C@H]2CCC(=O)N13] |
| 36 | (3S,7aR,11aR)-3-isopropyl-9-[4- (trifluoromethyl)phenyl]- 2,3,6,7,7a,8,10,11-octahydrooxazolo[2,3- j][1,6]naphthyridin-5-one | 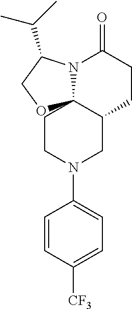 [SMILES: CC(C)[C@H]1CO[C@]23CCN(c4ccc(C(F)(F)F)cc4)C[C@H]2CCC(=O)N13] |
| 37 | (3S,7aR,11aR)-3-isopropyl-9-(2- phenylethyl)-2,3,6,7,7a,8,10,11- octahydrooxazolo[2,3-j][1,6]naphthyridin- 5-one | 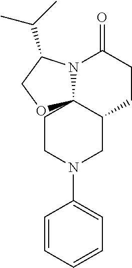 [SMILES: CC(C)[C@H]1CO[C@]23CCN(c4ccccc4)C[C@H]2CCC(=O)N13] |
| 38 | (3S,7aR,11aR)-3-isopropyl-9-[2-[4- (trifluoromethyl)phenyl]ethyl]- 2,3,6,7,7a,8,10,11-octahydrooxazolo[2,3- j][1,6]naphthyridin-5-one | 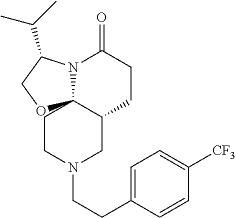 [SMILES: CC(C)[C@H]1CO[C@]23CCN(CCc4ccc(C(F)(F)F)cc4)C[C@H]2CCC(=O)N13] |
| 39 | (3S,7aR,11aR)-3-isopropyl-9-[3-[4- (trifluoromethyl)phenyl]propyl]- 2,3,6,7,7a,8,10,11-octahydrooxazolo[2,3- j][1,6]naphthyridin-5-one | 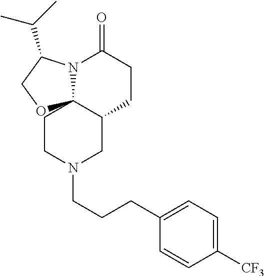 [SMILES: CC(C)[C@H]1CO[C@]23CCN(CCCc4ccc(C(F)(F)F)cc4)C[C@H]2CCC(=O)N13] |
| 40 | (3S,7aR,11aR)-3-isopropyl-9-(1- phenylethyl)-2,3,6,7,7a,8,10,11- octahydrooxazolo[2,3-j][1,6]naphthyridin- 5-one | 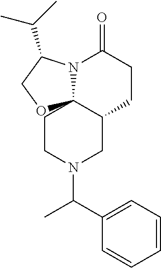 [SMILES: CC(C)[C@H]1CO[C@]23CCN(C(C)c4ccccc4)C[C@H]2CCC(=O)N13] |
| 41 | (3S,7aR,11aR)-9-[2,2-difluoro-2-[4- (trifluoromethyl)phenyl]ethyl]-3-isopropyl- 2,3,6,7,7a,8,10,11-octahydrooxazolo[2,3- j][1,6]naphthyridin-5-one | 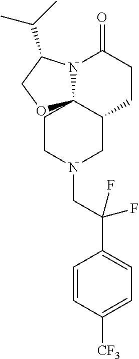 [SMILES: CC(C)[C@H]1CO[C@]23CCN(CC(F)(F)c4ccc(C(F)(F)F)cc4)C[C@H]2CCC(=O)N13] |
| 42 | (3S,7aR,11aR)-3-isopropyl-9-(2- phenylacetyl)-2,3,6,7,7a,8,10,11- octahydrooxazolo[2,3-j][1,6]naphthyridin- 5-one | 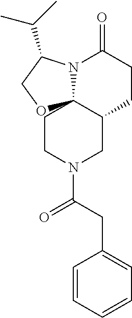 [SMILES: CC(C)[C@H]1CO[C@]23CCN(C(=O)Cc4ccccc4)C[C@H]2CCC(=O)N13] |
| 43 | (3S,7aR,11aR)-3-isopropyl-9-[2-[4- (trifluoromethyl)phenyl]acetyl]- 2,3,6,7,7a,8,10,11-octahydrooxazolo[2,3- j][1,6]naphthyridin-5-one | 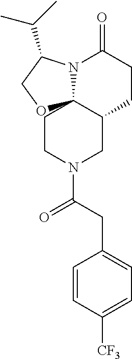 [SMILES: CC(C)[C@H]1CO[C@]23CCN(C(=O)Cc4ccc(C(F)(F)F)cc4)C[C@H]2CCC(=O)N13] |
| 44 | (3S,7aR,11aR)-3-isopropyl-9-[3-[4- (trifluoromethyl)phenyl]propanoyl]- 2,3,6,7,7a,8,10,11-octahydrooxazolo[2,3- j][1,6]naphthyridin-5-one | 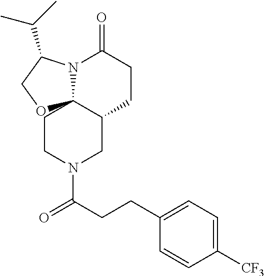 [SMILES: CC(C)[C@H]1CO[C@]23CCN(C(=O)CCc4ccc(C(F)(F)F)cc4)C[C@H]2CCC(=O)N13] |
| 45 | (3S,7aR,11aR)-3-isopropyl-9-[4-[4- (trifluoromethyl)phenyl]butanoyl]- 2,3,6,7,7a,8,10,11-octahydrooxazolo[2,3- j][1,6]naphthyridin-5-one | 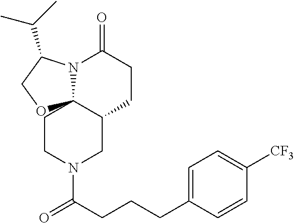 [SMILES: CC(C)[C@H]1CO[C@]23CCN(C(=O)CCCc4ccc(C(F)(F)F)cc4)C[C@H]2CCC(=O)N13] |
| 46 | (3S,7aR,11aR)-9-[2,2-difluoro-2-[4- (trifluoromethyl)phenyl]acetyl]-3- isopropyl-2,3,6,7,7a,8,10,11- octahydrooxazolo[2,3-j][1,6]naphthyridin- 5-one | 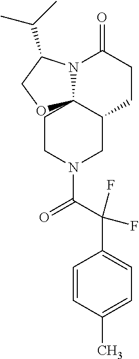 [SMILES: Cc1ccc(C(F)(F)C(=O)N2CC[C@]34OC[C@H](C(C)C)N3C(=O)CC[C@@H]4C2)cc1] |
| 47 | benzyl (3S,7aR,11aR)-3-isopropyl-5-oxo- 2,3,6,7,7a,8,10,11-octahydrooxazolo[2,3- j][1,6]naphthyridine-9-carboxylate | 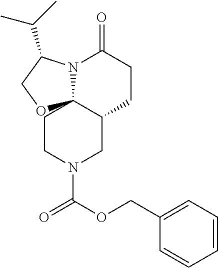 [SMILES: CC(C)[C@H]1CO[C@]23CCN(C(=O)OCc4ccccc4)C[C@H]2CCC(=O)N13] |
| 48 | [4-(trifluoromethyl)phenyl]methyl (3S,7aR,11aR)-3-isopropyl-5-oxo- 2,3,6,7,7a,8,10,11-octahydrooxazolo[2,3- j][1,6]naphthyridine-9-carboxylate | 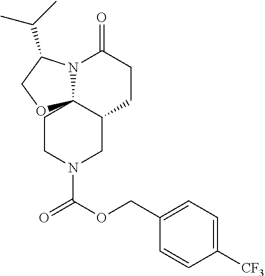 [SMILES: CC(C)[C@H]1CO[C@]23CCN(C(=O)OCc4ccc(C(F)(F)F)cc4)C[C@H]2CCC(=O)N13] |
| 49 | 2-[4-(trifluoromethyl)phenyl]ethyl (3S,7aR,11aR)-3-isopropyl-5-oxo- 2,3,6,7,7a,8,10,11-octahydrooxazolo[2,3- j][1,6]naphthyridine-9-carboxylate | 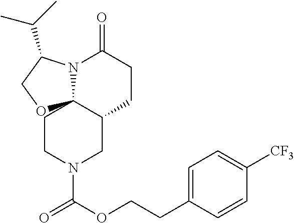 [SMILES: CC(C)[C@H]1CO[C@]23CCN(C(=O)OCCc4ccc(C(F)(F)F)cc4)C[C@H]2CCC(=O)N13] |
| 50 | 3-[4-(trifluoromethyl)phenyl]propyl (3S,7aR,11aR)-3-isopropyl-5-oxo- 2,3,6,7,7a,8,10,11-octahydrooxazolo[2,3- j][1,6]naphthyridine-9-carboxylate | 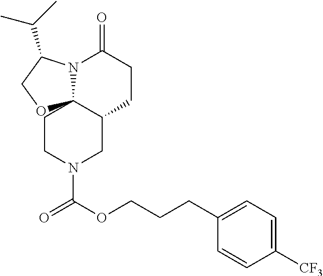 [SMILES: CC(C)[C@H]1CO[C@]23CCN(C(=O)OCCCc4ccc(C(F)(F)F)cc4)C[C@H]2CCC(=O)N13] |
| 51 | (3S,7aR,11aR)-3-isopropyl-9-[4- (trifluoromethyl)phenyl]sulfonyl- 2,3,6,7,7a,8,10,11-octahydrooxazolo[2,3- j][1,6]naphthyridin-5-one | 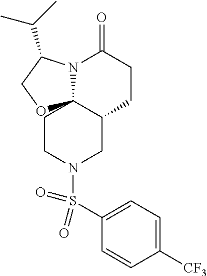 [SMILES: CC(C)[C@H]1CO[C@]23CCN(S(=O)(=O)c4ccc(C(F)(F)F)cc4)C[C@H]2CCC(=O)N13] |
| 52 | (3S,7aR,11aR)-3-isopropyl-9-[[4- (trifluoromethyl)phenyl]methylsulfonyl]- 2,3,6,7,7a,8,10,11-octahydrooxazolo[2,3- j][1,6]naphthyridin-5-one | 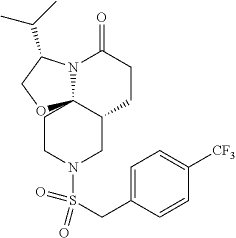 [SMILES: CC(C)[C@H]1CO[C@]23CCN(S(=O)(=O)Cc4ccc(C(F)(F)F)cc4)C[C@H]2CCC(=O)N13] |
| 53 | (3S,7aR,11aR)-3-isopropyl-9-[2-[4- (trifluoromethyl)phenyl]ethylsulfonyl]- 2,3,6,7,7a,8,10,11-octahydrooxazolo[2,3- j][1,6]naphthyridin-5-one | 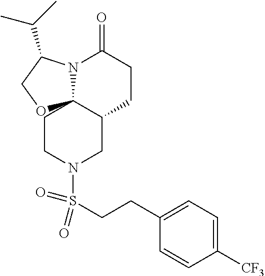 [SMILES: CC(C)[C@H]1CO[C@]23CCN(S(=O)(=O)CCc4ccc(C(F)(F)F)cc4)C[C@H]2CCC(=O)N13] |
| 54 | (3S,7aR,11aR)-3-isopropyl-9-[3-[4- (trifluoromethyl)phenyl]propylsulfonyl]- 2,3,6,7,7a,8,10,11-octahydrooxazolo[2,3- j][1,6]naphthyridin-5-one | 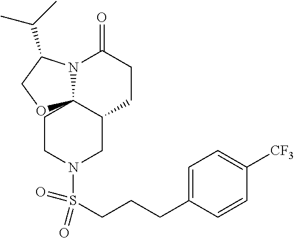 [SMILES: CC(C)[C@H]1CO[C@]23CCN(S(=O)(=O)CCCc4ccc(C(F)(F)F)cc4)C[C@H]2CCC(=O)N13] |
| 55 | (3S,7aR,11aR)-9-benzyl-3-[(1R)-1- methylpropyl]-2,3,6,7,7a,8,10,11- octahydrooxazolo[2,3-j][1,6]naphthyridin- 5-one | 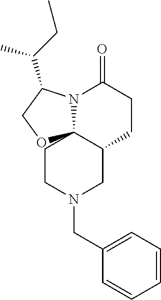 [SMILES: CC[C@@H](C)[C@H]1CO[C@]23CCN(Cc4ccccc4)C[C@H]2CCC(=O)N13] |
| 56 | (3S,7aR,11aR)-9-benzyl-3-tert-butyl- 2,3,6,7,7a,8,10,11-octahydrooxazolo[2,3- j][1,6]naphthyridin-5-one | 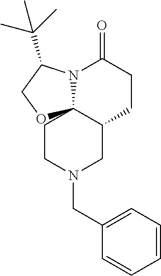 [SMILES: CC(C)(C)[C@H]1CO[C@]23CCN(Cc4ccccc4)C[C@H]2CCC(=O)N13] |
| 57 | (3S,7aR,11aR)-9-benzyl-3-cyclopropyl- 2,3,6,7,7a,8,10,11-octahydrooxazolo[2,3- j][1,6]naphthyridin-5-one | 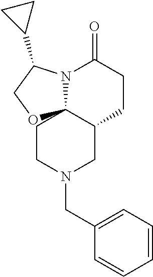 [SMILES: O=C1CC[C@@H]2CN(Cc3ccccc3)CC[C@@]23OC[C@H](C2CC2)N13] |
| 58 | (3S,7aR,11aR)-9-[[4- (trifluoromethyl)phenyl]methyl]-3-vinyl- 2,3,6,7,7a,8,10,11-octahydrooxazolo[2,3- j][1,6]naphthyridin-5-one | 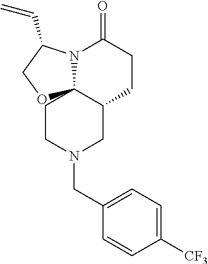 [SMILES: C=C[C@H]1CO[C@]23CCN(Cc4ccc(C(F)(F)F)cc4)C[C@H]2CCC(=O)N13] |
| 59 | (3R,7aR,11aR)-3-(trifluoromethyl)-9-[[4- (trifluoromethyl)phenyl]methyl]- 2,3,6,7,7a,8,10,11-octahydrooxazolo[2,3- j][1,6]naphthyridin-5-one | 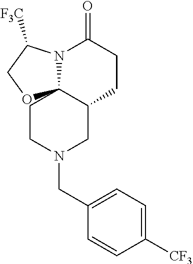 [SMILES: O=C1CC[C@@H]2CN(Cc3ccc(C(F)(F)F)cc3)CC[C@@]23OC[C@H](C(F)(F)F)N13] |
| 60 | (1R,6R,11S,16S)-4-benzyl-17-oxa-4,10- diazatetracyclo[8.7.0.01,6.011,16]hepta- decan-9-one | 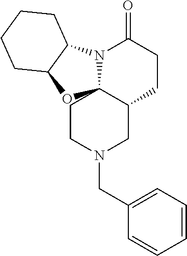 [SMILES: O=C1CC[C@@H]2CN(Cc3ccccc3)CC[C@@]23O[C@H]2CCCC[C@@H]2N13] |
| 61 | (4R,8aR,12aR)-4-isopropyl-10-[[4- (trifluoromethyl)phenyl]methyl]- 3,4,7,8,8a,9,11,12-octahydro-2H- [1,3]oxazino[2,3-j][1,6]naphthyridin-6-one | 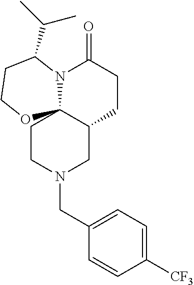 [SMILES: CC(C)[C@H]1CCO[C@]23CCN(Cc4ccc(C(F)(F)F)cc4)C[C@H]2CCC(=O)N13] |
| 62 | (3S,7aR,11aR)-9-benzyl-3-isopropyl- 2,3,6,7,7a,8,10,11-octahydrooxazolo[2,3- j][1,6]naphthyridin-5-one | 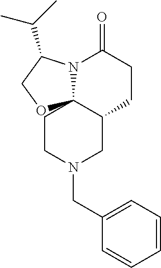 [SMILES: CC(C)[C@H]1CO[C@]23CCN(Cc4ccccc4)C[C@H]2CCC(=O)N13] |
| 63 | (1R,4S,8R)-10-benzyl-4-isopropyl-2-oxa- 5,10-diazatricyclo[6.4.0.01,5]dodecan-6- one | 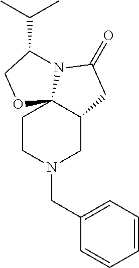 [SMILES: CC(C)[C@H]1CO[C@]23CCN(Cc4ccccc4)C[C@H]2CC(=O)N13] |
| 64 | (1R,4S,8R)-4-isopropyl-10-[[4- (trifluoromethyl)phenyl]methyl]-2-oxa- 5,10-diazatricyclo[6.4.0.01,5]dodecan-6- one | 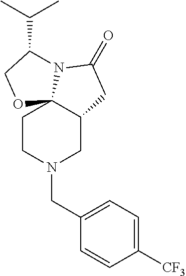 [SMILES: CC(C)[C@H]1CO[C@]23CCN(Cc4ccc(C(F)(F)F)cc4)C[C@H]2CC(=O)N13] |
| 65 | (3S,7aR,11aR)-9-benzyl-3-isopropyl- 2,3,6,7,7a,8,10,11-octahydrooxazolo[2,3- j][1,6]naphthyridine-5-thione | 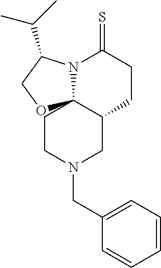 [SMILES: CC(C)[C@H]1CO[C@]23CCN(Cc4ccccc4)C[C@H]2CCC(=S)N13] |
| 66 | (1R,4S,9R)-4-isopropyl-11-[[4- (trifluoromethyl)phenyl]methyl]-2,8-dioxa- 5,11-diazatricyclo[7.4.0.01,5]tridecan-6- one | 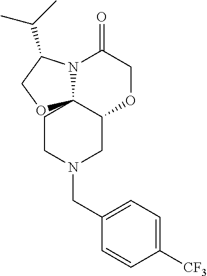 [SMILES: CC(C)[C@H]1CO[C@]23CCN(Cc4ccc(C(F)(F)F)cc4)C[C@H]2OCC(=O)N13] |
| 67 | (1R,5R,9R)-5-isopropyl-11-[[4- (trifluoromethyl)phenyl]methyl]-2-oxa- 6,11-diazatricyclo[7.4.0.01,6]tridecan-7- one | 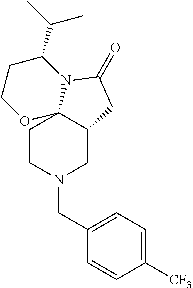 [SMILES: CC(C)[C@H]1CCO[C@]23CCN(Cc4ccc(C(F)(F)F)cc4)C[C@H]2CC(=O)N13] |
| 68 | (3S,7aR,11aR)-3-isopropyl-9-[[4- (trifluoromethyl)phenyl]methyl]- 3,6,7,7a,8,11-hexahydro-2H-oxazolo[2,3- j][1,6]naphthyridine-5,10-dione | 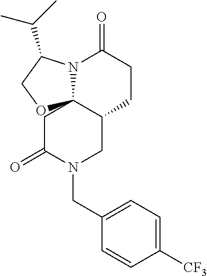 [SMILES: CC(C)[C@H]1CO[C@]23CC(=O)N(Cc4ccc(C(F)(F)F)cc4)C[C@H]2CCC(=O)N13] |
| 69 | (3S,7aS,11aR)-3-isopropyl-9-[[4- (trifluoromethyl)phenyl]methyl]- 3,6,7,7a,10,11-hexahydro-2H- oxazolo[2,3-j][1,6]naphthyridine-5,8- dione | 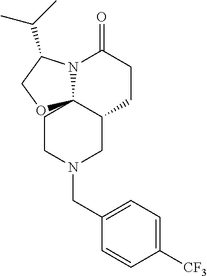 [SMILES: CC(C)[C@H]1CO[C@]23CCN(Cc4ccc(C(F)(F)F)cc4)C[C@H]2CCC(=O)N13] |
| 70 | (3S,7aR,9S,11aR)-9-[bis[[4- (trifluoromethyl)phenyl]methyl]amino]-3- isopropyl-3,6,7,7a,8,9,10,11-octahydro- 2H-oxazolo[2,3-j]quinolin-5-one | 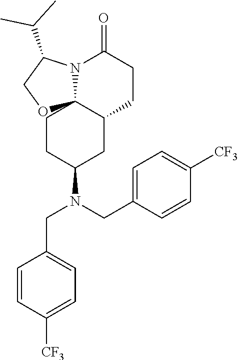 [SMILES: CC(C)[C@H]1CO[C@]23CC[C@H](N(Cc4ccc(C(F)(F)F)cc4)Cc4ccc(C(F)(F)F)cc4)C[C@H]2CCC(=O)N13] |
| 71 | (3S,7aR,9R,11aR)-9-[bis[[4- (trifluoromethyl)phenyl]methyl]amino]-3- isopropyl-3,6,7,7a,8,9,10,11-octahydro- 2H-oxazolo[2,3-j]quinolin-5-one | 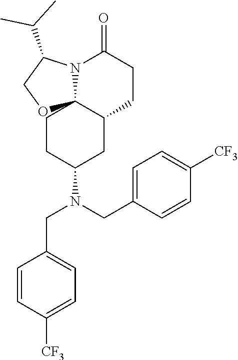 [SMILES: CC(C)[C@H]1CO[C@]23CC[C@@H](N(Cc4ccc(C(F)(F)F)cc4)Cc4ccc(C(F)(F)F)cc4)C[C@H]2CCC(=O)N13] |
| 72 | (3S,7aR,9S,11aR)-3-isopropyl-9-[[4- (trifluoromethyl)phenyl]methylamino]- 3,6,7,7a,8,9,10,11-octahydro-2H- oxazolo[2,3-j]quinolin-5-one | 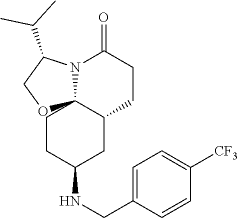 [SMILES: CC(C)[C@H]1CO[C@]23CC[C@H](NCc4ccc(C(F)(F)F)cc4)C[C@H]2CCC(=O)N13] |
| 73 | (3S,7aR,9R,11aR)-3-isopropyl-9-[[4- (trifluoromethyl)phenyl]methylamino]- 3,6,7,7a,8,9,10,11-octahydro-2H- oxazolo[2,3-j]quinolin-5-one | 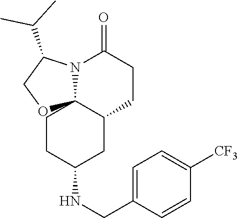 [SMILES: CC(C)[C@H]1CO[C@]23CC[C@@H](NCc4ccc(C(F)(F)F)cc4)C[C@H]2CCC(=O)N13] |
| 74 | (3S,7aR,9S,11aR)-3-isopropyl-9-[4- (trifluoromethyl)anilino]- 3,6,7,7a,8,9,10,11-octahydro-2H- oxazolo[2,3-j]quinolin-5-one | 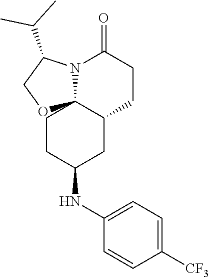 [SMILES: CC(C)[C@H]1CO[C@]23CC[C@H](Nc4ccc(C(F)(F)F)cc4)C[C@H]2CCC(=O)N13] |
| 75 | (3S,7aR,9S,11aR)-3-isopropyl-9-[4- (trifluoromethyl)anilino]- 3,6,7,7a,8,9,10,11-octahydro-2H- oxazolo[2,3-j]quinolin-5-one | 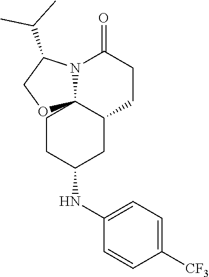 [SMILES: CC(C)[C@H]1CO[C@]23CC[C@@H](Nc4ccc(C(F)(F)F)cc4)C[C@H]2CCC(=O)N13] |
| 76 | (4S,9R)-4-isopropyl-11-[[4- (trifluoromethyl)phenyl]methyl]-2-oxa- 5,11-diazatricyclo[7.5.0.01,5]tetradecan- 6-one | 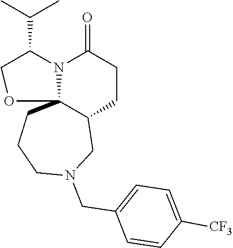 [SMILES: CC(C)[C@H]1CO[C@]23CCCN(Cc4ccc(C(F)(F)F)cc4)C[C@H]2CCC(=O)N13] |
| 77 | (1R,4S,9S)-4-isopropyl-10-[[4- (trifluoromethyl)phenyl]methyl]-2-oxa- 5,10-diazatricyclo[7.3.0.01,5]dodecan-6- one | 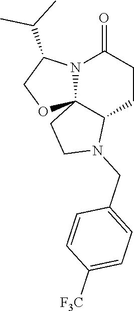 [SMILES: CC(C)[C@H]1CO[C@]23CCN(Cc4ccc(C(F)(F)F)cc4)[C@H]2CCC(=O)N13] |
| 78 | (3S,6R,7aR,11aR)-3-isopropyl-6-methyl- 9-[[4-(trifluoromethyl)phenyl]methyl]- 2,3,6,7,7a,8,10,11-octahydrooxazolo[2,3- j][1,6]naphthyridin-5-one | 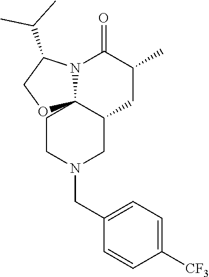 [SMILES: CC(C)[C@H]1CO[C@]23CCN(Cc4ccc(C(F)(F)F)cc4)C[C@H]2C[C@@H](C)C(=O)N13] |
| 79 | (3S,6R,7aR,11aR)-6-ethyl-3-isopropyl-9- [[4-(trifluoromethyl)phenyl]methyl]- 2,3,6,7,7a,8,10,11-octahydrooxazolo[2,3- j][1,6]naphthyridin-5-one | 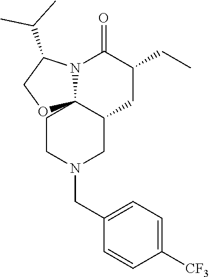 [SMILES: CC[C@@H]1C[C@@H]2CN(Cc3ccc(C(F)(F)F)cc3)CC[C@@]23OC[C@H](C(C)C)N3C1=O] |
| 80 | (3S,6R,7aR,11aR)-6-allyl-3-isopropyl-9- [[4-(trifluoromethyl)phenyl]methyl]- 2,3,6,7,7a,8,10,11-octahydrooxazolo[2,3- j][1,6]naphthyridin-5-one | 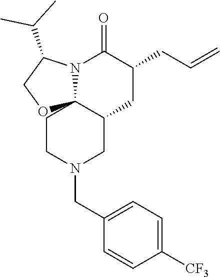 [SMILES: C=CC[C@@H]1C[C@@H]2CN(Cc3ccc(C(F)(F)F)cc3)CC[C@@]23OC[C@H](C(C)C)N3C1=O] |
| 81 | (3S,6R,7aR,11aR)-6-isobutyl-3- isopropyl-9-[[4- (trifluoromethyl)phenyl]methyl]- 2,3,6,7,7a,8,10,11-octahydrooxazolo[2,3- j][1,6]naphthyridin-5-one | 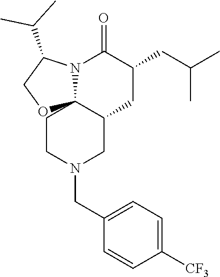 [SMILES: CC(C)C[C@@H]1C[C@@H]2CN(Cc3ccc(C(F)(F)F)cc3)CC[C@@]23OC[C@H](C(C)C)N3C1=O] |
| 82 | (3S,6S,7aR,11aR)-3-isopropyl-6-methyl- 9-[[4-(trifluoromethyl)phenyl]methyl]- 2,3,6,7,7a,8,10,11-octahydrooxazolo[2,3- j][1,6]naphthyridin-5-one | 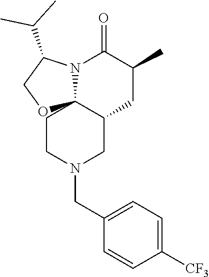 [SMILES: CC(C)[C@H]1CO[C@]23CCN(Cc4ccc(C(F)(F)F)cc4)C[C@H]2C[C@H](C)C(=O)N13] |
| 83 | (3S,6S,7aR,11aR)-6-ethyl-3-isopropyl-9- [[4-(trifluoromethyl)phenyl]methyl]- 2,3,6,7,7a,8,10,11-octahydrooxazolo[2,3- j][1,6]naphthyridin-5-one | 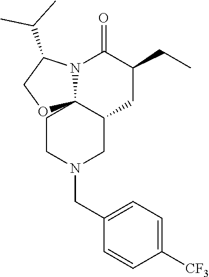 [SMILES: CC[C@H]1C[C@@H]2CN(Cc3ccc(C(F)(F)F)cc3)CC[C@@]23OC[C@H](C(C)C)N3C1=O] |
| 84 | (3S,6S,7aR,11aR)-6-allyl-3-isopropyl-9- [[4-(trifluoromethyl)phenyl]methyl]- 2,3,6,7,7a,8,10,11-octahydrooxazolo[2,3- j][1,6]naphthyridin-5-one | 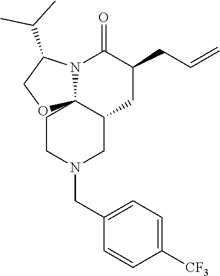 [SMILES: C=CC[C@H]1C[C@@H]2CN(Cc3ccc(C(F)(F)F)cc3)CC[C@@]23OC[C@H](C(C)C)N3C1=O] |
| 85 | (3S,7aR,11aR)-6-fluoro-3-isopropyl-9-[[4- (trifluoromethyl)phenyl]methyl]- 2,3,6,7,7a,8,10,11-octahydrooxazolo[2,3- j][1,6]naphthyridin-5-one | 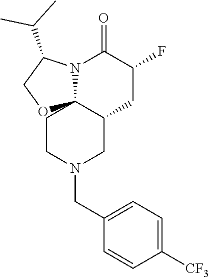 [SMILES: CC(C)[C@H]1CO[C@]23CCN(Cc4ccc(C(F)(F)F)cc4)C[C@H]2C[C@@H](F)C(=O)N13] |
| 86 | (3S,7aR,11aR)-6,6-difluoro-3-isopropyl- 9-[[4-(trifluoromethyl)phenyl]methyl]- 3,7,7a,8,10,11-hexahydro-2H- oxazolo[2,3-j][1,6]naphthyridin-5-one | 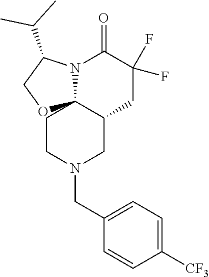 [SMILES: CC(C)[C@H]1CO[C@]23CCN(Cc4ccc(C(F)(F)F)cc4)C[C@H]2CC(F)(F)C(=O)N13] |

### 3-(1-benzyl-4-oxo-3-piperidyl) propanoic acid (intermediate 1)

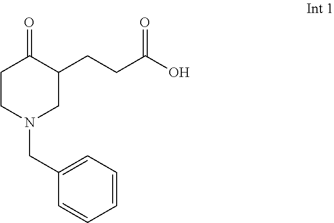 [SMILES: O=C(O)CCC1CN(Cc2ccccc2)CCC1=O]

Intermediate 1 was prepared according to the procedure described in Malaquin, S.; Jida, M.; Courtin, J.; Laconde, G.; Willand, N.; Deprez, B.; Deprez-Poulain, R. Tetrahedron Letters, 2013, 54, 562-567.

### Synthesis of methyl 3-(1-benzyl-4-oxo-3-piperidyl) propanoate (intermediate 2)

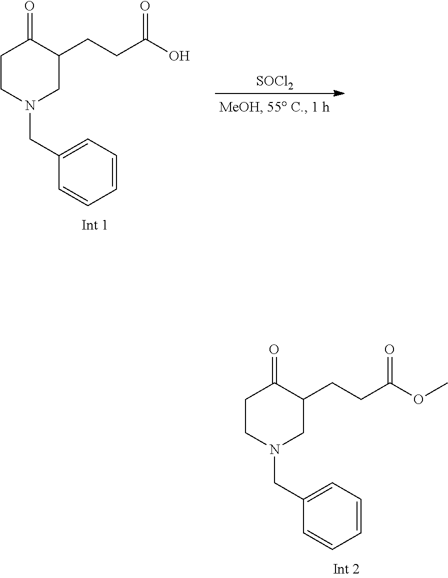 [SMILES: O=C(O)CCC1CN(Cc2ccccc2)CCC1=O ; "SOCl2" ; COC(=O)CCC1CN(Cc2ccccc2)CCC1=O]

To a solution of Intermediate 1 (19.3 g, 73.9 mmol) in methanol (200 mL), was added SOCl₂ (5.9 mL, 81.3 mmol) dropwise at room temperature. The mixture was then stirred at 55° C. for 1 h.

The solvent was removed under vacuum and the mixture was dissolved in 0.1 N HCl (100 mL), and stirred at room temperature for 1 h. A saturated aqueous solution of Na₂CO₃ was added until pH 10. The solution was extracted with ethyl acetate. The organic layer was dried over MgSO₄, and the solvent was removed under reduced pressure to give the crude product, which was purified by silica gel chromatography (cyclohexane/ethyl acetate: 70/30 to 0/100) to afford Intermediate 2 (6.73 g, 33%), as a colorless oil. [ES+MS] m/z 276 (MH⁺).

### Synthesis of methyl 2-(1-benzyl-4-oxo-3-piperidyl)acetate hydrochloride (intermediate 3)

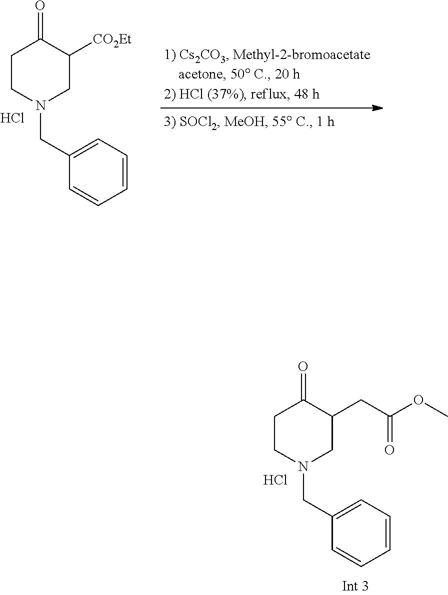 [SMILES: Cl ; CCOC(=O)C1CN(Cc2ccccc2)CCC1=O ; Cl ; COC(=O)CC1CN(Cc2ccccc2)CCC1=O]

Ethyl 1-benzyl-4-oxo-piperidine-3-carboxylate; hydrochloride (0.500 g, 1.68 mmol, 1 eq) and Cs₂CO₃ (1.45 g, 4.45 mmol, 2.7 eq) were dissolved in 3 mL of dry acetone. The reaction mixture was stirred and heated at 50° C. Methyl 2-bromoacetate (185 μL, 2.01 mmol, 1.2 eq) was dissolved in 2 mL of dry acetone before being added dropwise to the hot reaction mixture which was then stirred at reflux during 20 h. The reaction mixture was filtered and the solvent evaporated. A solution of 13 mL of HCl 37% dissolved in water (13 mL) was added.

The reaction mixture was reflux heated for 48 h. Solvent was removed under vacuum. The pH was then brought to 7 using 3M NaOH and sat. NaHCO₃. Solvent was removed under vacuum. The mixture was taken up in ethanol and filtered. Ethanol was then removed under vacuum to afford a dark brown oil. The brown oil obtained was dissolved in MeOH (2 mL), then SOCl₂ (26.6 μL, 0.365 mmol) was added dropwise at room temperature. The mixture was then stirred at 55° C. for 1 h. Solvent was removed under vacuum and the mixture was dissolved in 0.1N HCl (5 mL) and stirred at room temperature for 3 h. The solvent was removed under reduced pressure to afford intermediate 3. [ES+MS] m/z 262 (MH⁺).

### General Protocol 1: Synthesis of N-Boc Ketoester Via Stork Enamine Alkylation:

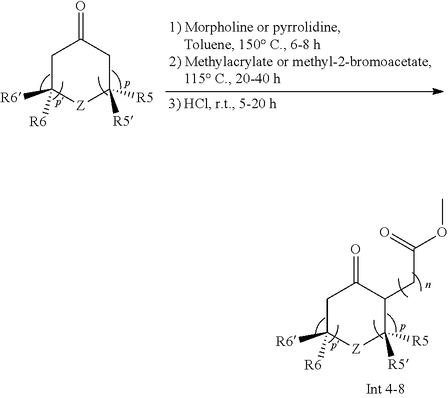

The appropriate ketone (1 eq) was dissolved in dry toluene (0.4N), then morpholine (1.5 eq) or pyrrolidine (5 eq) was added. The flask was equipped with a Dean-Stark apparatus and a condenser. The solution was heated at 150° C. for 6-8 h. The mixture was cooled down to room temperature, then methyl acrylate (2.5-5 eq) or methyl-2-bromoacetate (2.5 eq) was added. The solution was refluxed at 115° C. during 20-40 h. The mixture was evaporated until dryness. The brown oil obtained was dissolved in HCl (10 eq), and stirred at room temperature for 5-20 h. The solution was extracted with ethyl acetate. The layers were separated. The organic layer was dried over MgSO₄, filtered and concentrated under reduced pressure to give the crude product, which was purified to afford the appropriate ketoester.

| Intermediate | Structure | Physical data |
|---|---|---|
| 4 | 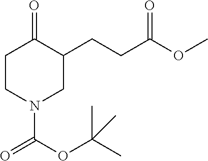 [SMILES: COC(=O)CCC1CN(C(=O)OC(C)(C)C)CCC1=O] | Tert-butyl 3-(3-methoxy-3-oxo-propyl)-4-oxo- piperidine-1-carboxylate Intermediate 4 was obtained from N-Boc piperidin-4-one, morpholine and methyl acrylate using general protocol 1; Purification by reverse phase chromatography combiflash using H₂O/MeOH (90/10 to 0/100); Yield: 72%; white solid; ¹H NMR (300 MHz, CDCl₃): δ 4.20-4.00 (m, 2H), 3.66 (s, 3H), 3.42-3.26 (m, 1H), 3.06-2.92 (m, 1H), 2.51-2.33 (m, 5H), 2.11-1.98 (m, 1H), 1.65-1.52 (m, 1H), 1.48 (s, 9H) ppm. ¹³C (75 MHz, CDCl₃): δ 209.1, 173.5, 154.6, 80.7, 51.8, 49.4, 48.4, 43.9, 40.9, 31.5, 28.5, 22.5 ppm. [ES+ MS] m/z 286 (MH⁺). |
| 5 | 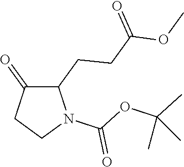 [SMILES: COC(=O)CCC1C(=O)CCN1C(=O)OC(C)(C)C] | Tert-butyl 3-(3-methoxy-3-oxo-propyl)-4-oxo- pyrrolidine-1-carboxylate Intermediate 5 was obtained from N-Boc pyrrolidin-3-one, morpholine and methyl acrylate using general protocol 1; Purification by reverse phase chromatography combiflash using H₂O/MeOH (90/10 to 0/100); Yield: 43%; colorless oil; ¹H NMR (300 MHz, CDCl₃): δ 3.96-3.98 (m, 1H), 3.73-3.65 (m, 1H), 3.63 (s, 3H), 3.57-3.44 (m, 1H), 2.67-2.23 (m, 4H), 2.20-2.01 (m, 2H), 1.47 (s, 9H) ppm. [ES+ MS] m/z 272 (MH⁺). |
| 6 | 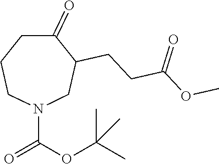 | Tert-butyl 3-(3-methoxy-3-oxo-propyl)-4-oxo-azepane- 1-carboxylate Intermediate 6 was obtained from N-Boc azapan-4-one, pyrrolidine and methyl acrylate using general protocol 1; Purification by reverse phase chromatography combiflash using H₂O/MeOH (90/10 to 0/100); Yield: 34%; colorless oil; ¹H NMR (300 MHz, CDCl₃): δ 4.02-3.79 (m, 2H), 3.64 (s, 3H), 3.11-2.93 (m, 2H), 2.91-2.73 (m, 1H), 2.72-2.55 (m, 2H), 2.52-2.42 (m, 1H), 2.40-2.24 (m, 2H), 1.82-1.61 (m, 3H), 1.42 (s, 9H) ppm. ¹³C (75 MHz, CDCl₃): δ 212.9, 173.4, 154.5, 80.3, 77.3, 51.5, 49.3, 42.8, 40.8, 31.6, 28.4, 26.6, 25.8, 25.6, 24.5, 24.4, 23.5 ppm. [ES+ MS] m/z 300 (MH⁺). |
| 7 | 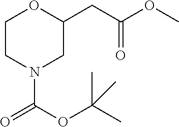 | Tert-butyl 3-(2-methoxy-2-oxo-ethyl)-4-oxo-piperidine- 1-carboxylate Intermediate 7 was obtained from N-Boc piperidin-4-one, morpholine and methyl-2-bromoacetate using general protocol 1; Purification by reverse phase chromatography combiflash using H₂O/MeOH (90/10 to 0/100);Yield: 9%; colorless oil; ¹H NMR (300 MHz, CDCl₃): δ 4.40-4.24 (m, 2H), 3.70 (s, 3H), 3.25-3.211 (m, 1H), 3.02-2.84 (m, 2H), 2.77-2.64 (m, 1H), 2.62-3.37 (m, 2H), 2.34-2.22 (m, 1H), 1.50 (s, 9H) ppm; ¹³C (75 MHz, CDCl₃): δ 207.5, 172.1, 154.5, 80.8, 52.0, 48.1, 46.4, 43.8, 41.0, 31.3, 28.5 ppm. [ES+ MS] m/z 272 (MH⁺). |
| 8 | 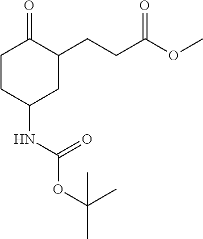 [SMILES: COC(=O)CCC1CC(NC(=O)OC(C)(C)C)CCC1=O] | Methyl 3-[5-(tert-butoxycarbonylamino)-2-oxo- cyclohexyl]propanoate Intermediate 8 was obtained from N-Boc cyclohexan-4-one, pyrrolidine and methyl acrylate using general protocol 1; Purification by flash chromatography column over silica gel (cyclohexane/ethyl acetate 100/0 to 40/60); Yield = 85%; Pale yellow oil; ¹H NMR (300 MHz, CDCl₃): δ 4.85 (s, 0.5H), 4.49 (d, J = 7.6 Hz, 0.5H), 4.00 (br s, 1H), 3.67 (s, 3H), 2.61- 2.25 (m, 7H), 2.17-1.98 (m, 4H), 1.48-1.43 (m, 9H) ppm. [ES+ MS] m/z 300 (MH⁺). |

### General Protocol 2: Synthesis of Lactams by Meyers Lactamization

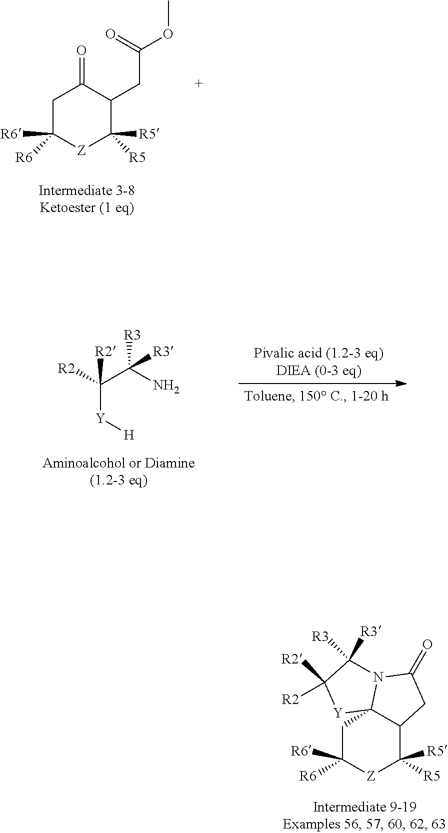

A solution of pivalic acid (1.2-3 eq) in toluene (0.2N) was added to the appropriate ketoester (1 eq). The appropriate aminoalcohol or diamine (1.2-3 eq) was added (when the amine is used as a chlorohydrate, DIEA (1.2-3 eq) was added). The mixture was refluxed at 150° C. (thermic or microwave irradiations) for 1-20 h. The solution was dissolved in H₂O, extracted with ethyl acetate or dichloromethane. The layers were separated. The organic layer was dried over MgSO₄, filtered and concentrated under vacuum to give the crude product, which was purified, to afford the corresponding desired product.

| Example | Structure | Physical data |
|---|---|---|
| 56 | 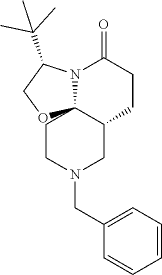 [SMILES: CC(C)(C)[C@H]1CO[C@]23CCN(Cc4ccccc4)C[C@H]2CCC(=O)N13] | (3S,7aR,11aR)-9-benzyl-3-tert-butyl-2,3,6,7,7a,8,10,11- octahydrooxazolo[2,3-j][1,6]naphthyridin-5-one Example 56 was obtained from intermediate 2 and (2S)-2- amino-3,3-dimethyl-butan-1-ol using general protocol 2; Purification by preparative HPLC using H₂O + 0.1% HCOOH/MeCN + 0.1% HCOOH (100/0 to 0/100); Yield: 40%; yellow oil; ¹H NMR (300 MHz, CD₂Cl₂): δ 7.36-7.19 (m, 5H), 4.11 (t, J = 6.9 Hz, 1H), 3.85 (d, J = 6.5 Hz, 2H), 3.53 (d, J = 13.3 Hz, 1H), 3.41 (d, J = 13.3 Hz, 1H), 2.77- 2.69 (m, 1H), 2.61-2.57 (m, 1H), 2.58-2.49 (m, 1H), 2.45- 2.27 (m, 3H), 2.20 (dt, J = 12.0 Hz, J = 3.4 Hz, 1H), 1.98 (dt, J = 13.3 Hz, J = 4.68 Hz, 1H), 1.90-1.82 (m, 1H), 1.73-1.52 (m, 2H), 0.92 (m, 9H) ppm. ¹³C (75 MHz, CD₂Cl₂): δ 172.7, 139.5, 129.1, 128.5, 127.2, 94.3, 64.8, 64.5, 62.7, 54.9, 50.9, 40.2, 34.8, 32.7, 31.0, 27.8, 20.9 ppm. HRMS (ESI) [M + H]⁺ calculated for [C₂₁H₃₁N₂O₂]⁺: 343.2386, found: 343.2394 |
| 57 | 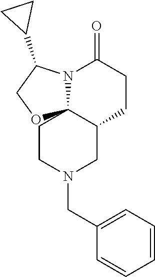 [SMILES: O=C1CC[C@@H]2CN(Cc3ccccc3)CC[C@@]23OC[C@H](C2CC2)N13] | (3S,7aR,11aR)-9-benzyl-3-cyclopropyl- 2,3,6,7,7a,8,10,11-octahydrooxazolo[2,3- j][1,6]naphthyridin-5-one Example 57 was obtained from intermediate 2 and (2S)-2- amino-2-cyclopropyl-ethanol; hydrochloride using general protocol 2; Purification by flash column chromatography column on silica gel (cyclohexane/ethyl acetate: 100/0 to 0/100), then by preparative HPLC using H₂O + 0.1% HCOOH/MeCN + 0.1% HCOOH (100/0 to 0/100); Yield: 27%; colorless oil. ¹H NMR (300 MHz, CDCl₃): δ 7.36-7.23 (m, 5H), 4.15 (dd, J = 8.5, 8.4 Hz, 1H), 3.86 (dd, J = 8.9, 6.8 Hz, 1H), 3.75-3.62 (m, 1H), 3.58 (d of AB system, J = 13.4 Hz, 1H), 3.44 (d, J = 13.4 Hz, 1H), 2.82-2.18 (m, 6H), 2.11-1.89 (m, 2H), 1.74-1.54 (m, 3H), 0.93-0.72 (m, 2H), 0.66-0.41 (m, 2H), 0.30-0.17 (m, 1H) ppm. ¹³C (75 MHz, CDCl₃): δ 169.4, 138.9, 128.8, 128.4, 127.1, 92.6, 68.0, 62.6, 59.9, 54.9, 50.8, 40.4, 31.8, 31.1, 22.5, 15.8, 4.6, 2.7 ppm. [ES+ MS] m/z 327 (MH⁺). |
| 60 | 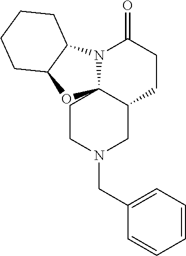 [SMILES: O=C1CC[C@@H]2CN(Cc3ccccc3)CC[C@@]23O[C@H]2CCCC[C@@H]2N13] | (1R,6R,11S,16S)-4-benzyl-17-oxa-4,10- diazatetracyclo[8.7.0.01,6.011,16]heptadecan-9-one Example 60 was obtained from intermediate 2 and (1S,2S)- 2-aminocyclohexanol; hydrochloride using general protocol 2; Purification by flash column chromatography column on silica gel (Cyclohexane/ethyl acetate: 1/0 to 0/1), then by preparative HPLC using H₂O + 0.1%/ HCOOH/MeCN + 0.1% HCOOH (100/0 to 0/100); Yield: 30%; yellow oil; ¹H NMR (300 MHz, CD₂Cl₂): δ 7.34-7.19 (m, 5H); 3.59-3.40 (m, 3H); 2.98 (td, 1H, J = 10.3 Hz, 2.9 Hz, 1H), 2.75-2.06 (m, 9H); 1.91-1.20 (m, 13H) ppm. ¹³C NMR (75 MHz, CD₂Cl₂) δ: 173.4, 129.1, 128.5, 127.3, 94.2, 81.7, 78.8, 64.2, 62.7, 62.2, 55.1, 54.5, 50.7, 40.8, 31.6, 31.1, 30.2, 29.9, 24.7, 24.5, 21.7 ppm. [ES+ MS] m/z 341 (MH⁺). |
| 62 | 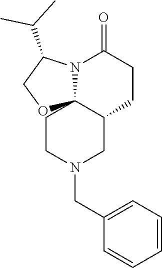 [SMILES: CC(C)[C@H]1CO[C@]23CCN(Cc4ccccc4)C[C@H]2CCC(=O)N13] | (3S,7aR,11aR)-9-benzyl-3-isopropyl-2,3,6,7,7a,8,10,11- octahydrooxazolo[2,3-j][1,6]naphthyridin-5-one Example 62 was obtained from intermediate 2 and L-valinol using general protocol 2; Purification by flash column chromatography column on silica gel Cyclohexane/ethyl acetate: 100/0 to 0/100); Yield: 75%; White powder; ¹H NMR (300 MHz, CDCl₃): δ 7.33-7.20 (m, 5H), 4.13-4.03 (m, 1H), 3.98 (dd, J = 8.6 Hz, 7.6 Hz, 1H), 3.77 (dd, J = 8.6 Hz, 6.2 Hz, 1H), 3.54 (d, J = 13.3 Hz, 1H), 3.41 (d, J = 13.1 Hz, 1H), 2.77-2.64 (m, 2H), 2.62-2.51 (m, 1H), 2.49-2.29 (m, 3H), 2.25-2.16 (m, 1H), 2.09-1.89 (m, 2H), 1.83-1.73 (m, 1H), 1.72-1.55 (m, 2H), 0.94 (d, J = 6.8 Hz, 3H), 0.90 (d, J = 6.8 Hz, 3H) ppm. ¹³C NMR (75 MHz, CDCl₃)δ 170.2, 138.9, 128.7, 128.3, 127.0, 92.7, 66.2, 62.5, 61.2, 54.8, 50.7, 40.4, 32.4, 32.4, 30.8, 21.9, 19.9, 18.7 ppm. HRMS (ESI,m/z): [M + H]⁺ calculated for C₂₀H₂₉N₂O₂, 329.2229; found 329.2227. |
| 63 | 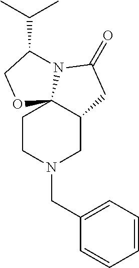 [SMILES: CC(C)[C@H]1CO[C@]23CCN(Cc4ccccc4)C[C@H]2CC(=O)N13] | (1R,4S,8R)-10-benzyl-4-isopropyl-2-oxa-5,10- diazatricyclo[6.4.0.01,5]dodecan-6-one Example 63 was obtained from intermediate 3 and L-valinol using general protocol 2; Purification by preparative HPLC using H₂O + 0.1% HCOOH/MeCN + 0.1% HCOOH (100/0 to 0/100); Yield: 17%; ¹H NMR (300 MHz, CD₂Cl₂): δ 7.28 (m, 5H), 4.06 (m, 1H), 3.93 (m, 1H), 3.54 (m, 3H), 2.87 (m, 1H), 2.65 (m, 2H), 2.45 (m, 1H), 2.31 (m, 2H), 2.19 (m, 1H), 2.07 (m, 1H), 1.93 (qd, J = 6.7 Hz, 1H), 1.64 (m, 1H), 0.97 (d, J = 6.7 Hz, 3H), 0.89 (d, J = 6.7 Hz, 3H) ppm. ¹³C NMR (75 MHz, CD₂Cl₂): δ 176.9, 139.3, 129.0, 128.6, 127.3, 97.7, 71.3, 62.7, 61.9, 53.5, 50.3, 43.0, 38.7, 35.1, 32.8, 20.4, 19.2 ppm. HRMS (ESI,m/z): [M + H]⁺ calculated for C₁₉H₂₆N₂O₂, 315.2073; found 315.2080. |

| Intermediate | Structure | Physical data |
|---|---|---|
| 9 | 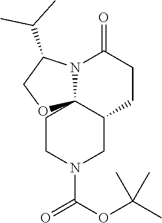 [SMILES: CC(C)[C@H]1CO[C@]23CCN(C(=O)OC(C)(C)C)C[C@H]2CCC(=O)N13] | Tert-butyl (3S,7aR,11aR)-3-isopropyl-5-oxo- 2,3,6,7,7a,8,10,11-octahydrooxazolo[2,3- j][1,6]naphthyridine-9-carboxylate Intermediate 9 was obtained from intermediate 4 and L- valinol using general protocol 2; Purification by flash chromatography column on silica gel (cyclohexane/ethyl acetate: 100/0 to 0/100); Yield: 88%; white solid; ¹H NMR (300 MHz, CDCl₃): δ 4.18-4.04 (m, 2H), 4.02-3.90 (m, 2H), 3.78 (dd, J = 8.7, 6.1 Hz, 1H), 3.30-3.11 (m, 1H), 2.97-2.78 (m, 1H), 2.62-2.50 (m, 1H), 2.39-2.34 (m, 1H), 2.09-1.93 (m, 1H), 1.89-1.54 (m, 5H), 1.44 (s, 9H), 0.92 (d, J = 6.9 Hz, 3H), 0.89 (d, J = 6.9 Hz, 3H) ppm. [ES+ MS] m/z 339 (MH⁺). |
| 10 | 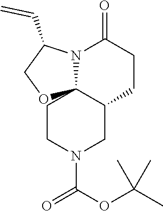 [SMILES: C=C[C@H]1CO[C@]23CCN(C(=O)OC(C)(C)C)C[C@H]2CCC(=O)N13] | Tert-butyl (3S,7aR,11aR)-5-oxo-3-vinyl- 2,3,6,7,7a,8,10,11-octahydrooxazolo[2,3- j][1,6]naphthyridine-9-carboxylate Intermediate 10 was obtained from intermediate 4 and (2S)-2-aminobut-3-en-1-ol; hydrochloride using general protocol 2; Purification by flash chromatography column on silica gel (cyclohexane/ethyl acetate: 100/0 to 0/100); Yield: 59%; colorless oil; [ES+ MS] m/z 323 (MH⁺). |
| 11 | 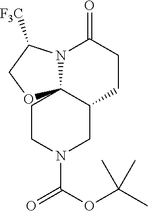 [SMILES: CC(C)(C)OC(=O)N1CC[C@]23OC[C@H](C(F)(F)F)N2C(=O)CC[C@@H]3C1] | Tert-butyl (3R,7aR,11aR)-5-oxo-3-(trifluoromethyl)- 2,3,6,7,7a,8,10,11-octahydrooxazolo[2,3- j][1,6]naphthyridine-9-carboxylate Intermediate 11 was obtained from intermediate 3 and (2R)-2-amino-3,3,3-trifluoro-propan-1-ol; hydrochloride using general protocol 2; Purification by flash chromatography column on silica gel (cyclohexane/ethyl acetate: 100/0 to 0/100); Yield: 47%; colorless oil; ¹H NMR (300 MHz, CDCl₃): δ 4.98-4.84 (m, 1H), 4.25-3.94 (m, 4H), 3.29-3.12 (m, 1H), 2.96-2.69 (m, 1H), 2.70-2.58 (m, 1H), 2.56-2.42 (m, 1H), 1.92-1.75 (m, 4H), 1.71-1.58 (m, 1H), 1.45 (m, 9H) ppm. ¹³C (75 MHz, CDCl₃): δ 170.6, 155.1, 124.2 (q, J = 280.2 Hz), 95.0, 80.2, 63.0 (q, J = 2.0 Hz), 56.3 (q, J = 33.9 Hz), 45.4, 41.2, 40.4, 30.6, 30.4, 28.6, 20.5 ppm. [ES+ MS] m/z 365 (MH⁺). |
| 12 | 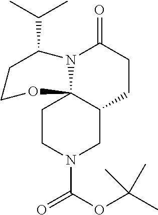 | Tert-butyl (4R,8aR,12aR)-4-isopropyl-6-oxo- 3,4,7,8,8a,9,11,12-octahydro-2H-[1,3]oxazino[2,3- j][1,6]naphthyridine-10-carboxylate Intermediate 12 was obtained from intermediate 3 and (3R)-3-amino-4-methyl-pentan-1-ol using general protocol 2; Purification by flash chromatography column on silica gel (cyclohexane/ethyl acetate: 100/0 to 0/100); Yield: 13%; colorless oil; ¹H NMR (300 MHz, CDCl₃): δ 4.64-4.54 (m, 1H), 4.10-3.76 (m, 3H), 3.73-3.63 (m, 1H), 3.40-3.22 (m, 1H), 2.92-2.73 (m, 1H), 2.62-2.30 (m, 3H), 2.04-1.59 (m, 7H), 1.44 (s, 9H), 0.98 (d, J = 6.3 Hz, 3H), 0.87 (d, J = 6.3 Hz, 3H) ppm. ¹³C (75 MHz, CDCl₃): δ 169.7, 155.0, 86.3, 79.8, 56.3, 51.7, 45.5, 42.3, 39.9, 32.3, 31.7, 30.3, 28.5, 26.6, 21.2, 20.7, 19.6 ppm. [ES+ MS] m/z 353 (MH⁺). |
| 13 | 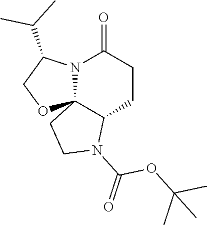 [SMILES: CC(C)[C@H]1CO[C@]23CCN(C(=O)OC(C)(C)C)[C@H]2CCC(=O)N13] | Tert-butyl (1S,4S,9R)-4-isopropyl-6-oxo-2-oxa-5,11- diazatricyclo[7.3.0.01,5]dodecane-11-carboxylate Intermediate 13 was obtained from intermediate 5 with L-valinol using general protocol 2; Yield: 78%; colorless oil; Purification by flash chromatography column on silica gel (cyclohexane/ethyl acetate: 100/0 to 0/100); ¹H NMR (300 MHz, CDCl₃): δ 4.14-4.00 (m, 2H), 3.76-3.57 (m, 2H), 3.54-3.31 (m, 2H), 2.59-2.41 (m, 1H), 2.39-1.92 (m, 4H), 1.86-1.57 (m, 2H), 1.40 (s, 9H), 0.92 (d, J = 6.8 Hz, 3H), 0.88 (d, J = 6.8 Hz, 3H) ppm. ¹³C (75 MHz, CDCl₃): δ 170.4, 154.2, 98.9, 80.0, 66.9, 60.7, 59.2, 43.9, 33.6, 33.0, 29.8, 28.5, 27.2, 25.2, 20.1, 18.6 ppm. [ES+ MS] m/z 325 (MH⁺). |
| 14 | 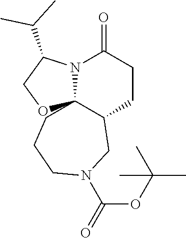 [SMILES: CC(C)[C@H]1CO[C@]23CCCN(C(=O)OC(C)(C)C)C[C@H]2CCC(=O)N13] | Tert-butyl (1R,4S,9R)-4-isopropyl-6-oxo-2-oxa-5,11- diazatricyclo[7.5.0.01,5]tetradecane-11-carboxylate Intermediate 14 was obtained from intermediate 6 and L- valinol, in mixture with other diastereoisomers (8/1/1) using general protocol 2; Purification by flash chromatography column on silica gel (cyclohexane/ethyl acetate: 100/0 to 0/100); Yield: 89%; colorless oil; ¹H NMR (300 MHz, CDCl₃): δ 4.14-3.86 (m, 2H), 3.84-3.53 (m, 2H), 3.44-3.30 (m, 3H), 2.61-2.26 (m, 2H), 2.00-1.53 (m, 8H), 1.44 (s, 9H), 0.94 (d, J = 6.9 Hz, 3H), 0.90 (d, J = 6.8 Hz, 3H) ppm. [ES+ MS] m/z 353 (MH⁺). |
| 15 | 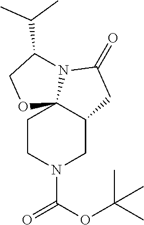 [SMILES: CC(C)[C@H]1CO[C@]23CCN(C(=O)OC(C)(C)C)C[C@H]2CC(=O)N13] | Tert-butyl (1R,4S,8R)-4-isopropyl-6-oxo-2-oxa-5,10- diazatricyclo[6.4.0.01,5]dodecane-10-carboxylate Intermediate 15 was obtained from intermediate 7 and L- valinol using general protocol 2; Purification by flash chromatography column on silica gel (cyclohexane/ethyl acetate: 100/0 to 0/100); Yield: 90%; colorless oil; ¹H NMR (300 MHz, CDCl₃): δ 4.21 (dd, J = 8.8, 7.6 Hz, 1H), 3.83 (dd, J = 8.8 Hz, 6.4 Hz, 1H), 3.70-3.35 (m, 5H), 2.63-2.47 (m, 3H), 2.10-1.88 (m, 2H), 1.68-1.53 (m, 1H), 1.46 (s, 9H), 1.03 (d, J = 6.7 Hz, 3H), 0.87 (d, J = 6.7 Hz, 3H) ppm. ¹³C (75 MHz, CDCl₃): δ 178.2, 155.6, 99.2, 80.2, 71.2, 61.8, 42.7, 41.5, 40.6, 39.7, 36.8, 33.9, 30.6, 28.7, 20.7, 19.1 ppm. [ES+ MS] m/z 325 (MH⁺). |
| 16 | 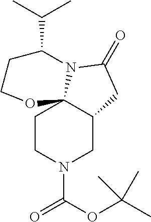 [SMILES: CC(C)[C@H]1CCO[C@]23CCN(C(=O)OC(C)(C)C)C[C@H]2CC(=O)N13] | Tert-butyl (1R,5R,9R)-5-isopropyl-7-oxo-2-oxa-6,11- diazatricyclo[7.4.0.01,6]tridecane-11-carboxylate Intermediate 20 was obtained from intermediate 7 and (3R)-3-amino-4-methyl-pentan-1-ol using general protocol 2; Purification by flash chromatography column on silica gel (cyclohexane/ethyl acetate: 100/0 to 0/100); Yield: 91%; colorless oil; [ES+ MS] m/z 339 (MH⁺). |
| 17 | 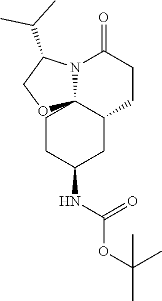 [SMILES: CC(C)[C@H]1CO[C@]23CC[C@H](NC(=O)OC(C)(C)C)C[C@H]2CCC(=O)N13] | Tert-butyl N-[(3S,7aR,9S,11aR)-3-isopropyl-5-oxo- 3,6,7,7a,8,9,10,11-octahydro-2H-oxazolo[2,3- j]quinolin-9-yl]carbamate Intermediate 17 was obtained from intermediate 8 and L-valinol using general protocol 2; Purification by flash chromatography column over silica gel (cyclohexane/ acetone 100/0 to 90/10); Yield: 41%, white powder; ¹H NMR (300 MHz, CDCl₃): δ 4.56-4.28 (m, 1H), 4.17- 3.96 (m, 2H), 3.32-3.61 (m, 2H), 2.67-2.53 (m, 1H), 2.48-2.28 (m, 1H), 2.11-1.85 (m, 5H), 1.84-1.68 (m, 4H), 1.46 (s, 10H), 0.96 (d, J = 6.9 Hz, 3H), 0.92 (d, J = 6.9 Hz, 3H) ppm. [ES+ MS] m/z 353 (MH⁺). |
| 18 | 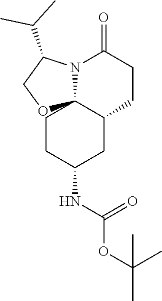 [SMILES: CC(C)[C@H]1CO[C@]23CC[C@@H](NC(=O)OC(C)(C)C)C[C@H]2CCC(=O)N13] | Tert-butyl N-[(3S,7aR,9R,11aR)-3-isopropyl-5-oxo- 3,6,7,7a,8,9,10,11-octahydro-2H-oxazolo[2,3- j]quinolin-9-yl]carbamate Intermediate 18 was obtained from intermediate 8 with L-valinol using general protocol 2; Purification by flash chromatography column over silica gel (cyclohexane/acetone 100/0 to 90/10); Yield: 29%; white powder; ¹H NMR (300 MHz, CDCl₃): 4.35 (d, J = 7.4 Hz, 1H), 4.05-3.96 (m, 1H), 3.95-3.89 (m, 2H), 3.84-3.64 (m, 1H), 3.00 (d sept, J = 3.2 Hz, J = 7.0 Hz, 1H), 2.51- 2.39 (m, 2H), 2.07-1.97 (m, 5H), 1.95-1.83 (m, 4H), 1.45 (s, 9H), 0.87 (d, J = 7.1 Hz, 3H), 0.72 (d, J = 7.0 Hz, 3H) ppm. [ES+ MS] m/z 353 (MH⁺). |
| 19 | 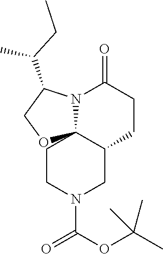 [SMILES: CC[C@@H](C)[C@H]1CO[C@]23CCN(C(=O)OC(C)(C)C)C[C@H]2CCC(=O)N13] | Tert-butyl (3S,7aR,11aR)-3-[(1R)-1-methylpropyl]-5- oxo-2,3,6,7,7a,8,10,11-octahydrooxazolo[2,3- j][1,6]naphthyridine-9-carboxylate Intermediate 19 was obtained from intermediate 4 with (2S,3R)-2-amino-3-methyl-pentan-1-ol using general protocol 2; Purification by flash chromatography column on silica gel (cyclohexane/ethyl acetate: 100/0 to 60/40); Yield: 28%; colorless oil; ¹H NMR (300 MHz, CDCl₃): δ 4.21-3.89 (m, 3H); 3.83-3.76 (m, 1H); 3.33-3.10 (m, 1H); 2.98-2.79 (m, 1H); 2.64-2.51 (m, 1H); 2.49-2.39 (m, 1H); 1.89-1.56 (m, 8H); 1.45 (s, 9H); 1.17-1.06 (m, 1H); 0.95- 0.88 (m, 6H) ppm. ¹³C (75 MHz, CDCl₃): δ 169.7, 155.1, 92.4, 80.0, 67.2, 60.8, 45.6, 40.3, 39.5, 32.1, 31.1, 30.4, 28.6, 25.9, 20.7, 16.0, 11.7 ppm. [ES+ MS] m/z 353 (MH⁺). |

### Synthesis of 4-(bromomethyl)-2-cyclopropyl-quinoline (intermediate 20) for use in General protocol 3

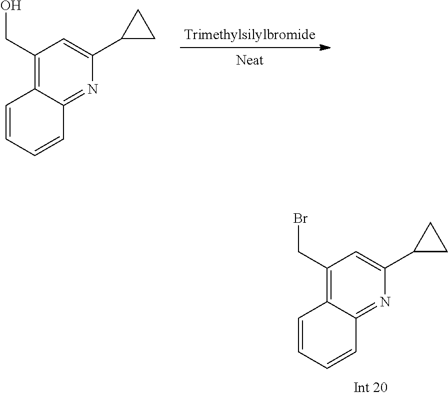 [SMILES: OCc1cc(C2CC2)nc2ccccc12 ; BrCc1cc(C2CC2)nc2ccccc12]

(2-cyclopropyl-4-quinolyl) methanol (1 eq) was dissolved in bromo (trimethyl) silane (5 eq). The mixture was stirred at room temperature overnight, and then at 90° C. for 8 h. The crude was then evaporated under reduced pressure, and purified through flash chromatography (cyclohexane/thyl acetate: 100/0 to 0/100) over silica gel to afford the desired product as a white powder; Yield=15%. [ES+MS] m/z 264, 262 (MH⁺).

General Protocol 3: Synthesis of N-Alkylated Lactams from N-Boc Lactams

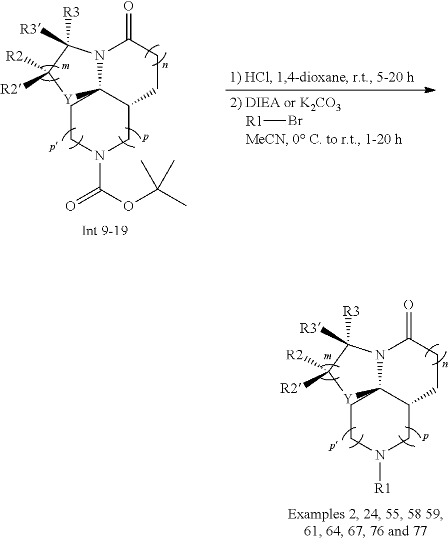

Step 1: The appropriate N-Boc lactam was dissolved in 1,4-dioxane (0.05 M), then HCl in solution in 1,4-dioxane (4N, 10 eq) was added. The mixture was stirred at room temperature for 5-20 h. After full conversion (as determined by TLC or LC/MS), the solvent was removed under vacuum and the crude product obtained was engaged into the next step without any further purification.

Step 2: The crude product of step 1 was dissolved in MeCN (0.1N). The solution was cooled down to 0° C., then DIEA or K₂CO₃ (1.5-3 eq) and the appropriate alkylbromide (1.5 eq) were added. The mixture was warmed up to room temperature and stirred for 1-20 h. The solvent was then removed under reduced pressure. The residue obtained was dissolved in H₂O and extracted with dichloromethane. The organic layer was dried over MgSO₄, filtered and concentrated under vacuum to give the crude product, which was purified to afford the corresponding desired lactams.

| Example | Structure | Physical data |
|---|---|---|
| 2 | 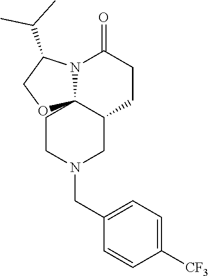 | (3S,7aR,11aR)-3-isopropyl-9-[[4- (trifluoromethyl)phenyl]methyl]-2,3,6,7,7a,8,10,11- octahydrooxazolo[2,3-j][1,6]naphthyridin-5-one Example 2 was obtained from intermediate 9 and 4- (trifluoromethyl)benyzyl bromide using general protocol 3; Purification by flash column chromatography over silica gel (cyclohexane/ethyl acetate:100/0 to 0/100); Yield: 53% over 2 steps; colorless oil; ¹H NMR (300 MHz, CD₂Cl₂): δ 7.57 (d, J = 8.1 Hz, 2H), 7.47 (d, J = 8.1 Hz, 2H), 4.07- 3.94 (m, 2H), 3.75 (dd, J = 8.0 Hz, 5.7 Hz, 1H), 3.58 (d, J = 13.9 Hz, 1H), 3.48 (d, J = 13.9 Hz, 1H), 2.73-2.61 (m, 2H), 2.57-2.45 (m, 2H), 2.41-2.28 (m, 2H), 2.27-2.18 (m, 1H), 2.03-1.90 (m, 2H), 1.82-1.74 (m, 1H), 1.69-1.56 (m, 2H), 0.91 (d, J = 6.8 Hz, 3H), 0.88 (d, J = 6.8 Hz, 3H) ppm. ¹³C NMR (75 MHz, CD₂Cl₂): δ 170.0, 144.0, 129.2 (q, J = 32.2 Hz), 129.2, 125.4 (q, J = 3.7 Hz), 124.8 (q, J = 272.1 Hz), 92.7, 66.4, 62.2, 61.5, 55.3, 51.0, 40.8, 32.8, 32.6, 31.2, 32.3, 19.9, 18.7 ppm. HRMS (ESI, m/z): [M + H]⁺ calcd. for C₂₁H₂₇N₂O₂F₃, 397.2103; found 396.2101. |
| 24 | 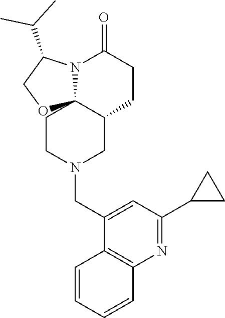 [SMILES: CC(C)[C@H]1CO[C@]23CCN(Cc4cc(C5CC5)nc5ccccc45)C[C@H]2CCC(=O)N13] | (3S,7aR,11aR)-9-[(2-cyclopropyl-4-quinolyl)methyl]-3- isopropyl-2,3,6,7,7a,8,10,11-octahydrooxazolo[2,3- j][1,6]naphthyridin-5-one Example 24 was obtained from intermediate 9 and intermediate 20 using general protocol 3; Purification by flash column chromatography over silica gel (cyclohexane/ethyl acetate:100/0 to 50/50); Yield: 50% over 2 steps; colorless oil; ¹H NMR (300 MHz, CDCl₃): δ 8.16 (dd, J = 1.0 Hz, 8.4 Hz, 1H), 8.0 (dd, J = 1.2 Hz, J = 8.5 Hz, 1H), 7.64 (ddd, J = 1.4 Hz, J = 6.8 Hz, J = 8.4 Hz, 1H), 7.44 (ddd, J = 1.3 Hz, J = 6.9 Hz, J = 8.3 Hz, 1H), 7.16 (s, 1H), 4.16-4.07 (m, 1H), 4.02 (dd, J = 7.6 Hz, J = 8.6 Hz, 1H), 3.85 (d, J = 1.9 Hz, 2H), 3.80 (dd, J = 6.1 Hz, J = 8.6 Hz, 1H), 2.83-2.70 (m, 2H), 2.66-2.52 (m, 2H), 2.46- 2.28 (m, 3H), 2.26-2.17 (m, 1H), 2.09-1.91 (m, 2H), 1.82 (dq, J = 2.2 Hz, J = 13.7 Hz, 1H), 1.75-.1.59 (m, 3H), 1.18-1.06 (m, 4H), 0.95 (d, J = 6.9 Hz, 3H), 0.91 (d, J = 6.9 Hz, 3H) ppm. ¹³C NMR (75 MHz, CDCl₃): δ 170.0, 163.0, 148.3, 143.5, 129.2, 129.0, 126.1, 124.8, 124.0, 119.4, 92.4, 66.1, 61.2, 59.7, 55.2, 50.8, 40.4, 32.3, 32.3, 30.7, 21.9, 19.8, 18.6, 18.0, 10.2, 10.2 ppm. [ES+ MS] m/z 420 (MH⁺). |
| 55 | 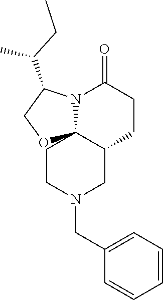 [SMILES: CC[C@@H](C)[C@H]1CO[C@]23CCN(Cc4ccccc4)C[C@H]2CCC(=O)N13] | (3S,7aR,11aR)-9-benzyl-3-[(1R)-1-methylpropyl]- 2,3,6,7,7a,8,10,11-octahydrooxazolo[2,3- j][1,6]naphthyridin-5-one Example 55 was obtained from intermediate 19 and benzyl bromide using general protocol 3; Purification by flash column chromatography over silica gel (cyclohexane/ethyl acetate:100/0 to 60/40); Yield: 60% over 2 steps; colorless oil; ¹H NMR (300 MHz, CDCl₃): δ 7.32-7.230 (m, 5H), 4.15 (dd, J = 14.9, 7.4 Hz, 1H), 4.00 (dd, J = 8.7, 7.4 Hz, 1H), 3.77 (dd, J = 8.7 Hz, 6.1 Hz, 1H), 3.55 (d, J = 13.2 Hz, 1H), 3.41 (d, J = 13.2 Hz, 1H), 2.77-2.52 (m, 3H), 2.49-2.32 (m, 3H), 2.26-2.16 (m, 1H), 2.03-1.92 (m,1H), 1.82-1.55 (m, 4H), 1.48-1.34 (m, 1H), 1.17-1.05 (m, 1H), 0.91 (t, J = 7.2 Hz, 3H), 0.92 (d, J = 6.9 Hz, 3H) ppm. ¹³C NMR (75 MHz, CDCl₃): δ 170.2, 128.7, 128.3, 127.1, 92.7, 66.9, 62.6, 60.5, 54.9, 50.7, 40.5, 39.6, 32.5, 30.9, 25.9, 22.0, 16.0, 11.7 ppm. [ES+ MS] m/z 343 (MH⁺). |
| 58 | 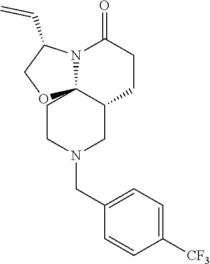 [SMILES: C=C[C@H]1CO[C@]23CCN(Cc4ccc(C(F)(F)F)cc4)C[C@H]2CCC(=O)N13] | (3S,7aR,11aR)-9-[[4-(trifluoromethyl)phenyl]methyl]-3- vinyl-2,3,6,7,7a,8,10,11-octahydrooxazolo[2,3- j][1,6]naphthyridin-5-one Example 58 was obtained from intermediate 10 and 4- (trifluoromethyl)benzyl bromide using general protocol 3; Purification by flash column chromatography over silica gel (cyclohexane/ethyl acetate: 100/0 to 0/100); Yield: 51% over 2 steps; colorless oil; ¹H NMR (300 MHz, CDCl₃): δ 7.59 (d, J = 8.1 Hz, 2H), 7.46 (d, J = 8.1 Hz, 2H), 5.80 (ddd, J = 16.4 Hz, 10.2 Hz, 6 Hz, 1H), 5.29 (td, J = 16.4 Hz, 1.3 Hz, 1H), 5.22 (td, J = 10.2 Hz, 1.3 Hz, 1H), 4.85-4.75 (m, 1H), 4.28 (dd, J = 8.8 Hz, 8.8 Hz, 1H), 3.78 (dd, J = 8.8 Hz, 7.4 Hz, 1H), 3.62 (d, J = 13.9 Hz, 1H), 3.48 (d, J = 13.9 Hz, 1H), 2.93-2.34 (m, 6H), 2.33-2.18 (m, 1H), 2.02-1.81 (m, 2H), 1.77-1.64 (m, 2H) ppm. ¹³C NMR (75 MHz, CDCl₃): δ 169.0, 143.1, 136.4, 129.5 (q, J = 33.2 Hz), 128.9, 125.4 (q, J = 3.7 Hz), 124.2 (q, J = 272.0 Hz), 117.1, 92.6, 67.7, 62.0, 57.3, 54.9, 50.7, 40.3, 31.2, 31.0, 22.7 ppm. [ES+ MS] m/z 381 (MH⁺). |
| 59 | 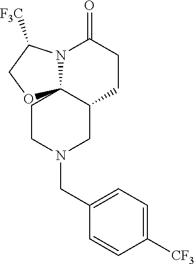 [SMILES: O=C1CC[C@@H]2CN(Cc3ccc(C(F)(F)F)cc3)CC[C@@]23OC[C@H](C(F)(F)F)N13] | (3R,7aR,11aR)-3-(trifluoromethyl)-9-[4- (trifluoromethyl)phenyl]methyl]-2,3,6,7,7a,8,10,11- octahydrooxazolo[2,3-j][1,6]naphthyridin-5-one Example 59 was obtained from intermediate 11 and 4- (trifluoromethyl)benzyl bromide using general protocol 3; Purification by flash column chromatography over silica gel (cyclohexane/ethyl acetate: 100/0 to 0/100); Yield: 65% over 2 steps; colorless oil; ¹H NMR (300 MHz, CDCl₃): δ 7.49 (d, J = 8.1 Hz, 2H), 7.35 (d, J = 8.1 Hz, 2H), 4.82 (septuplet, J = 7.0 Hz, 1H), 4.13 (dd, J = 9.5 Hz, 9.5 Hz, 1H), 4.03 (dd, J = 9.5 Hz, 6.4 Hz, 1H), 3.53 (d, J = 14.0 Hz, 1H), 3.37 (d, J = 14.0 Hz, 1H), 2.73-2.53 (m, 3H), 2.46-2.27 (m, 3H), 2.17 (dt, J = 12.0 Hz, 4.5 Hz, 1H), 2.00- 1.79 (m, 1H), 1.71-1.51 (m, 2H) ppm. ¹³C NMR (75 MHz, CDCl₃): δ 177.1, 143.1, 129.7 (q, J = 31.5 Hz), 129.0, 125.5 (q, J = 3.5 Hz), 124.4 (q, J = 282.6 Hz), 124.4 (q, J = 274.7 Hz), 95.1, 62.8 (q, J = 1.6 Hz), 62.1, 56.2 (q, J = 33.7 Hz), 54.8, 50.8, 40.7, 31.1, 30.9, 21.9 ppm. [ES+ MS] m/z 423 (MH⁺). |
| 61 | 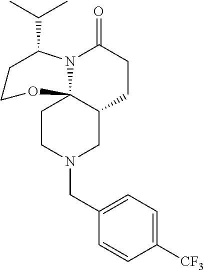 | (4R,8aR,12aR)-4-isopropyl-10-[4- (trifluoromethyl)phenyl]methyl]-3,4,7,8,8a,9,11,12- octahydro-2H-[1,3]oxazino[2,3-j][1,6]naphthyridin-6- one Example 61 was obtained from intermediate 12 and 4- (trifluoromethyl)benzyl bromide using general protocol 3; Purification by flash column chromatography over silica gel (cyclohexane/ethyl acetate: 100/0 to 0/100); Yield: 66% over 2 steps; colorless oil; ¹H NMR (300 MHz, CDCl₃): δ 7.55 (d, J = 8.1 Hz, 2H), 7.43 (d, J = 8.1 Hz, 2H), 4.65- 4.57 (m, 1H), 3.95-3.85 (m, 1H), 3.69-3.61 (m, 1H), 3.54 (d, J = 13.6 Hz, 1H), 3.43 (d, J = 13.6 Hz, 1H), 2.66-2.31 (m, 8H), 2.10-1.73 (m, 5H), 1.65-1.55 (m, 1H), 0.99 (d, J = 6.3 Hz, 3H), 0.89 (d, J = 6.7 Hz, 3H) ppm. ¹³C NMR (75 MHz, CDCl₃): δ 170.6, 143.2, 129.3 (q, J = 32.4 Hz), 128.8, 125.2 (q, J = 3.8 Hz), 124.1 (q, J = 271.2 Hz), 86.2, 61.9, 55.9, 54.8, 51.6, 50.1, 42.4, 32.2, 32.1, 30.7, 26.5, 22.0, 21.1, 19.5 ppm. [ES+ MS] m/z 411 (MH⁺). |
| 77 | 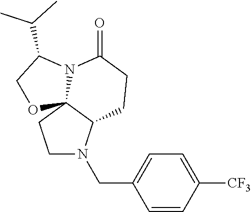 [SMILES: CC(C)[C@H]1CO[C@]23CCN(Cc4ccc(C(F)(F)F)cc4)[C@H]2CCC(=O)N13] | (1R,4S,9S)-4-isopropyl-10-[[4- (trifluoromethyl)phenyl]methyl]-2-oxa-5,10- diazatricyclo[7.3.0.01,5]dodecan-6-one Example 77 was obtained from intermediate 13 and 4- (trifluoromethyl)benzyl bromide using general protocol 3; Purification by flash column chromatography over silica gel (cyclohexane/ethyl acetate: 100/0 to 0/100); Yield: 60% over 2 steps; colorless oil; ¹H NMR (300 MHz, CDCl₃): δ 7.57 (d, J = 8.0 Hz, 2H), 7.43 (d, J = 8.0 Hz, 2H), 4.11-3.95 (m, 3H), 3.69 (dd, J = 8.5 Hz, 6.6 Hz, 1H), 3.40 (d, J = 13.7 Hz, 1H), 2.90-2.81 (m, 2H), 2.58-2.45 (m, 1H), 2.29-2.20 (m, 3H), 1.92-1.83 (m, 1H), 1.78-1.59 (m, 3H), 0.94 (d, J = 7.1 Hz, 3H), 0.88 (d, J = 8.0 Hz, 3H) ppm. ¹³C NMR (75 MHz, CDCl₃): δ 170.5, 143.0, 129.6 (q, J = 31.1 Hz), 128.9, 125.4 (q, J = 3.8 Hz), 124.5 (q, J = 275.6 Hz), 100.4, 67.0, 65.5, 60.6, 58.1, 50.8, 39.1, 31.0, 29.1, 23.6, 19.6, 17.6 ppm. [ES+ MS] m/z 383 (MH⁺). |
| 76 | 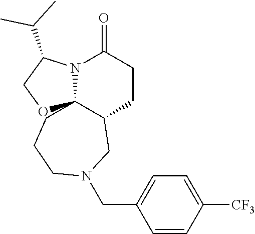 [SMILES: CC(C)[C@H]1CO[C@]23CCCN(Cc4ccc(C(F)(F)F)cc4)C[C@H]2CCC(=O)N13] | (1R,4S,9R)-4-isopropyl-11-[[4- (trifluoromethyl)phenyl]methyl]-2-oxa-5,11- diazatricyclo[7.5.0.01,5]tetradecan-6-one Example 76 was obtained from intermediate 14 and 4- (trifluoromethyl)benzyl bromide using general protocol 3; Purification by flash column chromatography over silica gel (cyclohexane/ethyl acetate: 100/0 to 0/100); Yield: 56% over 2 steps; colorless oil; ¹H NMR (300 MHz, CDCl₃): δ 7.56 (d, J = 7.9 Hz, 2H), 7.24 (d, J = 7.9 Hz, 2H), 4.10-3.96 (m, 2H), 3.70-3.60 (m, 3H), 2.99 (dd, J = 2.0 Hz, 14.2 Hz, 1H), 2.76-2.64 (m, 1H), 2.62-2.40 (m, 3H), 2.37-2.22 (m, 1H), 2.20-2.08 (m, 1H), 2.06-1.85 (m, 3H), 1.84-1.74 (m, 1H), 1.61-1.50 (m, 1H), 0.95 (d, J = 6.8 Hz, 3H), 0.85 (d, J = 6.8 Hz, 3H) ppm. ¹³C NMR (75 MHz, CDCl₃): δ 170.9, 144.0, 129.5 (q, J = 32.0 Hz), 129.2, 125.2 (q, J = 3.8 Hz), 124.4 (q, J = 272.0 Hz), 96.9, 66.9, 63.7, 61.3, 54.8, 53.4, 44.8, 33.8, 31.2, 30.8, 21.8, 20.6, 20.2, 19.4 ppm. [ES+ MS] m/z 411 (MH⁺). |
| 64 | 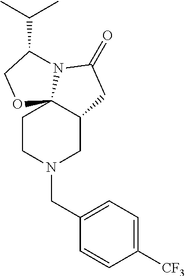 [SMILES: CC(C)[C@H]1CO[C@]23CCN(Cc4ccc(C(F)(F)F)cc4)C[C@H]2CC(=O)N13] | (1R,4S,8R)-4-isopropyl-10-[4- (trifluoromethyl)phenyl]methyl]-2-oxa-5,10- diazatricyclo[6.4.0.01,5]dodecan-6-one Example 64 was obtained from intermediate 15 and 4- (trifluoromethyl)benzyl bromide using general protocol 3; Purification by flash column chromatography over silica gel (cyclohexane/ethyl acetate: 100/0 to 0/100); Yield: 82% over 2 steps; colorless oil; ¹H NMR (300 MHz, CDCl₃): δ 7.56 (d, J = 8.0 Hz, 1H), 7.47 (d, J = 8.0 Hz, 1H), 4.10 (dd, J = 9.0, 7.1 Hz, 1H), 3.97 (dd, J = 9.0 Hz, 4.7 Hz, 1H), 3.69- 3.63 (m, 1H), 3.61 (d, J = 13.7 Hz, 1H), 3.57 (d, J = 13.7 Hz, 1H), 3.00-2.86 (m, 1H), 2.73-2.61 (m, 2H), 2.57-2.47 (m, 1H), 2.45-2.31 (m, 2H), 2.25 (td, J = 11.3 Hz, 2.8 Hz, 1H), 2.11 (td, J = 13.5, 4.0 Hz, 1H), 2.03-1.92 (m, 1H), 1.75-1.59 (m, 1H), 1.02 (d, J = 6.7 Hz, 3H), 0.92 (d, J = 6.7 Hz, 3H) ppm. ¹³C NMR (75 MHz, CDCl₃): δ 176.7, 142.8, 129.5 (q, J = 32.3 Hz), 129.9, 125.4 (q, J = 3.8 Hz), 124.6 (q, J = 271.6 Hz), 97.3, 71.2, 62.0, 61.5, 53.2, 50.0, 42.8, 38.5, 34.8, 32.5, 20.3, 19.2 ppm. [ES+ MS] m/z 383 (MH⁺). |
| 67 | 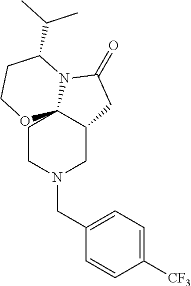 [SMILES: CC(C)[C@H]1CCO[C@]23CCN(Cc4ccc(C(F)(F)F)cc4)C[C@H]2CC(=O)N13] | (1R,5R,9R)-5-isopropyl-11-[[4- (trifluoromethyl)phenyl]methyl]-2-oxa-6,11- diazatricyclo[7.4.0.01,6]tridecan-7-one Example 67 was obtained from intermediate 16 and 4- (trifluoromethyl)benzyl bromide using general protocol 3; Purification by flash column chromatography over silica gel (cyclohexane/ethyl acetate: 100/0 to 0/100); Yield: 72% over 2 steps; colorless oil; ¹H NMR (300 MHz, CDCl₃): δ 7.56 (d, J = 8.1 Hz, 2H), 7.43 (d, J = 8.1 Hz, 2H), 3.97-3.86 (m, 1H), 3.84-3.73 (m, 1H), 3.68-3.58 (m, 1H), 3.57 (d, J = 13.9 Hz, 1H), 3.51 (d, J = 13.9 Hz, 1H), 2.61-2.42 (m, 4H), 2.38-2.27 (m, 2H), 2.26-2.14 (m, 2H), 1.94-1.69 (m, 4H), 0.95 (dd, J = 6.7 Hz, 1.0 Hz, 6H) ppm. ¹³C NMR (75 MHz, CDCl₃): δ 173.4, 142.8, 129.6 (q, J = 32.3 Hz), 129.0, 125.4 (q, J = 3.7 Hz), 124.2 (q, J = 271.8 Hz), 88.1, 61.1, 58.0, 53.5, 52.9, 49.6, 42.5, 34.2, 33.0, 31.9, 26.5, 20.5, 20.2 ppm; [ES+ MS] m/z 397 (MH⁺). |

### General Protocol 4: Deprotection of N-Boc Lactams

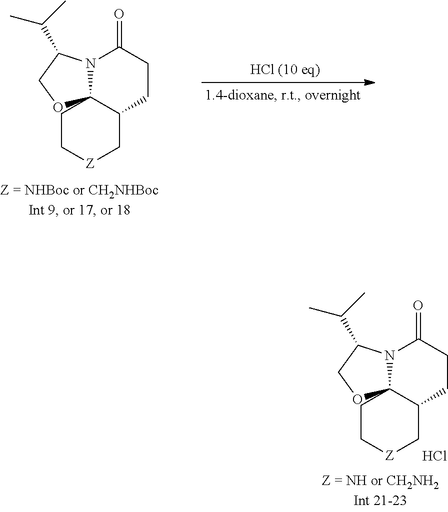

The appropriate N-Boc lactam was dissolved in 1,4-dioxane (0.05 M), then HCl solution in 1,4-dioxane (4N, 10 eq) was added. The mixture was stirred at room temperature for 5-48 h. until full conversion of the starting material (as determined by TLC or LC/MS), the solvent was removed under vacuum and the crude product obtained was engaged into the next step reaction without further purification.

| Intermediate | Structure | Physical data |
|---|---|---|
| 21 | 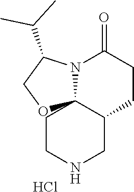 [SMILES: CC(C)[C@H]1CO[C@]23CCNC[C@H]2CCC(=O)N13 ; Cl] | (3S,7aR,11aR)-3-isopropyl-3,6,7,7a,8,9,10,11-octahydro- 2H-oxazolo[2,3-j][1,6]naphthyridin-5-one; hydrochloride Intermediate 21 was obtained from intermediate 9 using general protocol 4; Yield: quantitative; white powder. ¹H NMR (300 MHz, MeOD, d4): δ 4.18-4.04 (m, 2H), 3.99- 3.87 (m, 1H), 3.48-3.28 (m, 4H), 3.24-3.0 (m, 1H), 2.75 (ddd, J = 1.7 Hz, 7.3 Hz, 18.4 Hz, 1H), 2.26-1.84 (m, 6H), 0.97-0.92 (m, 6H) ppm. [ES+ MS] m/z 239 (MH⁺ − HCl). |
| 22 | 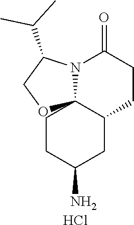 [SMILES: CC(C)[C@H]1CO[C@]23CC[C@H](N)C[C@H]2CCC(=O)N13 ; Cl] | (3S,7aR,9S,11aR)-9-amino-3-isopropyl- 3,6,7,7a,8,9,10,11-octahydro-2H-oxazolo[2,3-j]quinolin-5- one; hydrochloride Intermediate 22 was obtained from intermediate 17 using general protocol 4; Yield: quantitative; white solid. ¹H NMR (300 MHz, D₂O): δ 4.04 (dd, J = 7.2 Hz, J = 8.4 Hz, 1H), 3.92 (ddd, J = 1.7 Hz, J = 6.4 Hz; J = 14.5 Hz, 1H), 3.82 (dd, J = 6.4 Hz, J = 8.4 Hz, 1H), 3.46-3.31 (m, 1H), 2.52 (dd, J = 5.1 Hz, J = 18.5 Hz, 1H), 1.41-2.24 (m, 1H), 2.11- 1.42 (m, 10H), 0.82-0.75 (m, 6H) ppm. [ES+ MS] m/z 253 (MH⁺ − HCl). |
| 23 | 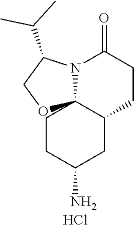 [SMILES: CC(C)[C@H]1CO[C@]23CC[C@@H](N)C[C@H]2CCC(=O)N13 ; Cl] | (3S,7aR,9R,11aR)-9-amino-3-isopropyl- 3,6,7,7a,8,9,10,11-octahydro-2H-oxazolo[2,3-j]quinolin-5- one; hydrochloride Intermediate 23 was obtained from intermediate 18 using general protocol 4; Yield: quantitative; white solid. ¹H NMR (300 MHz, D₂O): δ 4.06-3.94 (m, 3H), 3.51-3.38 (m, 1H), 2.71-2.55 (m, 1H), 2.52-2.30 (m, 2H), 2.17-1.74 (m, 7H), 1.64-1.44 (m, 2H), 0.78 (d, J = 7.1 Hz, 3H), 0.61 (d, J = 7.0 Hz, 3H) ppm. [ES+ MS] m/z 253 (MH⁺ − HCl). |

### Synthesis of (3S,7aR,11aR)-3-isopropyl-3,6,7,7a,8,9,10,11-octahydro-2H-oxazolo[2,3-j][1,6]naphthyridin-5-one; formic acid (Intermediate 24)

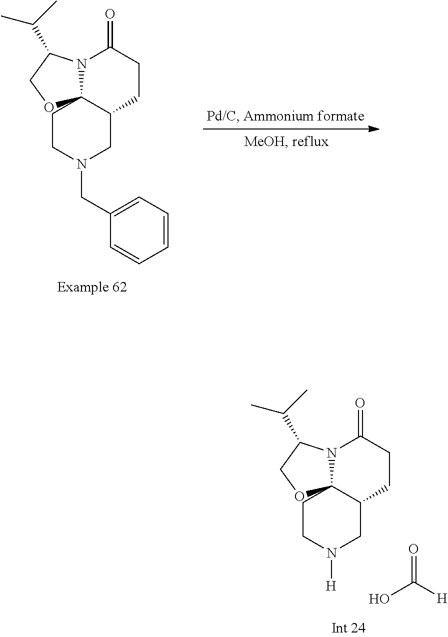 [SMILES: CC(C)[C@H]1CO[C@]23CCN(Cc4ccccc4)C[C@H]2CCC(=O)N13 ; CC(C)[C@H]1CO[C@]23CCNC[C@H]2CCC(=O)N13 ; O=CO]

Example 62 (655 mg, 1.99 mmol, 1 eq.) was dissolved in methanol (20 mL), then were added Pd/C 10% (127 mg, 1.20 mmol, 0.12 mmol, 10 mol %) and ammonium formate (629 mg, 9.97 mmol, 5 eq.). The mixture was refluxed during 30 minutes. The solution was filtered over celite, then the filtrate was concentrated under reduced pressure to afford intermediate 24 with a quantitative yield as a white powder. ¹H NMR (300 MHZ, CD₂Cl₂): δ 8.44 (brs, 1H), 4.13-3.99 (m, 2H), 3.77 (dd, J=8.3, 5.6 Hz, 1H), 3.35-3.11 (m, 3H), 2.99 (td, J=13.1, 3.2 Hz, 1H), 2.68-2.55 (m, 1H), 2.48-2.20 (m, 2H), 2.10 (td, J=14.4, 4.6 Hz, 1H), 2.01-1.70 (m, 4H), 0.92 (d, J=6.4 Hz, 3H), 0.90 (d, J=6.4 Hz, 3H) ppm. HRMS (ESI, m/z): [M+H]⁺ calcd. for C₁₃H₂₂N₂O₂, 239.1760; found 239.1759.

### General Protocol 5: Alkylation of Deprotected Lactams

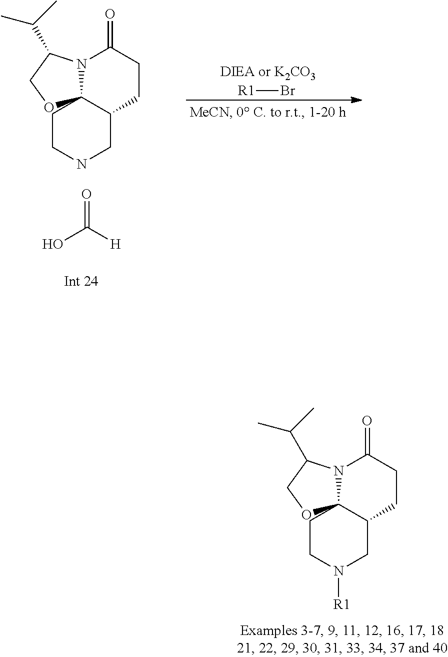

Intermediate 24 (1 eq) was dissolved in MeCN (0.1N). The solution was cooled down to 0 5° C., then DIEA or K₂CO₃ (1.5-3 eq) and the appropriate bromide (1-1.5 eq), and NaI (0-1 eq) were added. The mixture was warmed up to room temperature. The solution was stirred at room temperature or refluxed at 80° C. (if necessary). When the conversion of the intermediate 24 into the desired product was judged complete by LC/MS or TLC, the solvent was removed under reduced pressure. The residue obtained was dissolved in H₂O and extracted with dichloromethane. The organic layer was dried over MgSO₄, filtered and 10 concentrated under vacuum to give the crude product, which was purified to afford the corresponding desired lactams.

| Ex- | | |
|---|---|---|
| amples | Structure | Physical data |
| 3 | 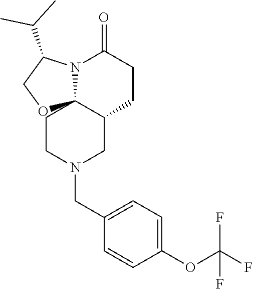 [SMILES: CC(C)[C@H]1CO[C@]23CCN(Cc4ccc(OC(F)(F)F)cc4)C[C@H]2CCC(=O)N13] | (3S,7aR,11aR)-3-isopropyl-9-[[4- (trifluoromethoxy)phenyl]methyl]-2,3,6,7,7a,8,10,11- octahydrooxazolo[2,3-j][1,6]naphthyridin-5-one Example 3 was obtained from intermediate 24 and 1- (bromomethyl)-4-(trifluoromethoxy)benzene using general protocol 5; Purification by flash chromatography column over silica (cyclohexane/ethyl acetate: 100/0 to 60/40); Yield: 77%; colorless oil; ¹H NMR (300 MHz, CD₂Cl₂): δ 7.38 (d, J = 8.6 Hz, 2H), 7.17 (d, J = 8.6 Hz, 2H), 4.07-3.94 (m, 2H), 3.75 (dd, J = 8.0 Hz, 5.7 Hz, 1H), 3.52 (d, J = 13.6 Hz, 1H), 3.42 (d, J = 13.6 Hz, 1H), 2.74-2.60 (m, 2H), 2.58-2.42 (m, 2H), 2.40-2.26 (m, 2H), 2.26-2.14 (m, 1H), 2.04-1.87 (m, 2H), 1.83-1.74 (m, 1H), 1.72-1.54 (m, 2H), 0.91 (d, J = 6.7 Hz, 3H), 0.88 (d, J = 6.7 Hz, 3H) ppm. ¹³C NMR (75 MHz, CD₂Cl₂): δ 170.0, 148.5, 138.6, 130.3, 121.1, 120.7, 92.8, 66.4, 61.9, 61.6, 55.3, 50.9, 40.8, 32.8, 32.7, 31.2, 32.3, 20.0, 18.8 ppm. HRMS (ESI, m/z): [M + H]⁺ calcd. for C₂₁H₂₈N₂O₃F₃, 413.2052; found 413.2056. |
| 4 | 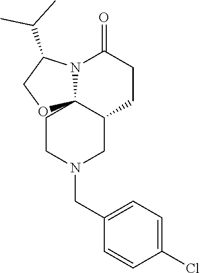 | (3S,7aR,11aR)-9-[(4-chlorophenyl)methyl]-3-isopropyl- 2,3,6,7,7a,8,10,11-octahydrooxazolo[2,3- jl[1,6]naphthyridin-5-one Example 4 was obtained from intermediate 24 and 11- chloro-4-(chloromethyl)benzene using general protocol 5; Purification by flash chromatography column over silica (cyclohexane/ethyl acetate: 100/0 to 30/70); Yield: 42%; colorless oil; ¹H NMR (300 MHz, CDCl₃): δ 7.30-7.21 (m, 4H), 4.14-4.05 (m, 1H), 3.98 (dd, J = 8.6 Hz, 7.6 Hz, 1H), 3.76 (dd, J = 8.6 Hz, 6.2 Hz, 1H), 3.49 (d, J = 13.5 Hz, 1H), 3.37 (d, J = 13.5 Hz, 1H), 2.74-2.61 (m, 2H), 2.60-2.52 (m, 1H), 2.50-2.29 (m, 3H), 2.26-2.15 (m, 1H), 2.08-1.89 (m, 2H), 1.84-1.76 (m, 1H), 1.72-1.56 (m, 2H), 0.94 (d, J = 6.8 Hz, 3H), 0.89 (d, J = 6.8 Hz, 3H) ppm. ¹³C NMR (75 MHz, CDCl₃): δ 170.2, 137.5, 132.7, 130.0, 128.5, 92.6, 66.2, 61.8, 61.3, 54.8, 50.7, 40.4, 32.4, 32.4, 30.8, 22.0, 19.9, 18.7 ppm. HRMS (ESI, m/z): [M + H]⁺ calcd. for C₂₀H₂₈N₂O₂Cl, |
| | | 363.1839; found 363.1872. |
| 5 | 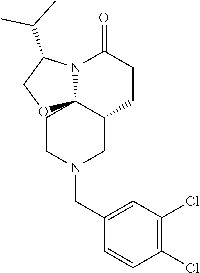 [SMILES: CC(C)[C@H]1CO[C@]23CCN(Cc4ccc(Cl)c(Cl)c4)C[C@H]2CCC(=O)N13] | (3S,7aR,11aR)-9-[(3,4-dichlorophenyl)methyl]-3- isopropyl-2,3,6,7,7a,8,10,11-octahydrooxazolo[2,3- j][1,6]naphthyridin-5-one Example 5 was obtained from intermediate 24 and 1,2- dichloro-4-(chloromethyl)benzene using general protocol 5; Purification by flash chromatography column over silica (cyclohexane/ethyl acetate: 100/0 to 30/70); Yield: 39%; [ES+ MS] m/z 397 (MH⁺). |
| 6 | 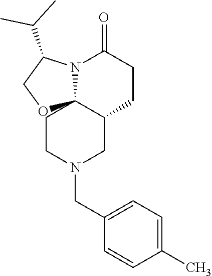 [SMILES: Cc1ccc(CN2CC[C@]34OC[C@H](C(C)C)N3C(=O)CC[C@@H]4C2)cc1] | (3S,7aR,11aR)-3-isopropyl-9-(p-tolylmethyl)- 2,3,6,7,7a,8,10,11-octahydrooxazolo[2,3- j][1,6]naphthyridin-5-one Example 6 was obtained from intermediate 24 and 1- (bromomethyl)-4-methyl-benzene using general protocol 5; Purification by flash chromatography column over silica (cyclohexane/ethyl acetate: 100/0 to 60/40); Yield: 28%; white powder; ¹H NMR (300 MHz, CDCl₃): δ 7.19 (d, J = 8.0 Hz, 2H), 7.11 (d, J = 8.0 Hz, 2H), 4.12-4.04 (m, 1H), 3.97 (dd, J = 8.6 Hz, 7.6 Hz, 1H), 3.76 (dd, J = 8.6 Hz, 6.2 Hz, 1H), 3.50 (d, J = 13.1 Hz, 1H), 3.36 (d, J = 13.1 Hz, 1H), 2.77-2.62 (m, 2H), 2.62-2.50 (m, 1H), 2.46-2.34 (m, 3H), 2.30 (s, 3H), 2.24-2.13 (m, 1H), 2.07-1.88 (m, 2H), 1.84-1.71 (m, 1H), 1.70-1.54 (m, 2H), 0.94 (d, J = 6.8 Hz, 3H), 0.89 (d, J = 6.8 Hz, 3H) ppm. ¹³C NMR (75 MHz, CDCl₃): δ 170.3, 136.7, 135.9, 129.0, 128.7, 92.8, 66.2, 62.3, 61.3, 54.8, 50.7, 40.5, 32.5, 32.4, 30.9, 22.0, 21.2, 19.9, 18.7 ppm. HRMS |
| | | (ESI, m/z): [M + H]⁺ calcd. for C₂₁H₃₁N₂O₂, 343.2386; found |
| | | 343.2398. |
| 7 | 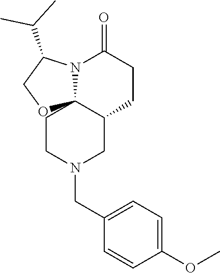 | (3S,7aR,11aR)-3-isopropyl-9-[(4- methoxyphenyl)methyl]-2,3,6,7,7a,8,10,11- octahydrooxazolo[2,3-j][1,6]naphthyridin-5-one Example 7 was obtained from intermediate 24 and 1- (bromomethyl)-4-methoxy-benzene using general protocol 5; Purification by flash chromatography column over silica (cyclohexane/ethyl acetate: 100/0 to 60/40); Yield: 45%; colorless oil. ¹H NMR (300 MHz, CDCl₃): δ 7.21 (d, J = 8.6 Hz, 2H), 6.85 (d, J = 8.6 Hz, 2H), 4.14-4.03 (m, 1H), 3.98 (dd, J = 8.6 Hz, 7.6 Hz, 1H), 3.80 (s, 3H), 3.79-3.73 (m, 1H), 3.48 (d, J = 13.0 Hz, 1H), 3.34 (d, J = 13.0 Hz, 1H), 2.76- 2.63 (m, 2H), 2.61-2.51 (m, 1H), 2.45-2.28 (m, 3H), 2.24- 2.13 (m, 1H), 2.07-1.89 (m, 2H), 1.82-1.71 (m, 1H), 1.70- 1.54 (m, 2H), 0.94 (d, J = 6.9 Hz, 3H), 0.90 (d, J = 6.8 Hz, 3H) ppm. ¹³C NMR (75 MHz, CDCl₃): δ 170.3, 158.8, 130.9, 129.9, 113.7, 92.8, 66.2, 61.9, 61.3, 55.4, 54.7, 50.6, 40.4, 32.5, 32.4, 30.9, 22.0, 19.9, 18.7 ppm. HRMS (ESI, m/z): |
| | | [M + H]⁺ calcd. for C₂₁H₃₁N₂O₃, 359.2335; found 359.2314. |
| 9 | 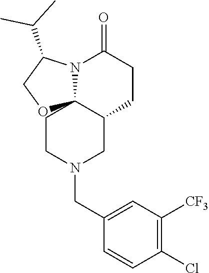 [SMILES: CC(C)[C@H]1CO[C@]23CCN(Cc4ccc(Cl)c(C(F)(F)F)c4)C[C@H]2CCC(=O)N13] | (3S,7aR,11aR)-9-[4-chloro-3- (trifluoromethyl)phenyl]methyl]-3-isopropyl- 2,3,6,7,7a,8,10,11-octahydrooxazolo[2,3- j][1,6]naphthyridin-5-one Example 9 was obtained from intermediate 24 and 4- (bromomethyl)-1-chloro-2-(trifluoromethyl)benzene using general protocol 5; Purification by flash chromatography over silica gel (cyclohexane/ethyl acetate: 100/0 to 0/100); [ES+ MS] m/z 431 (MH⁺). |
| 11 | 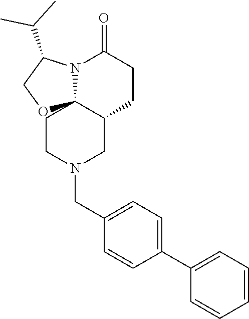 [SMILES: CC(C)[C@H]1CO[C@]23CCN(Cc4ccc(-c5ccccc5)cc4)C[C@H]2CCC(=O)N13] | (3S,7aR,11aR)-3-isopropyl-9-[(4-phenylphenyl)methyl]- 2,3,6,7,7a,8,10,11-octahydrooxazolo[2,3- j][1,6]naphthyridin-5-one Example 11 was obtained from intermediate 24 and 4- (bromomethyl)-1-(bromomethyl)-4-phenyl-benzene using general protocol 5; Purification by flash chromatography over silica gel (cyclohexane/ethyl acetate: 100/0 to 30/70); [ES+ MS] m/z 405 (MH⁺). |
| 12 | 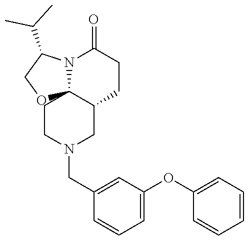 [SMILES: CC(C)[C@H]1CO[C@]23CCN(Cc4cccc(Oc5ccccc5)c4)C[C@H]2CCC(=O)N13] | (3S,7aR,11aR)-3-isopropyl-9-[(3- phenoxyphenyl)methyl]-2,3,6,7,7a,8,10,11- octahydrooxazolo[2,3-j][1,6]naphthyridin-5-one Example 12 was obtained from intermediate 24 and 1- (chloromethyl)-3-phenoxy-benzene using general protocol 5; Purification by flash chromatography over silica gel (cyclohexane/ethyl acetate: 100/0 to 0/100); [ES+ MS] m/z 421 (MH⁺). |
| 16 | 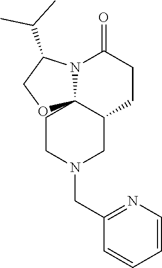 [SMILES: CC(C)[C@H]1CO[C@]23CCN(Cc4ccccn4)C[C@H]2CCC(=O)N13] | (3S,7aR,11aR)-3-isopropyl-9-(2-pyridylmethyl)- 2,3,6,7,7a,8,10,11-octahydrooxazolo[2,3- j][1,6]naphthyridin-5-one Example 16 was obtained from intermediate 24 and 2- (bromomethyl)pyridine; hydrobromide using general protocol 5; Purification by flash chromatography column over silica (DCM/MeOH: 100/0 to 90/10); Yield: 55%; [ES+ MS] m/z 330 (MH⁺). |
| 17 | 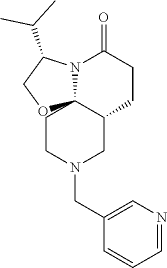 [SMILES: CC(C)[C@H]1CO[C@]23CCN(Cc4cccnc4)C[C@H]2CCC(=O)N13] | (3S,7aR,11aR)-3-isopropyl-9-(3-pyridylmethyl)- 2,3,6,7,7a,8,10,11-octahydrooxazolo[2,3- j][1,6]naphthyridin-5-one Example 16 was obtained from intermediate 24 and 3- (bromomethyl)pyridine; hydrobromide using general protocol 5; Purification by flash chromatography column over silica (DCM/MeOH: 100/0 to 95/05); Yield: 50%; [ES+ MS] m/z 330 (MH⁺). |
| 18 | 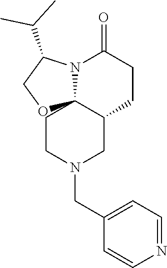 [SMILES: CC(C)[C@H]1CO[C@]23CCN(Cc4ccncc4)C[C@H]2CCC(=O)N13] | (3S,7aR,11aR)-3-isopropyl-9-(4-pyridylmethyl)- 2,3,6,7,7a,8,10,11-octahydrooxazolo[2,3- j][1,6]naphthyridin-5-one Example 18 was obtained from intermediate 24 and 4- (bromomethyl)pyridine; hydrobromide using general protocol 5; Purification by flash chromatography column over silica (DCM/MeOH: 100/0 to 95/05); Yield: 31%; ¹H NMR (300 MHz, CD₂Cl₂): δ 8.56 (d, J = 6.5 Hz, 2H), 7.27 (d, J= 6.5 Hz, 2H), 4.00 (m, 2H), 3.75 (m, 1H), 3.53 (d, J = 13.6 Hz, 1H), 3.42 (d, J = 13.6 Hz, 1H), 2.68 (m, 2H), 2.52 (m, 2H), 2.30 (m, 3H), 1.97 (m, 2H), 1.70 (m, 3H), 0.91 (d, J = 7.0 Hz, 3H), 0.88 (d, J= 7.0 Hz, 3H) ppm. ¹³C NMR (75 MHz, CD₂Cl₂): δ 170.0, 150.0, 148.7, 123.9, 92.6, 66.4, 61.5, 55.4, 51.1, 40.8, 32.8, 32.6, 31.1, 22.3, 20.0, 18.8 ppm. HRMS (ESI, m/z): [M + H]⁺ calcd. for C₁₉H₂₇N₃O₂, 330.2182; found 330.2180. |
| 21 | 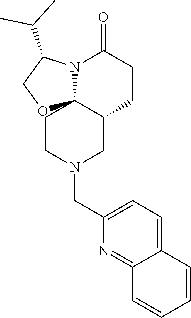 [SMILES: CC(C)[C@H]1CO[C@]23CCN(Cc4ccc5ccccc5n4)C[C@H]2CCC(=O)N13] | (3S,7aR,11aR)-3-isopropyl-9-(2-quinolylmethyl)- 2,3,6,7,7a,8,10,11-octahydrooxazolo[2,3- j][1,6]naphthyridin-5-one Example 21 was obtained from intermediate 24 and 2- (bromomethyl)quinoline using general protocol 5; Purification by flash chromatography over silica gel (cyclohexane/ethyl acetate: 100/0 to 0/100); [ES+ MS] m/z 380 (MH⁺). |
| 22 | 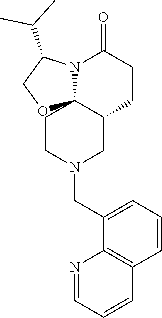 [SMILES: CC(C)[C@H]1CO[C@]23CCN(Cc4cccc5cccnc45)C[C@H]2CCC(=O)N13] | (3S,7aR,11aR)-3-isopropyl-9-(8-quinolylmethyl)- 2,3,6,7,7a,8,10,11-octahydrooxazolo[2,3- j][1,6]naphthyridin-5-one Example 22 was obtained from intermediate 24 and 8- (bromomethyl)quinoline using general protocol 5; Purification by flash chromatography over silica gel (cyclohexane/ethyl acetate: 100/0 to 0/100); [ES+ MS] m/z 380 (MH⁺). |
| 29 | 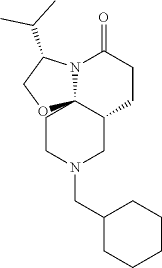 [SMILES: CC(C)[C@H]1CO[C@]23CCN(CC4CCCCC4)C[C@H]2CCC(=O)N13] | (3S,7aR, 11aR)-9-(cyclohexylmethyl)-3-isopropyl- 2,3,6,7,7a,8,10,11-octahydrooxazolo[2,3- j][1,6]naphthyridin-5-one Example 29 was obtained from intermediate 24 and bromomethylcyclohexane using general protocol 5; Purification by flash chromatography over silica gel (cyclohexane/ethyl acetate: 100/0 to 0/100);; Yield: 67%; [ES+ MS] m/z 335 (MH⁺). |
| 30 | 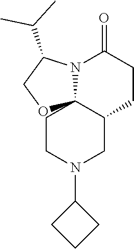 [SMILES: CC(C)[C@H]1CO[C@]23CCN(C4CCC4)C[C@H]2CCC(=O)N13] | (3S,7aR,11aR)-9-cyclobutyl-3-isopropyl- 2,3,6,7,7a,8,10,11-octahydrooxazolo[2,3- j][1,6]naphthyridin-5-one Example 30 was obtained from intermediate 24 and bromocyclobutane using general protocol 5; Purification by flash chromatography over silica gel (cyclohexane/ethyl acetate: 100/0 to 0/100); [ES+ MS] m/z 293 (MH⁺). |
| 31 | 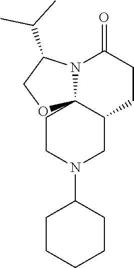 [SMILES: CC(C)[C@H]1CO[C@]23CCN(C4CCCCC4)C[C@H]2CCC(=O)N13] | (3S,7aR,11aR)-9-cyclohexyl-3-isopropyl- 2,3,6,7,7a,8,10,11-octahydrooxazolo[2,3- j][1,6]naphthyridin-5-one Example 31 was obtained from intermediate 24 and iodocyclohexane using general protocol 5; Purification by flash chromatography over silica gel (cyclohexane/ethyl acetate: 100/0 to 0/100); [ES+ MS] m/z 321 (MH⁺). |
| 33 | 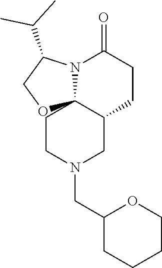 [SMILES: CC(C)[C@H]1CO[C@]23CCN(CC4CCCCO4)C[C@H]2CCC(=O)N13] | (3S,7aR,11aR)-3-isopropyl-9-(tetrahydropyran-2- ylmethyl)-2,3,6,7,7a,8,10,11-octahydrooxazolo[2,3- j][1,6]naphthyridin-5-one Example 31 was obtained from intermediate 24 and 2- (bromomethyl)tetrahydropyrane using general protocol 5; Purification by flash chromatography column (DCM/MeOH: 100/0 to 98/02); Yield: 56%; [ES+ MS] m/z 337 (MH⁺). |
| 34 | 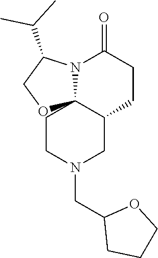 [SMILES: CC(C)[C@H]1CO[C@]23CCN(CC4CCCO4)C[C@H]2CCC(=O)N13] | (3S,7aR, 11aR)-3-isopropyl-9-(tetrahydrofuran-2- ylmethyl)-2,3,6,7,7a,8,10,11-octahydrooxazolo[2,3- j][1,6]naphthyridin-5-one Example 34 was obtained from intermediate 24 and 2- (bromomethyl)tetrahydrofurane using general protocol 5; Purification by flash chromatography column (DCM/MeOH: 100/0 to 98/2); Yield: 31%; [ES+ MS] m/z 323 (MH⁺). |
| 37 | 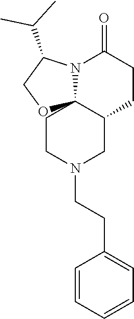 [SMILES: CC(C)[C@H]1CO[C@]23CCN(CCc4ccccc4)C[C@H]2CCC(=O)N13] | (3S,7aR,11aR)-3-isopropyl-9-(2-phenylethyl)- 2,3,6,7,7a,8,10,11-octahydrooxazolo[2,3- j][1,6]naphthyridin-5-one Example 37 was obtained from intermediate 24 and 2- bromoethylbenzene using general protocol 5; Purification by flash chromatography over silica gel (cyclohexane/ethyl acetate: 100/0 to 0/100);; Yield: 78%; [ES+ MS] m/z 343 (MH⁺). |
| 40 | 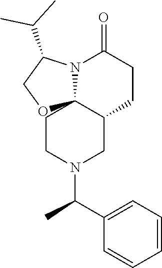 [SMILES: CC(C)[C@H]1CO[C@]23CCN([C@H](C)c4ccccc4)C[C@H]2CCC(=O)N13] | (3S,7aR,11aR)-3-isopropyl-9-[(1R)-1-phenylethyl]- 2,3,6,7,7a,8,10,11-octahydrooxazolo[2,3- j][1,6]naphthyridin-5-one + (3S,7aR, 11aR)-3-isopropyl-9-[(1S)-1-phenylethyl]- 2,3,6,7,7a,8,10,11-octahydrooxazolo[2,3- j][1,6]naphthyridin-5-one Example 40 was obtained from intermediate 24 and 1- bromoethylbenzene using general protocol 5; Purification by flash chromatography over silica gel (cyclohexane/ethyl acetate: 100/0 to 0/100);; Yield: 81%; yellow oil; [ES+ MS] m/z 343 (MH⁺). |
| | 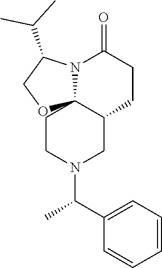 [SMILES: CC(C)[C@H]1CO[C@]23CCN([C@@H](C)c4ccccc4)C[C@H]2CCC(=O)N13] | |

### Synthesis of Aldehyde for Use in Reductive Amination Reaction (General Protocol 8)

General Protocol 6: Oxidation of Alcohol into Aldehyde

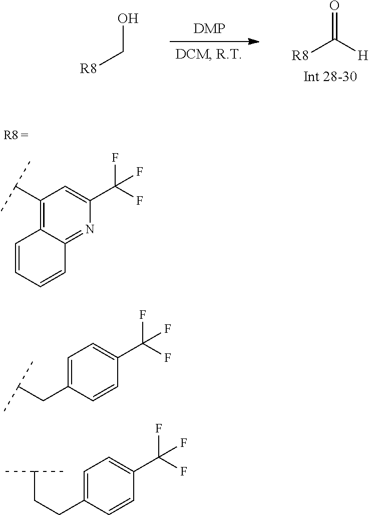

The appropriate alcohol (1 eq) was dissolved in DCM. Then DMP (1 eq) was added. The mixture was stirred until completion of the reaction. Treatment depends of the substituents

### General Protocol 7: Synthesis of Aldehyde Via Nucleophilic Aromatic Substitution

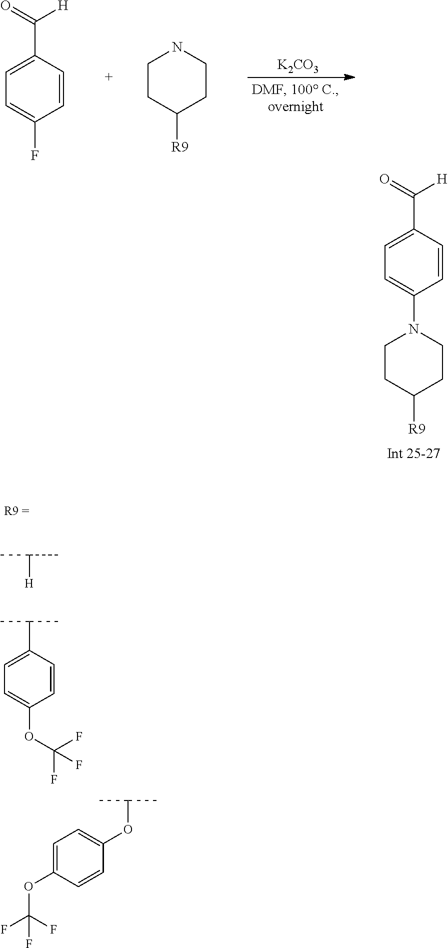

The appropriate amine (1 eq) and K₂CO₃ (1 eq) was dissolved in DMF. Then 4-fluorobenzaldehyde (1 eq) was added dropwise. The mixture was stirred at 100° C. overnight. The solvent was removed under reduced pressure; retreatment depend of the substituents.

| Inter- | | |
|---|---|---|
| mediates | Structures | Physichal data |
| 25 | 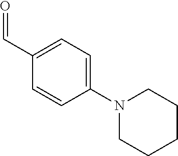 [SMILES: O=Cc1ccc(N2CCCCC2)cc1] | 4-(1-piperidyl)benzaldehyde Intermediate 25 was obtained from 4-fluorobenzaldehyde and piperidine following the general protocol 7. [ES+ MS] m/z 190 (MH⁺). |
| 26 | 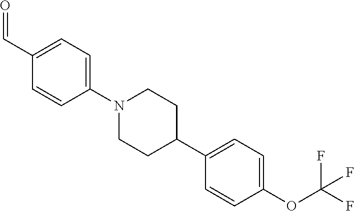 [SMILES: O=Cc1ccc(N2CCC(c3ccc(OC(F)(F)F)cc3)CC2)cc1] | 4-[4-[4-(trifluoromethoxy)phenyl]-1- piperidyl]benzaldehyde Intermediate 26 was obtained from 4-fluorobenzaldehyde and 4-[4-(trifluoromethoxy)phenyl]piperidine following the general protocol 7. Yield: 77%; [ES+ MS] m/z 350 (MH+). |
| 27 | 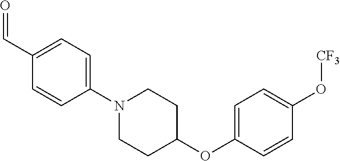 [SMILES: O=Cc1ccc(N2CCC(Oc3ccc(OC(F)(F)F)cc3)CC2)cc1] | 4-[4-[4-(trifluoromethoxy)phenoxy]-1- piperidyl]benzaldehyde Intermediate 27 was obtained from 4-fluorobenzaldehyde and 4-[4-(trifluoromethoxy)phenoxy]piperidine following the general protocol 7. [ES+ MS] m/z 366 (MH⁺). |
| 28 | 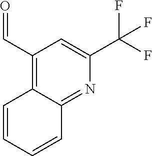 [SMILES: O=Cc1cc(C(F)(F)F)nc2ccccc12] | 2-(trifluoromethyl)quinoline-4-carbaldehyde Intermediate 28 was obtained from [2-(trifluoromethyl)-4- quinolyl]methanol following the general protocol 6. The reaction was heated at 60° C. After completion of the reaction water was added and extracted 4 times with DCM; organic layers were pooled, washed with brine, dried over MgSO₄, filtered and evaporated under vacuum. Yield: 94%; ¹H NMR (300 MHz, CDCl₃): δ 10.6 (s, 1H), 9.09 (dd, J = 1.4 Hz, J = 8.6 Hz, 1H), 8.36 (dd, J = 1.4 Hz, J = 8.4 Hz, 1H), 7.96 (ddd, J = 1.6 Hz, J = 7.0 Hz, J = 8.4 Hz, 1H), 7.90 (ddd, |
| | | J = 1.6 Hz, J = 7.0 Hz, J = 8.3 Hz, 1H) ppm. |
| 29 | 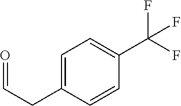 [SMILES: O=CCc1ccc(C(F)(F)F)cc1] | 2-[4-(trifluoromethyl)phenyl]acetaldehyde Intermediate 29 was obtained from 2-[4- (trifluoromethyl)phenyl]ethanol following the general protocol 6. After completion of the reaction the crude was purified through flash chromatography (from cyclohexane/ethyl actetate 100/0 to 50/50). Yield = 25%; ¹H NMR (300 MHz, |
| | | CDCl₃): δ 9.80 (t, J = 2.0 Hz, 1H), 7.65 (d, J = 8.2 Hz, 2H), |
| | | 7.36 (d, J = 8.2 Hz, 2H), 3.81 (d, J = 1.5 Hz, 2H) ppm. |
| 30 | 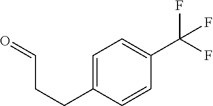 [SMILES: O=CCCc1ccc(C(F)(F)F)cc1] | 3-[4-(trifluoromethyl)phenyl]propanal Intermediate 30 was obtained from 3-[4- (trifluoromethyl)phenyl]propan-1-ol following the general protocol 6. After completion of the reaction. The crude was washed twice with an aqueous solution saturated with NaHCO₃, once with brine, dried over MgSO₄, filtered and evaporated under |
| | | reduced pressure. The crude was then purified through flash |
| | | chromatography (from cyclohexane/ethyl acetate 100/0 to |
| | | 70/30). Yield: 54%; ¹H NMR (300 MHz, CDCl₃): δ 9.84 (t, J= |
| | | 1.2 Hz, 1H), 7.57 (d, J = 8.0 Hz, 2H), 7.33 (d, J = 8.0 Hz, 2H), |
| | | 3.0 (t, J = 7.4 Hz, 2H), 2.84 (tt, J = 1.2 Hz, J = 7.4 Hz, 2H) |
| | | ppm. |

### General Protocol 8: Synthesis of Lactams Via Reductive Amination

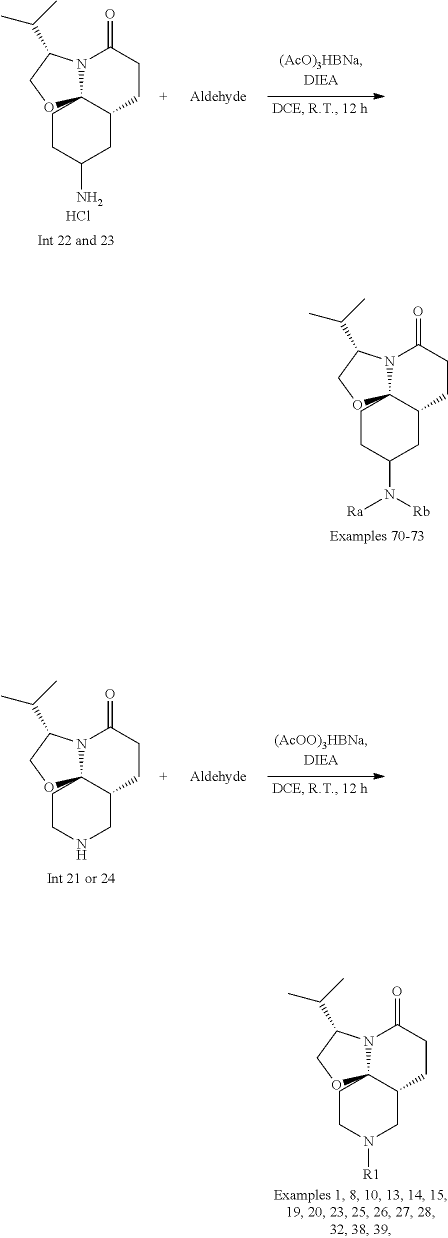

Appropriate carbonyl (ketone or aldehyde) (1 eq) was dissolved in 1,2-dichlorethane. Then the appropriate intermediate (21, 22, 23 or 24) (1-3 eq), DIEA or K₂CO₃ (2 eq), CH₃CO₂H (0.1-1 eq), and (CH₃COO)₃BHNa (3 eq) were added. The mixture was stirred (RT or reflux) overnight. The work-up was dependent on the substituent.

| Ex- | | |
|---|---|---|
| amples | Structure | Physical data |
| 15 | 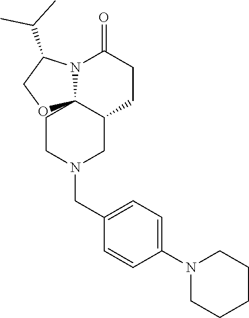 [SMILES: CC(C)[C@H]1CO[C@]23CCN(Cc4ccc(N5CCCCC5)cc4)C[C@H]2CCC(=O)N13] | (3S,7aR,11aR)-3-isopropyl-9-[[4-(1- piperidyl)phenyl]methyl]-2,3,6,7,7a,8,10,11- octahydrooxazolo[2,3-j][1,6]naphthyridin-5-one Example 15 was obtained from intermediate 21 and intermediate 25 using general protocol 8. The crude was evaporated under reduced pressure and purified through flash chromatography over silica gel (cyclohexane/ethyl acetate: 100/0 to 30/70); Yield: 77%; ¹H NMR (300 MHz, CDCl₃): δ 7.18 (d, J = 8.7 Hz, 2H), 6.90 (d, J = 8.7 Hz, 2H), 4..11 (m, 1H), 3.99 (dd, J = 7.6 Hz, J = 8.7 Hz, 1H); 3.78 (dd, J = 6.3 Hz, J = 8.7 Hz, 1H), 3.50 (d, J = 13.0 Hz, 1H), 3.34 (d, J = 13.0 Hz, 1H), 3.18-3.12 (m, 4H), 2.82-2.66 (m, 2H), 2.64-2.52 (m, 1H), 2.49-2.12 (m, 5H), 2.10-1.88 (m, 2H), 1.85-1.53 (m, 9H) 0.96 (d, J = 6.9 Hz, 3H), 0.92 (d, J = 6.9 Hz, 3H) ppm. ¹³C NMR (75 MHz, CDCl₃): δ 170.2, 151.3, 129.5, 116.2, 92.7, 66.1, 61.9, 61.2, 54.5, 50.8, 50.4, 40.3, 32.3, 30.8, 25.9, 24.3, 21.8, 18.8, 18.6 ppm. |
| 14 | 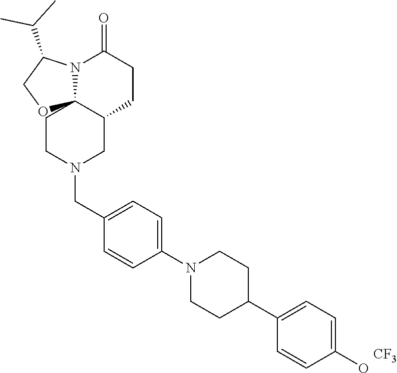 | (3S,7aR,11aR)-3-isopropyl-9-[[4-[4-[4- (trifluoromethoxy)phenyl]-1-piperidyl]phenyl]methyl]- 2,3,6,7,7a,8,10,11-octahydrooxazolo[2,3- j][1,6]naphthyridin-5-one Example 14 was obtained from intermediate 21 and intermediate 26 using general protocol 8. The crude was evaporated under reduced pressure and purified through flash chromatography column over silica gel (cyclohexane/ethyl acetate: 100/0 to 30/70); Yield: 53%; ¹H NMR (300 MHz, CDCl₃): δ 7.31-7.25 (m, 2H), 7.24-7.16 (m, 4H), 6.95 (d, J = 8.7 Hz, 2H), 4.16-4.08 (m, 1H), 4.00 (dd, J = 7.4 Hz, J = 8.6 Hz, 1H), 3.83-3.75 (m, 3H), 3.50 (d, J = 13.0 Hz, 1H), 3.36 (d, J = 13.1 Hz, 1H), 2.89-2.53 (m, 6H), 2.49-2.29 (m, 3H), 2.28-2.15 (m, 1H), 2.11-1.75 (m, 8H), 1.74-1.56 (m, 2H), 0.97 (d, J = 6.9 Hz, 3H), 0.92 (d, J = 6.9 Hz, 3H) ppm. ¹³C NMR (75 MHz, CDCl₃): δ 170.2, 150.8, 147.6 (q, J = 1.8 Hz), 144.7, 129.8, 129.5, 128.0, 121.0, 120.5 (q, J = 256 Hz), 116.4, 92.7, 66.1, 61.9, 61.2, 54.6, 50.6, 50.5, 50.5, 41.9, 40.3, 33.4, 32.3, 32.3, 30.8, 21.9, 19.8, 18.6 ppm. |
| 13 | 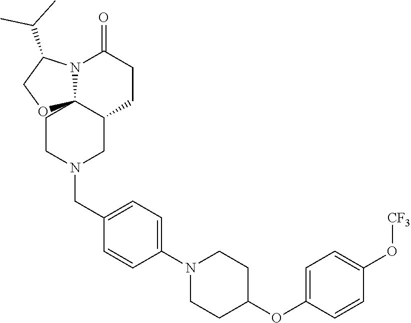 [SMILES: CC(C)[C@H]1CO[C@]23CCN(Cc4ccc(N5CCC(Oc6ccc(OC(F)(F)F)cc6)CC5)cc4)C[C@H]2CCC(=O)N13] | (3S,7aR,11aR)-3-isopropyl-9-[[4-[4-[4- (trifluoromethoxy)phenoxy]-1-piperidyl]phenyl]methyl]- 2,3,6,7,7a,8,10,11-octahydrooxazolo[2,3- j][1,6]naphthyridin-5-one Example 13 was obtained from intermediate 21 and intermediate 27 using general protocol 8. The crude was evaporated under reduced pressure and purified through reverse flash chromatography (MeCN/water 10/90 to 100/0). pure fraction were lyophilized; Yield: 81%; ¹H NMR (300 MHz, CDCl₃): δ 7.21 (d, J = 8.6 Hz, 2H), 7.17 (d, J = 8.7 Hz, 2H), 6.96-6.89 (m, 4H), 4.45 (sept, J = 3.7 Hz, 1H), 4.16-4.05 (m, 1H), 4.00 (t, J = 7.7 Hz, 1H), 3.79 (dd, J = 6.4 Hz, J = 8.8 Hz, 1H), 3.56-3.45 (m, 3H), 3.35 (d, J = 12.9 Hz, 1H), 3.11 (ddd, J = 3.4 Hz, 8.2 Hz, J = 11.7 Hz, 2H), 2.81-2.52 (m, 3H), 2.50-2.28 (m, 3H), 2.26-2.06 (m, 3 H), 2.0-1.88 (m, 4H), 1.80 (d, J = 13.6 Hz, 1H), 1.73-1.56 (m, 4H), 0.96 (d, J = 6.9 Hz, 3H), 0.92 (d, J = 6.9 Hz, 3H) ppm.¹³C NMR (75 MHz, CDCl₃): δ 170.2, 155.8, 150.31, 142.8, 129.8, 129.6, 122.5, 120.5 (d, J = 256 Hz), 116.8, 116.3, 92.7, 72.9, 66.1, 61.9, 61.2, 54.6, 50.5, 46.8, 40.3, 32.3, 32.3, 30.8, 30.5, |
| | | 21.9, 19.8, 18.6 ppm. |
| 38 | 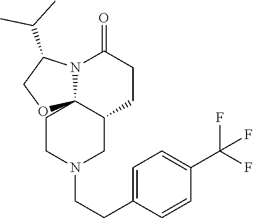 [SMILES: CC(C)[C@H]1CO[C@]23CCN(CCc4ccc(C(F)(F)F)cc4)C[C@H]2CCC(=O)N13] | (3S,7aR,11aR)-3-isopropyl-9-[2-[4- (trifluoromethyl)phenyl]ethyl]-2,3,6,7,7a,8,10,11- octahydrooxazolo[2,3-j][1,6]naphthyridin-5-one Example 38 was synthetized from intermediate 21 and intermediate 29 using general protocol 8; The crude was dissolved in DCM and washed twice with an aqueous solution of saturated NaHCO₃, once with brine, dried over MgSO₄, filtered and evaporated under reduced pressure. The residue was purified through flash chromatography (DCM/MeOH: 100/0 to 95/05). Yield: 31%; ¹H NMR (300 MHz, CDCl₃): δ 7.55 (d, J = 8.1 Hz, 2H), 7.33 (d, J = 8.1 Hz, 2H), 4.16 - 4.06 (m, 1H), 4.00 (t, J = 8.6 Hz, 1H), 3.79 (dd, J = 6.2 Hz, J = 8.2 Hz, 1H), 2.88-2.73 (m, 4H), 2.70-2.50 (m, 4H), 2.49-2.34 (m, 1H), 2.34-2.15 |
| | | (m, 2H), 2.10-1.95 (m, 1H), 1.91 (dd, J = 4.6 Hz, J = 12.9 |
| | | Hz), 1.86-1.57 (m, 4H), 0.96 (d, J = 6.9 Hz, 3H), 0.92 (d, J = |
| | | 6.9 Hz, 3H) ppm. ¹³C NMR (75 MHz, CDCl₃): δ 170.1, 144.7, |
| | | 129.0, 128.3 (q, J = 32 Hz), 125.2 (q, J = 4 Hz), 124.0 (q, J = |
| | | 272 Hz), 92.4, 66.1, 61.2, 59.3, 55.0, 50.6, 40.3, 33.5, 32.3, |
| | | 32.2, 30.8, 22.0, 19.8, 18.6 ppm. |
| 39 | 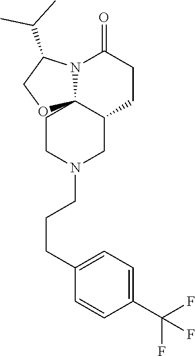 [SMILES: CC(C)[C@H]1CO[C@]23CCN(CCCc4ccc(C(F)(F)F)cc4)C[C@H]2CCC(=O)N13] | (3S,7aR,11aR)-3-isopropyl-9-[3-[4- (trifluoromethyl)phenyl]propyl]-2,3,6,7,7a,8,10,11- octahydrooxazolo[2,3-j][1,6]naphthyridin-5-one Example 39 was synthetized from intermediate 21 and intermediate 30 using general protocol 8; The crude was dissolved in DCM and washed twice with an aqueous solution of saturated NaHCO₃, once with brine, dried over MgSO4, filtered and evaporated under reduced pressure. The crude was then purified through flash chromatography (DCM/ethyl acetate: 100/0 to 70/30); Fractions containing the desired product were pooled, then repurified through flash chromatography (DCM/Acetone 100:0 to 60/40); Yield: 19%; ¹H NMR (300 MHz, CDCl₃): δ 7.55 (d, J = 8.1 Hz, 2H), 7.31 (d, J = 8.1 Hz, 2H), 4.17 - 4.06 (m, 1H), 4.01 (dd, J = 7.4 Hz, J = 8.5 Hz, 1H), 3.78 (dd, J = 6.3 Hz, J = 8.6 Hz, 1H), 2.81- 2.68 (m, 4H), 2.65-2.54 (m, 1H), 2.51-2.13 (m, 8H), 2.09- 1.91 (m, 2H), 1.88 - 1.61 (m, 5H), 0.97 (d, J = 6.9 Hz, 3H), 0.93 (d, J = 6.9 Hz, 3H) ppm. ¹³C NMR (75 MHz, CDCl₃): δ 170.1, 146.2, 128.8, 128.2 (q, J = 32 Hz), 125.2 (q, J = 3.5 Hz), 124.4 (q, J = 273 Hz), 92.4, 66.2, 61.2, 56.8, 54.8, 50.5, 40.2, 33.2, 32.4, 32.1, 30.8, 28.2, 22.1, 19.8, 18.6 ppm. |
| 25 | 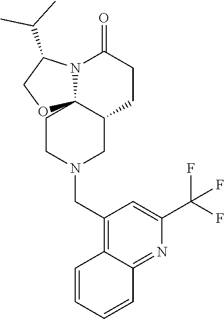 [SMILES: CC(C)[C@H]1CO[C@]23CCN(Cc4cc(C(F)(F)F)nc5ccccc45)C[C@H]2CCC(=O)N13] | (3S,7aR,11aR)-3-isopropyl-9-[[2-(trifluoromethyl)-4- quinolyl]methyl]-2,3,6,7,7a,8,10,11- octahydrooxazolo[2,3-j][1,6]naphthyridin-5-one Example 25 was synthetized from intermediate 21 and intermediate 28 using general protocol 8; An aqueous solution of NaHCO₃ was added, then the mixture was extracted three times with DCM. Organic layers were pooled, washed with brine, dried over MgSO₄, filtered and evaporated under reduced pressure. The residue was purified through reverse flash chromatography (MeCN/water: 10/90 to 100/0). Pure fractions were lyophilized; Yield: 69%. ¹H NMR (300 MHz, CDCl₃): δ 8.30-8.24 (m, 2H), 7.87-7.78 (m, 2H), 7.69 (ddd, J = 1.2 Hz, J = 6.9 Hz, J = 8.3 Hz, 1H), 4.17-4.08 (m, 1H), 4.06-3.93 (m, 3H), 3.81 (dd, J = 6.1 Hz, J = 8.6 Hz, 1H), 2.84-2.55 (m, 4H), 2.49-2.27 (m, 3H), 2.09-1.95 (m, 2H), 1.85 (dq, J = 2.6 Hz, J = 13.7 Hz, 1H), 1.76-1.61 (m, 2H), 0.96 (d, J = 6.9 Hz, 3H), 0.92 (d, J = 6.7 Hz, 3H) ppm. ¹³C NMR (75 MHz, CDCl₃): δ 170.0, 147.7 (q, J = 34 Hz), 147.4, 147.1, 130.8, 130.5, 128.3, 128.1, 123.9, 121.6 (q, J = 275 Hz), 116.5 (q, J = 2.4 Hz), 92.1, 66.2, 61.2, |
| | | 59.2, 55.3, 51.0, 40.3, 32.4, 32.2, 30.7, 21.9, 19.8, 18.6 ppm. |
| 23 | 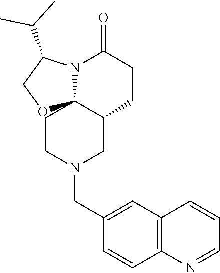 [SMILES: CC(C)[C@H]1CO[C@]23CCN(Cc4ccc5ncccc5c4)C[C@H]2CCC(=O)N13] | (3S,7aR,11aR)-3-isopropyl-9-(6-quinolylmethyl)- 2,3,6,7,7a,8,10,11-octahydrooxazolo[2,3- j][1,6]naphthyridin-5-one Example 23 was synthetized from intermediate 21 and quinoline-6-carbaldehyde using general protocol 8; Yield: 61%; ¹H NMR (300 MHz, CDCl₃): δ 8.92 (s, 1H), 8.16 (d, J = 8.0 Hz, 1H), 8.09 (d, J = 8.1 Hz, 1H), 7.85-7.66 (m, 2H), 7.42 (dd, J = 3.7 Hz, J = 7.4 Hz, 1H), 4.17-4.08 (m, 1H), 4.01 (dd, J = 7.4 Hz, J = 8.6 Hz, 1H), 3.85-3.56 (m, 3H), 2.90-2.53 (m, 4H), 2.45-2.23 (m, 3H), 2.09-1.94 (m, 2H), 1.84 (d, J = 12.0 Hz, 1H), 1.77-1.59 (m, 5H), 0.96 (d, J = 6.8 Hz, 3H), 0.92 (d, J = 6.8 Hz, 3H) ppm. ¹³C NMR (75 MHz, CDCl₃): δ 170.1, 150.1, 147.8, 137.2, 135.8, 130.5, 129.4, 128.1, 126.9, 121.2, 92.4, 66.1, 62.2, 61.2, 54.8, 50.7, 40.3, 32.3, 32.2, 30.7, 21.9, 19.8, 18.6. |
| 72 | 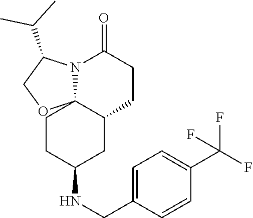 [SMILES: CC(C)[C@H]1CO[C@]23CC[C@H](NCc4ccc(C(F)(F)F)cc4)C[C@H]2CCC(=O)N13] | (3S,7aR,9S,11aR)-3-isopropyl-9-[[4- (trifluoromethyl)phenyl]methylamino]-3,6,7,7a,8,9,10,11- octahydro-2H-oxazolo[2,3-j]quinolin-5-one Example 72 was synthetized from intermediate 22 and 4- trifluoromethylbenzaldehyde using general protocol 8; The crude was evaporated under reduced pressure and purified through flash chromatography (DCM/MeOH: 100/0 to 90/10). Yield: 31%; ¹H NMR (300 MHz, CDCl₃): δ 7.60 (d, J = 8.0 Hz, 2H), 7.48 (d, J = 8.0 Hz, 2H), 4.14-4.04 (m, 1H), 4.0 (dd, J = 7.5 Hz, J = 8.4 Hz, 1H), 3.93 (s, 2H), 3.76 (dd, J = 6.2 Hz, J = 8.5 Hz, 1H), 2.83 (dt, J = 4.0 Hz, J = 11.2 Hz, 1H), 2.58 (br dd, J = 6.9 Hz, J = 17.9 Hz, 1H), 2.47-2.32 (m, 1H), 2.09- 1.76 (m, 8H), 1.74-1.61 (m, 2H), 1.56-1.42 (m, 1H), 0.95 (d, J = 6.9 Hz, 3H), 0.91 (d, J = 6.9 Hz, 3H) ppm. ¹³C NMR (75 |
| | | MHz, CDCl₃): δ 170.0, 142.6, 129.7 (q, J = 32 Hz), 128.6, |
| | | 125.5 (q, J = 3.7 Hz), 124.1 (q, J = 274 Hz), 93.4, 66.3, 61.3, |
| | | 50.2, 49.6, 39.8, 34.1, 32.6, 31.0, 30.0, 28.7, 22.7, 19.8, 18.7 |
| | | ppm. |
| 70 | 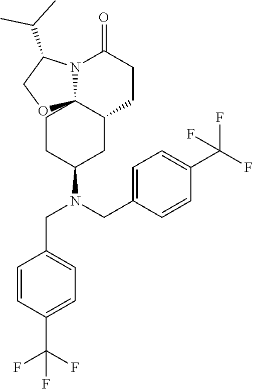 [SMILES: CC(C)[C@H]1CO[C@]23CC[C@H](N(Cc4ccc(C(F)(F)F)cc4)Cc4ccc(C(F)(F)F)cc4)C[C@H]2CCC(=O)N13] | (3S,7aR,9S,11aR)-9-[bis[[4- (trifluoromethyl)phenyl]methyl]amino]-3-isopropyl- 3,6,7,7a,8,9,10,11-octahydro-2H-oxazolo[2,3-j]quinolin-5- one Example 70 was synthetized from intermediate 22 and 4- trifluoromethylbenzaldehyde using general protocol 8; Yield: 13%. ¹H NMR (300 MHz, CDCl₃): δ 7.56 (d, J = 8.1 Hz, 4H), 7.46 (d, J = 8.1 Hz, 4H), 4.13-3.97 (m, 2H), 3.80-3.63 (m, 5H), 2.84-2.70 (m, 1H), 2.52 (ddd, J = 2.2 Hz, J = 7.1 Hz, J = 18.1 Hz, 1H), 2.43-2.29 (m, 1H), 2.13-1.54 (m, 9H), 0.94 (d, J = 7.0 Hz, 3H), 0.91 (d, J = 6.9 Hz, 3H) ppm. ¹³C NMR (75 MHz, CDCl₃): δ 169.9, 144.4, 129.2 (q, J = 32 Hz), 128.4, 125.2 (q, J = 3.7 Hz), 124.2 (q, J = 274 Hz), 93.7, 77.2, 66.4, 61.2, 53.7, 51.7, 40.2, 32.6, 31.0, 30.8, 29.2, 24.6, 22.4, 19.8, 18.7 ppm. |
| 73 | 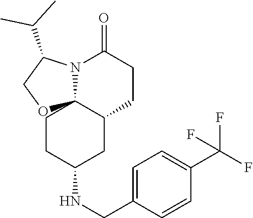 [SMILES: CC(C)[C@H]1CO[C@]23CC[C@@H](NCc4ccc(C(F)(F)F)cc4)C[C@H]2CCC(=O)N13] | (3S,7aR,9R,11aR)-3-isopropyl-9-[4- (trifluoromethyl)phenyl]methylamino]-3,6,7,7a,8,9,10,11- octahydro-2H-oxazolo[2,3-j]quinolin-5-one Example 73 was synthetized from intermediate 23 and 4- trifluoromethylbenzaldehyde using general protocol 8; The crude was evaporated under reduced pressure and purified through flash chromatography (from DCM/MeOH 100/0 to 90/10). Yield: 27%. ¹H NMR (300 MHz, CDCl₃): δ 7.60 (d, J = 8.1 Hz, 2H), 7.46 (d, J = 8.0 Hz, 2H), 4.05-3.96 (m, 1H), 3.93 (br d, J = 4.6 Hz, 2H), 3.89 (s, 2H), 2.89-2.75 (m, 1H), 2.55-2.34 (m, 2H), 2.09-1.71 (m, 8H), 1.51-1.36 (m, 2H), 0.87 (d, J = 7.1 Hz, 3H), 0.72 (d, J = 6.9 Hz, 3H) ppm. ¹³C NMR (75 MHz, CDCl₃): δ, 167.8, 144.0, 129.4 (q, J = 32 Hz), 128.3, 125.4 (q, J = 3.7 Hz), 124.2 (q, J = 274 Hz), 93.2, 62.5, |
| | | 60.4, 50.2, 50.0, 38.0, 34.3, 30.6, 29.3, 28.4, 24.6, 23.0, 19.5, |
| | | 14.7 ppm. |
| 71 | 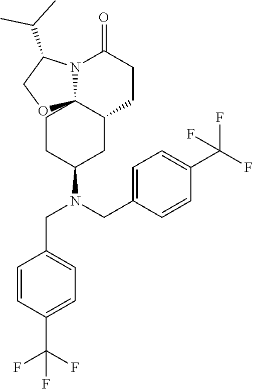 [SMILES: CC(C)[C@H]1CO[C@]23CC[C@H](N(Cc4ccc(C(F)(F)F)cc4)Cc4ccc(C(F)(F)F)cc4)C[C@H]2CCC(=O)N13] | (3S,7aR,9R,11aR)-9-[bis[[4- (trifluoromethyl)phenyl]methyl]amino]-3-isopropyl- 3,6,7,7a,8,9,10,11-octahydro-2H-oxazolo[2,3-j]quinolin-5- one Example 71 was synthetized from intermediate 23 and 4- trifluoromethylbenzaldehyde using general protocol 8; Yield: 15%. ¹H NMR (300 MHz, CDCl₃): δ 7.56 (d, J = 8.0 Hz, 4H), 7.45 (d, J = 8.0 Hz, 4H), 4.03-3.97 (m, 1H), 3.95-3.87 (m, 2H), 3.76-3.61 (m, 4H), 2.99 (d sept, J = 3.0 Hz, J = 6.8 Hz, 1H), 2.89-2.75 (m, 1H), 2.51-2.29 (m, 2H), 2.14-2.00 (m, 2H), 1.98-1.59 (m, 6H), 1.44-1.25 (m, 1H), 0.87 (d, J = 7.1 Hz, 3H), 0.72 (d, J = 6.9 Hz, 3H) ppm. ¹³C NMR (75 MHz, CDCl₃): δ 167.6, 144.4, 129.3 (q, J = 32 Hz), 128.4, 125.2 (q, J = 3.7 Hz), 124.1 (q, J= 274 Hz), 93.2, 77.2, 62.5, 60.4, 53.7, 51.4, 38.2, 30.4, 28.8, 28.6, 24.6, 24.3, 22.6, 19.5, 14.7 ppm. |
| 1 | 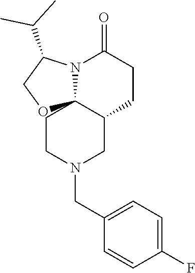 [SMILES: CC(C)[C@H]1CO[C@]23CCN(Cc4ccc(F)cc4)C[C@H]2CCC(=O)N13] | (3S,7aR,11aR)-9-[(4-fluorophenyl)methyl]-3-isopropyl- 2,3,6,7,7a,8,10,11-octahydrooxazolo[2,3- j][1,6]naphthyridin-5-one Example 1 was obtained from intermediate 24 and 4- fluorobenzaldehyde using general protocol 8. A saturated aqueous solution of Na₂CO₃ and DCM were added. The organic layer was dried over MgSO₄ and the solvents were removed under vacuum. The product was then purified by flash column chromatography (cyclohexane/ethyl acetate: 100/0 to 30/70). [ES+ MS] m/z 347 (MH⁺). |
| 8 | 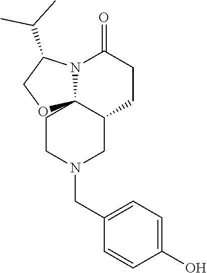 [SMILES: CC(C)[C@H]1CO[C@]23CCN(Cc4ccc(O)cc4)C[C@H]2CCC(=O)N13] | (3S,7aR,11aR)-9-[(4-hydroxyphenyl)methyl]-3-isopropyl- 2,3,6,7,7a,8,10,11-octahydrooxazolo[2,3- j][1,6]naphthyridin-5-one Example 8 was obtained from intermediate 24 and 4- hydroxybenzaldehyde using general protocol 8. The solvent was removed under vacuum, the mixture was then taken up in CH₂Cl₂ and washed with a saturated aqueous solution of Na₂CO₃. The organic layer was dried over MgSO₄, filtered and concentrated under vacuum. The product was purified by flash chromatography column over silica gel using cyclohexane/ethyl acetate (100/0 to 60/40); Yield: 13%; colorless oil. ¹H NMR (300 MHz, CDCl₃): δ 7.13 (d, J = 8.5 Hz, 2H), 6.77 (d, J = 8.5 Hz, 2H), 4.14-4.05 (m, 1H), 3.99 (dd, J = 8.6, 7.0 Hz, 1H), 3.77 (dd, J = 8.6, 6.3 Hz, 1H), 3.46 (d, J = 13.1 Hz, 1H), 3.33 (d, J = 13.1 Hz, 1H), 2.78-2.62 (m, 2H), 2.62-2.52 (m, 1H), 2.46-2.28 (m, 3H), 2.22-2.13 (m, 1H), 2.08-1.88 (m, 2H), 1.83-1.74 (m, 1H), 1.70-1.55 (m, 2H), 0.94 |
| | | (d, J = 6.9 Hz, 3H), 0.90 (d, J = 6.9 Hz, 3H) ppm. ¹³C NMR |
| | | (75 MHz, CDCl₃): δ 170.8, 155.5, 130.2, 130.1, 115.3, 92.8, |
| | | 66.2, 62.0, 61.4, 54.6, 50.5, 40.4, 32.4, 32.3, 30.8, 21.9, 19.9, |
| | | 18.7 ppm. HRMS (ESI, m/z): [M + H]⁺ calcd. for C₂₀H₂₉N₂O₃, |
| | | 345.2178; found 345.2191. |
| 10 | 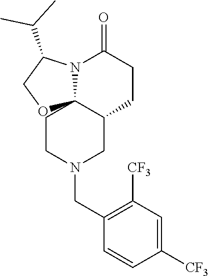 [SMILES: CC(C)[C@H]1CO[C@]23CCN(Cc4ccc(C(F)(F)F)cc4C(F)(F)F)C[C@H]2CCC(=O)N13] | (3S,7aR,11aR)-9-[2,4- bis(trifluoromethyl)phenyl]methyl]-3-isopropyl- 2,3,6,7,7a,8,10,11-octahydrooxazolo[2,3- j][1,6]naphthyridin-5-one Example 10 was obtained from intermediate 24 and 2,4- bis(trifluoromethyl)benzaldehyde using general protocol 8. The solvent was removed under vacuum, the mixture was then taken up in CH₂Cl₂ and washed with a saturated aqueous solution of Na₂CO₃. The organic layer was dried over MgSO₄, filtered and concentrated under vacuum. The product was purified by flash chromatography column over silica gel using cyclohexane/ethyl acetate (100/0 to 20/80); Yield: 13%; [ES+ MS] m/z 465 (MH⁺). |
| 19 | 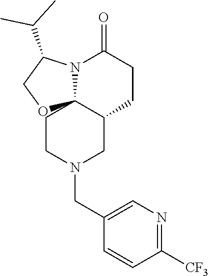 [SMILES: CC(C)[C@H]1CO[C@]23CCN(Cc4ccc(C(F)(F)F)nc4)C[C@H]2CCC(=O)N13] | (3S,7aR,11aR)-3-isopropyl-9-[[6-(trifluoromethyl)-3- pyridyl]methyl]-2,3,6,7,7a,8,10,11-octahydrooxazolo[2,3- j][1,6]naphthyridin-5-one Example 19 was obtained from intermediate 24 and 6- (trifluoromethyl)pyridine-3-carbaldehyde using general protocol 8. The solvent was removed under vacuum, the mixture was then taken up in CH₂Cl₂ and washed with a saturated aqueous solution of Na₂CO₃. The organic layer was dried over MgSO₄, filtered and concentrated under vacuum. The product was purified by flash chromatography column over silica gel using cyclohexane/ethyl acetate (100/0 to 0/100); Yield: 59%; white powder. ¹H NMR (300 MHz, CDCl₃): δ 8.66 (brs, 1H), 7.85 (dd, J= 8.0, 1.3 Hz, 1H), 7.64 (d, J = 8.0 Hz, 1H), 4.14-4.06 (m, 1H), 3.99 (dd, J = 8.5, 8.0 Hz, 1H), 3.78 (dd, J = 8.5, 6.2 Hz, 1H), 3.62 (d, J = 14.1 Hz, 1H), 3.52 (d, J = 14.1 Hz, 1H), 2.75-2.52 (m, 4H), 2.48-2.25 (m, 3H), 2.07-1.91 (m, 2H), 1.85-1.77 (m, 1H), 1.75-1.59 (m, 2H), |
| | | 0.95 (d, J = 6.8 Hz, 3H), 0.91 (d, J = 6.8 Hz, 3H) ppm. ¹³C |
| | | NMR (75 MHz, CDCl₃): δ 170.1, 129.2, 147.2 (q, J = 33.5 Hz), |
| | | 137.9, 137.5, 121.5 (q, J = 272.0 Hz), 120.3 (q, J = 3.7 Hz), |
| | | 92.3, 66.3, 61.3, 59.4, 55.0, 50.8, 40.3, 32.4, 32.3, 30.7, 21.9, |
| | | 19.9, 18.7 ppm. HRMS (ESI, m/z): [M + H]⁺ calcd. for |
| | | C₂₀H₂₇N₃O₂F₃, 398.2055; found 398.2044. |
| 20 | 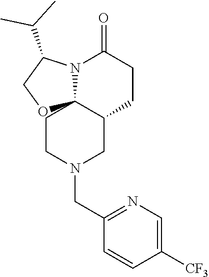 [SMILES: CC(C)[C@H]1CO[C@]23CCN(Cc4ccc(C(F)(F)F)cn4)C[C@H]2CCC(=O)N13] | (3S,7aR,11aR)-3-isopropyl-9-[[5-(trifluoromethyl)-2- pyridyl]methyl]-2,3,6,7,7a,8,10,11-octahydrooxazolo[2,3- j][1,6]naphthyridin-5-one Example 20 was obtained from intermediate 24 and 5- (trifluoromethyl)pyridine-2-carbaldehyde using general protocol 8. The solvent was removed under vacuum, the mixture was then taken up in CH₂Cl₂ and washed with a saturated aqueous solution of Na₂CO₃. The organic layer was dried over MgSO₄, filtered and concentrated under vacuum. The product was purified by flash chromatography column over silica gel using cyclohexane/ethyl acetate (100/0 to 0/100); Yield: 70%; light brown solid. ¹H NMR (300 MHz, CDCl₃): δ 8.77 (m, 1H); 7.91 (dd, J = 8.30 Hz, J = 2.15 Hz, 2H), 7.64 (d, J = 8.1 Hz, 2H), 4.07-3.95 (m, 2H), 3.78-3.63 (m, 3H), 2.74-2.50 (m, 4H), 2.43-2.28 (m, 3H), 2.04-1.92 (m, 2H), 1.84-1.77 (m, 1H), 1.74-1.61 (m, 2H), 0.93-0.88 (m, 6H) ppm. [ES+ MS] m/z 398 (MH⁺). |
| 26 | 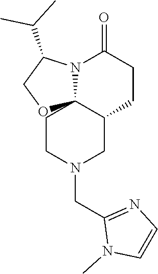 [SMILES: CC(C)[C@H]1CO[C@]23CCN(Cc4nccn4C)C[C@H]2CCC(=O)N13] | 3-isopropyl-9-[(1-methylimidazol-2-yl)methyl]- 2,3,6,7,7a,8,10,11-octahydrooxazolo[2,3- j][1,6]naphthyridin-5-one Example 26 was obtained from intermediate 24 and 1- methylimidazole-2-carbaldehyde using general protocol 8. The mixture was quenched with water, an aqueous solution of NaOH (30%) and brine. The solution was stirred at room temperature for 1 h, extracted with DCM. The combined organic layers were dried over MgSO₄, filtered and concentrated under vacuum. Purification by flash chromatography over silica gel (DCM/MeOH: 100/0 to 98/2). Yield: 25%; [ES+ MS] m/z 333 (MH⁺). |
| 27 | 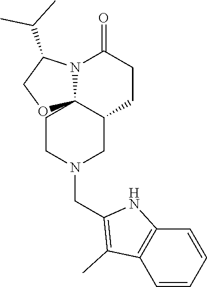 [SMILES: Cc1c(CN2CC[C@]34OC[C@H](C(C)C)N3C(=O)CC[C@@H]4C2)[nH]c2ccccc12] | (3S,7aR,11aR)-3-isopropyl-9-[(3-methyl-1H-indol-2- yl)methyl]-2,3,6,7,7a,8,10,11-octahydrooxazolo[2,3- j][1,6]naphthyridin-5-one Example 27 was obtained from intermediate 24 and 3- methyl-1H-indole-2-carbaldehyde using general protocol 8. The solvent was removed under vacuum, the mixture was then taken up in CH₂Cl₂ and washed with a saturated aqueous solution of Na₂CO₃. The organic layer was dried over MgSO₄, filtered and concentrated under vacuum. The product was purified by flash chromatography column over silica gel (cyclohexane/ethyl acetate: 100/0 to 60/40); Yield: 40%; [ES+ MS] m/z 382 (MH⁺). |
| 28 | 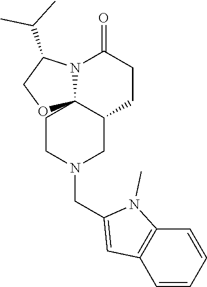 [SMILES: CC(C)[C@H]1CO[C@]23CCN(Cc4cc5ccccc5n4C)C[C@H]2CCC(=O)N13] | (3S,7aR,11aR)-3-isopropyl-9-[(1-methylindol-2- yl)methyl]-2,3,6,7,7a,8,10,11-octahydrooxazolo[2,3- j][1,6]naphthyridin-5-one Example 28 was obtained from intermediate 24 and 1- methylindole-2-carbaldehyde using general protocol 8. The mixture was extracted with DCM. The organic layer was dried on MgSO₄, filtered and concentrated under vacuum to give the crude product. Purification by flash chromatography column over silica gel (cyclohexane/ethyl acetate: 100/0 to 0/100); Yield: 73%; visqueous yellow oil. [ES+ MS] m/z 382 (MH⁺). |
| 32 | 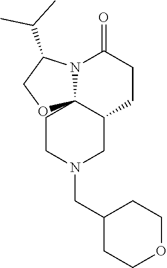 [SMILES: CC(C)[C@H]1CO[C@]23CCN(CC4CCOCC4)C[C@H]2CCC(=O)N13] | (3S,7aR,11aR)-3-isopropyl-9-(tetrahydropyran-4- ylmethyl)-2,3,6,7,7a,8,10,11-octahydrooxazolo[2,3- j][1,6]naphthyridin-5-one Example 32 was obtained from intermediate 24 and tetrahydropyran-4-carbaldehyde using general protocol 8. The solvent was removed under vacuum, the mixture was then taken up in CH₂Cl₂ and washed with a saturated aqueous solution of Na₂CO₃. The organic layer was dried over MgSO₄, filtered and concentrated under vacuum. The product was purified by flash chromatography column over silica gel (cyclohexane/ethyl acetate: 100/0 to 60/40); Yield: 22%; [ES+ MS] m/z 337 (MH⁺). |

### Synthesis of Intermediates 31-34

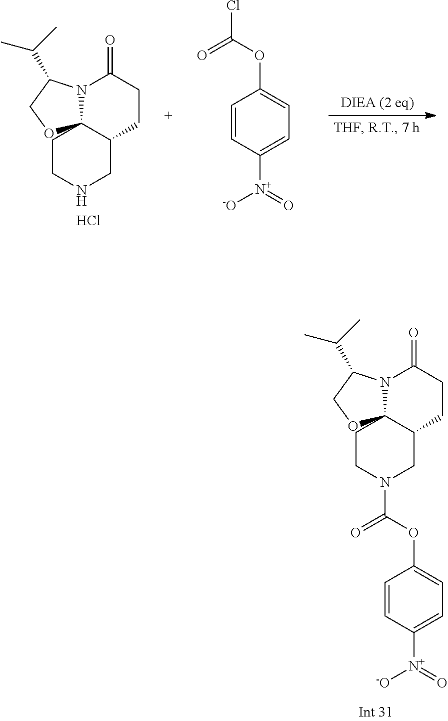

(4-nitrophenyl) carbonochloridate (0.49 mmol, 1.4 mmol) dissolved in THF was added dropwise to a solution of intermediate 21 (0.36 mmol, 1 eq), DIEA (0.73 mmol, 2 eq) dissolved in THF. The mixture was stirred at room temperature for 7 h. Ethyl acetate was added, washed twice with water, once with brine, dried over MgSO₄, filtered and evaporated under vacuum. The intermediate was purified by reverse phase chromatography.

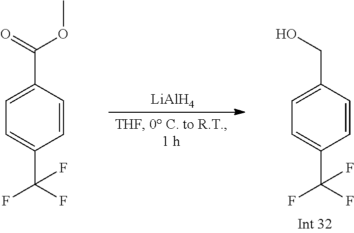

At 0° C. methyl 4-(trifluoromethyl)benzoate (0.98 mmol, 1 eq) was dissolved in THF. Then LiAlH₄ (1.96 mmol, 2 eq) was added. The solution was stirred at room temperature for 1 h. Ethyl acetate was added and washed with an aqueous solution of HCl 1N twice, dried over MgSO₄, filtered and evaporated under vacuum. The intermediate 32 was purified by chromatography.

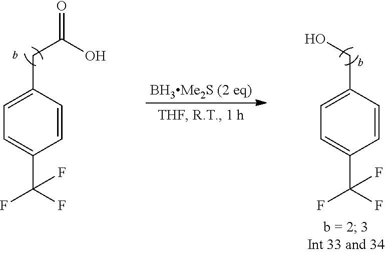

At room temperature the appropriate carboxylic acid (1 eq) was dissolved in THF. Then borane; methylsulfanylmethane (2 eq) was added. The mixture was stirred until completion of the reaction at room temperature. Ethyl acetate was added and washed with water twice, dried over MgSO₄, filtered and evaporated under vacuum.

| Intermediate | Structure | Physical data |
|---|---|---|
| 32 | 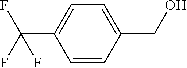 [SMILES: OCc1ccc(C(F)(F)F)cc1] | [4-(trifluoromethyl)phenyl]methanol Intermediate 32 was obtained as a colorless oil. Yield = 97%. ¹H NMR (300 MHz, CDCl₃): δ 7.64 (d, J = 8.1 Hz, 2H), 7.49 (d, J = 8.1 Hz, 2H), 4.78 (s, 2H), 2.05 (s, 1H) ppm. |
| 33 | 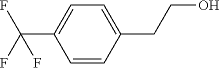 [SMILES: OCCc1ccc(C(F)(F)F)cc1] | 2-[4-(trifluoromethyl)phenyl]ethanol Intermediate 33 was obtained as a colourless oil. Yield = 87%. ¹H NMR (300 MHz, CDCl₃): δ 7.56 (d, J = 8.1 Hz, 2H), 7.35 (d, J = 8.1 Hz, 2H), 3.89 (dd, J = 6.3 Hz, J = 11.7 Hz, 2H), 2.92 (t, J = 6.5 |
| | | Hz, 2H), 1.51 (t, J = 5.4 Hz, 1H) ppm. |
| 34 | 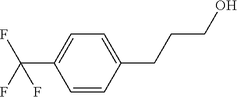 [SMILES: OCCCc1ccc(C(F)(F)F)cc1] | 3-[4-(trifluoromethyl)phenyl]propan-1-ol Intermediate 34 was obtained as a colorless oil. Yield = 96%. ¹H NMR (300 MHz, CDCl₃): 7.56 (d, J = 8.2 Hz, 2H), 7.34 (d, J = 8.3 Hz, 2H), 3.71 (t, J = 6.3 Hz, 2H), 2.80 (t, J = 7.6 Hz, 2H), 1.98-1.87 (m, 2H), 1.38 (s, 1H) ppm. |
| 31 | 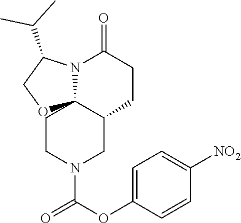 [SMILES: CC(C)[C@H]1CO[C@]23CCN(C(=O)Oc4ccc([N+](=O)[O-])cc4)C[C@H]2CCC(=O)N13] | (4-nitrophenyl)(3S,7aR,11aR)-3-isopropyl-5- oxo-2,3,6,7,7a,8,10,11-octahydrooxazolo[2,3- j][1,6]naphthyridine-9-carboxylate Intermediate 34 was obtained as a white powder Yield = 80%. ¹H NMR (300 MHz, CDCl₃): δ 8.28- 8.21 (m, 2H), 7.30 (d, J = 8.1 Hz, 2H), 4.28-4.10 (m, 3H), 4.07-3.98 (m, 1H), 3.87-3.79 (dd, J = 6 Hz, J = 8.8 Hz, 1H), 3.60-3.47 (dd, J = 3.1 Hz, J = 13.70 Hz, 0.55H), 3.47-3.33 (dd, J = 3.1 Hz, J = 13.70 Hz, 0.45H), 3.30-3.15 (dt, J = 3.1 Hz, J = 13.2 Hz, 0.45H), 3.15-3.01 (dt, J = 3.1 Hz, J = 13.2 Hz, 0.55H), 2.71-2.56 (m, 1H), 2.56-2.40 (m, 1H), 2.09-1.80 (m, 5H), 1.8-1.7 (m, 1H), 0.99-0.89 (t, J = 7.4 Hz, 6H) ppm. |

### Synthesis Example 47

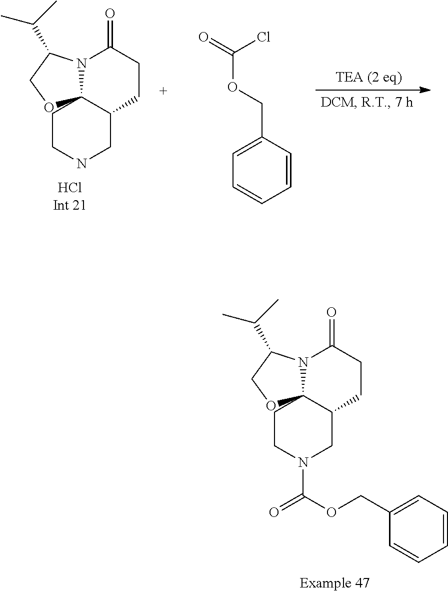 [SMILES: CC(C)[C@H]1CO[C@]23CCNC[C@H]2CCC(=O)N13 ; O=C(Cl)OCc1ccccc1 ; CC(C)[C@H]1CO[C@]23CCN(C(=O)OCc4ccccc4)C[C@H]2CCC(=O)N13]

In a round bottom flask charged with (3S,7aR,11aR)-3-isopropyl-3,6,7,7a,8,9,10,11-octahydro-2H-oxazolo[2,3-j][1,6]naphthyridin-5-one; hydrochloride (intermediate 21) (1 eq) was added DCM, TEA (2 eq) and benzyl carbonochloridate (1 eq). The mixture was stirred until completion of the reaction. Crude was purified to afford the desired product.

### General Protocol 9: Carbamate Synthesis

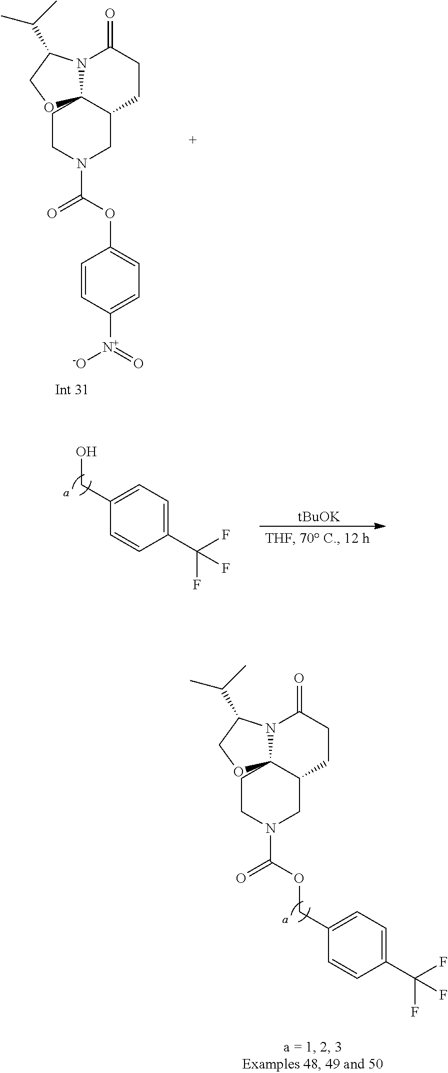

Intermediate 31 (1 eq) was dissolved in THF, then t-BuOK (1 eq) and the appropriate alcohol (1 eq) were added. The mixture was stirred at reflux overnight. Dichloromethane was added and the organic layer was washed three times with a saturated aqueous solution of NaHCO₃, once with brine, dried over MgSO₄ filtered and then concentrated under reduced pressure. The residue was purified by reverse phase chromatography to afford the desired product.

| Example | Structure | Physical data |
|---|---|---|
| 47 | 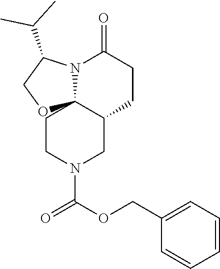 [SMILES: CC(C)[C@H]1CO[C@]23CCN(C(=O)OCc4ccccc4)C[C@H]2CCC(=O)N13] | Benzyl(3S,7aR,11aR)-3-isopropyl-5-oxo- 2,3,6,7,7a,8,10,11-octahydrooxazolo[2,3- j][1,6]naphthyridine-9-carboxylate Example 47 was obtained from intermediate 21 and benzyl carbonochloridate using general protocol 9. Purification by flash chromatography over silica gel (cyclohexane/ethyl acetate: 100/0 to 0/100); [ES+ MS] m/z 373 (MH⁺). |
| 48 | 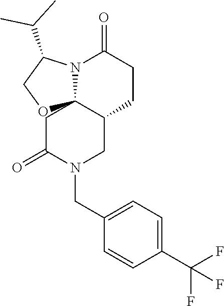 [SMILES: CC(C)[C@H]1CO[C@]23CC(=O)N(Cc4ccc(C(F)(F)F)cc4)C[C@H]2CCC(=O)N13] | [4-(trifluoromethyl)phenyl]methyl(3S)-3-isopropyl- 5-oxo-2,3,6,7,7a,8,10,11-octahydrooxazolo[2,3- j][1,6]naphthyridine-9-carboxylate Example 48 was synthetized from intermediate 31 and intermediate 32 using general protocol 9. Purification by reverse flash chromatography (MeCN/Water 10/90 to 100/0); Yield: 76%; ¹H NMR (300 MHz, CDCl₃): δ 7.64 (d, J = 8.1 Hz, 2H), 7.48 (d, J = 8.1 Hz, 2H), 5.27-5.10 (m, 2H), 4.25-3.95 (m, 4H), 3.81 (dd, J = 6.0 Hz, J = 8.9 Hz, 1H), 3.45-3.20 (m, 1H), 3.15- 2.85 (m, 1H), 2.65-2.36 (m, 2H), 2.0 (oct, J = 6.1 Hz, 1H), 1.89-1.60 (m, 5H), 0.95 (d, J = 7.0 Hz, 3H), 0.93 (d, J = 6.8 Hz, 3H) ppm. ¹³C NMR (75 MHz, CDCl₃): δ 169.5, 155.2, 140.6, 130.3 (q, J = 32 Hz), 127.9, 125.5 (q, J = 3.6 Hz), 124.1 (q, J = 271 Hz), 92.0, 66.4, 66.3, 61.5, 45.3, 41.1, 39.8, 32.1, 31.6, 30.0, 20.5, 19.6, 18.6 ppm. |
| 49 | 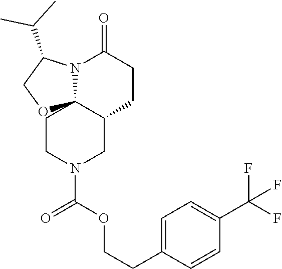 [SMILES: CC(C)[C@H]1CO[C@]23CCN(C(=O)OCCc4ccc(C(F)(F)F)cc4)C[C@H]2CCC(=O)N13] | 2-[4-(trifluoromethyl)phenyl]ethyl(3S,7aR,11aR)-3- isopropyl-5-oxo-2,3,6,7,7a,8,10,11- octahydrooxazolo[2,3-j][1,6]naphthyridine-9- carboxylate Example 49 was synthetized from intermediate 31 and intermediate 33 using general protocol 9. Purification by reverse flash chromatography (MeCN/Water 10/90 to 100/0); Yield: 35%. ¹H NMR (300 MHz, CDCl₃): δ 7.55 (d, J = 8.0 Hz, 2H), 7.33 (d, J = 8.0 Hz, 2H), 4.39-4.26 (m, 2H), 4.17-4.02 (m, 2H), 3.97 (dd, J = 7.2 Hz, J = 8.8 Hz, 1H), 3.77 (dd, J = 6.1 Hz, J = 8.8 Hz, 1H), 3.32-3.13 (m, 1H), 3.00 (t, J = 6.6 Hz, 2H), 2.90- 2.80 (m, 1H), 2.60-2.20 (m, 2H), 2.06-1.92 (m, 1H), 1.87- 1.68 (m, 1H), 1.8-1.5 (m, 4H), 1.48-1.22 (m, 1H), 0.97- 0.82 (m, 6H) ppm. ¹³C NMR (75 MHz, CDCl₃): δ 169.5, 155.4, 142.2, 129.6, 128.9 (q, J = 32.5 Hz), 125.4 (q, J = 4 Hz), 124.1 (q, J = 272 Hz), 92.0, 66.3, 65.3, 61.4, 45.0, |
| | | 40.9, 39.7, 35.3, 32.1, 31.6, 30.0, 20.3, 19.6, 18.6 ppm. |
| 50 | 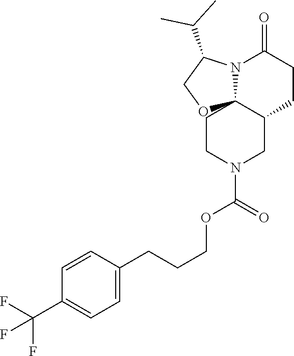 [SMILES: CC(C)[C@H]1CO[C@]23CCN(C(=O)OCCCc4ccc(C(F)(F)F)cc4)C[C@H]2CCC(=O)N13] | 3-[4-(trifluoromethyl)phenyl]propyl(3S,7aR,11aR)-3- isopropyl-5-oxo-3,6,7,7a,8,9,10,11-octahydro-2H- oxazolo[2,3-j]quinoline-9-carboxylate Example 50 was synthetized from intermediate 31 and intermediate 34 using general protocol 9. Purification by reverse flash chromatography (MeCN/Water 10/90 to 100/0); Yield: 18%. ¹H NMR (300 MHz, CDCl₃): δ 7.54 (d, J = 8 Hz, 2H), 7.29 (d, J = 8.0 Hz, 2H), 4.20-4.04 (m, 4H), 4.04-3.90 (m, 2H), 3.90-3.70 (m, 2H), 3.34-3.12 (m, 1H), 3.00-2.84 (m, 1H), 2.75 (t, J = 7.6 Hz, 2H), 2.63-2.50 (m, 1H), 2.50-2.35 (m, 1H), 2.07-1.90 (m, 3H), 1.80-1.70 (m, 4H), 0.98-0.87 (m, 7H) ppm. ¹³C NMR (75 MHz, CDCl₃): δ 169.5, 155.6, 145.4, 128.7, 128.5 (q, J = 32 Hz), 125.4 (q, J = 3.7 Hz), 124 (q, J = 272 Hz), 92.1, 66.3, 64.8, 61.4, 45.1, 40.9, 39.8, 32.2, 32.1, 31.6, 30.3, 30.1, 20.5, 18.6 ppm. |

### General Protocol 10: Amide Synthesis

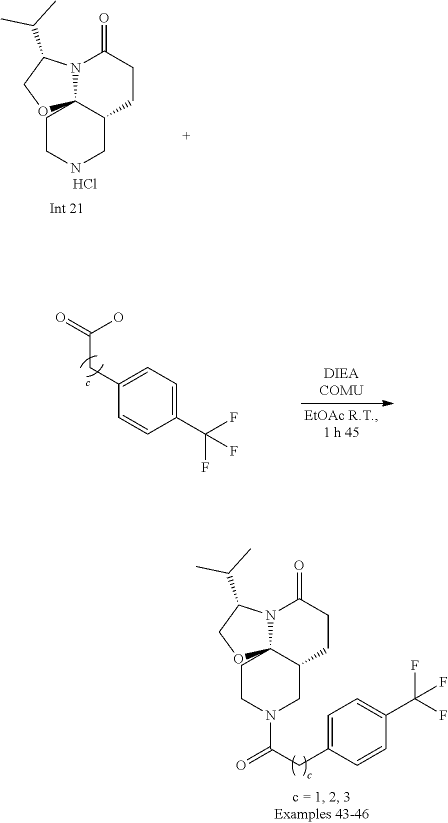

Appropriate carboxylic acid (1.35 eq) was dissolved in ethyl acetate, then COMU (2 eq), DIEA (1 eq) were added. Then mixture was stirred for 15 min at room temperature. Intermediate 21 (1 eq) and DIEA (1.2 eq) were then dissolved in ethyl acetate and added dropwise to the first solution. The mixture was stirred at room temperature until completion of the reaction. Ethyl acetate was added, washed twice with an aqueous solution of HCl 1N, twice with a saturated aqueous solution of NaHCO₃, once with brine, dried over MgSO₄, filtered and evaporated under vacuum. The residue was purified by reverse phase chromatography to afford the desired product

### Synthesis of 2,2-difluoro-2-[4-(trifluoromethyl)phenyl]acetic acid (intermediate 35)

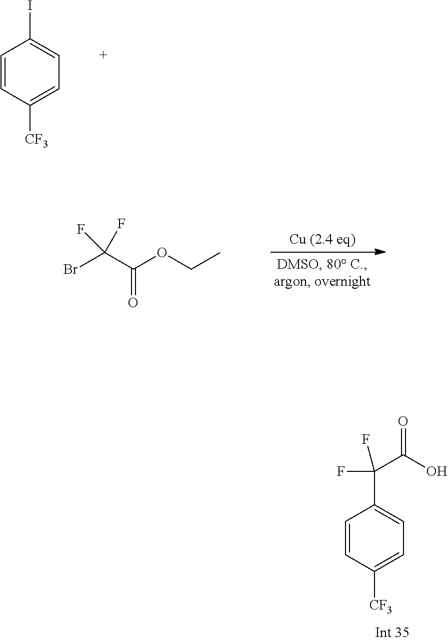

To a solution of copper (2.6 eq) in DMSO, was added dropwise under argon at room temperature 1-iodo-4-(trifluoromethyl)benzene (1 eq) followed by ethyl 2-bromo-2,2-difluoro-acetate (1 eq). The mixture was stirred overnight at 80° C. under argon. The solution was filtered through a celite plug and eluted with diethyl-ether. The filtrate was evaporated; the remaining DMSO was then freeze-dried. The crude was dissolved in ethyl acetate and washed twice with an aqueous solution of HCl 1N, once with brine, dried over MgSO₄, filtered and evaporated under reduced pressure to afford the desired product intermediate 35. Yield=65%, ¹H NMR (300 MHZ, CDCl₃): δ 9.97 (s, 1H), 7.80 (d, J=8.9 Hz, 2H), 7.76 (d, J=8.9 Hz, 2H) ppm. [ES+MS] m/z 239 (MH⁺).

### Synthesis Example 42

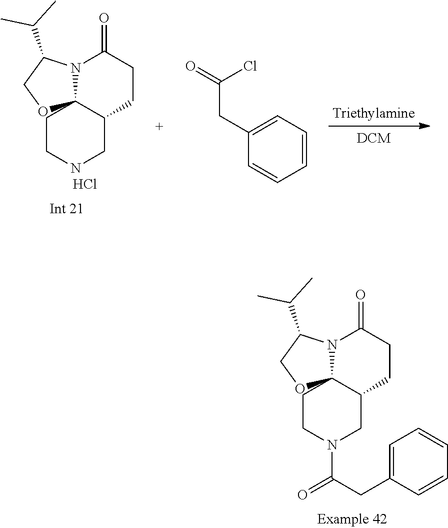 [SMILES: CC(C)[C@H]1CO[C@]23CCNC[C@H]2CCC(=O)N13 ; O=C(Cl)Cc1ccccc1 ; Cl ; CC(C)[C@H]1CO[C@]23CCN(C(=O)Cc4ccccc4)C[C@H]2CCC(=O)N13]

To a solution of (3S,7aR,11aR)-3-isopropyl-3,6,7,7a,8,9,10,11-octahydro-2H-oxazolo[2,3-j][1,6]naphthyridin-5-one; formic acid (80 mg, 0.281 mmol) in DCM (2 mL) were added 2-phenylacetyl chloride (32.7 μL, 0.281 mmol) and K₂CO₃ (117 mg, 0.844 mmol). The mixture was stirred for 2 h at rt, then 14 h at 60° C. and 50 h at rt. 1 eq. of 2-phenylacetyl chloride and triethylamine were added and the mixture was further stirred at 60° C. for 4 h. The solvent was removed under vacuum; the mixture was then taken up in DCM and washed with saturated Na₂CO₃. The organic phase was dried over magnesium sulfate and the DCM was removed under vacuum. Product was purified throughflash chromatography over silica gel (cyclohexane/ethyl acetate: 100/0 to 0/100); to afford 15.4 mg of the desired product (yield=16%).

| Example | Structure | Physical data |
|---|---|---|
| 42 | 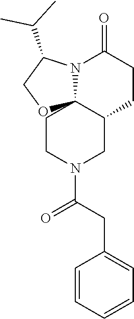 | (3S,7aR,11aR)-3-isopropyl-9-(2-phenylacetyl)- 2,3,6,7,7a,8,10,11-octahydrooxazolo[2,3- j][1,6]naphthyridin-5-one Example 42 was obtained from intermediate 21 and 2- phenylacetyl chloride. [ES+ MS] m/z 343 (MH⁺). |
| 43 | 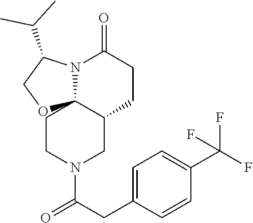 [SMILES: CC(C)[C@H]1CO[C@]23CCN(C(=O)Cc4ccc(C(F)(F)F)cc4)C[C@H]2CCC(=O)N13] | (3S)-3-isopropyl-9-[2-[4-(trifluoromethyl)phenyl]acetyl]- 2,3,6,7,7a,8,10,11-octahydrooxazolo[2,3- j][1,6]naphthyridin-5-one Example 43 was obtained from intermediate 21 and 2-[4- (trifluoromethyl)phenyl]acetic acid using general protocol 10. Purification by reverse flash chromatography (MeCN/Water 10/90 to 100/0); Yield: 69%; ¹H NMR (300 MHz, CDCl₃): δ 7.61 (d, J = 7.5 Hz, 2H), 7.40 (d, J = 7.8 Hz, 2H), 4.70-4.55 (m, 1H), 4.20-4.07 (m, 1H), 4.00 (dt, J = 3.2 Hz, J = 8.7 Hz, 1H), 3.89-3.67 (m, 4H), 3.57 (dd, J = 2.5 Hz, J = 13.8 Hz, 0.5 H), 3.23 (dt, J = 3.0 Hz, J = 13.8 Hz, 0.5 H), 3.07 (dd, J = 2.7 Hz, J = 13.5 Hz, 0.5 H), 2.85-2.70 (m, 0.5 H), 3.07 (dd, J = 2.7 Hz, J = 13.5 Hz, 0.5 H), 2.85-2.70 (m, 0.5 H), 2.66- 2.33 (m, 2H), 2.06 (oct, J = 6.9 Hz, 1H) 1.91-1.58 (m, 5H), |
| | | 1.57-1.45 (m, 0.5 H), 0.97-0.85 (m, 6H) ppm. ¹³C NMR (75 |
| | | MHz, CDCl₃): δ 169.4, 169.1, 169.0, 139.0, 129.4 (q, J = 31 |
| | | Hz), 129.3, 129.1, 125.7 (q, J = 4 Hz), 124.0 (q, J = 271 Hz), |
| | | 91.90, 91.87, 66.4, 66.3, 61.5, 47.3, 43.2, 43.1, 40.6, 40.2, |
| | | 40.0, 39.8, 39.0, 32.3, 32.1, 31.4, 30.0, 29.9, 20.4, 20.3, 19.6, |
| | | 18.6 ppm. |
| 44 | 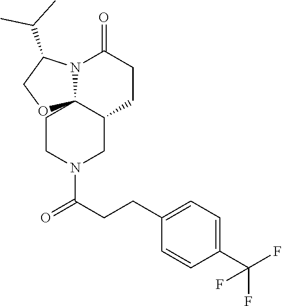 [SMILES: CC(C)[C@H]1CO[C@]23CCN(C(=O)CCc4ccc(C(F)(F)F)cc4)C[C@H]2CCC(=O)N13] | (3S)-3-isopropyl-9-[3-[4- (trifluoromethyl)phenyl]propanoyl]-2,3,6,7,7a,8,10,11- octahydrooxazolo[2,3-j][1,6]naphthyridin-5-one Example 44 was synthetized from intermediate 21 and 3-[4- (trifluoromethyl)phenyl]propanoic acid using general protocol 10. Purification by reverse flash chromatography (MeCN/Water 10/90 to 100/0); Yield: 64%; ¹H NMR (300 MHz, CDCl₃): δ 7.56 (d, J = 8 Hz, 2H), 7.35 (q, J = , 2H), 4.70-4.55 (m, 1H), 4.20-4.06 (m, 1H), 3.87-3.75 (m, 1H), 3.75-3.62 (m, 1H), 3.62-3.48 (m, 0.5H), 3.26-3.12 (dt, J = 3.0 Hz, J = 13.1 Hz, 0.5H), 3.11-2.98 (m, 2H), 2.80-2.57 (m, 2H), 2.57-2.35 (m, 2H), 2.10-1.90 (m, 1H), 1.88-1.51 (m, 7H), 0.98-0.88 (m, 6H) ppm. ¹³C NMR (75 MHz, CDCl₃): δ 170.4, 170.3, 169.59, 169.1, 145.3, 128.9, 128.6 (q, J = 32 Hz), 128.8, 125.4 (q, J = 4 Hz), 124.0 (q, J = 272 Hz), 92.0, 66.3, 61.5, 61.4, 46.7, 42.9, 42.6, 40.0, 39.7, 38.8, 34.3, 32.5, 32.1, 32.0, 31.5, 31.1, 31.0, 29.9, 29.8, 20.3, 19.7, 19.6, 18.6, 18.5 ppm. |
| 45 | 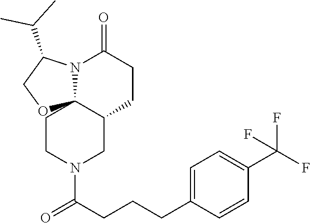 [SMILES: CC(C)[C@H]1CO[C@]23CCN(C(=O)CCCc4ccc(C(F)(F)F)cc4)C[C@H]2CCC(=O)N13] | (3S)-3-isopropyl-9-[4-[4- (trifluoromethyl)phenyl]butanoyl]-2,3,6,7,7a,8,10,11- octahydrooxazolo[2,3-j][1,6]naphthyridin-5-one Example 45 was synthetized from intermediate 21 and 4-[4- (trifluoromethyl)phenyl]butanoic acid using general protocol 10. Purification by reverse flash chromatography (MeCN/Water 10/90 to 100/0); Yield: 73%; ¹H NMR (300 MHz, CDCl₃): δ 7.53 (d, 7.8 Hz, 2H), 7.29 (d, J = 8 Hz, 2H), 4.66-4.53 (m, 1H), 4.17-4.05 (m, 1H), 4.04-3.95 (m, 1H), 3.86-3.73 (m, 1H), 3.71-3.46 (m, 1H), 3.26-3.12 (m, 0.45H), 3.05-3.92 (m, 0.55H), 2.73 (t, J = 7.3 Hz, 2H), 2.61-2.2 (m, 4H), 2.09-1.90 (m, 3H), 1.87-1.66 (m, 4H), 0.96-0.87 (m, 6H) ppm. ¹³C NMR (75 MHz, CDCl₃): δ 169.5, 155.6, 145.4, 128.7, 128.5 (q, J = 32 Hz), 125.4 (q, J = 3.7 Hz), 124 (q, J = |
| | | 272 Hz), 92.1, 66.3, 64.8, 61.4, 45.1, 40.9, 39.8, 32.2, 32.1, |
| | | 31.6, 30.3, 30.1, 20.5, 18.6 ppm. |
| 46 | 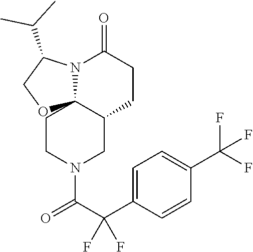 [SMILES: CC(C)[C@H]1CO[C@]23CCN(C(=O)C(F)(F)c4ccc(C(F)(F)F)cc4)C[C@H]2CCC(=O)N13] | (3S)-9-[2,2-difluoro-2-[4-(trifluoromethyl)phenyl]acetyl]- 3-isopropyl-2,3,6,7,7a,8,10,11-octahydrooxazolo[2,3- j][1,6]naphthyridin-5-one Example 46 was synthetized from intermediate 21 and intermediate 35 using general protocol 10. The Crude was purified by reverse flash chromatography (from water/MeCN 90/10 to 0/100), Yield = 65%. ¹H NMR (300 MHz, CDCl₃): δ 7.76 (d, J = 8.2 Hz, 2H), 7.68 (d, J = 8.3 Hz, 2H), 4.54 (d, J = 13.3 Hz, 1H), 4.18-4.09 (m, 1H), 4.07-3.94 (m, 2H), 3.84- 3.74 (m, 1H), 3.51 (dd, J = 2.2 Hz, J = 13.7 Hz, 0.5 H), 3.28- 3.11 (m, 1H), 2.99-2.83 (m, 0.5 H), 2.60 (dd, J = 7.2 Hz, J = 17.5 Hz, 1H), 2.51-2.35 (m, 1H), 2.09-1.61 (m, 6H), 0.99-0.84 (m, 6H) ppm. ¹³C NMR (75 MHz, CDCl₃): δ 169.4, 169.3, 162.0 (t, J = 31 Hz), 137.2 (t, J = 35 Hz) 132.9 (q, J = 32 Hz), 126.0 (q, J = 5.3 Hz), 125.7 (t, J = 3.6 Hz), 123.5 (q, J = 274 Hz), 119.0, 118.1, 115.6, 91.7, 91.7, 66.4, 61.5, 46.6 |
| | | (t, J = 4.4 Hz), 44.8, 43.0 (t, J = 5.5 Hz), 40.6, 40.1, 39.7, |
| | | 32.4, 32.2, 31.3, 30.1, .30.0, 20.4, 20.1, 19.6, 18.6 ppm. |

### General Protocol 11: Sulfonamides

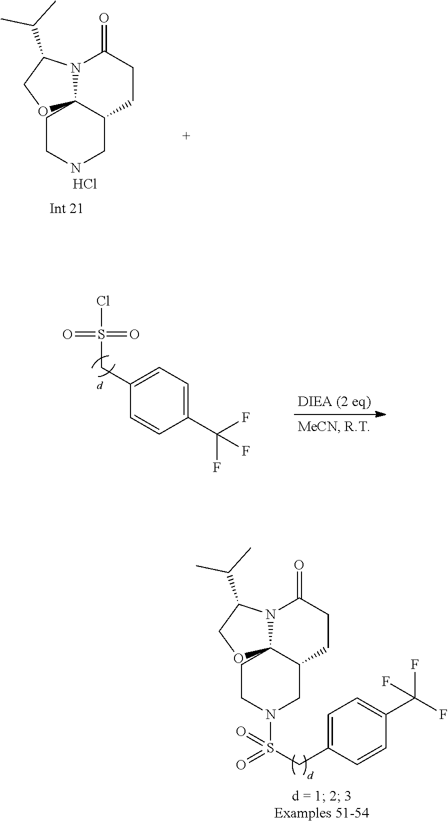

In a round bottom flask charged with intermediate 21 (1 eq), suspended in MeCN was added DIEA (2 eq). The mixture was stirred until complete solubilization. Then the appropriate sulfonyl chloride (1 eq) was added. The mixture was stirred at room temperature until completion of the reaction followed by TLC. Then the solvent was removed under reduced pressure, the crude was solubilized in ethyl acetate and washed twice with water, once with brine, dried over MgSO₄, filtered and evaporated under reduced pressure.

| Example | Structure | Physical data |
|---|---|---|
| 51 | 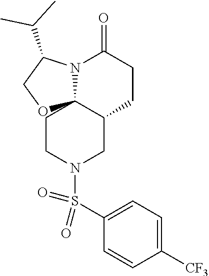 [SMILES: CC(C)[C@H]1CO[C@]23CCN(S(=O)(=O)c4ccc(C(F)(F)F)cc4)C[C@H]2CCC(=O)N13] | (3S,7aR,11aR)-3-isopropyl-9-[4- (trifluoromethyl)phenyl]sulfonyl-2,3,6,7,7a,8,10,11- octahydrooxazolo[2,3-j][1,6]naphthyridin-5-one Example 51 was obtained from intermediate 24 and 4- (trifluoromethyl)benzenesulfonyl chloride using general protocol 11; Purification by flash chromatography over silica gel (cyclohexane/ethyl acetate: 100/0 to 0/100); [ES+ MS] m/z 447 (MH⁺). |
| 52 | 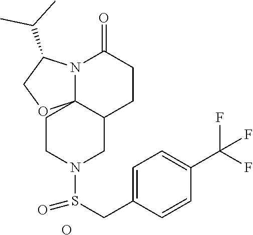 | (3S)-3-isopropyl-9-[[4- (trifluoromethyl)phenyl]methylsulfonyl]- 2,3,6,7,7a,8,10,11-octahydrooxazolo[2,3- j][1,6]naphthyridin-5-one Example 52 was synthetized from intermediate 21 and [4- (trifluoromethyl)phenyl]methanesulfonyl chloride using general protocol 11; The crude was evaporated under reduced pressure and purified through flash chromatography (cyclohexane/ethyl acetate: 100/0 to 60/40); Yield = 68%; ¹H NMR (300 MHz, CDCl₃): δ 7.68 (d, J = 8.1 Hz, 2H), 7.52 (d, J = 8.0 Hz, 2H), 4.26 (s, 2H), 4.17-4.06 (m, 1H), 3.97 (dd, J = 7.3 Hz, J = 8.8 Hz, 1H), 3.78 (dd, J = 5.8 Hz, J = 8.8 Hz, 1H), 3.64-3.50 (m, 2H), 3.13 (dd, J = 3.1 Hz, J = 12.5 Hz, 1H), 2.81 (dt, J = 2.9 Hz, J = 12.5 Hz, 1H), 2.60 (ddd, J = 2.5 Hz, J = 9.0 Hz, J = |
| | | 18.4 Hz, 1H), 2.51-2.36 (m, 1H), 2.16-1.85 (m, 3H), |
| | | 1.83-1.69 (m, 2H), 1.62 (br d, J = 13.5 Hz, 1H), 0.94 (d, |
| | | J = 6.9 Hz, 3H), 0.91 (d, J = 6.9 Hz, 3H) ppm. ¹³C NMR (75 |
| | | MHz, CDCl₃): δ 169.5, 132.8, 131.1 (q, J = 33 Hz), 131.0, |
| | | 125.8 (q, J = 3.8 Hz), 123.8 (q, J = 273 Hz), 91.4, 66.4, |
| | | 61.5, 56.6, 47.4, 43.5, 39.4, 32.2, 32.0, 29.9, 21.2, 19.6, |
| | | 18.6 ppm. |
| 53 | 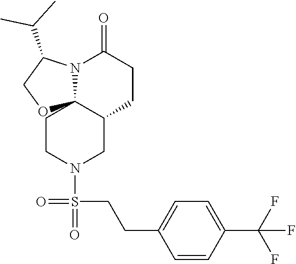 [SMILES: CC(C)[C@H]1CO[C@]23CCN(S(=O)(=O)CCc4ccc(C(F)(F)F)cc4)C[C@H]2CCC(=O)N13] | (3S,7aR,11aR)-3-isopropyl-9-[2-[4- (trifluoromethyl)phenyl]ethylsulfonyl]- 2,3,6,7,7a,8,10,11-octahydrooxazolo[2,3- j][1,6]naphthyridin-5-one Example 53 was synthetized from intermediate 21 and 2- [4-(trifluoromethyl)phenyl]ethanesulfonyl chloride using general protocol 11. The crude was evaporated under reduced pressure and purified through flash chromatography (cyclohexane/ethyl acetatec 100/0 to 50/50); Yield = 57%. ¹H NMR (300 MHz, CDCl₃): δ 7.60 (d, J = 8.1 Hz, 2H), 7.35 (d, J = 8.0 Hz, 2H), 4.19-4.09 (m, 1H), 3.98 (dd, J = 7.4 Hz, J = 8.9 Hz, 1H), 3.83-3.65 (m, 3H), 3.28 (dd, J = 3.0 Hz, J = 12.5 Hz, 1H), 3.18 (br s, 4H), 2.97 (dt, J = 3.0 Hz, J = 12.5 Hz, 1H), 2.63 (ddd, J = 2.5 Hz, J = 8.8 Hz, J = 18.3 Hz, 1H), 2.25-2.09 (m, 1H), 2.08-1.95 (m, 2H), 1.90-1.66 (m, 4H), 0.96 (d, J = 6.9 |
| | | Hz, 3H), 0.93 (d, J = 6.8 Hz, 3H) ppm. ¹³C NMR (75 MHz, |
| | | CDCl₃): δ 169.6, 142.0, 129.5 (q, J = 32 Hz), 128.8, 125.8 |
| | | (q, J = 3.8 Hz), 124.0 (q, J = 273 Hz), 91.4, 66.4, 61.5, |
| | | 50.6, 47.1, 43.1, 39.4, 32.1, 32.0, 29.7, 29.2, 20.3, 19.6, |
| | | 18.6 ppm. |
| 54 | 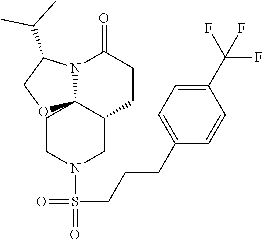 [SMILES: CC(C)[C@H]1CO[C@]23CCN(S(=O)(=O)CCCc4ccc(C(F)(F)F)cc4)C[C@H]2CCC(=O)N13] | (3S,7aR,11aR)-3-isopropyl-9-[3-[4- (trifluoromethyl)phenyl]propylsulfonyl]- 2,3,6,7,7a,8,10,11-octahydrooxazolo[2,3- j][1,6]naphthyridin-5-one Example 54 was synthetized from intermediate 21 and 3- [4-(trifluoromethyl)phenyl]propane-1-sulfonyl chloride using general protocol 11. The crude was evaporated under reduced pressure and purified through flash chromatography (cyclohexane/ethyl acetate: 100/0 to 50/50); Yield: 39%; ¹H NMR (300 MHz, CDCl₃): δ 7.57 (d, J = 8.1 Hz, 2H), 7.31 (d, J = 8.1 Hz, 2H), 4.13 (dq, J = 1.5 Hz, J = 7.4 Hz, 1H), 4.00 (dd, J = 7.3 Hz, J = 8.7 Hz, 1H), 3.80 (dd, J = 5.8 Hz, J = 8.8 Hz, 1H), 3.75-3.60 (m, 2H), 3.24 (dd, J = 3.0 Hz, J = 12.2 Hz, 1H), 3.0-2.80 (m, 5H), 2.62 (ddd, J = 2.2 Hz, J = 8.8 Hz, J = 18.5 Hz, 1H), 2.53- |
| | | 2.38 (m, 1H), 2.23-2.08 (m, 3H), 2.07-1.92 (m, 3H), |
| | | 1.88-1.64 (m, 4H), 0.95 (d, J = 6.9 Hz, 3H), 0.92 (J = 6.9 |
| | | Hz, 3H) ppm. ¹³C NMR (75 MHz, CDCl₃): δ 169.6, 144.2, |
| | | 128.9 (q, J = 31 Hz), 128.8, 125.6 (q, J = 3.7 Hz), 124.0 |
| | | (q, J = 274 Hz), 91.5, 66.4, 61.5, 48.5, 47.1, 43.1, 39.4, |
| | | 34.0, 31.1, 32.0, 30.0, 24.4, 20.3, 19.6, 18.6 ppm. |

### General Protocol 12: Buchwlad Synthesis

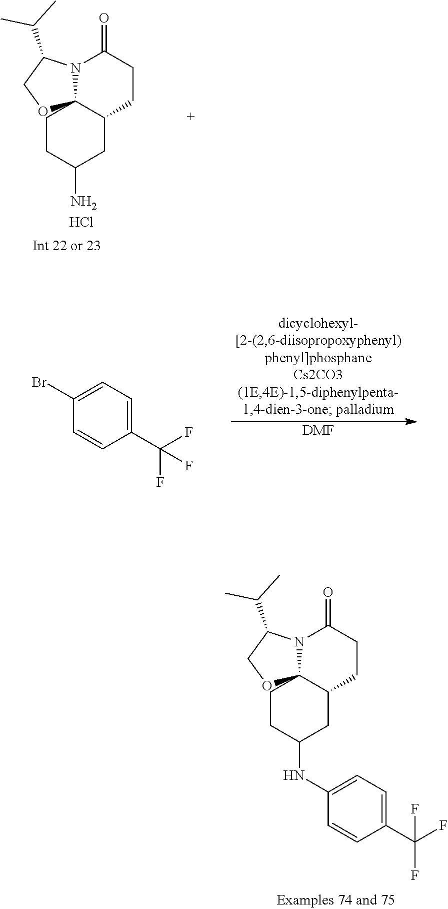

In a tube charged with the appropriate amine (1 eq), 1-bromo-4-(trifluoromethyl)benzene (1 eq), Pd₂dba₃ (5% mol), Ruphos (10% mol), Cs₂CO₃ (2.5 eq); dissolved in DMF. The tube was purged with argon and then heated at 120° C. for 5 h. The crude was filtered through a celite plug, eluted with MeOH until complete elution of UV visible product. The solvent was removed under reduced pressure. The crude was purified by flash chromatography (cyclohexane/ethyl acetate 100/0 to 70/30).

| Example | Structure | Physical data |
|---|---|---|
| 74 | 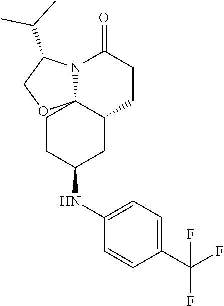 [SMILES: CC(C)[C@H]1CO[C@]23CC[C@H](Nc4ccc(C(F)(F)F)cc4)C[C@H]2CCC(=O)N13] | (3S,7aR,9S,11aR)-3-isopropyl-9-[4- (trifluoromethyl)anilino]-3,6,7,7a,8,9,10,11- octahydro-2H-oxazolo[2,3-j]quinolin-5-one Example 74 was synthetized from intermediate 22 and 1-bromo-4-(trifluromethyl)benzene using general protocol 12. Yield = 51%; ¹H NMR (300 MHz, CDCl₃): δ 7.40 (d, J = 8.4 Hz, 2H), 6.58 (d, J = 8.5 Hz, 2H), 4.18- 4.08 (m, 1H), 4.03 (dd, J = 7.5 Hz, J = 8.5 Hz, 1H), 3.80 (dd, J = 6.2 Hz, J = 8.5 Hz, 1H), 3.57 (br t, J = 11.2 Hz, 1H), 2.65 (ddd, J = 1.7 Hz, J = 8.0 Hz, J = 18.3 Hz, 1H), 2.53-2.38 (m, 1H), 2.13-1.93 (m, 5H), 1.91-1.76 (m, 4H), 1.65 (br s, 1H), 1.48 (ddd, J = 3.9 Hz, J = 12.5 Hz, J = 24.6 Hz, 1H), 0.98 (d, J = 6.9 Hz, 3H), 0.94 (d, J = 6.9 Hz, 3H) ppm. ¹³C NMR (75 MHz, CDCl₃): δ 169.8, 149.5, 126.7 (q, J = 3.7 Hz), 124.9 (q, J = 274 Hz), 118.7 (q, J = 34. Hz), 112.0, 93.3, 66.4, 61.3, 46.0, 40.0, 34.7, 32.6, 31.0, 30.4, 29.3, 22.7, 19.8, 18.7 ppm. |
| 75 | 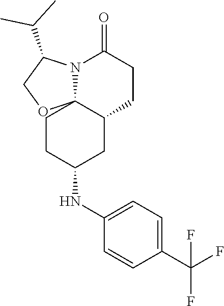 [SMILES: CC(C)[C@H]1CO[C@]23CC[C@@H](Nc4ccc(C(F)(F)F)cc4)C[C@H]2CCC(=O)N13] | (3S,7aR,9R,11aR)-3-isopropyl-9-[4- (trifluoromethyl)anilino]-3,6,7,7a,8,9,10,11- octahydro-2H-oxazolo[2,3-j]quinolin-5-one Example 75 was synthetized from intermediate 23 and 1-bromo-4-(trifluoromethyl)benzene using general protocol 12. Yield = 18%; ¹H NMR (300 MHz, CDCl₃): δ 7.40 (d, J = 8.5 Hz, 2H), 6.58 (d, J = 8.6 Hz, 2H), 4.08- 4.00 (m, 1H), 3.95 (br d, J = 4.7 Hz, 2H), 3.87-3.52 (m, 2H), 302 (d sept, J = 3.3 Hz, J = 7.2 Hz, 1H), 2.56-2.44 (m, 2H), 2.18-1.39 (m, 12H), 0.88 (d, J = 7.1 Hz, 3H), 0.74 (d, J = 6.9 Hz, 3H) ppm. ¹³C NMR (75 MHz, CDCl₃): δ 167.5, 149.5, 126.7 (q, J = 3.7 Hz), 124.9 (q, J = 274 Hz), 118.8 (q, J = 33 Hz), 112.1, 92.9, 62.6, 60.4, 45.8, 38.1, 34.2, 30.6, 29.2, 28.5, 24.6, 22.9, 19.5, 14.7 ppm. |

### Synthesis of (3S,7aR,11aR)-9-[2,2-difluoro-2-[4-(trifluoromethyl)phenyl]ethyl]-3-isopropyl-2,3,6,7,7a,8,10,11-octahydrooxazolo[2,3-j][1,6]naphthyridin-5-one (Example 41)

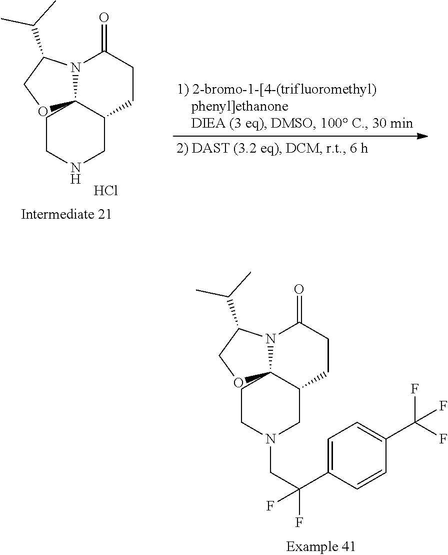 [SMILES: CC(C)[C@H]1CO[C@]23CCNC[C@H]2CCC(=O)N13 ; Cl ; CC(C)[C@H]1CO[C@]23CCN(CC(F)(F)c4ccc(C(F)(F)F)cc4)C[C@H]2CCC(=O)N13]

### Step 1:

In a tube were added (3S,7aR,11aR)-3-isopropyl-3,6,7,7a,8,9,10,11-octahydro-2H-oxazolo[2,3-j][1,6]naphthyridin-5-one; hydrochloride (275 mg, 1 mmol, 1 eq), 2-bromo-1-[4-(trifluoromethyl)phenyl]ethanone (267 mg, 1 mmol, 1 eq) dissolved in DMF (4 mL; on molecular sieves). Then 0.5 mL of DIEA was added. The mixture was stirred 30 min under microwave irradiation at 100° C.

The DMF was removed under vacuum. Ethyl acetate was added, and washed once with an aqueous solution of HCl 1N, once with a saturated aqueous solution of NaHCO₃ and once with brine. The organic layer was dried over MgSO₄, filtered and evaporated under reduced pressure The crude was introduce in the next step without any other treatment.

### Step 2:

The crude product of step 1 (50 mg, 0.12 mmol, 1 eq) was dissolved in 500 μL of dry dichloromethane. DAST (50 μL, 0.38 mmol, 3.2 eq) was added dropwise at 0° C. The mixture was stirred at room temperature for 6 h. The solution turned from light yellow to dark orange.

The reaction was quenched at 0° C. with an aqueous solution of saturated NaHCO₃; The aqueous layer was extracted three times with dichloromethane, organic fractions were pooled and washed once with brine, dried over MgSO₄, filtered and evaporated under reduced pressure. The crude was purified with flash chromatography (cyclohexane/ethyl acetate: 100/0 to 0:100). Fractions with desired product were pooled and evaporated under reduced pressure. This crude was purified with flash chromatography (dichloromethane/acetone: 100/0 to 80/20) to afford a fraction of the desired product at 60% purity. This fraction was then purified with reverse flash chromatography (water/MeCN: 90/10 to 0/100) to afford the desired product example 41. Yield=3%. ¹H NMR (300 MHZ, MeOD): δ 7.80 (d, J=8.4 Hz, 2H), 7.76 (d, J=8.4 Hz, 2H), 4.05-3.93 (m, 2H), 3.87-3.76 (m, 1H), 3.04 (ddd, J=3.0 Hz, J=12.2 Hz, J=15.6 Hz, 1H), 2.80 (dd, J=3.0 Hz, J=11.4 Hz, 1H), 2.71-2.57 (m, 2H), 2.56-2.43 (m, 2H), 2.39-2.23 (m, 1H), 2.07 (dd, J=3.7 Hz, J=8.7 Hz, 1H), 2.01-1.92 (m, 1H), 1.89-1.71 (m, 2H), 1.53 (br d, J=15.1 Hz, 1H), 1.49-1.41 (m, 1H), 1.31 (br s, 1H), 0.92 (d, J=6.9 Hz, 3H), 0.91 (d, J=6.9 Hz, 3H) ppm. 13C NMR (75 MHZ, MeOD, d4): δ 172.6, 140.4, 131.5 (q, J=33 Hz), 127.7 (t, J=6.2 Hz), 126.2 (q, J=3.8 Hz), 124.0 (q, J=274 Hz), 121.6 (t, J=234 Hz), 93.2, 67.1, 63.7 (t, J=30 Hz), 62.7, 57.1, 52.5, 41.7, 33.6, 33.0, 31.3, 30.7, 22.6, 20.2, 18.9 ppm.

### General Protocol 13: Functionalization of Lactam Ring

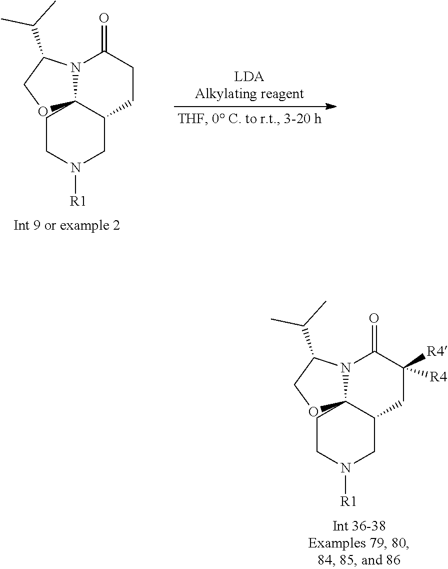

Intermediate 9 or Example 2 (1 eq) was dissolved in dry THF (0.15 M). The solution was cooled down to 0° C. then LDA (1.2-3 eq) was added dropwise. The mixture was stirred during 1 h, then a solution alkylating reagent (1.2-2 eq) in dry THF was added dropwise. The resulting mixture was warmed up to room temperature and stirred for 3-20 h. The solution was quenched with H₂O, extracted with diethyl ether. The organic layer was washed with a saturated aqueous solution of NH₄Cl, dried over MgSO₄, filtered, and concentrated under vacuum. The crude product obtained was purified by flash chromatography column over silica gel (cyclohexane/ethyl acetate: 100/0 to 0/100) to afford the corresponding desired product.

| Intermediate | structure | Physical data |
|---|---|---|
| 36 | 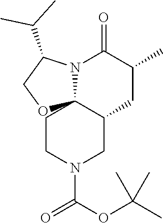 [SMILES: CC(C)[C@H]1CO[C@]23CCN(C(=O)OC(C)(C)C)C[C@H]2C[C@@H](C)C(=O)N13] | Tert-butyl(3S,6R,7aR,11aR)-3-isopropyl-6-methyl-5-oxo- 2,3,6,7,7a,8,10,11-octahydrooxazolo[2,3- j][1,6]naphthyridine-9-carboxylate Intermediate 36 was obtained from intermediate 9 and iodomethane using general protocol 13; Yield: 37%; colorless oil; ¹H NMR (300 MHz, CDCl₃): δ 4.17-3.97 (m, 4H), 3.71 (d, J = 8.1 Hz, 6.0 Hz, 1H), 3.29-3.11 (m, 1H), 2.94-2.77 (m, 1H), 2.53-2.38 (m, 1H), 2.01-1.50 (m, 6H), 1.45 (s, 9H), 1.26 (d, J = 7.6 Hz, 3H), 0.91 (d, J = 6.9 Hz, 3H), 0.88 (d, J = 6.9 Hz, 3H) ppm. ¹³C NMR (75 MHz, CDCl₃): δ 173.5, 155.1, 92.5, 79.9, 66.9, 61.2, 45.7, 41.4, 40.4, 37.1, 32.9, 31.7, 30.4, 28.5, 19.9, 19.8, 18.8 ppm; [ES+ MS] m/z 353 (MH⁺). |
| 37 | 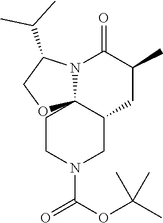 [SMILES: CC(C)[C@H]1CO[C@]23CCN(C(=O)OC(C)(C)C)C[C@H]2C[C@H](C)C(=O)N13] | Tert-butyl(3S,6S,7aR,11aR)-3-isopropyl-6-methyl-5-oxo- 2,3,6,7,7a,8,10,11-octahydrooxazolo[2,3- j][1,6]naphthyridine-9-carboylate Intermediate 37 was obtained from intermediate 9 and iodomethane using general protocol 13; Yield: 38%; colorless oil. ¹H NMR (300 MHz, CDCl₃): δ 4.13-4.01 (m, 2H), 3.92 (d, J = 8.8, 7.1 Hz, 2H), 3.78 (d, J = 8.8, 5.5 Hz, 1H), 3.27-3.09 (m, 1H), 2.96-2.77 (m, 1H), 2.58-2.46 (m, 1H), 2.08-1.56 (m, 6H), 1.44 (s, 9H), 1.21 (d, J = 7.2 Hz, 3H), 0.89 (d, J = 6.6 Hz, 3H), 0.87 (d, J = 6.6 Hz, 3H) ppm. ¹³C NMR (75 MHz, CDCl₃): δ 173.1, 155.2, 92.6, 79.9, 66.3, 61.5, 44.8, 41.2, 40.4, 37.8, 33.5, 31.9, 31.9, 28.5, 19.7, 18.7, 18.2 ppm. [ES+ MS] m/z 353 (MH⁺). |
| 38 | 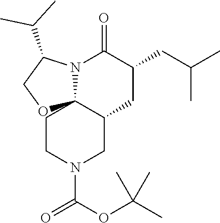 [SMILES: CC(C)C[C@@H]1C[C@@H]2CN(C(=O)OC(C)(C)C)CC[C@@]23OC[C@H](C(C)C)N3C1=O] | Tert-butyl(3S,6R,7aR,11aR)-6-isobutyl-3-isopropyl-5- oxo-2,3,6,7,7a,8,10,11-octahydrooxazolo[2,3- j][1,6]naphthyridine-9-carboxylate Intermediate 38 was obtained from intermediate 9 and 1- bromo-2-methyl-propane using general protocol 13; Yield: 19%; colorless oil; [ES+ MS] m/z 395 (MH⁺). |

| Example | structure | Physical data |
|---|---|---|
| 79 | 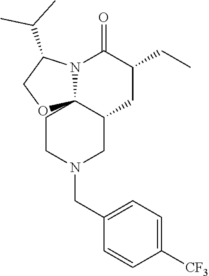 [SMILES: CC[C@@H]1C[C@@H]2CN(Cc3ccc(C(F)(F)F)cc3)CC[C@@]23OC[C@H](C(C)C)N3C1=O] | (3S,6R,7aR,11aR)-6-ethyl-3-isopropyl-9-[[4- (trifluoromethyl)phenyl]methyl]-2,3,6,7,7a,8,10,11- octahydrooxazolo[2,3-j][1,6]naphthyridin-5-one Example 79 was obtained from example 2 and bromoethane using general protocol 13; Yield: 21%; colorless oil; ¹H NMR (300 MHz, CDCl₃): δ 7.59 (d, J = 7.4 Hz, 2H), 7.46 (d, J = 7 .4 Hz, 2H), 4.21-4.09 (m, 1H), 4.05 (dd, J = 8.5, 8.5 Hz, 1H), 3.76 (dd, J = 8.5 Hz, 6.7 Hz, 1H), 3.60 (d, J = 13.2 Hz, 1H), 3.48 (d, J = 13.2 Hz, 1H), 2.80-2.61 (m, 2H), 2.57-2.34 (m, 2H), 2.32-2.08 (m, 2H), 2.06-1.54 (m, 8H), 0.99 (t, J = 7.4 Hz, 3H), 0.95 (d, J = 6.8 Hz, 3H), 0.91 (d, J = 6.8 Hz, 3H) ppm. ¹³C NMR (75 MHz, CDCl₃): δ 173.1, 143.1, 129.6, 128.9, 127.6 (q, J = 271 Hz), 125.3, 92.4, 66.6, 62.2, 61.1, 55.2, 50.9, 43.4, 40.4, 33.0, 32.6, 28.0, 26.4, 20.0, 18.8, 10.8 ppm. [ES+ MS] m/z 425 (MH⁺). |
| 83 | 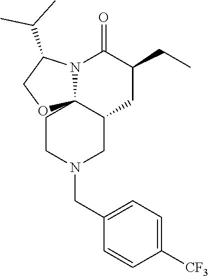 [SMILES: CC[C@H]1C[C@@H]2CN(Cc3ccc(C(F)(F)F)cc3)CC[C@@]23OC[C@H](C(C)C)N3C1=O] | (3S,6S,7aR,11aR)-6-ethyl-3-isopropyl-9-[[4- (trifluoromethyl)phenyl]methyl]-2,3,6,7,7a,8,10,11- octahydrooxazolo[2,3-j][1,6]naphthyridin-5-one Example 83 was obtained from example 2 and bromoethane using general protocol 13; Yield: 11%; colorless oil; ¹H NMR (300 MHz, CDCl₃): δ 7.65-7.38 (m, 4H), 4.15-4.07 (m, 1H), 3.93 (dd, J = 8.7, 7.0 Hz, 1H), 3.78 (dd, J = 8.7 Hz, 6.1 Hz, 1H), 3.70-3.45 (m, 2H), 2.81-2.36 (m, 5H), 2.08-1.83 (m, 4H), 1.83-1.59 (m, 2H), 1.56-1.38 (m, 2H), 0.99-0.87 (m, 9H) ppm. ¹³C NMR (75 MHz, CDCl₃): δ 172.9, 143.0, 130.2, 129.3, 126.4 (q, J = 271 Hz), 125.6, 92.6, 66.3, 62.1, 61.6, 54.7, 50.8, 40.3, 38.3, 32.5, 32.1, 26.7, 25.1, 19.9, 18.9, 12.1 ppm. [ES+ MS] m/z 425 (MH⁺). |
| 80 | 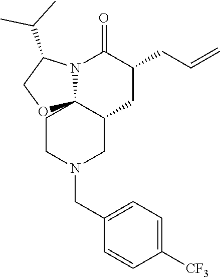 [SMILES: C=CC[C@@H]1C[C@@H]2CN(Cc3ccc(C(F)(F)F)cc3)CC[C@@]23OC[C@H](C(C)C)N3C1=O] | (3S,6R,7aR,11aR)-6-allyl-3-isopropyl-9-[[4- (trifluoromethyl)phenyl]methyl]-2,3,6,7,7a,8,10,11- octahydrooxazolo[2,3-j][1,6]naphthyridin-5-one Example 80 was obtained from example 2 and allyl bromide using general protocol 13; Yield: 30%; colorless oil; ¹H NMR (300 MHz, CDCl₃): δ 7.57 (d, J = 8.1 Hz, 2H), 7.43 (d, J = 8.1 Hz, 2H), 5.86-.570 (m, 1H), 5.21-5.10 (m, 2H), 4.17-4.08 (m, 1H), 4.04 (dd, J = 15.6, 6.9 Hz, 1H), 3.78 (dd, J = 8.5, 6.9 Hz, 1H), 3.57 (d, J = 13.8 Hz, 1H), 3.45 (d, J = 13.8 Hz, 1H), 2.73-2.61 (m, 2H), 2.59-2.41 (m, 4H), 2.29-2.14 (m, 2H), 2.03-1.88 (m, 2H), 1.85-1.75 (m, 1H), 1.73-1.58 (m, 2H), 0.93 (d, J = 6.8 Hz, 3H), 0.88 (d, J = 6.8 Hz, 3H) ppm. ¹³C NMR (75 MHz, CDCl₃): δ 172.3, 143.2, 135.3, 129.4 (q, J = 32.4 Hz), 128.9, 125.3 (q, J = 3.6 Hz), 124.5 (q, J = 274 Hz), 117.8, 92.5, 66.7, 62.1, 61.2, 55.2, 50.8, 42.0, 40.4, 37.7, 33.0, 32.3, 28.2, 20.0, 18.8 ppm. [ES+ MS] m/z 437 (MH⁺). |
| 84 | 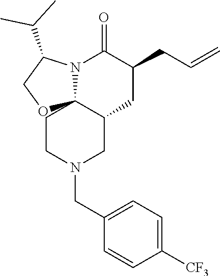 [SMILES: C=CC[C@H]1C[C@@H]2CN(Cc3ccc(C(F)(F)F)cc3)CC[C@@]23OC[C@H](C(C)C)N3C1=O] | (3S,6S,7aR,11aR)-6-allyl-3-isopropyl-9-[[4- (trifluoromethyl)phenyl]methyl]-2,3,6,7,7a,8,10,11- octahydrooxazolo[2,3-j][1,6]naphthyridin-5-one Example 84 was obtained from example 2 and allyl bromide using general protocol 13; Yield: 12%; colorless oil; ¹H NMR (300 MHz, CDCl₃): δ 7.57 (d, J = 8.5 Hz, 2H), 7.43 (d, J = 8.7 Hz, 2H), 5.88-5.72 (m, 1H), 5.11-5.01 (m, 2H), 4.14-4.04 (m, 1H), 3.93 (dd, J = 8.8 Hz, 7.2 Hz, 1H), 3.78 (dd, J = 8.8 Hz, 5.9 Hz, 1H), 3.56 (d, J = 14.0 Hz, 1H), 3.48 (d, J = 14.0 Hz, 1H), 2.73-2.39 (m, 6H), 2.32-2.16 (m, 1H), 2.06-1.88 (m, 2H), 1.79-1.71 (m, 1H), 1.70-1.47 (m, 3H), 0.93 (d, J = 6.8 Hz, 3H), 0.89 (d, J = 6.8 Hz, 3H) ppm. ¹³C NMR (75 MHz, CDCl₃): δ 172.2, 143.2, 136.6, 129.4 (q, J = 32.2 Hz), 128.9, 125.4 (q, J = 3.6 Hz), 124.5 (q, J = 271 Hz), 117.1, 92.5, 66.2, 62.1, 61.5, 54.9, 50.5, 38.3, 38.2, 36.6, 32.5, 31.9, 26.4, 19.8, 18.7 ppm. [ES+ MS] m/z 437 (MH⁺). |
| 85 | 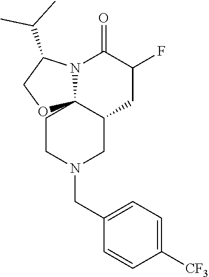 | (3S,7aR,11aR)-6-fluoro-3-isopropyl-9-[[4- (trifluoromethyl)phenyl]methyl]-2,3,6,7,7a,8,10,11- octahydrooxazolo[2,3-j][1,6]naphthyridin-5-one Example 85 was obtained from example 2 and N- (benzenesulfonyl)-N-fluoro-benzenesulfonamide using general protocol 13; Yield: 18%; colorless oil; ¹H NMR (300 MHz, CDCl₃ ): δ 7.51 (d, J = 7.7 Hz, 2H), 7.36 (d, J = 7.7 Hz, 2H), 4.93 (ddd, J = 48.4 Hz, 6.7 Hz, 2.3 Hz, 1H), 4.16-4.05 (m, 1H), 3.97 (d, J = 8.1 Hz, 1H), 3.74 (dd, J = 8.8 Hz, 6.2 Hz, 1H), 3.47 (d, J = 13.7 Hz, 1H), 3.42 (d, J = 13.7 Hz, 1H), 2.81-2.40 (m, 4H), 2.25-2.07 (m, 1H), 2.03-1.69 (m, 5H), 0.87 (d, J = 6.8 Hz, 3H), 0.83 (d, J = 6.8 Hz, 3H) ppm; ¹³C NMR (75 MHz, CDCl₃): δ 166.1 (d, J = 21.4 Hz), 142.9, 129.6 (q, J = 31.7 Hz), 128.9, 125.4 (q, J = 3.0 Hz), 124.6 (q, J = 271 Hz), 92.8, 84.8 (d, J = 175.5 Hz), 66.2, 61.9, 61.1, 54.3, 50.3, 36.8, 32.2, 32.1, 29.7 (d, J = 22.3 Hz), 19.8, 18.7 ppm. [ES+ MZ] m/z 415 (MH⁺). |
| 86 | 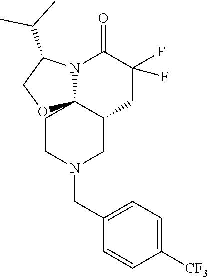 [SMILES: CC(C)[C@H]1CO[C@]23CCN(Cc4ccc(C(F)(F)F)cc4)C[C@H]2CC(F)(F)C(=O)N13] | (3S,7aR,11aR)-6,6-difluoro-3-isopropyl-9-[[4- (trifluoromethyl)phenyl]methyl]-3,7,7a,8,10,11- hexahydro-2H-oxazolo[2,3-j][1,6]naphthyridin-5-one Example 97 was obtained from example 2 and N- (benzenesulfonyl)-N-fluoro-benzenesulfonamide using general protocol 13; Yield: 23%; colorless oil; ¹H NMR (300 MHz, CDCl₃): δ 7.51 (d, J = 8.1 Hz, 2H), 7.36 (d, J = 7.5 Hz, 2H), 4.10-3.98 (m, 2H), 3.83-3.71 (m, 1H), 3.53 (d, J = 13.6 Hz, 1H), 3.42 (d, J = 13.6 Hz, 1H), 2.96-2.62 (m, 2H), 2.62- 2.42 (m, 2H), 2.28-2.12 (m, 2H), 2.02-1.78 (m, 4H), 0.89 (d, J = 6.9 Hz, 3H), 0.84 (d, J = 6.9 Hz, 3H) ppm. ¹³C NMR (75 MHz, CDCl₃): δ 166.2 (dd, J = 32.6, 29.5 Hz), 142.8, 129.8 (q, J = 31.8 Hz), 129.0, 125.4 (q, J = 3.0 Hz), 124.5 (q, J = 271.6 Hz), 113.5 (dd, J = 248.5, 248.5 Hz), 92.6, 67.4, 61.8, 61.8, 54.1, 50.4, 38.3 (d, J = 6.64 Hz), 34.3 (dd, J = 23.4 Hz), 32.7, 31.7, 19.9, 18.8 ppm. [ES+ MS] m/z 433 (MH⁺). |

### General Protocol 14: Synthesis of Examples 78, 81 and 82

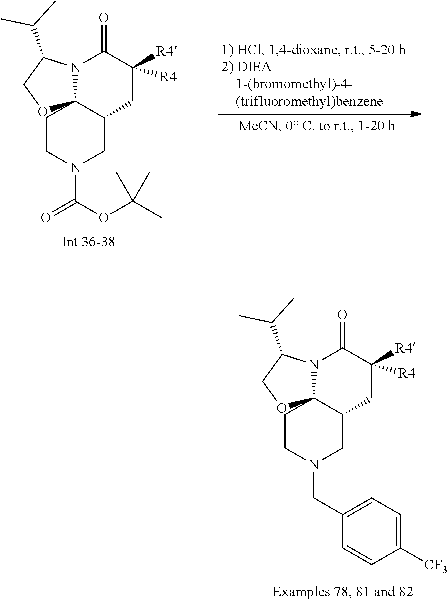

Step 1: The appropriate intermediate was dissolved in 1,4-dioxane (0.05 M), then HCl in solution in 1,4-dioxane (4N, 10 eq) was added. The mixture was stirred at room temperature for 5-20 h. When the conversion of the starting material was complete (as determined by TLC or LC/MS), the solvent was removed under vacuum and the crude product obtained was engaged into the next step without any further purification.

Step 2: The crude product of step 1 was dissolved in MeCN (0.1N). The solution was cooled down to 0° C., then DIEA (1.5-3 eq) and 1-(bromomethyl)-4-(trifluoromethyl)benzene (1.5 eq) were added. The mixture was warmed up to room temperature and stirred for 1-20 h. The solvent was removed under reduced pressure. The residue obtained was dissolved in H₂O and extracted with dichloromethane. The organic layer was dried over MgSO₄, filtered and concentrated under vacuum to give the crude product, which was purified by flash chromatography column over silica gel (cyclohexane/ethyl acetate: 100/0 to 0/100) to afford the corresponding trifluoromethylbenzyl substituted lactams.

| Example | Structure | Physical data |
|---|---|---|
| 78 | 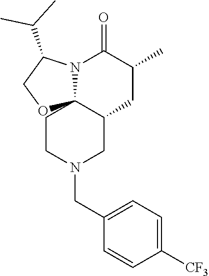 [SMILES: CC(C)[C@H]1CO[C@]23CCN(Cc4ccc(C(F)(F)F)cc4)C[C@H]2C[C@@H](C)C(=O)N13] | (3S,6R,7aR,11aR)-3-isopropyl-6-methyl-9-[[4- (trifluoromethyl)phenyl]methyl]-2,3,6,7,7a,8,10,11- octahydrooxazolo[2,3-j][1,6]naphthyridin-5-one Example 78 was obtained from intermediate 36 using general protocol 14; Yield: 70% over 2 steps; colorless oil; ¹H NMR (300 MHz, CDCl₃): δ 7.57 (d, J = 8.1 Hz, 2H), 7.44 (d, J = 8.1 Hz, 2H), 4.14-4.09 (m, 1H), 4.05 (dd, J = 14.0, 5.8 Hz, 1H), 3.78-3.71 (m, 1H), 3.57 (d, J = 14.0 Hz, 1H), 3.45 (d, J = 14.0 Hz, 1H), 2.75-2.61 (m, 2H), 2.51-2.31 (m, 2H), 2.28-2.15 (m, 1H), 2.14-1.69 (m, 5H), 1.69-1.59 (m, 1H), 1.31 (d, J = 7.2 Hz, 3H), 0.93 (d, J = 6.8 Hz, 3H), 0.88 (d, J = 6.8 Hz, 3H) ppm. ¹³C NMR (75 MHz, CDCl₃): δ 173.9, 143.2, 129.4 (q, J = 32.0 Hz), 128.9, 125.3 (q, J = 3.6 Hz), 124.4 (q, J = 273 Hz), 92.7, 66.7, 62.2, 61.0, 55.2, 50.9, 40.6, 33.1, 33.1, 32.3, 31.7, 20.0, 19.9, 18.8 ppm. [ES+ MS] m/z 411 (MH⁺). |
| 82 | 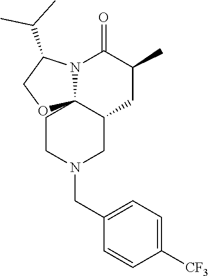 [SMILES: CC(C)[C@H]1CO[C@]23CCN(Cc4ccc(C(F)(F)F)cc4)C[C@H]2C[C@H](C)C(=O)N13] | (3S,6S,7aR,11aR)-3-isopropyl-6-methyl-9-[[4- (trifluoromethyl)phenyl]methyl]-2,3,6,7,7a,8,10,11- octahydrooxazolo[2,3-j][1,6]naphthyridin-5-one Example 82 was obtained from intermediate 37 using general protocol 14. Yield: 82% over 2 steps; colorless oil; ¹H NMR (300 MHz, CDCl₃): δ 7.57 (d, J = 8.1 Hz, 2H), 7.44 (d, J = 8.1 Hz, 2H), 4.13-4.03 (m, 1H), 3.93 (dd, J = 8.8, 7.2 Hz, 1H), 3.78 (dd, J = 8.8, 5.6 Hz, 1H), 3.59 (d, J = 13.8 Hz, 1H), 3.45 (d, J = 13.8 Hz, 1H), 2.76-2.43 (m, 5H), 2.31-2.19 (m, 1H), 2.06-1.90 (m, 2H), 1.80-1.72 (m, 1H), 1.72-1.62 (m, 1H), 1.42-1.31 (m, 1H), 1.21 (d, J = 7.0 Hz, 3H), 0.93 (d, J = 6.7 Hz, 3H), 0.89 (d, J = 6.7 Hz, 3H) ppm. ¹³C NMR (75 MHz, CDCl₃): δ 173.5, 143.2, 129.4 (q, J = 32.0 Hz), 128.9, 125.3 (q, J = 3.6 Hz), 124.6 (q, J = 271 Hz), 92.7, 66.2, 62.1, 61.4, 54.8, 50.7, 37.8, 33.8, 32.6, 32.1, 29.9, 19.8, 18.8, 18.2 ppm. [ES+ MS] m/z 411 (MH⁺). |
| 81 | 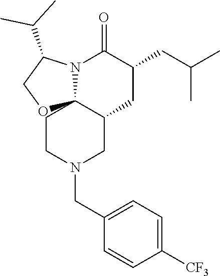 [SMILES: CC(C)C[C@@H]1C[C@@H]2CN(Cc3ccc(C(F)(F)F)cc3)CC[C@@]23OC[C@H](C(C)C)N3C1=O] | (3S,6R,7aR,11aR)-6-isobutyl-3-isopropyl-9-[[4- (trifluoromethyl)phenyl]methyl]-2,3,6,7,7a,8,10,11- octahydrooxazolo[2,3-j][1,6]naphthyridin-5-one Example 81 was obtained from intermediate 38 using general protocol 14; Yield: 82% over 2 steps; colorless oil; ¹H NMR (300 MHz, CDCl₃ ): δ 7.57 (d, J = 8.1 Hz, 2H), 7.43 (d, J = 8.1 Hz, 2H), 4.17-4.05 (m, 1H), 4.04 (dd, J = 16.4 Hz, 8.0 Hz, 1H), 3.73 (dd, J = 8.4, 6.7 Hz, 1H), 3.56 (d, J = 13.8 Hz, 1H), 3.45 (d, J = 13.8 Hz, 1H), 2.74-2.62 (m, 2H), 2.52-2.46 (m, 1H), 2.44-2.32 (m, 1H), 2.28-2.15 (m, 1H), 2.11-1.67 (m, 7H), 1.67-1.55 (m, 1H), 1.43-1.31 (m, 1H), 0.96 (d, J = 6.4 Hz, 3H), 0.91 (d, J = 6.8 Hz, 3H), 0.90 (d, J = 6.4 Hz, 3H), 0.88 (d, J = 6.8 Hz, 3H) ppm. ¹³C NMR (75 MHz, CDCl₃): δ 174.0, 143.2, 136.6, 129.5 (q, J = 32.1 Hz), 129.0, 125.4 (q, J = 3.5 Hz), 124.4 (q, J = 272.4 Hz), 92.5, 66.6, 62.3, 61.1, 55.4, 50.9, 44.0, 40.5, 40.5, 33.1, 32.2, 29.3, 25.3, 23.8, 21.3, 20.0, 18.9 ppm. [ES+ MS] m/z 453 (MH⁺). |

### Synthesis of example 68 and 69

### Step 1: Oxidation of Intermediate 9

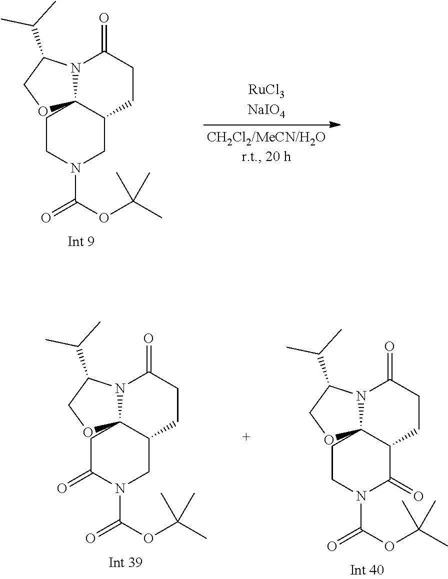 [SMILES: CC(C)[C@H]1CO[C@]23CCN(C(=O)OC(C)(C)C)C[C@H]2CCC(=O)N13 ; CC(C)[C@H]1CO[C@]23CC(=O)N(C(=O)OC(C)(C)C)C[C@H]2CCC(=O)N13 ; CC(C)[C@H]1CO[C@]23CCN(C(=O)OC(C)(C)C)C(=O)[C@H]2CCC(=O)N13]

Intermediate 9 (50 mg, 0.148 mmol, 1 eq) was dissolved in CH₂Cl₂ (2 mL), MeCN (2 mL) and H₂O (3 mL), then RuCl₃ (6.13 mg, 0.029 mmol, 20 mol %) and NaIO₄ (126 mg, 0.591 mmol, 4 eq) were added. The solution was stirred at room temperature overnight. The mixture was extracted with dichloromethane. The organic layer was dried over MgSO₄, filtered, then concentrated under reduced pressure to give the crude product, which was purified by flash chromatography over silica gel column (cyclohexane/ethyl acetate: 100/0 to 0/100) to afford the intermediates 39 and 40 (35 mg, 67%) as a colorless oil, in proportion (1/1).

### Tert-butyl (3S,7aR,11aR)-3-isopropyl-5,10-dioxo-3,6,7,7a,8,11-hexahydro-2H-oxazolo[2,3-j][1,6]naphthyridine-9-carboxylate (Intermediate 39)

[ES+MS] m/z 353 (MH⁺).

### Tert-butyl (3S,7aS,11aR)-3-isopropyl-5,8-dioxo-3,6,7,7a, 10,11-hexahydro-2H-oxazolo[2,3-j][1,6]naphthyridine-9-carboxylate (Intermediate 40)

[ES+MS] m/z 353 (MH⁺).

Steps 2 and 3: conversion of intermediates 39 and 40 into examples 68 and 69

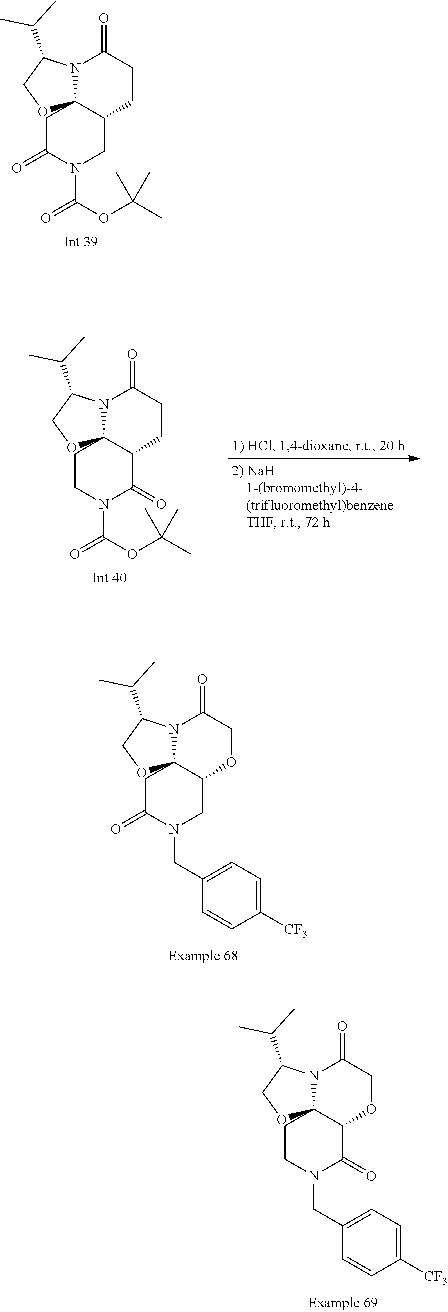 [SMILES: CC(C)[C@H]1CO[C@]23CC(=O)N(C(=O)OC(C)(C)C)C[C@H]2CCC(=O)N13 ; CC(C)[C@H]1CO[C@]23CCN(C(=O)OC(C)(C)C)C(=O)[C@H]2CCC(=O)N13 ; CC(C)[C@H]1CO[C@]23CC(=O)N(Cc4ccc(C(F)(F)F)cc4)C[C@H]2OCC(=O)N13 ; CC(C)[C@H]1CO[C@]23CCN(Cc4ccc(C(F)(F)F)cc4)C(=O)[C@H]2OCC(=O)N13]

Step 2: The mixture of intermediate 39 and 40 (35 mg, 0.099 mmol) was dissolved in 1,4-dioxane (2 mL), then HCl in solution in 1,4-dioxane (4N, 0.25 mL, 1 mmol, 10 eq) was added. The mixture was stirred at room temperature for 20 h. When the conversion of the starting material was judged completed by TLC or LC/MS, the solvent was removed under vacuum and the crude product obtained was engaged into the next step reaction without further purification.

Step 3: The crude product of step 1 was dissolved in THF (3 mL), then NaH (60% in suspension in oil, 96 mg, 2.49 mmol, 6 eq) and 1-(bromomethyl)-4-(trifluoromethyl)benzene (36 mg, 0.151 mmol, 1.5 eq) were added. The mixture was stirred at to room temperature for 72 h. The solvent was removed under reduced pressure. The residue obtained was dissolved in H₂O and extracted with dichloromethane. The organic layer was dried over MgSO₄, filtered and concentrated under vacuum to give the crude product, which was purified by preparative HPLC using H₂O+0.1% HCOOH/MeCN+0.1% HCOOH gradient (100/0 to 0/100), to afford example 68 and example 69.

### (3S,7aR,11aR)-3-isopropyl-9-[[4-(trifluoromethyl)phenyl]methyl]-3,6,7,7a,8,11-hexahydro-2H-oxazolo[2,3-j][1,6]naphthyridine-5,10-dione (example 68)

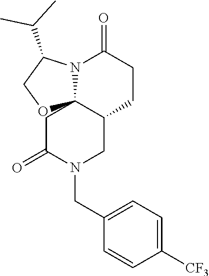 [SMILES: CC(C)[C@H]1CO[C@]23CC(=O)N(Cc4ccc(C(F)(F)F)cc4)C[C@H]2CCC(=O)N13]

Yield: 38%; colorless oil; ¹H NMR (300 MHZ, CDCl₃): δ 7.58 (d, J=8.1 Hz, 2H), 7.34 (d, J=8.1 Hz, 2H), 4.81 (d, J=15.0 Hz, 1H), 4.49 (d, J=15.0 Hz, 1H), 4.13-4.05 (m, 1H), 3.97 (dd, J=8.9, 7.1 Hz, 1H), 3.87 (dd, J=8.9 Hz, 6.2 Hz, 1H), 3.69 (dd, J=12.5, 4.3 Hz, 1H), 3.02 (dd, J=12.5, 1.0 Hz, 1H), 2.89 (d, J=18.0 Hz, 1H), 2.70 (d, J=18.0 Hz, 1H), 2.66-2.38 (m, 2H), 2.17-2.04 (m, 1H), 1.94-1.67 (m, 3H), 0.93 (d, J=2.2 Hz, 3H), 0.91 (d, J=2.2 Hz, 3H) ppm. ¹³C NMR (75 MHZ, CDCl₃): δ 165.8, 164.5, 140.7, 130.0 (q, J=32.3 Hz), 128.2, 125.8 (q, J=3.8 Hz), 124.9 (q, J=271.7 Hz), 92.0, 66.2, 61.7, 49.6, 47.9, 40.8, 37.8, 31.1, 30.1, 21.4, 19.5, 18.2 ppm. [ES+MS] m/z 411 (MH⁺).

### (3S,7aS,11aR)-3-isopropyl-9-[[4-(trifluoromethyl)phenyl]methyl]-3,6,7,7a, 10,11-hexahydro-2H-oxazolo[2,3-j][1,6]naphthyridine-5,8-dione (example 69)

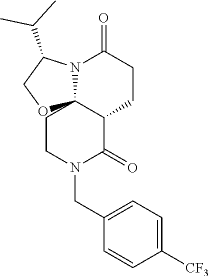 [SMILES: CC(C)[C@H]1CO[C@]23CCN(Cc4ccc(C(F)(F)F)cc4)C(=O)[C@H]2CCC(=O)N13]

Yield: 24%; colorless oil; ¹H NMR (300 MHZ, CDCl₃): δ 7.57 (d, J=8.0 Hz, 2H), 7.32 (d, J=8.1 Hz, 2H), 4.68 (d, J=15.0 Hz, 1H), 4.61 (d, J=15.0 Hz, 1H), 4.19-4.04 (m, 2H), 3.70 (dd, J=8.2, 5.9 Hz, 1H), 3.46-3.33 (m, 1H), 3.13-3.03 (m, 1H), 2.69-2.37 (m, 4H), 2.14-2.01 (m, 2H), 1.96-1.78 (m, 2H), 0.96 (d, J=2.2 Hz, 3H), 0.90 (d, J=6.8 Hz, 3H) ppm. 13C NMR (75 MHZ, CDCl₃): δ 170.0, 169.7, 140.7, 129.9 (q, J=32.2 Hz), 128.0, 125.8 (q, J=3.8 Hz), 124.2 (q, J=271.7 Hz), 92.2, 67.2, 61.2, 49.7, 47.3, 43.2, 33.6, 31.0, 28.7, 23.9, 20.0, 19.1 ppm. [ES+MS] m/z 411 (MH⁺).

### Synthesis of 4,4-dimethoxy-1-[[4-(trifluoromethyl)phenyl]methyl]piperidin-3-ol (Intermediate 41)

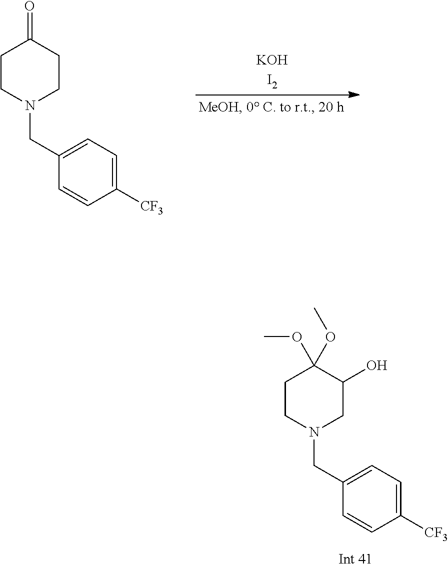 [SMILES: O=C1CCN(Cc2ccc(C(F)(F)F)cc2)CC1 ; COC1(OC)CCN(Cc2ccc(C(F)(F)F)cc2)CC1O]

1-[[4-(trifluoromethyl)phenyl]methyl]piperidin-4-one (1.54 g, 5.99 mmol, 1 eq) was dissolved in MeOH (12 mL), then KOH (840 mg, 15 mmol, 2.5 eq) was added. The reaction was placed at 0° C. and let stirred at this temperature for 30 min. Then a solution of 12 (2.28 g, 8.98 mmol, 1.5 eq) in MeOH (5 mL) was added dropwise over 1 h. The mixture was warmed up to room temperature and stirred overnight (20 h). The mixture was concentrated under reduced pressure. The oil obtained was extracted with ethyl acetate. The organic layer was dried over MgSO₄, filtered, concentrated under vacuum to afford the crude product, which was purified by flash chromatography column on silica gel (cyclohexane/ethyl acetate: 100/0 to 0/100) to afford the intermediate 41 (1.715 g, 90%), as a brown oil; ¹H NMR (300 MHz, CDCl₃): δ 7.60 (d, J=8.1 Hz, 2H), 7.48 (d, J=8.1 Hz, 2H), 3.85-3.65 (m, 3H), 3.26 (s, 3H), 3.21 (s, 3H), 2.94-2.75 (m, 2H), 2.64-2.58 (m, 1H), 2.35-2.18 (m, 3H) ppm. [ES+MS] m/z 320 (MH⁺).

### Synthesis of Methyl 2-[[4,4-dimethoxy-1-[[4-(trifluoromethyl)phenyl]methyl]-3-piperidyl]oxy]acetate (Intermediate 42)

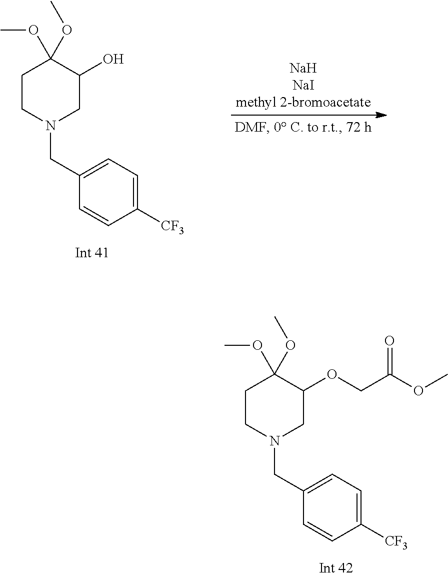

Intermediate 41 (1.6 g, 5.01 mmol, 1 eq) was dissolved in DMF (12 mL). The solution was cooled down to 0° C., then NaH (60% in suspension in oil, 576 mg, 15.0 mmol, 3 eq) was added. The solution was stirred for 0.5 h. A solution of methylbromoacetate (1.2 mL, 12.5 mmol, 2.5 eq) was added dropwise. The reaction was warmed to room temperature and let stirred for 72 h. The mixture was evaporated until dryness. The residue obtained was dissolved H₂O and extracted with ethyl acetate. The organic layer was dried over MgSO₄, filtered, and concentrated under vacuum to give the crude product. The crude product was purified by flash chromatography column on silica gel (cyclohexane/ethyl acetate: 100/0 to 0/100) to afford the intermediate 42 (326 mg, 17%) as a colorless oil; [ES+MS] m/z 392 (MH⁺).

### Synthesis of (1R,4S,9R)-4-isopropyl-11-[[4-(trifluoromethyl)phenyl]methyl]-2,8-dioxa-5,11-diazatricyclo[7.4.0.01,5]tridecan-6-one (example 66)

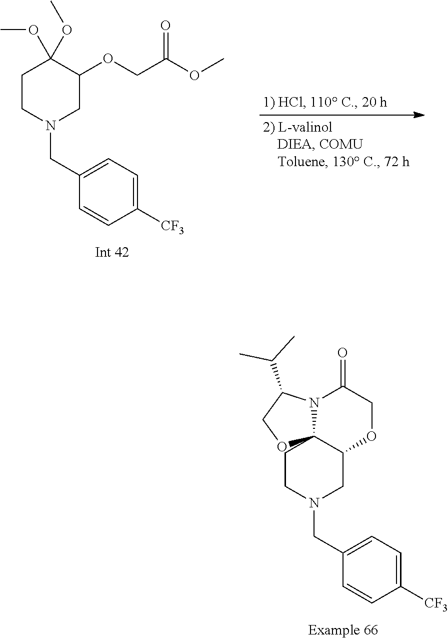

Step 1: Intermediate 42 (300 mg, 0.767 mmol, 1 eq) was dissolved in aqueous solution of HCl (0.1N, 50 mL, 5 mmol, 6 eq). The solution was refluxed at 110° C. for 20 h. H₂O was removed under vacuum, the brown oil obtained was used for the next step reaction without any further purification.

Step 2: The crude product for the first step was dissolved in DMF (2 mL), then DIEA (525 μL, 3.07 mmol, 4 eq) and COMU (493 mg, 1.15 mmol, 1.5 eq) were added. The mixture was stirred at room temperature for 15 min, then was added a solution of L-valinol (119 mg, 1.15 mmol, 1.5 eq) in DMF (2 mL). The resulting solution was refluxed at 130° C. for 72 h. The solvent was removed under vacuum. The residue obtained was dissolved in H₂O, extracted with ethyl acetate. The combined organic layers were dried over MgSO₄, filtered and concentrated under vacuum to give the crude product. The crude product was purified by flash chromatography column over silica gel (cyclohexane/ethyl acetate: 100/0 to 0/100) to afford example 66 (10 mg, 3%) as an yellow oil; ¹H NMR (300 MHZ, CDCl₃): δ 7.57 (d, J=8.1 Hz, 2H), 7.45 (d, J=8.1 Hz, 2H), 4.46 (d, J=16.9 Hz, 1H), 4.11 (d, J=16.9 Hz, 1H), 4.10-4.02 (m, 2H), 3.86-3.77 (m, 1H), 3.66 (d, J=13.6 Hz, 1H), 3.57 (d, J=13.6 Hz, 1H), 3.23 (dd, J=4.3, 2.2 Hz, 1H), 3.11-3.03 (m, 1H), 2.79-2.74 (m, 1H), 2.46 (dd, J=12.7, 2.2 Hz, 2H), 2.27-2.20 (m, 2H), 1.98-1.89 (m, 1H), 1.84-1.78 (m, 1H), 0.97 (d, J=6.8 Hz, 3H), 0.92 (d, J=6.8 Hz, 3H) ppm. ¹³C NMR (75 MHZ, CDCl₃): δ 167.5, 141.8, 129.6 (q, J=31.2 Hz), 129.4, 125.4 (q, J=3.6 Hz), 124.4 (q, J=270.0 Hz), 89.1, 75.5, 68.0, 67.5, 62.0, 61.4, 54.0, 49.5, 32.9, 32.0, 20.0, 18.9 ppm. [ES+MS] m/z 399 (MH⁺).

### Synthesis of (3S)-9-benzyl-3-isopropyl-2,3,6,7,7a,8,10,11-octahydrooxazolo[2,3-j][1,6]naphthyridine-5-thione (example 65)

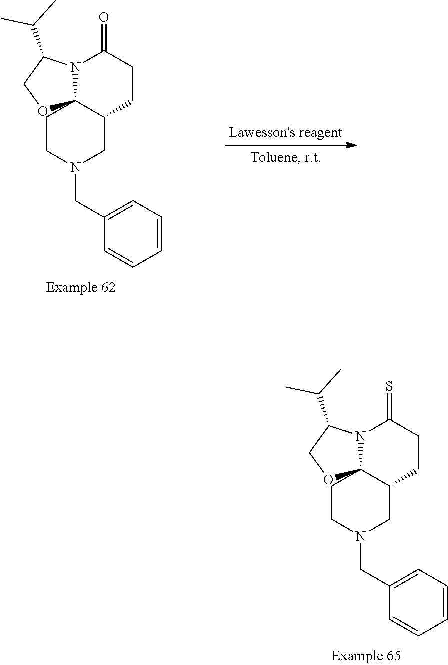 [SMILES: CC(C)[C@H]1CO[C@]23CCN(Cc4ccccc4)C[C@H]2CCC(=O)N13 ; CC(C)[C@H]1CO[C@]23CCN(Cc4ccccc4)C[C@H]2CCC(=S)N13]

Lawesson's Reagent (36.9 mg, 0.0913 mmol) was added to a solution of example 62 (50.0 mg, 0.152 mmol) dissolved in 5 mL of toluene under Argon. The reaction mixture was stirred for 48 h at room temperature. The conversion of example 62 was not complete (LC-MS), Lawesson's reagent (12.3 mg, 0.0304 mmol, 0.2 eq.) was added again to the mixture. When the conversion of the starting material example 71 was judged complete by LC-MS, cyclohexane was added to the reaction mixture and then a precipitate was formed. The mixture was filtered and the filtrate was recovered. The solvent was removed under reduced pressure to give example 65 (42.3 mg, 81%), as a yellow oil. [ES+MS] m/z 345 (MH⁺).

### General Protocol 15: Synthesis of Examples 35 and 36 by Chan-Lam Coupling

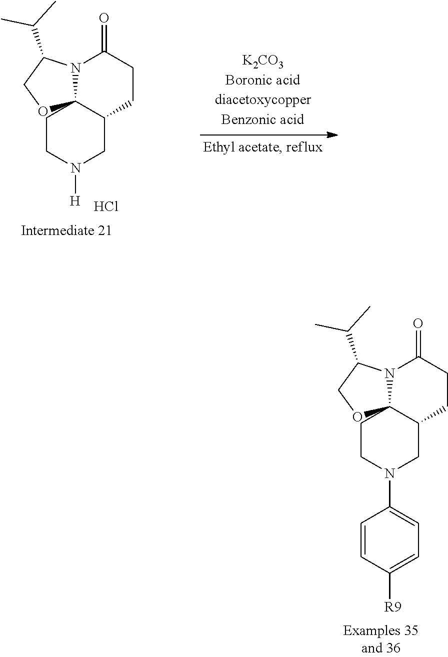

To a solution of diacetoxycopper (0.2 eq), dipotassium; carbonate (1 eq) and benzoic acid (1 eq) in ethyl acetate stirred at room temperature were intermediate 21 (1 eq) and the appropriate boronic acid (1 eq). The reaction was refluxed at 80° C. until complete conversion of the starting material intermediate 21.

| Example | structure | Physical data |
|---|---|---|
| 35 | 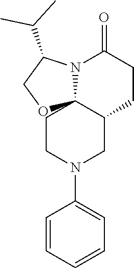 [SMILES: CC(C)[C@H]1CO[C@]23CCN(c4ccccc4)C[C@H]2CCC(=O)N13] | (3S,7aR,11aR)-3-isopropyl-9-phenyl-2,3,6,7,7a,8,10,11- octahydrooxazolo[2,3-j][1,6]naphthyridin-5-one Example 35 was obtained from phenyl boronic acid using general protocol 15; Purification by flash chromatography over silica gel (cyclohexane/ethyl acetate: 100/0 to 30/70); Yield: 32%; [ES+ MS] m/z 315 (MH⁺). |
| 36 | 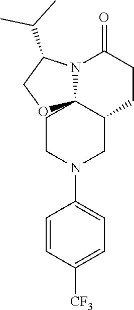 [SMILES: CC(C)[C@H]1CO[C@]23CCN(c4ccc(C(F)(F)F)cc4)C[C@H]2CCC(=O)N13] | (3S,7aR,11aR)-3-isopropyl-9-[4-(trifluoromethyl)phenyl]- 2,3,6,7,7a,8,10,11-octahydrooxazolo[2,3- j][1,6]naphthyridin-5-one Example 35 was obtained from [4- (trifluoromethyl)phenyl]boronic acid using general protocol 15; Purification by flash chromatography over silica gel (cyclohexane/ethyl acetate: 100/0 to 0/100); [ES+ MS] m/z 383 (MH⁺). |

### Part B: Activity of the Compounds According to the Invention

### Determination of MICs

The minimum inhibitory concentration (MIC) of compounds against Mtb was determined using the resazurin microtiter assay (REMA) in 96-well plates. Briefly, mycobacteria were grown to mid-log phase in complete Middlebrook 7H9 media and diluted to an OD₆₀₀ of 0.001. The bacterial suspension was then added to the wells of a 96-well plate (200 μL to the first column of wells and 100 μL to all other wells). Test compounds were then spiked into the first well, and serially diluted down the plate using a multichannel pipette. Plates were incubated for 8-10 doubling times (37° C., 6 days) and bacterial viability was determined by subsequent addition of resazurin (10 μL of 0.025% (w/v) resazurin), incubation (37° C., overnight) and measuring resorufin production [Ex 530 nm, Em 590 nm] using a fluorescence microplate reader. The MIC of compounds against Mtb was considered to be the lowest compound concentration where resazurin turnover was less than 2% of the background fluorescence.

For the MIC analysis of non-Mtb mycobacteria, experiments were performed in a similar manner as for Mtb, but incubation times were adjusted according to the bacterial generation time (5 days for M. avium and M. marinum and 2 days for M. abssessus and M. smegmatis). In addition, M. marinum was cultured at 30° C., rather than 37° C. and their viability was determined by visual inspection of bacterial growth in the microtitre plates. For the other bacteria, MIC was determined as the lowest compound concentration where resazurin turnover was less than 10% of the background fluorescence.

### Biochemical Evaluation of MBP-Ndh Inhibition

To define the activity of compounds on purified MBP-Ndh, a biochemical assay was setup to monitor MBP-Ndh dependent oxidation of NADH in the presence of an electron acceptor menadione, similar to methods described previously (Murugesan, D. et al. 2-Mercapto-Quinazolinones as Inhibitors of Type II NADH Dehydrogenase and Mycobacterium tuberculosis: Structure-Activity Relationships, Mechanism of Action and Absorption, Distribution, Metabolism, and Excretion Characterization. ACS Infect. Dis. 4, (2018)). Briefly, to evaluate the IC₅₀ of the compounds, the compounds were transferred to a black 384-well plate with transparent bottom using Echo liquid handling acoustic technology (Labcyte) and backfilled to 500 nL with DMSO. Using a Viafill liquid dispenser (INTEGRA Biosciences) a 45 μL mixture was added to these wells containing a Mtb recombinant MBP tagged Ndh-2 enzyme (either 70 nM recombinant MBP-Ndh produced in E. coli-; 0.66 nM recombinant MBP-Ndh produced in M. smegmatis, or 30 nM recombinant MBP-NdhA produced in M. smegmatis, concentration adjusted to have similar rate of NADH oxidation) and NADH (500 μM) in 50 mM HEPES buffer (pH 7.1 with K₂HPO₄). Following a pre-incubated (25° C., 15 min), enzyme activity was initiated by the addition of menadione (5 μL, 100 μM final concentration (dispensed into wells using ENVISION, Perkin Elmer), and the kinetics of NADH oxidation monitoring at 340 nm (measured every 60 sec, ENVISION plate reader, Perkin Elmer). The rate of NADH oxidation was calculated as the slope of the linear decrease in 340 nm signal using Microsoft Excel, and enzyme inhibition parameters determined using Graphpad Prism v. 9. IC₅₀ are reported as mean and standard deviation of at least 3 independent experiments.

The results are presented in Tables 2 and 3 below.

TABLE 2

| | | MIC | IC50 |
|---|---|---|---|
| | | (μM) | (μM) |
| | | H37Rv- | purified |
| IUPAC name | Structure | WT | Ndh |
| (3S,7aR,11aR)-9-[(4- fluorophenyl)methyl]-3-isopropyl- 2,3,6,7,7a,8,10,11- octahydrooxazolo[2,3- j][1,6]naphthyridin-5-one | 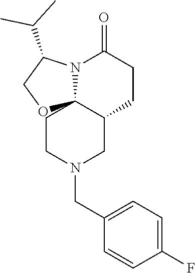 [SMILES: CC(C)[C@H]1CO[C@]23CCN(Cc4ccc(F)cc4)C[C@H]2CCC(=O)N13] | +++ | n.t. |
| (3S,7aR,11aR)-3-isopropyl-9-[[4- (trifluoromethyl)phenyl]methyl]- 2,3,6,7,7a,8,10,11- octahydrooxazolo[2,3- j][1,6]naphthyridin-5-one | 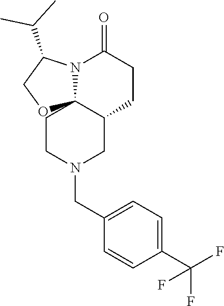 [SMILES: CC(C)[C@H]1CO[C@]23CCN(Cc4ccc(C(F)(F)F)cc4)C[C@H]2CCC(=O)N13] | +++ | *** |
| (3S,7aR,11aR)-3-isopropyl-9-[[4- (trifluoromethoxy)phenyl]methyl]- 2,3,6,7,7a,8,10,11- octahyooxazolo[2,3- j][1,6]naphthyridin-5-one | 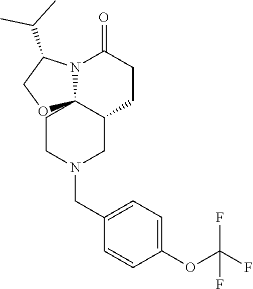 [SMILES: CC(C)[C@H]1CO[C@]23CCN(Cc4ccc(OC(F)(F)F)cc4)C[C@H]2CCC(=O)N13] | +++ | *** |
| (3S,7aR,11aR)-9-[(4- chlorophenyl)methyl]-3-isopropyl- 2,3,6,7,7a,8,10,11- octahydrooxazolo[2,3- j][1,6]naphthyridin-5-one | 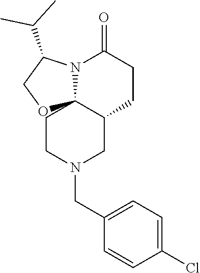 [SMILES: CC(C)[C@H]1CO[C@]23CCN(Cc4ccc(Cl)cc4)C[C@H]2CCC(=O)N13] | +++ | *** |
| (3S,7aR,11aR)-9-[(3,4- dichlorophenyl)methyl]-3- isopropyl-2,3,6,7,7a,8,10,11- octahydrooxazolo[2,3- j][1,6]naphthyridin-5-one | 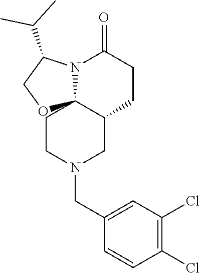 [SMILES: CC(C)[C@H]1CO[C@]23CCN(Cc4ccc(Cl)c(Cl)c4)C[C@H]2CCC(=O)N13] | +++ | *** |
| (3S,7aR,11aR)-3-isopropyl-9-(p- tolylmethyl)-2,3,6,7,7a,8,10,11- octahydrooxazolo[2,3- j][1,6]naphthyridin-5-one | 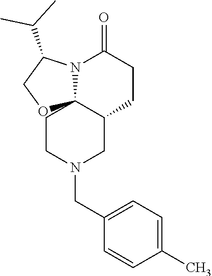 [SMILES: Cc1ccc(CN2CC[C@]34OC[C@H](C(C)C)N3C(=O)CC[C@@H]4C2)cc1] | ++ | n.t. |
| (3S,7aR,11aR)-3-isopropyl-9-[(4- methoxyphenyl)methyl]- 2,3,6,7,7a,8,10,11- octahydrooxazolo[2,3- j][1,6]naphthyridin-5-one | 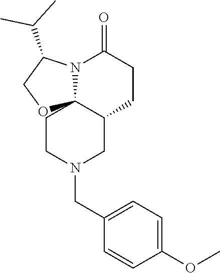 [SMILES: COc1ccc(CN2CC[C@]34OC[C@H](C(C)C)N3C(=O)CC[C@@H]4C2)cc1] | ++ | ** |
| (3S,7aR,11aR)-9-[(4- hydroxyphenyl)methyl]-3- isopropyl-2,3,6,7,7a,8,10,11- octahydrooxazolo[2,3- j][1,6]naphthyridin-5-one | 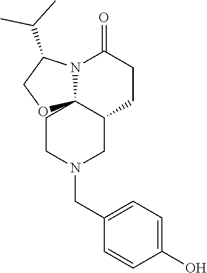 [SMILES: CC(C)[C@H]1CO[C@]23CCN(Cc4ccc(O)cc4)C[C@H]2CCC(=O)N13] | + | n.t. |
| (3S,7aR,11aR)-9-[[4-chloro-3- (trifluoromethyl)phenyl]methyl]-3- isopropyl-2,3,6,7,7a,8,10,11- octahydrooxazolo[2,3- j][1,6]naphthyridin-5-one | 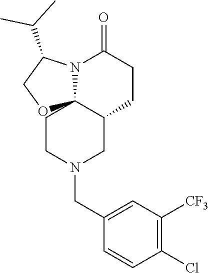 [SMILES: CC(C)[C@H]1CO[C@]23CCN(Cc4ccc(Cl)c(C(F)(F)F)c4)C[C@H]2CCC(=O)N13] | +++ | *** |
| (3S,7aR,11aR)-9-[[2,4- bis(trifluoromethyl)phenyl]methyl]- 3-isopropyl-2,3,6,7,7a,8,10,11- octahydrooxazolo[2,3- j][1,6]naphthyridin-5-one | 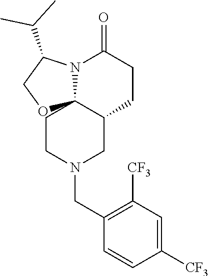 [SMILES: CC(C)[C@H]1CO[C@]23CCN(Cc4ccc(C(F)(F)F)cc4C(F)(F)F)C[C@H]2CCC(=O)N13] | +++ | n.t. |
| (3S,7aR,11aR)-3-isopropyl-9-[(4- phenylphenyl)methyl]- 2,3,6,7,7a,8,10,11- octahydrooxazolo[2,3- j][1,6]naphthyridin-5-one | 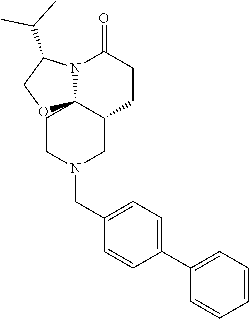 [SMILES: CC(C)[C@H]1CO[C@]23CCN(Cc4ccc(-c5ccccc5)cc4)C[C@H]2CCC(=O)N13] | +++ | n.t. |
| (3S,7aR,11aR)-3-isopropyl-9-[(3- phenoxyphenyl)methyl]- 2,3,6,7,7a,8,10,11- octahydrooxazolo[2,3- j][1,6]naphthyridin-5-one | 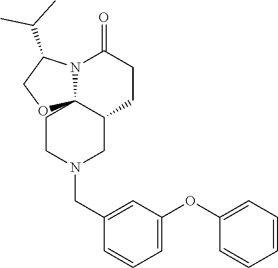 [SMILES: CC(C)[C@H]1CO[C@]23CCN(Cc4cccc(Oc5ccccc5)c4)C[C@H]2CCC(=O)N13] | +++ | n.t. |
| (3S,7aR,11aR)-3-isopropyl-9-[[4- [4-[4-(trifluoromethoxy)phenoxy]- 1-piperidyl]phenyl]methyl]- 2,3,6,7,7a,8,10,11- octahydrooxazolo[2,3- j][1,6]naphthyridin-5-one | 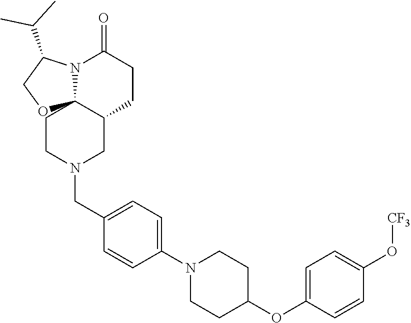 [SMILES: CC(C)[C@H]1CO[C@]23CCN(Cc4ccc(N5CCC(Oc6ccc(OC(F)(F)F)cc6)CC5)cc4)C[C@H]2CCC(=O)N13] | +++ | n.t. |
| (3S,7aR,11aR)-3-isopropyl-9-[[4- [4-[4-(trifluoromethoxy)phenyl]-1- piperidyl]phenyl]methyl]- 2,3,6,7,7a,8,10,11- octahydrooxazolo[2,3- j][1,6]naphthyridin-5-one | 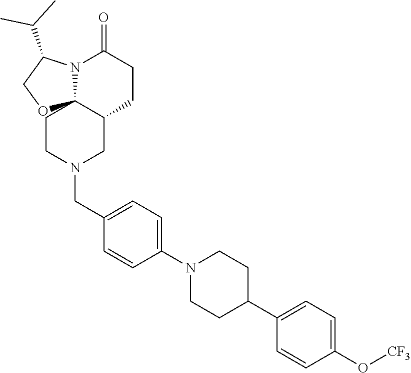 [SMILES: CC(C)[C@H]1CO[C@]23CCN(Cc4ccc(N5CCC(c6ccc(OC(F)(F)F)cc6)CC5)cc4)C[C@H]2CCC(=O)N13] | +++ | n .t. |
| (3S,7aR,11aR)-3-isopropyl-9-[[4- (1-piperidyl)phenyl]methyl]- 2,3,6,7,7a,8,10,11- octahydrooxazolo[2,3- j][1,6]naphthyridin-5-one | 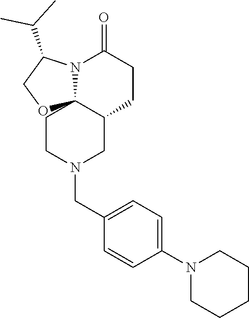 [SMILES: CC(C)[C@H]1CO[C@]23CCN(Cc4ccc(N5CCCCC5)cc4)C[C@H]2CCC(=O)N13] | ++ | n.t. |
| (3S,7aR,11aR)-3-isopropyl-9-(2- pyridylmethyl)-2,3,6,7,7a,8,10,11- octahydrooxazolo[2,3- j][1,6]naphthyiridn-5-one | 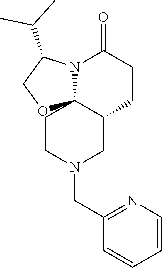 [SMILES: CC(C)[C@H]1CO[C@]23CCN(Cc4ccccn4)C[C@H]2CCC(=O)N13] | ++ | ** |
| (3S,7aR,11aR)-3-isopropyl-9-(3- pyridylmethyl)-2,3,6,7,7a,8,10,11- octahydrooxazolo[2,3- j][1,6]naphthyridin-5-one | 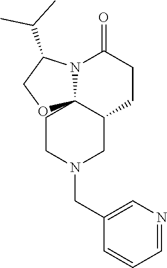 [SMILES: CC(C)[C@H]1CO[C@]23CCN(Cc4cccnc4)C[C@H]2CCC(=O)N13] | ++ | * |
| (3S,7aR,11aR)-3-isopropyl-9-(4- pyridylmethyl)-2,3,6,7,7a,8,10,11- octahydrooxazolo[2,3- j][1,6]naphthyridin-5-one | 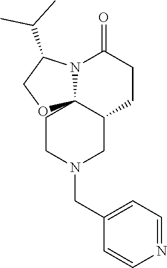 [SMILES: CC(C)[C@H]1CO[C@]23CCN(Cc4ccncc4)C[C@H]2CCC(=O)N13] | + | n.t. |
| (3S,7aR,11aR)-3-isopropyl-9-[[6- (trifluoromethyl)-3-pyridyl]methyl]- 2,3,6,7,7a,8,10,11- octahydrooxazolo[2,3- j][1,6]naphthyridin-5-one | 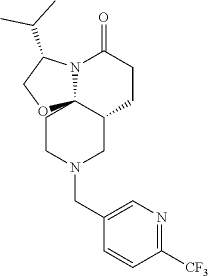 [SMILES: CC(C)[C@H]1CO[C@]23CCN(Cc4ccc(C(F)(F)F)nc4)C[C@H]2CCC(=O)N13] | +++ | *** |
| (3S,7aR,11aR)-3-isopropyl-9-[[5- (trifluoromethyl)-2-pyridyl]methyl]- 2,3,6,7,7a,8,10,11- octahydrooxazolo[2,3- j][1,6]naphthyridin-5-one | 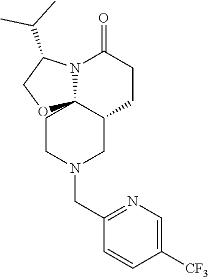 [SMILES: CC(C)[C@H]1CO[C@]23CCN(Cc4ccc(C(F)(F)F)cn4)C[C@H]2CCC(=O)N13] | +++ | ** |
| (3S,7aR,11aR)-3-isopropyl-9-(2- quinolylmethyl)- 2,3,6,7,7a,8,10,11- octahydrooxazolo[2,3- j][1,6]naphthyridin-5-one | 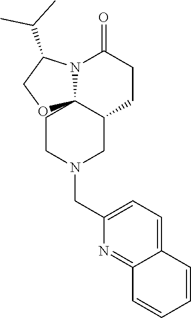 [SMILES: CC(C)[C@H]1CO[C@]23CCN(Cc4ccc5ccccc5n4)C[C@H]2CCC(=O)N13] | +++ | ** |
| (3S,7aR,11aR)-3-isopropyl-9-(8- quinolylmethyl)- 2,3,6,7,7a,8,10,11- octahydrooxazolo[2,3- j][1,6]naphthyridin-5-one | 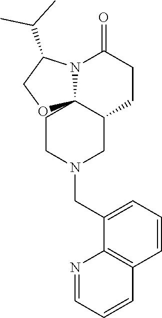 [SMILES: CC(C)[C@H]1CO[C@]23CCN(Cc4cccc5cccnc45)C[C@H]2CCC(=O)N13] | ++ | * |
| (3S,7aR,11aR)-3-isopropyl-9-(6- quinolylmethyl)- 2,3,6,7,7a,8,10,11- octahydrooxazolo[2,3- j][1,6]naphthyridin-5-one | 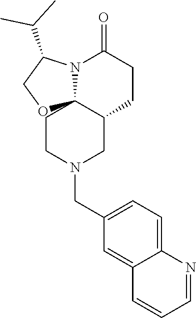 [SMILES: CC(C)[C@H]1CO[C@]23CCN(Cc4ccc5ncccc5c4)C[C@H]2CCC(=O)N13] | ++ | n.t. |
| (3S,7aR,11aR)-9-[(2-cyclopropyl- 4-quinolyl)methyl]-3-isopropyl- 2,3,6,7,7a,8,10,11- octahydrooxazolo[2,3- j][1,6]naphthyridin-5-one | 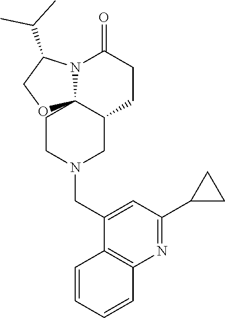 [SMILES: CC(C)[C@H]1CO[C@]23CCN(Cc4cc(C5CC5)nc5ccccc45)C[C@H]2CCC(=O)N13] | +++ | n.t. |
| (3S,7aR,11aR)-3-isopropyl-9-[[2- (trifluoromethyl)-4- quinolyl]methyl]- 2,3,6,7,7a,8,10,11- octahydrooxazolo[2,3- j][1,6]naphthyridin-5-one | 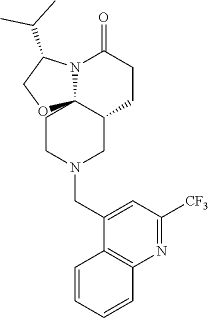 [SMILES: CC(C)[C@H]1CO[C@]23CCN(Cc4cc(C(F)(F)F)nc5ccccc45)C[C@H]2CCC(=O)N13] | +++ | |
| (3S,7aR,11aR)-3-isopropyl-9-[(1- methylimidazol-2-yl)methyl]- 2,3,6,7,7a,8,10,11- octahydrooxazolo[2,3- j][1,6]naphthyridin-5-one | 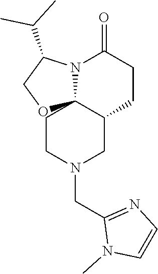 [SMILES: CC(C)[C@H]1CO[C@]23CCN(Cc4nccn4C)C[C@H]2CCC(=O)N13] | ++ | n.t. |
| (3S,7aR,11aR)-3-isopropyl-9-[(3- methyl-1H-indol-2-yl)methyl]- 2,3,6,7,7a,8,10,11- octahydrooxazolo[2,3- j][1,6]naphthyridin-5-one | 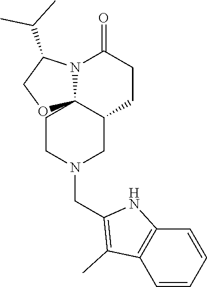 [SMILES: Cc1c(CN2CC[C@]34OC[C@H](C(C)C)N3C(=O)CC[C@@H]4C2)[nH]c2ccccc12] | +++ | n.t. |
| (3S,7aR,11aR)-3-isopropyl-9-[(1- methylindol-2-yl)methyl]- 2,3,6,7,7a,8,10,11- ocathydrooxazolo[2,3- j][1,6]naphthyridin-5-one | 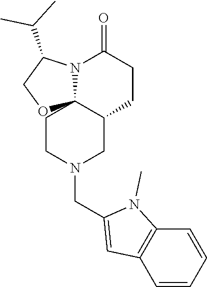 [SMILES: CC(C)[C@H]1CO[C@]23CCN(Cc4cc5ccccc5n4C)C[C@H]2CCC(=O)N13] | +++ | *** |
| (3S,7aR,11aR)-9- (cyclohexylmethyl)-3-isopropyl- 2,3,6,7,7a,8,10,11- octahydrooxazolo[2,3- j][1,6]naphthyridin-5-one | 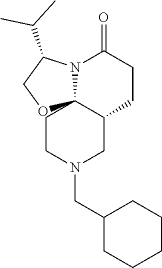 [SMILES: CC(C)[C@H]1CO[C@]23CCN(CC4CCCCC4)C[C@H]2CCC(=O)N13] | +++ | ** |
| (3S,7aR,11aR)-9-cyclobutyl-3- isopropyl-2,3,6,7,7a,8,10,11- octahydrooxazolo[2,3- j][1,6]naphthyridin-5-one | 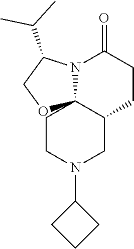 [SMILES: CC(C)[C@H]1CO[C@]23CCN(C4CCC4)C[C@H]2CCC(=O)N13] | * | n.t. |
| (3S,7aR,11aR)-9-cyclohexyl-3- isopropyl-2,3,6,7,7a,8,10,11- octahydrooxazolo[2,3- j][1,6]naphthyridin-5-one | 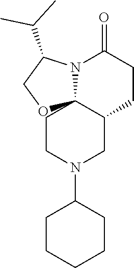 [SMILES: CC(C)[C@H]1CO[C@]23CCN(C4CCCCC4)C[C@H]2CCC(=O)N13] | + | n.t. |
| (3S,7aR,11aR)-3-isopropyl-9- (tetrahydropyran-4-ylmethyl)- 2,3,6,7,7a,8,10,11- octahydrooxazolo[2,3- j][1,6]naphthyridin-5-one | 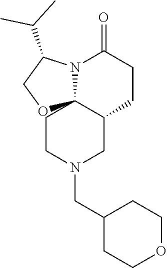 [SMILES: CC(C)[C@H]1CO[C@]23CCN(CC4CCOCC4)C[C@H]2CCC(=O)N13] | + | n.t. |
| (3S,7aR,11aR)-3-isopropyl-9- (tetrahydropyran-2-ylmethyl)- 2,3,6,7,7a,8,10,11- octahydrooxazolo[2,3- j][1,6]naphthyridin-5-one | 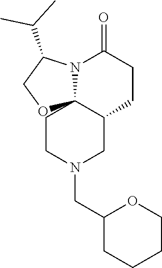 [SMILES: CC(C)[C@H]1CO[C@]23CCN(CC4CCCCO4)C[C@H]2CCC(=O)N13] | ++ | n.t. |
| (3S,7aR,11aR)-3-isopropyl-9- (tetrahydrofuran-2-ylmethyl)- 2,3,6,7,7a,8,10,11- octahydrooxazolo[2,3- j][1,6]naphthyridin-5-one | 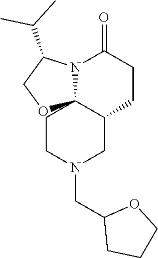 [SMILES: CC(C)[C@H]1CO[C@]23CCN(CC4CCCO4)C[C@H]2CCC(=O)N13] | + | n.t. |
| (3S,7aR,11aR)-3-isopropyl-9- phenyl-2,3,6,7,7a,8,10,11- octahydrooxazolo[2,3- j][1,6]naphthyridin-5-one | 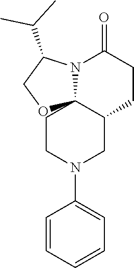 [SMILES: CC(C)[C@H]1CO[C@]23CCN(c4ccccc4)C[C@H]2CCC(=O)N13] | ++ | ** |
| (3S,7aR,11aR)-3-isopropyl-9-[4- (trifluoromethyl)phenyl]- 2,3,6,7,7a,8,10,11- octahydrooxazolo[2,3- j][1,6]naphthyridin-5-one | 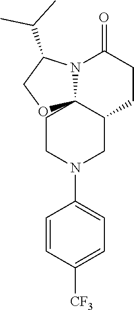 [SMILES: CC(C)[C@H]1CO[C@]23CCN(c4ccc(C(F)(F)F)cc4)C[C@H]2CCC(=O)N13] | ++ | n.t. |
| (3S,7aR,11aR)-3-isopropyl-9-(2- phenylethyl)-2,3,6,7,7a,8,10,11- octahydrooxazolo[2,3- j][1,6]naphthyridin-5-one | 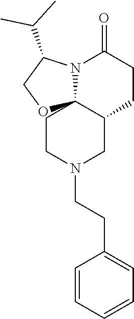 [SMILES: CC(C)[C@H]1CO[C@]23CCN(CCc4ccccc4)C[C@H]2CCC(=O)N13] | ++ | ** |
| (3S,7aR,11aR)-3-isopropyl-9-[2- [4-(trifluoromethyl)phenyl]ethyl]- 2,3,67,7a,8,10,11- octahydrooxazolo[2,3- j][1,6]naphthyridin-5-one | 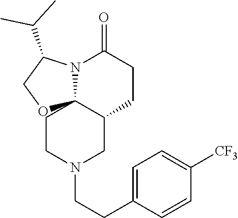 [SMILES: CC(C)[C@H]1CO[C@]23CCN(CCc4ccc(C(F)(F)F)cc4)C[C@H]2CCC(=O)N13] | +++ | ** |
| (3S,7aR,11aR)-3-isopropyl-9-[3- [4-(trifluoromethyl)phenyl]propyl]- 2,3,6,7,7a,8,10,11- octahydrooxazolo[2,3- j][1,6]naphthyridin-5-one | 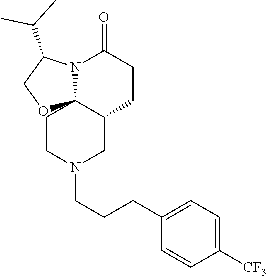 [SMILES: CC(C)[C@H]1CO[C@]23CCN(CCCc4ccc(C(F)(F)F)cc4)C[C@H]2CCC(=O)N13] | +++ | |
| (3S,7aR,11aR)-9-[2,2-difluoro-2- [4-(trifluoromethyl)phenyl]ethyl]-3- isopropyl-2,3,6,7,7a,8,10,11- octahydrooxazolo[2,3- j][1,6]naphthyridin-5-one | 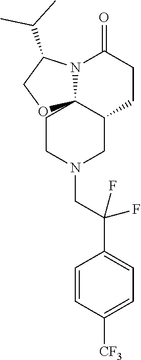 [SMILES: CC(C)[C@H]1CO[C@]23CCN(CC(F)(F)c4ccc(C(F)(F)F)cc4)C[C@H]2CCC(=O)N13] | +++ | n.t. |
| (3S,7aR,11aR)-3-isopropyl-9-(2- phenylacetyl)-2,3,6,7,7a,8,10,11- octahydrooxazolo[2,3- j][1,6]naphthyridin-5-one | 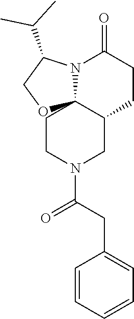 [SMILES: CC(C)[C@H]1CO[C@]23CCN(C(=O)Cc4ccccc4)C[C@H]2CCC(=O)N13] | + | * |
| (3S,7aR,11aR)-3-isopropyl-9-[2- [4-(trifluoromethyl)phenyl]acetyl]- 2,3,6,7,7a,8,10,11- octahydrooxazolo[2,3- j][1,6]naphthyridin-5-one | 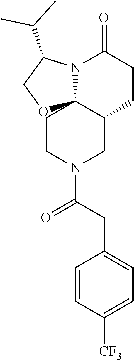 [SMILES: CC(C)[C@H]1CO[C@]23CCN(C(=O)Cc4ccc(C(F)(F)F)cc4)C[C@H]2CCC(=O)N13] | + | n.t. |
| (3S,7aR,11aR)-3-isopropyl-9-[3- [4- (trifluoromethyl)phenyl]propanyl]- 2,3,6,7,7a,8,10,11- ocathydrooxazolo[2,3- j][1,6]naphthyridin-5-one | 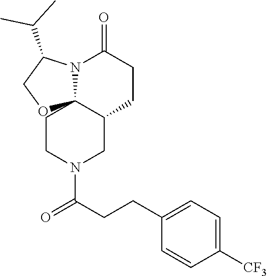 [SMILES: CC(C)[C@H]1CO[C@]23CCN(C(=O)CCc4ccc(C(F)(F)F)cc4)C[C@H]2CCC(=O)N13] | +(+) | n.t. |
| (3S,7aR,11aR)-3-isopropyl-9-[4- [4- (trifluoromethyl)phenyl]butanoyl]- 2,3,67,7a,8,10,11- ocathydrooxazolo[2,3- j][1,6]naphthyridin-5-one | 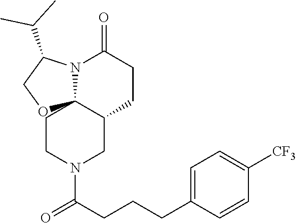 [SMILES: CC(C)[C@H]1CO[C@]23CCN(C(=O)CCCc4ccc(C(F)(F)F)cc4)C[C@H]2CCC(=O)N13] | +(+) | n.t. |
| (3S,7aR,11aR)-9-[2,2-difluoro-2- [4-(trifluoromethyl)phenyl]acetyl]- 3-isopropyl-2,3,6,7,7a,8,10,11- octahydrooxazolo[2,3- j][1,6]naphthyridin-5-one | 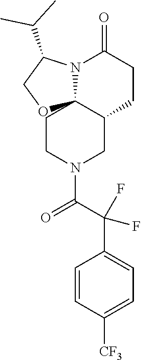 [SMILES: CC(C)[C@H]1CO[C@]23CCN(C(=O)C(F)(F)c4ccc(C(F)(F)F)cc4)C[C@H]2CCC(=O)N13] | + | n.t. |
| benzyl (3S,7aR,11aR)-3- isopropyl-5-oxo- 2,3,6,7,7a,8,10,11- octahydrooxazolo[2,3- j][1,6]naphthyridine-9-carboxylate | 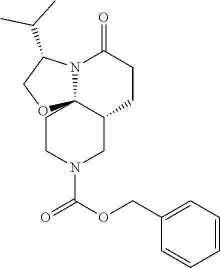 [SMILES: CC(C)[C@H]1CO[C@]23CCN(C(=O)OCc4ccccc4)C[C@H]2CCC(=O)N13] | ++ | ** |
| [4-(trifluoromethyl)phenyl]methyl (3S,7aR,11aR)-3-isopropyl-5-oxo- 2,3,6,7,7a,8,10,11- octahydrooxazolo[2,3- j][1,6]naphthyridine-9-carboxylate | 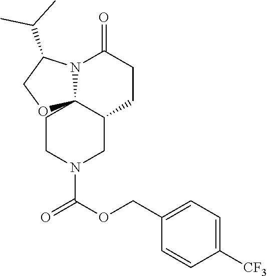 [SMILES: CC(C)[C@H]1CO[C@]23CCN(C(=O)OCc4ccc(C(F)(F)F)cc4)C[C@H]2CCC(=O)N13] | ++ | n.t. |
| 2-[4-(trifluoromethyl)phenyl]ethyl (3S,7aR,11aR)-3-isopropyl-5-oxo- 2,3,6,7,7a,8,10,11- ocathydrooxazolo[2,3- j][1,6]naphthyridine-9-carboxylate | 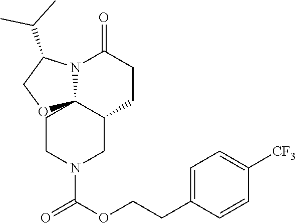 [SMILES: CC(C)[C@H]1CO[C@]23CCN(C(=O)OCCc4ccc(C(F)(F)F)cc4)C[C@H]2CCC(=O)N13] | ++ | n.t. |
| 3-[4-(trifluoromethyl)phenyl]propyl (3S,7aR,11aR)-3-isopropyl-5-oxo- 2,3,6,7,7a,8,10,11- ocathydrooxazolo[2,3- j][1,6]naphthyridine-9-carboxylate | 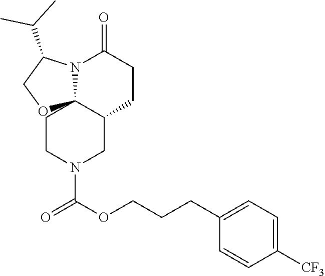 [SMILES: CC(C)[C@H]1CO[C@]23CCN(C(=O)OCCCc4ccc(C(F)(F)F)cc4)C[C@H]2CCC(=O)N13] | ++ | n.t. |
| (3S,7aR,11aR)-3-isopropyl-9-[4- (trifluoromethyl)phenyl]sulfonyl- 2,3,6,7,7a,8,10,11- octahydrooxazolo[2,3- j][1,6]naphthyridin-5-one | 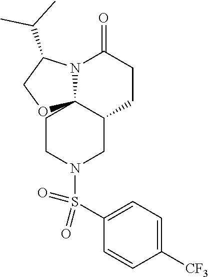 [SMILES: CC(C)[C@H]1CO[C@]23CCN(S(=O)(=O)c4ccc(C(F)(F)F)cc4)C[C@H]2CCC(=O)N13] | ++ | ** |
| (3S,7aR,11aR)-3-isopropyl-9-[[4- (trifluoromethyl)phenyl]methylsulfo- nyl]-2,3,6,7,7a,8,10,11- octahydrooxazolo[2,3- j][1,6]naphthyridin-5-one | 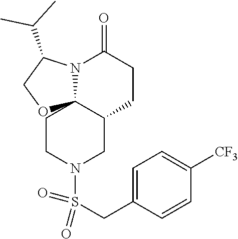 [SMILES: CC(C)[C@H]1CO[C@]23CCN(S(=O)(=O)Cc4ccc(C(F)(F)F)cc4)C[C@H]2CCC(=O)N13] | +++ | n.t. |
| (3S,7aR,11aR)-3-isopropyl-9-[2- [4- (trifluoromethyl)phenyl]ethylsulfonyl]- 2,3,6,7,7a,8,10,11- octahydrooxazolo[2,3- j][1,6]naphthyridin-5-one | 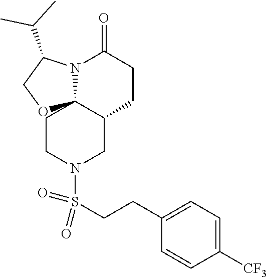 [SMILES: CC(C)[C@H]1CO[C@]23CCN(S(=O)(=O)CCc4ccc(C(F)(F)F)cc4)C[C@H]2CCC(=O)N13] | +++ | |
| (3S,7aR,11aR)-3-isopropyl-9-[3- [4- (trifluoromethyl)phenyl]propylsulfo- nyl]-2,3,6,7,7a,8,10,11- ocathydrooxazolo[2,3- j][1,6]naphthyridin-5-one | 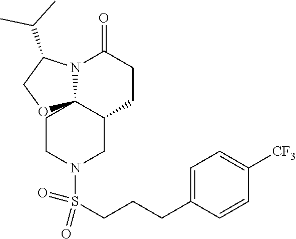 [SMILES: CC(C)[C@H]1CO[C@]23CCN(S(=O)(=O)CCCc4ccc(C(F)(F)F)cc4)C[C@H]2CCC(=O)N13] | +++ | |
| (3S,7aR,11aR)-9-benzyl-3-[(1R)- 1-methylpropyl]- 2,3,6,7,7a,8,10,11- octahydrooxazolo[2,3- j][1,6]naphthyridin-5-one | 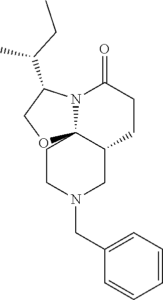 [SMILES: CC[C@@H](C)[C@H]1CO[C@]23CCN(Cc4ccccc4)C[C@H]2CCC(=O)N13] | +(+) | n.t. |
| (3S,7aR,11aR)-9-benzyl-3-tert- butyl-2,3,6,7,7a,8,10,11- octahydrooxazolo[2,3- j][1,6]naphthyridin-5-one | 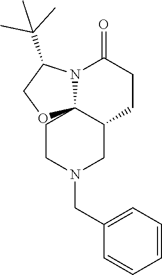 [SMILES: CC(C)(C)[C@H]1CO[C@]23CCN(Cc4ccccc4)C[C@H]2CCC(=O)N13] | + | * |
| (3S,7aR,11aR)-9-benzyl-3- cyclopropyl-2,3,6,7,7a,8,10,11- octahydrooxazolo[2,3- j][1,6]naphthyridin-5-one | 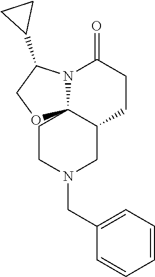 [SMILES: O=C1CC[C@@H]2CN(Cc3ccccc3)CC[C@@]23OC[C@H](C2CC2)N13] | + | n.t. |
| (3S,7aR,11aR)-9-[[4- (trifluoromethyl)phenyl]methyl]-3- vinyl-2,3,67,7a,8,10,11- octahydrooxazolo[2,3- j][1,6]naphthyridin-5-one | 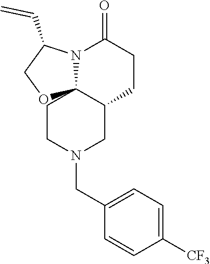 [SMILES: C=C[C@H]1CO[C@]23CCN(Cc4ccc(C(F)(F)F)cc4)C[C@H]2CCC(=O)N13] | + | n.t. |
| (3R,7aR,11aR)-3- (trifluoromethyl)-9-[[4- (trifluoromethyl)phenyl]methyl]- 2,3,6,7,7a,8,10,11- octahydrooxazolo[2,3- j][1,6]naphthyridin-5-one | 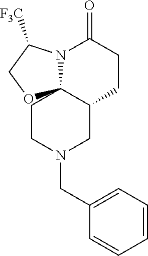 [SMILES: O=C1CC[C@@H]2CN(Cc3ccccc3)CC[C@@]23OC[C@H](C(F)(F)F)N13] | + | n.t. |
| (4R,8aR,12aR)-4-isopropyl-10- [[4- (trifluoromethyl)pehnyl]methyl]- 3,4,7,8,8a,9,11,12-octahydro-2H- [1,3]oxazino[2,3- j][1,6]naphthyridin-6-one | 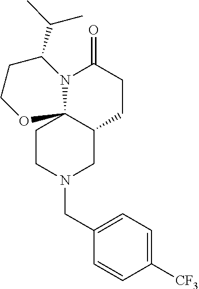 [SMILES: CC(C)[C@H]1CCO[C@]23CCN(Cc4ccc(C(F)(F)F)cc4)C[C@H]2CCC(=O)N13] | ++ | n.t. |
| (1S,6R,11S,16S)-4-benzyl- 4,10,17- triazatetracyclo[8.7.0.01,6.011,16] heptadecan-9-one | 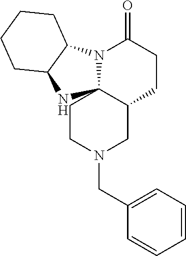 [SMILES: O=C1CC[C@@H]2CN(Cc3ccccc3)CC[C@@]23N[C@H]2CCCC[C@@H]2N13] | + | n.t. |
| (3S,7aR,11aR)-9-benzyl-3- isopropyl-2,3,6,7,7a,8,10,11- octahydrothiazolo[2,3- j][1,6]naphthyridin-5-one | 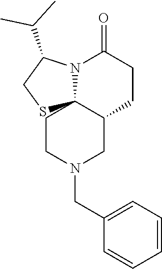 [SMILES: CC(C)[C@H]1CS[C@]23CCN(Cc4ccccc4)C[C@H]2CCC(=O)N13] | | * |
| (1R,4S,8R)-10-benzyl-4- isopropyl-2-oxa-5,10- diazatricyclo[6.4.0.01,5]dodecan- 6-one | 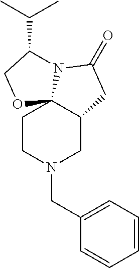 [SMILES: CC(C)[C@H]1CO[C@]23CCN(Cc4ccccc4)C[C@H]2CC(=O)N13] | ++ | n.t. |
| (1R,4S,8R)-4-isopropyl-10-[[4- (trifluoromethyl)phenyl]methyl]-2- oxa-5,10- diazatricyclo[6.4.0.01,5]dodecan- 6-one | 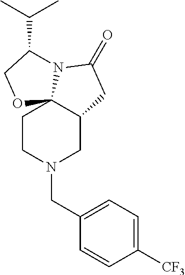 [SMILES: CC(C)[C@H]1CO[C@]23CCN(Cc4ccc(C(F)(F)F)cc4)C[C@H]2CC(=O)N13] | +++ | n.t. |
| (3S,7aR,11aR)-9-benzyl-3- isopropyl-2,3,6,7,7a,8,10,11- octahydrooxazolo[2,3- j][1,6]naphthyridine-5-thione | 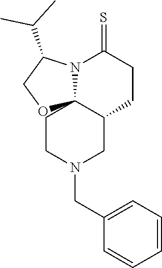 [SMILES: CC(C)[C@H]1CO[C@]23CCN(Cc4ccccc4)C[C@H]2CCC(=S)N13] | ++ | ** |
| (1R,4S,9R)-4-isopropyl-11-[[4- (trifluoromethyl)phenyl]methyl]- 2,8-dioxa-5,11- diazatricyclo[7.4.0.01,5]tridecan- 6-one | 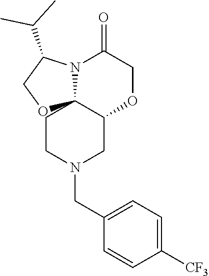 [SMILES: CC(C)[C@H]1CO[C@]23CCN(Cc4ccc(C(F)(F)F)cc4)C[C@H]2OCC(=O)N13] | ++ | |
| (1R,4S,8R)-4-isopropyl-10-[[4- (trifluoromethyl)phenyl]methyl]-2- oxa-5,10- diazatricyclo[6.4.0.01,5]dodecan- 6-one | 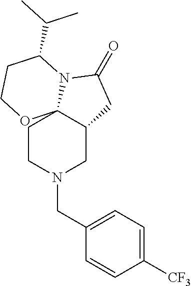 [SMILES: CC(C)[C@H]1CCO[C@]23CCN(Cc4ccc(C(F)(F)F)cc4)C[C@H]2CC(=O)N13] | + | n.t. |
| (3S,7aR,11aR)-3-isopropyl-9-[[4- (trifluoromethyl)phenyl]methyl]- 3,6,7,7a,8,11-hexahydro-2H- oxazolo[2,3-j][1,6]naphthyridine- 5,10-dione | 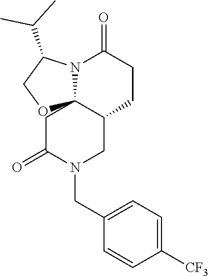 [SMILES: CC(C)[C@H]1CO[C@]23CC(=O)N(Cc4ccc(C(F)(F)F)cc4)C[C@H]2CCC(=O)N13] | + | |
| (3S,7aS,11aR)-3-isopropyl-9-[[4- (trifluoromethyl)phenyl]methyl]- 3,6,7,7a,10,11-hexahydro-2H- oxazolo[2,3-j][1,6]naphthyridine- 5,8-dione | 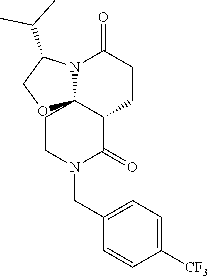 [SMILES: CC(C)[C@H]1CO[C@]23CCN(Cc4ccc(C(F)(F)F)cc4)C(=O)[C@H]2CCC(=O)N13] | + | |
| (3S,7aR,9S,11aR)-9-[bis[[4- (trifluoromethyl)phenyl]methyl]ami- no]-3-isopropyl- 3,6,7,7a,8,9,10,11-octahydro-2H- oxazolo[2,3-j]quinolin-5-one | 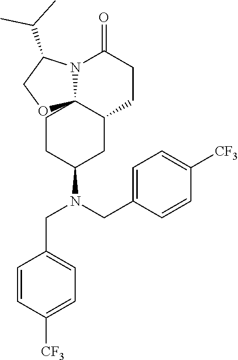 [SMILES: CC(C)[C@H]1CO[C@]23CC[C@H](N(Cc4ccc(C(F)(F)F)cc4)Cc4ccc(C(F)(F)F)cc4)C[C@H]2CCC(=O)N13] | +++ | |
| (3S,7aR,9R,11aR)-9-[bis[[4- (trifluoromethyl)phenyl]methyl]ami- no]-3-isopropyl- 3,6,7,7a,8,9,10,11-octahydro-2H- oxazolo[2,3-j]quinolin-5-one | 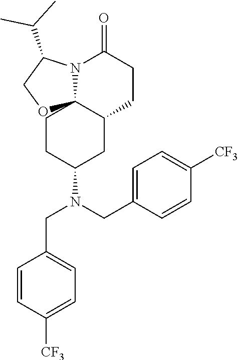 [SMILES: CC(C)[C@H]1CO[C@]23CC[C@@H](N(Cc4ccc(C(F)(F)F)cc4)Cc4ccc(C(F)(F)F)cc4)C[C@H]2CCC(=O)N13] | +++ | |
| (3S,7aR,9S,11aR)-3-isopropyl-9- [[4- (trifluoromethyl)phenyl]methylami- no]-3,6,7,7a,8,9,10,11-octahydro- 2H-oxazolo[2,3-j]quinolin-5-one | 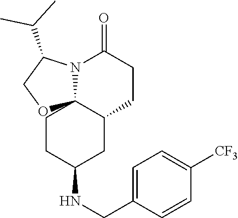 [SMILES: CC(C)[C@H]1CO[C@]23CC[C@H](NCc4ccc(C(F)(F)F)cc4)C[C@H]2CCC(=O)N13] | +++ | |
| (3S,7aR,9R,11aR)-3-isopropyl-9- [[4- (trifluoromethyl)phenyl]methylami- no]-3,6,7,7a,8,9,10,11-octahydro- 2H-oxazolo[2,3-j]quinolin-5-one | 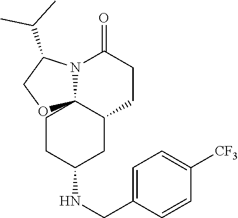 [SMILES: CC(C)[C@H]1CO[C@]23CC[C@@H](NCc4ccc(C(F)(F)F)cc4)C[C@H]2CCC(=O)N13] | +(+) | |
| (3S,7aR,9S,11aR)-3-isopropyl-9- [4-(trifluoromethyl)anilino]- 3,6,7,7a,8,9,10,11-octahydro-2H- oxazolo[2,3-j]quinolin-5-one | 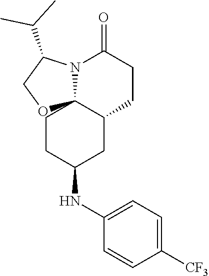 [SMILES: CC(C)[C@H]1CO[C@]23CC[C@H](Nc4ccc(C(F)(F)F)cc4)C[C@H]2CCC(=O)N13] | ++(+) | |
| (3S,7aR,9S,11aR)-3-isopropyl-9- [4-(trifluoromethyl)anilino]- 3,6,7,7a,8,9,10,11-octahydro-2H- oxazolo[2,3-j]quinolin-5-one | 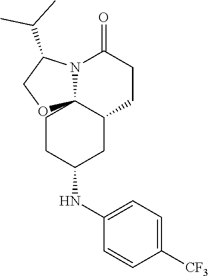 [SMILES: CC(C)[C@H]1CO[C@]23CC[C@@H](Nc4ccc(C(F)(F)F)cc4)C[C@H]2CCC(=O)N13] | | |
| (4S,9R)-4-isopropyl-11-[[4- (trifluoromethyl)phenyl]methyl]-2- oxa-5,11- diazatricyclo[7,5,0.01,5]tetradecan- 6-one | 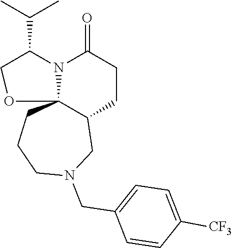 [SMILES: CC(C)[C@H]1CO[C@]23CCCN(Cc4ccc(C(F)(F)F)cc4)C[C@H]2CCC(=O)N13] | +++ | |
| (1R,4S,9S)-4-isopropyl-10-[[4- (trifluoromethyl)phenyl]methyl]-2- oxa-5,10- diazatricyclo[7.3.0.01,5]dodecan- 6-one | 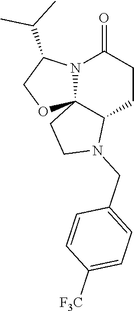 [SMILES: CC(C)[C@H]1CO[C@]23CCN(Cc4ccc(C(F)(F)F)cc4)[C@H]2CCC(=O)N13] | +++ | n.t. |
| (3S,5R,7aR,11aR)-3-isopropyl-6- methyl-9-[[4- (trifluoromethyl)phenyl]methyl]- 2,3,6,7,7a,8,10,11- octahydrooxazolo[2,3- j][1,6]naphthyridin-5-one | 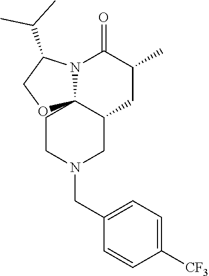 [SMILES: CC(C)[C@H]1CO[C@]23CCN(Cc4ccc(C(F)(F)F)cc4)C[C@H]2C[C@@H](C)C(=O)N13] | +++ | n.t. |
| (3S,6R,7aR,11aR)-6-ethyl-3- isopropyl-9-[[4- (trifluoromethyl)phenyl]methyl]- 2,3,6,7,7a,8,10,11- octahydrooxazolo[2,3- j][1,6]naphthyridin-5-one | 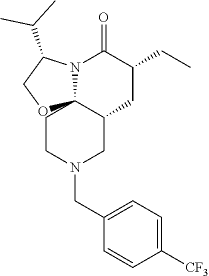 [SMILES: CC[C@@H]1C[C@@H]2CN(Cc3ccc(C(F)(F)F)cc3)CC[C@@]23OC[C@H](C(C)C)N3C1=O] | +++ | n.t. |
| (3S,6R,7aR,11aR)-6-allyl-3- isopropyl-9-[[4- (trifluoromethyl)phenyl]methyl]- 2,3,6,7,7a,8,10,11- octahydrooxazolo[2,3- j][1,6]naphthyridin-5-one | 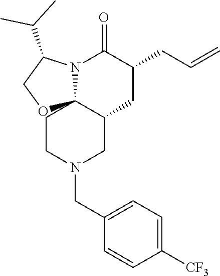 [SMILES: C=CC[C@@H]1C[C@@H]2CN(Cc3ccc(C(F)(F)F)cc3)CC[C@@]23OC[C@H](C(C)C)N3C1=O] | ++ | n.t. |
| (3S,6R,7aR,11aR)-6-isobutyl-3- isopropyl-9-[[4- (trifluoromethyl)phenyl]methyl]- 2,3,6,7,7a,8,10,11- octahydrooxazolo[2,3- j][1,6]naphthyridin-5-one | 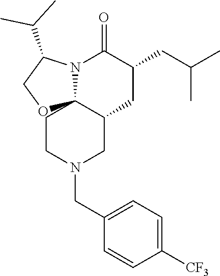 [SMILES: CC(C)C[C@@H]1C[C@@H]2CN(Cc3ccc(C(F)(F)F)cc3)CC[C@@]23OC[C@H](C(C)C)N3C1=O] | + | n.t. |
| (3S,6S,7aR,11aR)-3-isopropyl-6- methyl-9-[[4- (trifluoromethyl)phenyl]methyl]- 2,3,6,7,7a,8,10,11- octahydrooxazolo[2,3- j][1,6]naphthyridin-5-one (dia1) | 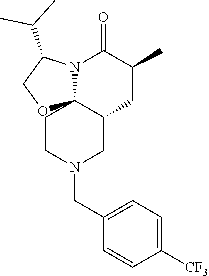 [SMILES: CC(C)[C@H]1CO[C@]23CCN(Cc4ccc(C(F)(F)F)cc4)C[C@H]2C[C@H](C)C(=O)N13] | ++ | n.t. |
| (3S,6S,7aR,11aR)-6-ethyl-3- isopropyl-9-[[4- (trifluoromethyl)phenyl]methyl]- 2,3,6,7,7a,8,10,11- octahydrooxazolo[2,3- j][1,6]naphthyridin-5-one | 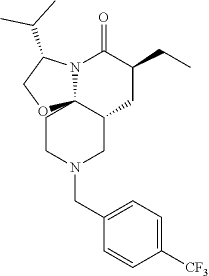 [SMILES: CC[C@H]1C[C@@H]2CN(Cc3ccc(C(F)(F)F)cc3)CC[C@@]23OC[C@H](C(C)C)N3C1=O] | + | n.t. |
| (3S,6S,7aR,11aR)-6-allyl-3- isopropyl-9-[[4- (trifluoromethyl)phenyl]methyl]- 2,3,6,7,7a,8,10,11- octahydrooxazolo[2,3- j][1,6]naphthyridin-5-one | 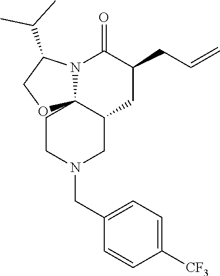 [SMILES: C=CC[C@H]1C[C@@H]2CN(Cc3ccc(C(F)(F)F)cc3)CC[C@@]23OC[C@H](C(C)C)N3C1=O] | + | n.t. |
| (3S,7aR,11aR)-6-fluoro-3- isopropyl-9-[[4- (trifluoromethyl)phenyl]methyl]- 2,3,6,7,7a,8,10,11- octahydrooxazolo[2,3- j][1,6]naphthyridin-5-one | 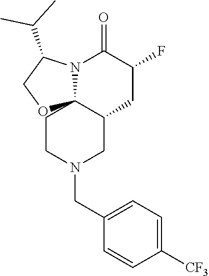 [SMILES: CC(C)[C@H]1CO[C@]23CCN(Cc4ccc(C(F)(F)F)cc4)C[C@H]2C[C@@H](F)C(=O)N13] | +++ | n.t. |
| (3S,7aR,11aR)-6,6-difluoro-3- isopropyl-9-[[4- (trifluoromethyl)phenyl]methyl]- 3,7,7a,8,10,11-hexahydro-2H- oxazolo[2,3-j][1,6]naphthyridin-5- one | 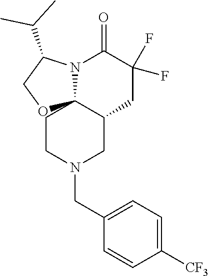 [SMILES: CC(C)[C@H]1CO[C@]23CCN(Cc4ccc(C(F)(F)F)cc4)C[C@H]2CC(F)(F)C(=O)N13] | +++ | n.t. |

MIC activity range: +++ indicates <1 μM, ++ indicates between 1-10 μM, + indicates >10 μM
IC₅₀ activity range: *** indicates <100 nM, ** indicates between 0.1-1 μM, * indicates >1 μM

TABLE 3

| Example | 2 | 3 | 4 | 5 | 9 | 10 | 24 | 25 | 28 | 38 | 39 | 70 |
|---|---|---|---|---|---|---|---|---|---|---|---|---|
| MIC M. tuberculosis | +++ | +++ | +++ | +++ | +++ | +++ | +++ | +++ | +++ | +++ | +++ | +++ |
| MIC M. abscessus R | ++ | ++ | | | ++ | | | | | | | |
| MIC M. abscessus S | ++ | ++ | | | ++ | | | | | | | |
| MIC M. smegmatis | ++ | ++ | | | ++ | | | | +++ | | | |
| MIC M. marinum | +++ | +++ | | | +++ | | | | | | | |
| MIC M. avium | +++ | +++ | | | +++ | | | | | | | |
| MIC BCG | ++ | ++ | | | ++ | | | | | | | |
| IC₅₀ Ndh-M. tb | *** | *** | *** | *** | *** | | | | *** | | | |
| IC₅₀ NdhA-M. tb | *** | *** | *** | | | | | | | | | |

MIC activity range:
+++ indicates <1 μM,
++ indicates between 1-10 μM,
+ indicates >10 μM
IC₅₀ activity range:
*** indicates <100 nM,
** indicates between 0.1-1 μM,
* indicates >1 μM

1. A compound of formula (I): 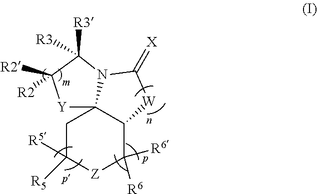
In which:
W is:
O; or
CR₄R_{4′}
X is:
O; or
S
Y is:
O; or
N
Z is:
NR₁; or
CHNRaRb;
p has a value of 0 or 1;
p′ has a value of 1 or 2;
m and n, identical or different have a value of 1 or 2; with at least one W being CR₄R₄;
R₁ is chosen from:
linear or branched —(C₁-C₆)alkyl-(C₆-C₁₀)aryl, said alkyl being optionally substituted by one or more halogen atoms and said aryl being optionally substituted by one or more:
halogen atom,
—OH,
linear or branched —(C₁-C₆)alkyl,
linear or branched —(C₁-C₆)halogenoalkyl,
linear or branched —(C₁-C₆)alkoxy,
linear or branched —(C₁-C₆)halogenoalkoxy,
—(C₆-C₁₀)aryl,
—O—(C₆-C₁₀)aryl;
heterocycle comprising 3 to 10 members and having at least one heteroatom chosen from O, N, or S; said heterocycle being optionally substituted by one or more:
—(C₆-C₁₀)aryl optionally substituted by one or more linear or branched —(C₁-C₆) halogenoalkoxy; and
—O—(C₆-C₁₀)aryl optionally substituted by one or more linear or branched —(C₁-C₆) halogenoalkoxy;
—(C₆-C₁₀)aryl optionally substituted by one or more linear or branched —(C₁-C₆)halogenoalkyl or —(C₁-C₆) halogenoalkoxy;
linear or branched —(C₁-C₆)alkyl-(C₆-C₁₀) heteroaryl, said heteroaryl having at least one heteroatom chosen from O, N, or S and being optionally substituted by one or more:
linear or branched —(C₁-C₆)alkyl,
linear or branched —(C₁-C₆)halogenoalkyl,
—(C₃-C₆)cycloalkyl;
linear or branched —(C₁-C₆)alkyl-(C₃-C₆)cycloalkyl;
—(C₃-C₆)cycloalkyl;
linear or branched —(C₁-C₆)alkyl-heterocycle, said heterocycle comprising 3 to 10 members and having at least one heteroatom chosen from O, N, or S;
—S(O)₂—(C₆-C₁₀)aryl, said aryl being optionally substituted by one or more linear or branched —(C₁-C₆)halogenoalkyl;
—S(O)₂—(C₁-C₆)alkyl-(C₆-C₁₀)aryl, said aryl being optionally substituted by one or more linear or branched —(C₁-C₆)halogenoalkyl;
—C(O)—R′—(C₆-C₁₀)aryl, said aryl being optionally substituted by one or more linear or branched —(C₁-C₆)halogenoalkyl and
with R′ chosen from:
linear or branched —(C₁-C₆)alkyl, said alkyl being optionally substituted by one or more halogen atoms,
linear or branched —O—(C₁-C₆)alkyl, said alkyl being optionally substituted by one or more halogen atoms
Ra and Rb, identical or different, are chosen from H, —(C₆-C₁₀)aryl and linear or branched —(C₁-C₆)alkyl-(C₆-C₁₀)aryl, said aryl being optionally substituted by one or more linear or branched —(C₁-C₆)halogenoalkyl;
R₂ and R₂′, are —H;
R₃ and R₃′, identical or different are chosen from:
—H;
a linear or branched —(C₁-C₆)alkyl;
a linear or branched —(C₂-C₆)alkenyl;
a linear or branched —(C₁-C₆)halogenoalkyl; or
a —(C₃-C₆)cycloalkyl;
or R₂ or R₂′ is linked together with R₃ or R₃′ to form a cycloalkyl or aryl; said cycloalkyl and aryl comprising 3 to 6 members;
R₄ and R₄′, identical or different are chosen from:
—H;
a halogen atom;
a linear or branched —(C₁-C₆)alkyl; and
a linear or branched —(C₂-C₆)alkenyl;
R₅ and R₅′ identical or different are chosen from H, linear or branched —(C₁-C₃)alkyl or form together ═O;
R₆ and R₆′, different, identical or different are chosen from H, linear or branched —(c₁-C₃)alkyl or form together ═O;
or its pharmaceutically acceptable salts or optical isomers.

2. A compound according to claim 1, wherein Z is NR₁ with R₁ chosen from:
linear or branched —(C₁-C₃)alkyl-phenyl, said alkyl being optionally substituted by one or more halogen atoms, in particular fluorine, and said phenyl being optionally substituted by one or more:
halogen atom,
—OH,
linear or branched —(C₁-C₃)alkyl, in particular methyl,
linear or branched —(C₁-C₅)halogenoalkyl, in particular trifluoromethyl,
linear or branched —(C₁-C₃)alkoxy, in particular methoxy;
linear or branched —(C₁-C₅)halogenoalkoxy, in particular trifluoromethoxy;
phenyl,
—O-phenyl;
an heterocycle comprising 3 to 10 members and having at least one heteroatom chosen from N; said heterocycle being optionally substituted by one or more:
phenyl optionally substituted by one or more linear or branched —(C₁-C₅)halogenoalkoxy, in particular trifluoromethoxy; and
—O-phenyl optionally substituted by one or more linear or branched —(C₁-C₃)halogenoalkoxy, in particular trifluoromethoxy;
phenyl optionally substituted by one or more linear or branched —(C₁-C₅)halogenoalkyl or —(C₁-C₅)halogenoalkoxy, in particular trifluoromethyl or trifluoromethoxy;
linear or branched —(C₁-C₃)alkyl-(C₆-C₁₀) heteroaryl, said heteroaryl having at least one heteroatom chosen from N and being optionally substituted by one or more:
linear or branched —(C₁-C₃)alkyl, in particular methyl,
linear or branched —(C₁-C₅)halogenoalkyl, in particular trifluoromethyl,
—(C₃-C₆)cycloalkyl;
linear or branched —(C₁-C₃)alkyl-(C₃-C₆)cycloalkyl;
—(C₃-C₆)cycloalkyl;
linear or branched —(C₁-C₃)alkyl-heterocycle, said heterocycle comprising 3 to 10 members and having at least one heteroatom chosen from O or N;
—S(O)₂-phenyl, said phenyl being optionally substituted by one or more linear or branched —(C₁-C₅)halogenoalkyl, in particular trifluoromethyl;
—S(O)₂—(C₁-C₃)alkyl-phenyl, said phenyl being optionally substituted by one or more linear or branched —(C₁-C₅)halogenoalkyl, in particular trifluoromethyl;
—C(O)—R′-phenyl, said phenyl being optionally substituted by one or more linear or branched —(C₁-C₅)halogenoalkyl, in particular trifluoromethoxy,
with R′ chosen from:
linear or branched —(C₁-C₃)alkyl, said alkyl being optionally substituted by one ore more halogen atoms,
linear or branched —O—(C₁-C₃)alkyl.

3. A compound according to claim 1 wherein Z is CHNRaRb with Ra and Rb, identical or different, chosen from H, phenyl and linear or branched —(C₁-C₃)alkylphenyl, said phenyl being optionally substituted by one or more linear or branched —(C₁-C₃)halogenoalkyl, in particular by one trifluoromethyl.

4. A compound according to claim 1, wherein:
R₂ and R₂′ are —H; and/or
R₃ and R₃′, identical or different are chosen from:
—H;
a linear or branched —(C₁-C₆)alkyl;
a linear or branched —(C₂-C₃)alkenyl;
a linear or branched —(C₁-C₅)halogenoalkyl, in particular trifluoromethyl; or
a —(C₃-C₆)cycloalkyl;
or R₂ or R₂′ is linked together with R₃ or R₃′ to form a cycloalkyl or aryl; said cycloalkyl and aryl comprising 3 to 6 members; and/or
R₄ and R₄′, identical or different are chosen from:
—H;
a halogen atom;
a linear or branched —(C₁-C₃)alkyl; and
a linear or branched —(C₂-C₃)alkenyl; and/or
R₅ and R₅′ are H or form together ═O; and/or
R₆ and Re are H or form together ═O.

5. A compound according to claim 1, wherein said compound if chosen from:
(3S,7aR,11aR)-9-[(4-fluorophenyl)methyl]-3-isopropyl-2,3,6,7,7a,8,10,11-octahydrooxazolo[2,3-j][1,6]naphthyridin-5-one;
(3S,7aR,11aR)-3-isopropyl-9-[[4-(trifluoromethyl)phenyl]methyl]-2,3,6,7,7a,8,10,11-octahydrooxazolo[2,3-j][1,6]naphthyridin-5-one;
1 (3S,7aR,11aR)-3-isopropyl-9-[[4-(trifluoromethoxy)phenyl]methyl]-2,3,6,7,7a,8,10,11-octahydrooxazolo[2,3-j][1,6]naphthyridin-5-one;
(3S,7aR,11aR)-9-[(4-chlorophenyl)methyl]-3-isopropyl-2,3,6,7,7a,8,10,11-octahydrooxazolo[2,3-j][1,6]naphthyridin-5-one;
(3S,7aR,11aR)-9-[(3,4-dichlorophenyl)methyl]-3-isopropyl-2,3,6,7,7a,8,10,11-octahydrooxazolo[2,3-j][1,6]naphthyridin-5-one;
(3S,7aR,11aR)-3-isopropyl-9-(p-tolylmethyl)-2,3,6,7,7a,8,10,11-octahydrooxazolo[2,3-j][1,6]naphthyridin-5-one;
(3S,7aR,11aR)-3-isopropyl-9-[(4-methoxyphenyl)methyl]-2,3,6,7,7a,8,10,11-octahydrooxazolo[2,3-j][1,6]naphthyridin-5-one;
1 (3S,7aR,11aR)-9-[(4-hydroxyphenyl)methyl]-3-isopropyl-2,3,6,7,7a,8,10,11-octahydrooxazolo[2,3-j][1,6]naphthyridin-5-one;
(3S,7aR,11aR)-9-[[4-chloro-3-(trifluoromethyl)phenyl]methyl]-3-isopropyl-2,3,6,7,7a,8,10,11-octahydrooxazolo[2,3-j][1,6]naphthyridin-5-one;
(3S,7aR,11aR)-9-[[2,4-bis(trifluoromethyl)phenyl]methyl]-3-isopropyl-2,3,6,7,7a,8,10,11-octahydrooxazolo[2,3-j][1,6]naphthyridin-5-one;
(3S,7aR,11aR)-3-isopropyl-9-[(4-phenylphenyl)methyl]-2,3,6,7,7a,8,10,11-octahydrooxazolo[2,3-j][1,6]naphthyridin-5-one;
(3S,7aR,11aR)-3-isopropyl-9-[(3-phenoxyphenyl)methyl]-2,3,6,7,7a,8,10,11-octahydrooxazolo[2,3-j][1,6]naphthyridin-5-one;
(3S,7aR,11aR)-3-isopropyl-9-[[4-[4-[4-(trifluoromethoxy)phenoxy]-1-piperidyl]phenyl]methyl]-2,3,6,7,7a,8,10,11-octahydrooxazolo[2,3-j][1,6]naphthyridin-5-one;
(3S,7aR,11aR)-3-isopropyl-9-[[4-[4-[4-(trifluoromethoxy)phenyl]-1-piperidyl]phenyl]methyl]-2,3,6,7,7a,8,10,11-octahydrooxazolo[2,3-j][1,6]naphthyridin-5-one;
(3S,7aR,11aR)-3-isopropyl-9-[[4-(1-piperidyl)phenyl]methyl]-2,3,6,7,7a,8,10,11-octahydrooxazolo[2,3-j][1,6]naphthyridin-5-one;
(3S,7aR,11aR)-3-isopropyl-9-(2-pyridylmethyl)-2,3,6,7,7a,8,10,11-octahydrooxazolo[2,3-j][1,6]naphthyridin-5-one;
(3S,7aR,11aR)-3-isopropyl-9-(3-pyridylmethyl)-2,3,6,7,7a,8,10,11-octahydrooxazolo[2,3-j][1,6]naphthyridin-5-one;
1 (3S,7aR,11aR)-3-isopropyl-9-(4-pyridylmethyl)-2,3,6,7,7a,8,10,11-octahydrooxazolo[2,3-j][1,6]naphthyridin-5-one;
(3S,7aR,11aR)-3-isopropyl-9-[[6-(trifluoromethyl)-3-pyridyl]methyl]-2,3,6,7,7a,8,10,11-octahydrooxazolo[2,3-j][1,6]naphthyridin-5-one;
1 (3S,7aR,11aR)-3-isopropyl-9-[[5-(trifluoromethyl)-2-pyridyl]methyl]-2,3,6,7,7a,8,10,11-octahydrooxazolo[2,3-j][1,6]naphthyridin-5-one;
(3S,7aR,11aR)-3-isopropyl-9-(2-quinolylmethyl)-2,3,6,7,7a,8,10,11-octahydrooxazolo[2,3-j][1,6]naphthyridin-5-one;
(3S,7aR,11aR)-3-isopropyl-9-(8-quinolylmethyl)-2,3,6,7,7a,8,10,11-octahydrooxazolo[2,3-j][1,6]naphthyridin-5-one;
(3S,7aR,11aR)-3-isopropyl-9-(6-quinolylmethyl)-2,3,6,7,7a,8,10,11-octahydrooxazolo[2,3-j][1,6]naphthyridin-5-one;
(3S,7aR,11aR)-9-[(2-cyclopropyl-4-quinolyl)methyl]-3-isopropyl-2,3,6,7,7a,8,10,11-octahydrooxazolo[2,3-j][1,6]naphthyridin-5-one;
(3S,7aR,11aR)-3-isopropyl-9-[[2-(trifluoromethyl)-4-quinolyl]methyl]-2,3,6,7,7a,8,10,11-octahydrooxazolo[2,3-j][1,6]naphthyridin-5-one;
(3S,7aR,11aR)-3-isopropyl-9-[(1-methylimidazol-2-yl)methyl]-2,3,6,7,7a,8,10,11-octahydrooxazolo[2,3-j][1,6]naphthyridin-5-one;
(3S,7aR,11aR)-3-isopropyl-9-[(3-methyl-1H-indol-2-yl)methyl]-2,3,6,7,7a,8,10,11-octahydrooxazolo[2,3-j][1,6]naphthyridin-5-one;
1 (3S,7aR,11aR)-3-isopropyl-9-[(1-methylindol-2-yl)methyl]-2,3,6,7,7a,8,10,11-octahydrooxazolo[2,3-j][1,6]naphthyridin-5-one;
(3S,7aR,11aR)-9-(cyclohexylmethyl)-3-isopropyl-2,3,6,7,7a,8,10,11-octahydrooxazolo[2,3-j][1,6]naphthyridin-5-one;
(3S,7aR,11aR)-9-cyclobutyl-3-isopropyl-2,3,6,7,7a,8,10,11-octahydrooxazolo[2,3-j][1,6]naphthyridin-5-one;
(3S,7aR,11aR)-9-cyclohexyl-3-isopropyl-2,3,6,7,7a,8,10,11-octahydrooxazolo[2,3-j][1,6]naphthyridin-5-one;
(3S,7aR,11aR)-3-isopropyl-9-(tetrahydropyran-4-ylmethyl)-2,3,6,7,7a,8,10,11-octahydrooxazolo[2,3-j][1,6]naphthyridin-5-one;
(3S,7aR,11aR)-3-isopropyl-9-(tetrahydropyran-2-ylmethyl)-2,3,6,7,7a,8,10,11-octahydrooxazolo[2,3-j][1,6]naphthyridin-5-one;
(3S,7aR,11aR)-3-isopropyl-9-(tetrahydrofuran-2-ylmethyl)-2,3,6,7,7a,8,10,11-octahydrooxazolo[2,3-j][1,6]naphthyridin-5-one;
(3S,7aR,11aR)-3-isopropyl-9-phenyl-2,3,6,7,7a,8,10,11-octahydrooxazolo[2,3-j][1,6]naphthyridin-5-one;
(3S,7aR,11aR)-3-isopropyl-9-[4-(trifluoromethyl)phenyl]-2,3,6,7,7a,8,10,11-octahydrooxazolo[2,3-j][1,6]naphthyridin-5-one;
(3S,7aR,11aR)-3-isopropyl-9-(2-phenylethyl)-2,3,6,7,7a,8,10,11-octahydrooxazolo[2,3-j][1,6]naphthyridin-5-one;
(3S,7aR,11aR)-3-isopropyl-9-[2-[4-(trifluoromethyl)phenyl]ethyl]-2,3,6,7,7a,8,10,11-octahydrooxazolo[2,3-j][1,6]naphthyridin-5-one;
1 (3S,7aR,11aR)-3-isopropyl-9-[3-[4-(trifluoromethyl)phenyl]propyl]-2,3,6,7,7a,8,10,11-octahydrooxazolo[2,3-j][1,6]naphthyridin-5-one;
(3S,7aR,11aR)-3-isopropyl-9-(1-phenylethyl)-2,3,6,7,7a,8,10,11-octahydrooxazolo[2,3-j][1,6]naphthyridin-5-one;
1 (3S,7aR,11aR)-9-[2,2-difluoro-2-[4-(trifluoromethyl)phenyl]ethyl]-3-isopropyl-2,3,6,7,7a,8,10,11-octahydrooxazolo[2,3-j][1,6]naphthyridin-5-one;
(3S,7aR,11aR)-3-isopropyl-9-(2-phenylacetyl)-2,3,6,7,7a,8,10,11-octahydrooxazolo[2,3-j][1,6]naphthyridin-5-one;
(3S,7aR,11aR)-3-isopropyl-9-[2-[4-(trifluoromethyl)phenyl]acetyl]-2,3,6,7,7a,8,10,11-octahydrooxazolo[2,3-j][1,6]naphthyridin-5-one;
(3S,7aR,11aR)-3-isopropyl-9-[3-[4-(trifluoromethyl)phenyl]propanoyl]-2,3,6,7,7a,8,10,11-octahydrooxazolo[2,3-j][1,6]naphthyridin-5-one;
(3S,7aR,11aR)-3-isopropyl-9-4-[[4-(trifluoromethyl)phenyl]butanoyl]-2,3,6,7,7a,8,10,11-octahydrooxazolo[2,3-j][1,6]naphthyridin-5-one;
(3S,7aR,11aR)-9-[2,2-difluoro-2-[4-(trifluoromethyl)phenyl]acetyl]-3-isopropyl-2,3,6,7,7a,8,10,11-octahydrooxazolo[2,3-j][1,6]naphthyridin-5-one;
benzyl (3S,7aR,11aR)-3-isopropyl-5-oxo-2,3,6,7,7a,8,10,11-octahydrooxazolo[2,3-j][1,6]naphthyridine-9-carboxylate;
[4-(trifluoromethyl)phenyl]methyl (3S,7aR,11aR)-3-isopropyl-5-oxo-2,3,6,7,7a,8,10,11-octahydrooxazolo[2,3-j][1,6]naphthyridine-9-carboxylate;
2-[4-(trifluoromethyl)phenyl]ethyl (3S,7aR,11aR)-3-isopropyl-5-oxo-2,3,6,7,7a,8,10,11-octahydrooxazolo[2,3-j][1,6]naphthyridine-9-carboxylate;
3-[4-(trifluoromethyl)phenyl]propyl (3S,7aR,11aR)-3-isopropyl-5-oxo-2,3,6,7,7a,8,10,11-octahydrooxazolo[2,3-j][1,6]naphthyridine-9-carboxylate;
(3S,7aR,11aR)-3-isopropyl-9-[4-(trifluoromethyl)phenyl]sulfonyl-2,3,6,7,7a,8,10,11-octahydrooxazolo[2,3-j][1,6]naphthyridin-5-one;
(3S,7aR,11aR)-3-isopropyl-9-[[4-(trifluoromethyl)phenyl]methylsulfonyl]-2,3,6,7,7a,8,10,11-octahydrooxazolo[2,3-j][1,6]naphthyridin-5-one;
(3S,7aR,11aR)-3-isopropyl-9-[2-[4-(trifluoromethyl)phenyl]ethylsulfonyl]-2,3,6,7,7a,8,10,11-octahydrooxazolo[2,3-j][1,6]naphthyridin-5-one;
1 (3S,7aR,11aR)-3-isopropyl-9-[3-[4-(trifluoromethyl)phenyl]propylsulfonyl]-2,3,6,7,7a,8,10,11-octahydrooxazolo[2,3-j][1,6]naphthyridin-5-one;
(3S,7aR,11aR)-9-benzyl-3-[(1S)-1-methylpropyl]-2,3,6,7,7a,8,10,11-octahydrooxazolo[2,3-j][1,6]naphthyridin-5-one;
(3S,7aS,11aS)-9-benzyl-3-[(1S)-1-methylpropyl]-2,3,6,7,7a,8,10,11-octahydrooxazolo[2,3-j][1,6]naphthyridin-5-one;
(3S,7aR,11aR)-9-benzyl-3-[(1R)-1-methylpropyl]-2,3,6,7,7a,8,10,11-octahydrooxazolo[2,3-j][1,6]naphthyridin-5-one;
(3S,7aR,11aR)-9-benzyl-3-tert-butyl-2,3,6,7,7a,8,10,11-octahydrooxazolo[2,3-j][1,6]naphthyridin-5-one;
(7aR,11aR)-9-benzyl-3,3-dimethyl-6,7,7a,8,10,11-hexahydro-2H-oxazolo[2,3-j][1,6]naphthyridin-5-one;
1 (7aS,11aS)-9-benzyl-3,3-dimethyl-6,7,7a,8,10,11-hexahydro-2H-oxazolo[2,3-j][1,6]naphthyridin-5-one;
(7aR,11aR)-9-benzylspiro[6,7,7a,8,10,11-hexahydro-2H-oxazolo[2,3-j][1,6]naphthyridine-3,1′-cyclohexane]-5-one;
(7aS,11aS)-9-benzylspiro[6,7,7a,8,10,11-hexahydro-2H-oxazolo[2,3-j][1,6]naphthyridine-3,1′-cyclohexane]-5-one;
1 (3S,7aR,11aR)-9-benzyl-3-cyclopropyl-2,3,6,7,7a,8,10,11-octahydrooxazolo[2,3-j][1,6]naphthyridin-5-one;
(3S,7aR,11aR)-9-[[4-(trifluoromethyl)phenyl]methyl]-3-vinyl-2,3,6,7,7a,8,10,11-octahydrooxazolo[2,3-j][1,6]naphthyridin-5-one;
(3S,7aR,11aR)-9-benzyl-3-ethyl-2,3,6,7,7a,8,10,11-octahydrooxazolo[2,3-j][1,6]naphthyridin-5-one;
(3S,7aS,11aS)-9-benzyl-3-ethyl-2,3,6,7,7a,8,10,11-octahydrooxazolo[2,3-j][1,6]naphthyridin-5-one;
(3R,7aR,11aR)-3-(trifluoromethyl)-9-[[4-(trifluoromethyl)phenyl]methyl]-2,3,6,7,7a,8,10,11-octahydrooxazolo[2,3-j][1,6]naphthyridin-5-one
(1R,6R,11S,16S)-4-benzyl-17-oxa-4,10-diazatetracyclo[8.7.0.01,6.011, 16]heptadecan-9-one;
(1S,6S,11R,16R)-4-benzyl-17-oxa-4,10-diazatetracyclo[8.7.0.01,6.011, 16]heptadecan-9-one;
(1R,6R, 11S, 16S)-4-[[4-(trifluoromethoxy)phenyl]methyl]-17-oxa-4,10-diazatetracyclo[8.7.0.01,6.011, 16]heptadecan-9-one;
(1S,6S,11R,16R)-4-[[4-(trifluoromethoxy)phenyl]methyl]-17-oxa-4,10-diazatetracyclo[8.7.0.01,6.011, 16]heptadecan-9-one;
(8aR,12aR)-10-[[4-(trifluoromethyl)phenyl]methyl]spiro[2,4,7,8,8a,9,11,12-octahydro-[1,3]oxazino[2,3-j][1,6]naphthyridine-3,1′-cyclobutane]-6-one;
(8aS,12aS)-10-[[4-(trifluoromethyl)phenyl]methyl]spiro[2,4,7,8,8a,9,11,12-octahydro-[1,3]oxazino[2,3-j][1,6]naphthyridine-3,1′-cyclobutane]-6-one;
(3R,8aR,12aR)-3-isopropyl-10-[[4-(trifluoromethyl)phenyl]methyl]-3,4,7,8,8a,9,11,12-octahydro-2H-[1,3]oxazino[2,3-j][1,6]naphthyridin-6-one;
(3S,8aS,12aS)-3-isopropyl-10-[[4-(trifluoromethyl)phenyl]methyl]-3,4,7,8,8a,9,11,12-octahydro-2H-[1,3]oxazino[2,3-j][1,6]naphthyridin-6-one;
(3R,8aR,12aR)-3-ethyl-10-[[4-(trifluoromethyl)phenyl]methyl]-3,4,7,8,8a,9,11,12-octahydro-2H-[1,3]oxazino[2,3-j][1,6]naphthyridin-6-one;
(3S,8aS,12aS)-3-ethyl-10-[[4-(trifluoromethyl)phenyl]methyl]-3,4,7,8,8a,9,11,12-octahydro-2H-[1,3]oxazino[2,3-j][1,6]naphthyridin-6-one;
1 (4R,8aR,12aR)-4-isopropyl-10-[[4-(trifluoromethyl)phenyl]methyl]-3,4,7,8,8a,9,11,12-octahydro-2H-[1,3]oxazino[2,3-j][1,6]naphthyridin-6-one;
(1S,6R,11S,16S)-4-benzyl-4,10,17-triazatetracyclo[8.7.0.01,6.011, 16]heptadecan-9-one;
1 (1R,4S,8R)-10-benzyl-4-isopropyl-2-oxa-5,10-diazatricyclo[6.4.0.01,5]dodecan-6-one;
(1R,4S,8R)-4-isopropyl-10-[[4-(trifluoromethyl)phenyl]methyl]-2-oxa-5,10-diazatricyclo[6.4.0.01,5]dodecan-6-one;
(3S,7aR,11aR)-9-benzyl-3-isopropyl-2,3,6,7,7a,8,10,11-octahydrooxazolo[2,3-j][1,6]naphthyridine-5-thione;
(1R,4S,9R)-4-isopropyl-11-[[4-(trifluoromethyl)phenyl]methyl]-2,8-dioxa-5,11-diazatricyclo[7.4.0.01,5]tridecan-6-one;
1 (1R,4S,8R)-4-isopropyl-10-[[4-(trifluoromethyl)phenyl]methyl]-2-oxa-5,10-diazatricyclo[6.4.0.01,5]dodecan-6-one;
(1R,4R,9R)-4-isopropyl-11-[[4-(trifluoromethyl)phenyl]methyl]-2-oxa-6,11-diazatricyclo[7.4.0.01,6]tridecan-7-one;
1 (1S,4S,9S)-4-isopropyl-11-[[4-(trifluoromethyl)phenyl]methyl]-2-oxa-6,11-diazatricyclo[7.4.0.01,6]tridecan-7-one;
(1R,4R,9R)-4-ethyl-11-[[4-(trifluoromethyl)phenyl]methyl]-2-oxa-6,11-diazatricyclo[7.4.0.01,6]tridecan-7-one;
1 (1R,4R,9R)-4-ethyl-11-[[4-(trifluoromethyl)phenyl]methyl]-2-oxa-6,11-diazatricyclo[7.4.0.01,6]tridecan-7-one;
(3S,7aR,11aR)-3-isopropyl-9-[[4-(trifluoromethyl)phenyl]methyl]-3,6,7,7a,8,11-hexahydro-2H-oxazolo[2,3-j][1,6]naphthyridine-5,10-dione;
(3S,7aS,11aR)-3-isopropyl-9-[[4-(trifluoromethyl)phenyl]methyl]-3,6,7,7a, 10,11-hexahydro-2H-oxazolo[2,3-j][1,6]naphthyridine-5,8-dione;
(3S,7aR,9S,11aR)-9-[bis[[4-(trifluoromethyl)phenyl]methyl]amino]-3-isopropyl-3,6,7,7a,8,9,10,11-octahydro-2H-oxazolo[2,3-j]quinolin-5-one;
1 (3S,7aR,9R, 11aR)-9-[bis[[4-(trifluoromethyl)phenyl]methyl]amino]-3-isopropyl-3,6,7,7a,8,9,10,11-octahydro-2H-oxazolo[2,3-j]quinolin-5-one;
(3S,7aR,9S, 11aR)-3-isopropyl-9-[[4-(trifluoromethyl)phenyl]methylamino]-3,6,7,7a,8,9,10,11-octahydro-2H-oxazolo[2,3-j]quinolin-5-one;
1 (3S,7aR,9R, 11aR)-3-isopropyl-9-[[4-(trifluoromethyl)phenyl]methylamino]-3,6,7,7a,8,9,10,11-octahydro-2H-oxazolo[2,3-j]quinolin-5-one;
(3S,7aR,9S, 11aR)-3-isopropyl-9-[4-(trifluoromethyl) anilino]-3,6,7,7a,8,9,10,11-octahydro-2H-oxazolo[2,3-j]quinolin-5-one;
(3S,7aR,9S, 11aR)-3-isopropyl-9-[4-(trifluoromethyl) anilino]-3,6,7,7a,8,9,10,11-octahydro-2H-oxazolo[2,3-j]quinolin-5-one;
1 (4S,9R)-4-isopropyl-11-[[4-(trifluoromethyl)phenyl]methyl]-2-oxa-5,11-diazatricyclo[7.5.0.01,5]tetradecan-6-one;
(1R,4S,9S)-4-isopropyl-10-[[4-(trifluoromethyl)phenyl]methyl]-2-oxa-5,10-diazatricyclo[7.3.0.01,5]dodecan-6-one;
(3S,6R,7aR,11aR)-3-isopropyl-6-methyl-9-[[4-(trifluoromethyl)phenyl]methyl]-2,3,6,7,7a,8,10,11-octahydrooxazolo[2,3-j][1,6]naphthyridin-5-one;
1 (3S,6R,7aR,11aR)-6-ethyl-3-isopropyl-9-[[4-(trifluoromethyl)phenyl]methyl]-2,3,6,7,7a,8,10,11-octahydrooxazolo[2,3-j][1,6]naphthyridin-5-one;
(3S,6R,7aR,11aR)-6-allyl-3-isopropyl-9-[[4-(trifluoromethyl)phenyl]methyl]-2,3,6,7,7a,8,10,11-octahydrooxazolo[2,3-j][1,6]naphthyridin-5-one;
(3S,6R,7aR,11aR)-6-isobutyl-3-isopropyl-9-[[4-(trifluoromethyl)phenyl]methyl]-2,3,6,7,7a,8,10,11-octahydrooxazolo[2,3-j][1,6]naphthyridin-5-one;
(3S,6S,7aR,11aR)-3-isopropyl-6-methyl-9-[[4-(trifluoromethyl)phenyl]methyl]-2,3,6,7,7a,8,10,11-octahydrooxazolo[2,3-j][1,6]naphthyridin-5-one;
(3S,6S,7aR,11aR)-6-ethyl-3-isopropyl-9-[[4-(trifluoromethyl)phenyl]methyl]-2,3,6,7,7a,8,10,11-octahydrooxazolo[2,3-j][1,6]naphthyridin-5-one;
(3S,6S,7aR,11aR)-6-allyl-3-isopropyl-9-[[4-(trifluoromethyl)phenyl]methyl]-2,3,6,7,7a,8,10,11-octahydrooxazolo[2,3-j][1,6]naphthyridin-5-one;
(3S,7aR,11aR)-6-fluoro-3-isopropyl-9-[[4-(trifluoromethyl)phenyl]methyl]-2,3,6,7,7a,8,10,11-octahydrooxazolo[2,3-j][1,6]naphthyridin-5-one;
(3S,7aR,11aR)-6,6-difluoro-3-isopropyl-9-[[4-(trifluoromethyl)phenyl]methyl]-3,7,7a,8,10,11-hexahydro-2H-oxazolo[2,3-j][1,6]naphthyridin-5-one;
or its pharmaceutically acceptable salts or optical isomers.

6. A compound according to claim 1, wherein:
X is O; and/or
Y is O; and/or
m has a value of 1; and/or n has a value of 1 and W is CR₄R_{4′}; and/or
p and p′ have a value of 1; and/or
Z is NR₁ with R₁ chosen from:
benzyl, said benzyl being substituted by one or more halogen atom, linear-(C₁-C₆)halogenoalkyl, linear-(C₁-C₆)halogenoalkoxy,
linear or branched —CH₃—(C₆-C₁₀) heteroaryl, said heteroaryl having at least one heteroatom chosen from O, N, or S, in particular said heteroaryl being a piperidine, and being optionally substituted by one or more linear-(C₁-C₆)halogenoalkyl (C₁-C₃)alkyl or —(C₃-C₆)cycloalkyl,
linear or branched —(C₁-C₆)alkyl-(C₆-C₁₀)aryl, said aryl being in particular phenyl, and said phenyl being optionally substituted by linear-(C₁-C₃)halogenoalkyl; or
Z is CHNRaRb with Ra and Rb, identical being linear or branched —(C₁-C₃)alkylphenyl, said phenyl being optionally substituted by one or more linear or branched —(C₁-C₃) halogenoalkyl, in particular by one trifluoromethyl; and/or
R₂ and R₂′ are H; and/or
R₃ and R₃′ are different and chosen from H and branched —(C₁-C₆)alkyl; and/or
R₄ and R₄′ are identical and are H; and/or
R₅ and R₅′ are H; and/or
R₆ and R₆′ are H.

7. A compound according to claim 1, wherein said compound if chosen from:
1 (3S,7aR,11aR)-3-isopropyl-9-[[4-(trifluoromethyl)phenyl]methyl]-2,3,6,7,7a,8,10,11 octahydrooxazolo[2,3-j][1,6]naphthyridin-5-one
(3S,7aR,11aR)-3-isopropyl-9-[[4-(trifluoromethoxy)phenyl]methyl]-2,3,6,7,7a,8,10,11-octahydrooxazolo[2,3-j][1,6]naphthyridin-5-one
(3S,7aR,11aR)-9-[(4-chlorophenyl)methyl]-3-isopropyl-2,3,6,7,7a,8,10,11-octahydrooxazolo[2,3-j][1,6]naphthyridin-5-one
(3S,7aR,11aR)-9-[(3,4-dichlorophenyl)methyl]-3-isopropyl-2,3,6,7,7a,8,10,11-octahydrooxazolo[2,3-j][1,6]naphthyridin-5-one
(3S,7aR,11aR)-9-[[4-chloro-3-(trifluoromethyl)phenyl]methyl]-3-isopropyl-2,3,6,7,7a,8,10,11-octahydrooxazolo[2,3-j][1,6]naphthyridin-5-one
(3S,7aR,11aR)-9-[[2,4-bis(trifluoromethyl)phenyl]methyl]-3-isopropyl-2,3,6,7,7a,8,10,11-octahydrooxazolo[2,3-j][1,6]naphthyridin-5-one
(3S,7aR,11aR)-9-[(2-cyclopropyl-4-quinolyl)methyl]-3-isopropyl-2,3,6,7,7a,8,10,11-octahydrooxazolo[2,3-j][1,6]naphthyridin-5-one
(3S,7aR,11aR)-3-isopropyl-9-[[2-(trifluoromethyl)-4-quinolyl]methyl]-2,3,6,7,7a,8,10,11-octahydrooxazolo[2,3-j][1,6]naphthyridin-5-one
(3S,7aR,11aR)-3-isopropyl-9-[(1-methylindol-2-yl)methyl]-2,3,6,7,7a,8,10,11-octahydrooxazolo[2,3-j][1,6]naphthyridin-5-one
(3S,7aR,11aR)-3-isopropyl-9-[2-[4-(trifluoromethyl)phenyl]ethyl]-2,3,6,7,7a,8,10,11-octahydrooxazolo[2,3-j][1,6]naphthyridin-5-one
(3S,7aR,11aR)-3-isopropyl-9-[3-[4-(trifluoromethyl)phenyl]propyl]-2,3,6,7,7a,8,10,11-octahydrooxazolo[2,3-j][1,6]naphthyridin-5-one
(3S,7aR,9S, 11aR)-9-[bis[[4-(trifluoromethyl)phenyl]methyl]amino]-3-isopropyl-3,6,7,7a,8,9,10,11-octahydro-2H-oxazolo[2,3-j]quinolin-5-one or its pharmaceutically acceptable salts or optical isomers.

8. A pharmaceutical composition comprising a compound according to claim 1.

9. (canceled)

10. A method for the prevention and/or treatment of a mycobacterial infection or for the treatment of a disease caused by infection with a Mycobacterium, said method comprising administering a therapeutically effective amount of a compound according to claim 1 or a pharmaceutical composition which includes the compound to a subject in need thereof.

11. The method according to claim 10, wherein the mycobacterial infection is a Mycobacterium tuberculosis infection.

12. The method according to claim 10, for the prevention and/or treatment of tuberculosis.

13. The method according to claim 10, wherein said compound or pharmaceutical composition is administered in combination with at least one other anti-mycobacterial agent.

14. A combination of (a) a compound of Formula (I) as defined in claim 1 and (b) at least one other anti-mycobacterial agent.

15. The method of claim 10, wherein said at least one other anti-mycobacterial agent is an anti-tuberculosis agent.

16. The pharmaceutical composition of claim 8, wherein said at least one other anti-mycobacterial agent is an anti-tuberculosis agent.

17. The combination of claim 15, wherein said at least one other anti-mycobacterial agent is an anti-tuberculosis agent.
